(19) United States
(12) Patent Application Publication
(10) Pub. No.: US 2012/0180165 A1
(43) Pub. Date: Jul. 12, 2012

(54) Plants Having Enhanced Yield-Related Traits and a Method for Making the Same

(75) Inventors: Yves Hatzfeld, Lille (FR); Christophe Reuzeau, Tocan Saint Apre (FR); Valerie Frankard, Waterloo (BE); Marieke Louwers, Gent (BE)

(73) Assignee: BASF PLANT SCIENCE COMPANY GMBH, Ludwigshafen (DE)

(21) Appl. No.: 13/497,854

(22) PCT Filed: Sep. 22, 2010
(86) PCT No.: PCT/EP10/63931

§ 371 Date: Mar. 23, 2012

Related U.S. Application Data
(60) Provisional application No. 61/249,290, filed on Oct. 7, 2009.
(60) Provisional application No. 61/252,208, filed on Oct. 16, 2009.
(60) Provisional application No. 61/249,282, filed on Oct. 7, 2009.
(60) Provisional application No. 61/252,236, filed on Oct. 16, 2009.
(60) Provisional application No. 61/252,183, filed on Oct. 16, 2009.

(30) Foreign Application Priority Data
Sep. 25, 2009 (EP) 09171331.3
Sep. 25, 2009 (EP) 09171353.7
Oct. 9, 2009 (EP) 09172707.3
Oct. 9, 2009 (EP) 09172713.1
Oct. 16, 2009 (EP) 09173350.1

(51) Int. Cl. A01H 5/00; A01H 5/10; C07K 14/415; A01H 5/04; C12N 15/29; C12N 15/82; A01H 5/06

(52) U.S. Cl. 800/290; 800/298; 435/320.1; 800/320; 800/320.1; 800/320.3; 800/320.2; 536/23.6; 530/370

(57) ABSTRACT

The present invention relates generally to the field of molecular biology and concerns a method for improving various plant growth characteristics by modulating expression in a plant of a nucleic acid encoding a GDH (Glutamate DeHydrogenase) polypeptide. The present invention also concerns plants having modulated expression of a nucleic acid encoding a GDH polypeptide, which plants have improved growth characteristics relative to corresponding wild type plants or other control plants. The invention also provides constructs useful in the methods of the invention. The present invention relates generally to the field of molecular biology and concerns a method for enhancing various economically important yield-related traits in plants. More specifically, the present invention concerns a method for enhancing yield-related traits in plants by modulating expression in a plant of a nucleic acid encoding a FLA-like (Fasciclin-like) polypeptide. The present invention also concerns plants having modulated expression of a nucleic acid encoding a FLA-like polypeptide, which plants have enhanced yield-related traits relative to control plants. The invention also provides constructs comprising FLA-like-encoding nucleic acids, useful in performing the methods of the invention. The present invention relates generally to the field of molecular biology and concerns a method for enhancing yield-related traits in plants by modulating expression in a plant of a nucleic acid encoding a SAUR polypeptide. The present invention also concerns plants having modulated expression of a nucleic acid encoding a SAUR polypeptide, which plants have enhanced yield-related traits relative to corresponding wild type plants or other control plants. The invention also provides constructs useful in the methods of the invention. Furthermore, the present invention also relates to a SAUR-based protein complex. It further relates to the use of the complex to enhance yield-related traits, and to a method for stimulating the complex formation, by overexpressing at least two members of the complex. The present invention relates generally to the field of molecular biology and concerns a method for enhancing yield traits in plants by modulating expression in a plant of a nucleic acid encoding a dehydroascorbate reductase (DHAR) polypeptide. The present invention also concerns plants having modulated expression of a nucleic acid encoding a DHAR polypeptide, which plants have enhancing yield traits relative to corresponding wild type plants or other control plants. The invention also provides constructs useful in the methods of the invention.

The present invention relates generally to the field of molecular biology and concerns a method for improving various plant growth characteristics by modulating expression in a plant of a nucleic acid encoding a GDH (Glutamate DeHydrogenase) polypeptide. The present invention also concerns plants having modulated expression of a nucleic acid encoding a GDH polypeptide, which plants have improved growth characteristics relative to corresponding wild type plants or other control plants. The invention also provides constructs useful in the methods of the invention.

The present invention relates generally to the field of molecular biology and concerns a method for enhancing various economically important yield-related traits in plants. More specifically, the present invention concerns a method for enhancing yield-related traits in plants by modulating expression in a plant of a nucleic acid encoding a FLA-like (Fasciclin-like) polypeptide. The present invention also concerns plants having modulated expression of a nucleic acid encoding a FLA-like polypeptide, which plants have enhanced yield-related traits relative to control plants. The invention also provides constructs comprising FLA-like-encoding nucleic acids, useful in performing the methods of the invention.

The present invention relates generally to the field of molecular biology and concerns a method for enhancing yield-related traits in plants by modulating expression in a plant of a nucleic acid encoding a SAUR polypeptide. The present invention also concerns plants having modulated expression of a nucleic acid encoding a SAUR polypeptide, which plants have enhanced yield-related traits relative to corresponding wild type plants or other control plants. The invention also provides constructs useful in the methods of the invention. Furthermore, the present invention also relates to a SAUR-based protein complex. It further relates to the use of the complex to enhance yield-related traits, and to a method for stimulating the complex formation, by overexpressing at least two members of the complex.

The present invention relates generally to the field of molecular biology and concerns a method for enhancing yield traits in plants by modulating expression in a plant of a nucleic acid encoding a dehydroascorbate reductase (DHAR) polypeptide. The present invention also concerns plants having modulated expression of a nucleic acid encoding a DHAR polypeptide, which plants have enhancing yield traits relative to corresponding wild type plants or other control plants. The invention also provides constructs useful in the methods of the invention.

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the abovementioned factors may therefore contribute to increasing crop yield.

Seed yield is a particularly important trait, since the seeds of many plants are important for human and animal nutrition. Crops such as corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain.

Another important trait for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. The ability to engineer early vigour into plants would be of great importance in agriculture. For example, poor early vigour has been a limitation to the introduction of maize (Zea mays L.) hybrids based on Corn Belt germplasm in the European Atlantic.

A further important trait is that of improved abiotic stress tolerance. Abiotic stress is a primary cause of crop loss worldwide, reducing average yields for most major crop plants by more than 50% (Wang et al., Planta (2003) 218: 1-14). Abiotic stresses may be caused by drought, salinity, extremes of temperature, chemical toxicity and oxidative stress. The ability to improve plant tolerance to abiotic stress would be of great economic advantage to farmers worldwide and would allow for the cultivation of crops during adverse conditions and in territories where cultivation of crops may not otherwise be possible.

Crop yield may therefore be increased by optimising one of the above-mentioned factors.

Depending on the end use, the modification of certain yield traits may be favoured over others. For example for applications such as forage or wood production, or bio-fuel resource, an increase in the vegetative parts of a plant may be desirable, and for applications such as flour, starch or oil production, an increase in seed parameters may be particularly desirable. Even amongst the seed parameters, some may be favoured over others, depending on the application. Various mechanisms may contribute to increasing seed yield, whether that is in the form of increased seed size or increased seed number.

One approach to increasing yield (seed yield and/or biomass) in plants may be through modification of the inherent growth mechanisms of a plant, such as the cell cycle or various signalling pathways involved in plant growth or in defence mechanisms.

It has now been found that various growth characteristics may be improved in plants by modulating expression in a plant of a nucleic acid encoding a GDH (Glutamate dehydrogenase) in a plant.

It has also now been found that various yield-related traits may be improved (herein also refer to as enhanced) in plants by modulating expression in a plant of a nucleic acid encoding a FLA-like polypeptide in a plant.

It has also now been found that various yield-related traits may be improved in plants by modulating expression in a plant of a nucleic acid encoding a SAUR polypeptide in a plant or modulating expression in a plant of a SAUR-based protein complex.

It has also now been found that various yield related traits may be improved in plants by modulating expression in a plant of a nucleic acid encoding a DHAR polypeptide in a plant.

### BACKGROUND

### 1. Glutamate Dehydrogenase (GDH) Polypeptides

Glutamate dehydrogenase catalyses the reversible deamination of glutamate into 2-oxoglutarate. It exists at least in 3 forms, depending on the coenzyme used: NAD (EC1.4.1.2), NAD(P) (EC1.4.1.3) or NADP (EC1.4.1.4). In plants, existence of only the NAD-GDH form has been reported, although there are indications of the occurrence of an NADP-GDH form. Plant GDH exists as hexamers of alpha and beta subunits in 7 isoforms, going from isoform 1 (6× betasubunit) to isoform 7 (6× alpha subunit) (Turano et al., Plant Physiol. 113, 1329-1341, 1997). Alpha and beta subunits are related to each other on amino acid sequence level and usually have a sequence identity between 75 and 85%. GDH isoform 7 has a high glutamate deaminating activity, with a minor aminating activity, whereas GDH isoform 1 has only a deaminating activity (Turano et al., 1997). Glutamate dehydrogenase activity in vivo is primarily located in mitochondria; the reaction goes mainly in the direction of Glutamate deamination and 2-oxoglutarate production, rather than in the direction of Glutamate synthesis:

L-Glutamate+H₂O+NAD+⇄2-oxoglutarate+NH₃+NADH+H⁺

Downregulation or overexpression of the beta subunit in tobacco (transgenic lines having GDH activity ranging between 0.5 to 34 times of normal activity levels) had little effect on leaf ammonium or the leaf free amino acid pool, except for a large decrease of Asp in leaves; furthermore, plant growth and development was not affected (Purnell et al., Planta 222, 167-180, 2005). No plant phenotype was described for plants overexpressing the alpha subunit (Skopelitis et al., Plant Physiol. 145, 1726-1734, 2007). On the other hand, strong overexpression of gdhA from Escherichia coli (encoding an NADPH-GDH) resulted in increased biomass, as well as in altered metabolite levels (Ameziane et al., Plant and Soil 221, 47-57, 2000). Also other studies reported changes in metabolite levels upon overexpression of gdhA from Escherichia coli in corn (Guthrie et al., J. Anim. Sci. 82, 1693-1698, 2004), or upon overexpressing NADP-dependent glutamate dehydrogenase (gdhA) from Aspergillus nidulans (Kisaka and Kida, Plant Science 164, 35-42, 2003).

### 2. Fasciclin-Like AGP (FLA) Polypeptides

Cell-to-cell interactions and communication provide key structural, positional, and environment signals during plant development. In plant cells, such signals must traverse the cell wall that surrounds the plasma membrane. Plant cell walls are primarily composed of the polysaccharides cellulose, crosslinking glycans, pectins, and some proteins) that together form a complex interactive network known as the extracellular matrix (ECM). The nature of the interactions changes during development and is influenced by biotic and abiotic stresses, resulting in altered wall composition and structure. Cell wall proteins, which generally comprise less than 10% of the dry weight of the primary wall, are recognized as critical components in maintaining the physical and biological functions of the plant ECM. Most ECM proteins belong to large families that include enzymes such as the hydrolases, proteases, glycosidases, peroxidases, and esterases, expansins, wall-associated kinases, and hydroxyproline (Hyp)-rich glycoproteins (Arabidopsis Genome Initiative [AGI], 2000). Arabinogalactan proteins (AGPs) are a class of Hyp-rich glycoproteins that are highly glycosylated and are abundant in the plant cell wall and plasmamembrane. Type II arabinogalactan (AG) polysaccharide chains predominate and are O-glycosidically linked to Hyp residues in the protein backbone, resulting in the total mass of the molecule consisting of 90% to 99% carbohydrate. There is increasing evidence that Hyp (arabino) galactosylation depends on the clustered, noncontiguous arrangement of the Hyp residues. In contrast, blocks of contiguous Hyp residues, such as those that occur in extensins, are arabinosylated with short oligosaccharides. In Arabidopsis, the glycosylphosphatidylinositol (GPI)-anchored AGPs can be divided into four subclasses, the classical AGPs, those with Lys-rich domains, and AG peptides with short protein backbones. The fasciclin-like AGPs (FLAs) constitute a fourth distinct subclass of AGPs. Proteins with variable numbers of fasciclin domains, generally one to four, were first identified in fruitfly (Drosophila melanogaster) and have since been identified in proteins from animals, yeast, bacteria, algae, lichens, and higher plants Johnson et al., 2003 Plant physiology 133, 1911-1925). Fasciclin domains are 110 to 150 amino acids long and have low sequence similarity. This low sequence similarity may account for the lack of a single consensus sequence for fasciclin domains. However, all fasciclin domains contain two highly conserved regions (H1 and H2) of approximately 10 amino acids each. Proteins containing fasciclin domains, from a broad spectrum of organisms, have been shown to function as adhesion molecules. Fasciclin 1 (Fas1) from fruitfly is capable of promoting cell adhesion through homophilic interactions. A multiple sequence alignment of all the fasciclin domains of FLAs from Arabidopsis and a consensus sequence (smart00554) identified the conserved regions common to all fasciclin domains, called H1 and H2 (Johnson et al., 2003). Most of the Arabidopsis FLAs contain other conserved residues such as Leu and Ile near the H1 domain that are thought to be involved in either maintaining the structure of the fasciclin domain and/or cell adhesion (Johnson et al., 2003).

A number of mutations in Arabidopsis FLAs have been characterized. The haploin sufficient mutant, rat1 (resistant to Agrobacterium transformation), is resistant to transient and stable transformation of root segments by tumorigenic and non-tumorigenic Agrobacterium strains. This mutant has a T-DNA insertion upstream of the start codon of AGP17. A root-specific non-classical (chimeric) AGP from Arabidopsis, AtAGP30, has been implicated in root regeneration and seed germination. The other AGP mutant, sos5/fla4, displays a salt overly sensitive phenotype with increased cell expansion under high salt conditions (Gaspar et al; 2004; Shi 2003 Plant Cell. 2003 January; 15(1):19-32.).

### 3. Small Auxin-Up RNA (SAUR) Polypeptides

The early auxin-responsive genes, which are specifically induced within minutes of auxin application, have been broadly grouped into three major classes: auxin/indoleacetic acid (Aux/IAA), GH3, and small auxin-up RNA (SAUR) gene families. SAURs can be induced by cycloheximide, a translational inhibitor, indicating that their transcription is regulated by a short-lived repressor. Following the initial identification of SAUR genes from Soybean, members of this class have been isolated from mung bean, pea, Arabidopsis, tobacco, and, more recently, maize. SAURs are represented as a large multigene family in the Arabidopsis genome comprising more than 70 members. The SAURs encode highly unstable mRNAs with a very high turnover that are induced within minutes by auxin application. The instability of SAUR mRNAs has been attributed due to the presence of a conserved downstream (DST) element in their 3′-untranslated regions. There is evidence that the SAURs are regulated at the posttranscriptional and posttranslational levels, too. Recently, the calcium-dependent in vitro binding of SAUR proteins with calmodulin has been demonstrated which provides a link between the Ca2+/calmodulin second messenger system and auxin signaling (Jain et al. 2006 Genomics 88, 360-371; Hagen and Guilfoyle Plant Molecular Biology 49: 373-385, 2002). The phylogenetic analysis of SAURs from rice and Arabidopsis was performed to understand the possible mechanisms of gene family expansion. Recently Kant et al. Plant Physiol. 2009 online publication characterized the role of the rice SAUR 39 protein in SAUR39 as a negative regulator of auxin synthesis and transport in rice.

### 4. Dehydroascorbate Reductase (DHAR) Polypeptides

The role of dehydroascorbate reductase (DHAR) is widely related to ascorbate-glutathione cycle and to regeneration of ascorbic acid (ASC) from oxidized ascorbate. This enzyme is critical for maintaining proper redox state of ascorbic acid, and therefore of the cell, and has an important role in defensive processes against oxidative damage generated by drought stress (Secenji, M. et al. 2008—Transcriptional changes in ascorbate-glutathione cycle under drought conditions. Acta Biologica Szegediensis, 52(1):93-94).

U.S. Pat. No. 6,903,246 discloses DAHR genes from Triticum aestivum and their use to modulate ascorbic acid levels in plants. Lee, Y. P. et al. (Enhanced tolerance to oxidative stress in transgenic tobacco plants expressing three antioxidant enzymes in chloroplasts. Plant Cell Rep. 26: 591-8, 2007) discloses the role of simultaneous expression of DHAR, SOD and APX in chloroplast of tobacco, which increases tolerance to oxidative stress. The association of DAHR and plant response to stress was also disclosed by Ushimaru, T. et al. (Transgenic Arabidopsis plants expressing the rice dehydroascorbate reductase gene are resistant to salt stress. J. Plant Physiol. 163: 1179-84, 2006). In this study it was observed that the expression of cytosolic rice DHAR with 35S promoter in Arabidopsis contributes to increased ascorbate content of the plant, which leads to increased salt stress tolerance. Kwon, S. Y. et al. (Enhanced stress-tolerance of transgenic tobacco plants expressing a human dehydroascorbate reductase gene. J. Plant Physiol. 160: 347-53, 2003) discloses the over expression of human DHAR in chloroplast of tobacco and the resulting increased resistance to oxidative stress, cold and salt stress. Zou, L. et al. (Cloning and mapping of genes involved in tomato ascorbic acid biosynthesis and metabolism. Plant Sci. 170 (1), 120-127, 2006) discloses cloning and mapping of several tomato genes involved in ascorbic acid biosynthesis and metabolism and amongst them two DHAR genes in order to screen for candidate genes linked to tomato ascorbic acid biosynthesis and metabolism. Kato, Y. et al. (Purification and characterization of dehydroascorbate reductase from rice. Plant and Cell Physiology, 38, No. 2 173-178, 1997) disclose a method for enzymatic assay to evaluate DHAR activity.

### SUMMARY

### 1. Glutamate Dehydrogenase (GDH) Polypeptides

Surprisingly, it has now been found that modulating expression of a nucleic acid encoding a GDH polypeptide gives plants having enhanced yield-related traits, in particular increased yield and improved early vigour, relative to control plants.

According to one embodiment, there is provided a method for improving yield related traits of a plant relative to control plants, comprising modulating expression of a nucleic acid encoding a GDH polypeptide in a plant.

### 2. Fasciclin-Like AGP (FLAs) Polypeptides

Surprisingly, it has now been found that modulating expression of a nucleic acid encoding a FLA-like polypeptide gives plants having enhanced yield-related traits relative to control plants.

According to one embodiment, there is provided a method for enhancing yield-related traits in plants relative to control plants, comprising modulating expression in a plant of a nucleic acid encoding a FLA-like polypeptide.

### 3. Small Auxin-Up RNA (SAUR) Polypeptides

Surprisingly, it has now been found that modulating expression of a nucleic acid encoding a SAUR polypeptide gives plants having enhanced yield-related traits relative to control plants.

According to one embodiment, there is provided a method for enhancing (improving) yield-related traits in plants relative to control plants, comprising modulating expression in a plant of a nucleic acid encoding a SAUR polypeptide or modulating expression in a plant of a SAUR-based protein complex.

In order to decipher the biological networks influencing yield-traits in plants a SAUR protein centred approach was undertaken to study SAUR interacting proteins in Arabidopsis thaliana. The interactome and the regulon of SAUR proteins were used to make a selection of genes that act together with SAUR proteins in enhancing yield related traits of plants, referred to herein as SYNP (SAUR Yield Network protein) proteins.

Surprisingly, a subset of proteins belonging to the SYNP proteins group of proteins could be identified.

### 4. Dehydroascorbate Reductase (DHAR) Polypeptides

Surprisingly, it has now been found that modulating expression of a nucleic acid encoding a DHAR polypeptide gives plants having enhanced yield-related traits, in particular increased yield relative to control plants.

According one embodiment, there is provided a method for improving yield related traits in plants relative to control plants, comprising modulating expression in a plant of a nucleic acid encoding a DHAR polypeptide.

### DEFINITIONS

The following definitions will be used throughout the present specification.

### Polypeptide(s)/Protein(s)

The terms “polypeptide” and “protein” are used interchangeably herein and refer to amino acids in a polymeric form of any length, linked together by peptide bonds.

### Polynucleotide(s)/Nucleic Acid(s)/Nucleic Acid Sequence(s)/Nucleotide Sequence(s)

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

### Homologue(s)

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide and may range from 1 to 10 amino acids; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds) and Table 1 below).

TABLE 1
Examples of conserved amino acid substitutions

| | Conservative | | Conservative |
|---|---|---|---|
| Residue | Substitutions | Residue | Substitutions |
| Ala | Ser | Leu | Ile; Val |
| Arg | Lys | Lys | Arg; Gln |
| Asn | Gln; His | Met | Leu; Ile |
| Asp | Glu | Phe | Met; Leu; Tyr |
| Gln | Asn | Ser | Thr; Gly |
| Cys | Ser | Thr | Ser; Val |
| Glu | Asp | Trp | Tyr |
| Gly | Pro | Tyr | Trp; Phe |
| His | Asn; Gln | Val | Ile; Leu |
| Ile | Leu, Val | | |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

### Derivatives

“Derivatives” include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

### Orthologue(s)/Paralogue(s)

Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

### Domain, Motif/Consensus Sequence/Signature

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

The term “motif” or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7).

### Reciprocal BLAST

Typically, this involves a first BLAST involving BLASTing a query sequence (for example using any of the sequences listed in Table A of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived. The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

### Hybridisation

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore medium stringency hybridisation conditions may sometimes be needed to identify such nucleic acid molecules.

The Tₘ is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tₘ may be calculated using the following equations, depending on the types of hybrids:

1) DNA-DNA hybrids (Meinkoth and Wahl, Anal. Biochem., 138: 267-284, 1984):

Tₘ=81.5° C.+16.6×log₁₀[Na⁺]^{a}+0.41×%[G/C^{b}]−500×[L^{c}]⁻¹−0.61×% formamide

2) DNA-RNA or RNA-RNA hybrids:

Tₘ=79.8° C.+18.5(log₁₀[Na⁺]^{a})+0.58(% G/C^{b})+11.8(% G/C^{b})²−820/L^{c}

3) oligo-DNA or oligo-RNAs hybrids:

For <20 nucleotides: Tₘ=2(Iₙ)

For 20-35 nucleotides: Tₘ=22+1.46(Iₙ)
^{a}or for other monovalent cation, but only accurate in the 0.01-0.4 M range.
^{b}only accurate for % GC in the 30% to 75% range.
^{c}L=length of duplex in base pairs.
^{d}oligo, oligonucleotide; I_{n,}=effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3^{rd} Edition, Cold Spring Harbor Laboratory Press, CSH, New York or to Current Protocols in Molecular Biology, John Wiley & Sons, N.Y. (1989 and yearly updates).

### Splice Variant

The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be manmade. Methods for predicting and isolating such splice variants are well known in the art (see for example Foissac and Schiex (2005) BMC Bioinformatics 6: 25).

### Allelic Variant

Alleles or allelic variants are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

### Endogenous Gene

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene. The isolated gene may be isolated from an organism or may be manmade, for example by chemical synthesis.

### Gene Shuffling/Directed Evolution

Gene shuffling or directed evolution consists of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547).

### Construct

Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

### Regulatory Element/Control Sequence/Promoter

The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid used in the methods of the present invention, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). Generally by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a lower level than a strong promoter, in particular at a level that is in all instances below that obtained when under the control of a 35S CaMV promoter.

### Operably Linked

The term “operably linked” as used herein refers to a functional linkage between the promoter sequence and the gene of interest, such that the promoter sequence is able to initiate transcription of the gene of interest.

### Constitutive Promoter

A “constitutive promoter” refers to a promoter that is transcriptionally active during most, but not necessarily all, phases of growth and development and under most environmental conditions, in at least one cell, tissue or organ. Table 2a below gives examples of constitutive promoters.

TABLE 2a
Examples of constitutive promoters

| Gene Source | Reference |
|---|---|
| Actin | McElroy et al, Plant Cell, 2: 163-171, 1990 |
| HMGP | WO 2004/070039 |
| CAMV 35S | Odell et al, Nature, 313: 810-812, 1985 |
| CaMV 19S | Nilsson et al., Physiol. Plant. 100:456-462, 1997 |
| GOS2 | de Pater et al, Plant J November; 2(6):837-44, |
| | 1992, WO 2004/065596 |
| Ubiquitin | Christensen et al, Plant Mol. Biol. 18: 675-689, |
| | 1992 |
| Rice cyclophilin | Buchholz et al, Plant Mol Biol. 25(5): 837-43, |
| | 1994 |
| Maize H3 histone | Lepetit et al, Mol. Gen. Genet. 231:276-285, 1992 |
| Alfalfa H3 histone | Wu et al. Plant Mol. Biol. 11:641-649, 1988 |
| Actin 2 | An et al, Plant J. 10(1); 107-121, 1996 |
| 34S FMV | Sanger et al., Plant. Mol. Biol., 14, 1990: 433-443 |
| Rubisco small subunit | U.S. Pat. No. 4,962,028 |
| OCS | Leisner (1988) Proc Natl Acad Sci USA 85(5): |
| | 2553 |
| SAD1 | Jain et al., Crop Science, 39 (6), 1999: 1696 |
| SAD2 | Jain et al., Crop Science, 39 (6), 1999: 1696 |
| nos | Shaw et al. (1984) Nucleic Acids Res. |
| | 12(20):7831-7846 |
| V-ATPase | WO 01/14572 |
| Super promoter | WO 95/14098 |
| G-box proteins | WO 94/12015 |

### Ubiquitous Promoter

A ubiquitous promoter is active in substantially all tissues or cells of an organism.

### Developmentally-Regulated Promoter

A developmentally-regulated promoter is active during certain developmental stages or in parts of the plant that undergo developmental changes.

### Inducible Promoter

An inducible promoter has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to exposure to various pathogens.

### Organ-Specific/Tissue-Specific Promoter

An organ-specific or tissue-specific promoter is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”.

Examples of root-specific promoters are listed in Table 2b below:

TABLE 2b
Examples of root-specific promoters

| Gene Source | Reference |
|---|---|
| RCc3 | Plant Mol Biol. 1995 January; 27(2):237-48 |
| Arabidopsis PHT1 | Koyama et al. J Biosci Bioeng. 2005 |
| | January; 99(1):38-42.; Mudge et al. |
| | (2002, Plant J. 31:341) |
| Medicago phosphate | Xiao et al., 2006, Plant Biol (Stuttg). |
| transporter | 2006 July; 8(4):439-49 |
| Arabidopsis Pyk10 | Nitz et al. (2001) Plant Sci 161(2): |
| | 337-346 |
| root-expressible genes | Tingey et al., EMBO J. 6: 1, 1987. |
| tobacco auxin-inducible | Van der Zaal et al., Plant Mol. Biol. |
| gene | 16, 983, 1991. |
| β-tubulin | Oppenheimer, et al., Gene 63: 87, 1988. |
| tobacco root-specific genes | Conkling, et al., Plant Physiol. 93: 1203, |
| | 1990. |
| B. napus G1-3b gene | U.S. Pat. No. 5,401,836 |
| SbPRP1 | Suzuki et al., Plant Mol. Biol. 21: 109-119, |
| | 1993. |
| LRX1 | Baumberger et al. 2001, Genes & Dev. |
| | 15:1128 |
| BTG-26 Brassica napus | US 20050044585 |
| LeAMT1 (tomato) | Lauter et al. (1996, PNAS 3:8139) |
| The LeNRT1-1 (tomato) | Lauter et al. (1996, PNAS 3:8139) |
| class I patatin gene (potato) | Liu et al., Plant Mol. Biol. 17 (6): 1139- |
| | 1154 |
| KDC1 (Daucus carota) | Downey et al. (2000, J. Biol. Chem. |
| | 275:39420) |
| TobRB7 gene | W Song (1997) PhD Thesis, North Carolina |
| | State University, Raleigh, NC USA |
| OsRAB5a (rice) | Wang et al. 2002, Plant Sci. 163:273 |
| ALF5 (Arabidopsis) | Diener et al. (2001, Plant Cell 13:1625) |
| NRT2;1Np (N. | Quesada et al. (1997, Plant Mol. Biol. |
| plumbaginifolia) | 34:265) |

A seed-specific promoter is transcriptionally active predominantly in seed tissue, but not necessarily exclusively in seed tissue (in cases of leaky expression). The seed-specific promoter may be active during seed development and/or during germination. The seed specific promoter may be endosperm/aleurone/embryo specific. Examples of seed-specific promoters (endosperm/aleurone/embryo specific) are shown in Table 2c to Table 2f below. Further examples of seed-specific promoters are given in Qing Qu and Takaiwa (Plant Biotechnol. J. 2, 113-125, 2004), which disclosure is incorporated by reference herein as if fully set forth.

TABLE 2c
Examples of seed-specific promoters

| Gene source | Reference |
|---|---|
| seed-specific genes | Simon et al., Plant Mol. Biol. 5: 191, 1985; |
| | Scofield et al., J. Biol. Chem. 262: 12202, |
| | 1987.; Baszczynski et al., Plant Mol. Biol. |
| | 14: 633, 1990. |
| Brazil Nut albumin | Pearson et al., Plant Mol. Biol. 18: 235- |
| | 245, 1992. |
| legumin | Ellis et al., Plant Mol. Biol. 10: 203-214, |
| | 1988. |
| glutelin (rice) | Takaiwa et al., Mol. Gen. Genet. 208: |
| | 15-22, 1986; Takaiwa et al., FEBS Letts. |
| | 221: 43-47, 1987. |
| zein | Matzke et al Plant Mol Biol, 14(3): 323- |
| | 32 1990 |
| napA | Stalberg et al, Planta 199: 515-519, 1996. |
| wheat LMW and HMW | Mol Gen Genet 216:81-90, 1989; |
| glutenin-1 | NAR 17:461-2, 1989 |
| wheat SPA | Albani et al, Plant Cell, 9: 171-184, 1997 |
| wheat α, β, γ-gliadins | EMBO J. 3:1409-15, 1984 |
| barley ltr1 promoter | Diaz et al. (1995) Mol Gen Genet |
| | 248(5):592-8 |
| barley B1, C, D, hordein | Theor Appl Gen 98:1253-62, 1999; Plant J |
| | 4:343-55, 1993; Mol Gen Genet |
| | 250:750-60, 1996 |
| barley DOF | Mena et al, The Plant Journal, 116(1): |
| | 53-62, 1998 |
| blz2 | EP99106056.7 |
| synthetic promoter | Vicente-Carbajosa et al., Plant J. 13: |
| | 629-640, 1998. |
| rice prolamin NRP33 | Wu et al, Plant Cell Physiology 39(8) |
| | 885-889, 1998 |
| rice a-globulin Glb-1 | Wu et al, Plant Cell Physiology 39(8) |
| | 885-889, 1998 |
| rice OSH1 | Sato et al, Proc. Natl. Acad. Sci. USA, 93: |
| | 8117-8122, 1996 |
| rice α-globulin REB/OHP-1 | Nakase et al. Plant Mol. Biol. 33: 513- |
| | 522, 1997 |
| rice ADP-glucose pyrophos- | Trans Res 6:157-68, 1997 |
| phorylase | |
| maize ESR gene family | Plant J 12:235-46, 1997 |
| sorghum α-kafirin | DeRose et al., Plant Mol. Biol 32:1029- |
| | 35, 1996 |
| KNOX | Postma-Haarsma et al, Plant Mol. Biol. |
| | 39:257-71, 1999 |
| rice oleosin | Wu et al, J. Biochem. 123:386, 1998 |
| sunflower oleosin | Cummins et al., Plant Mol. Biol. 19: 873- |
| | 876, 1992 |
| PRO0117, putative rice 40S | WO 2004/070039 |
| ribosomal protein | |
| PRO0136, rice alanine | unpublished |
| aminotransferase | |
| PRO0147, trypsin inhibitor | unpublished |
| ITR1 (barley) | |
| PRO0151, rice WSI18 | WO 2004/070039 |
| PRO0175, rice RAB21 | WO 2004/070039 |
| PRO005 | WO 2004/070039 |
| PRO0095 | WO 2004/070039 |
| α-amylase (Amy32b) | Lanahan et al, Plant Cell 4:203-211, 1992; |
| | Skriver et al, Proc Natl Acad Sci USA |
| | 88:7266-7270, 1991 |
| cathepsin β-like gene | Cejudo et al, Plant Mol Biol 20:849-856, |
| | 1992 |
| Barley Ltp2 | Kalla et al., Plant J. 6:849-60, 1994 |
| Chi26 | Leah et al., Plant J. 4:579-89, 1994 |
| Maize B-Peru | Selinger et al., Genetics 149;1125-38, 1998 |

TABLE 2d
examples of endosperm-specific promoters

| Gene source | Reference |
|---|---|
| glutelin (rice) | Takaiwa et al. (1986) Mol Gen Genet |
| | 208:15-22; Takaiwa et al. (1987) FEBS |
| | Letts. 221:43-47 |
| zein | Matzke et al., (1990) Plant Mol Biol |
| | 14(3): 323-32 |
| wheat LMW and HMW | Colot et al. (1989) Mol Gen Genet |
| glutenin-1 | 216:81-90, Anderson et al. (1989) |
| | NAR 17:461-2 |
| wheat SPA | Albani et al. (1997) Plant Cell 9:171-184 |
| wheat gliadins | Rafalski et al. (1984) EMBO 3:1409-15 |
| barley ltr1 promoter | Diaz et al. (1995) Mol Gen Genet |
| | 248(5):592-8 |
| barley B1, C, D, hordein | Cho et al. (1999) Theor Appl Genet |
| | 98:1253-62; Muller et al. (1993) Plant |
| | J 4:343-55; Sorenson et al. (1996) Mol |
| | Gen Genet 250:750-60 |
| barley DOF | Mena et al, (1998) Plant J 116(1): 53-62 |
| blz2 | Onate et al. (1999) J Biol Chem |
| | 274(14):9175-82 |
| synthetic promoter | Vicente-Carbajosa et al. (1998) Plant J |
| | 13:629-640 |
| rice prolamin NRP33 | Wu et al, (1998) Plant Cell Physiol |
| | 39(8) 885-889 |
| rice globulin Glb-1 | Wu et al. (1998) Plant Cell Physiol |
| | 39(8) 885-889 |
| rice globulin REB/OHP-1 | Nakase et al. (1997) Plant Molec Biol |
| | 33: 513-522 |
| rice ADP-glucose pyrophos- | Russell et al. (1997) Trans Res 6:157-68 |
| phorylase | |
| maize ESR gene family | Opsahl-Ferstad et al. (1997) Plant J |
| | 12:235-46 |
| sorghum kafirin | DeRose et al. (1996) Plant Mol Biol |
| | 32:1029-35 |

TABLE 2e
Examples of embryo specific promoters:

| Gene source | Reference |
|---|---|
| rice OSH1 | Sato et al, Proc. Natl. Acad. Sci. USA, 93: 8117-8122, 1996 |
| KNOX | Postma-Haarsma et al, Plant Mol. Biol. 39:257-71, 1999 |
| PRO0151 | WO 2004/070039 |
| PRO0175 | WO 2004/070039 |
| PRO005 | WO 2004/070039 |
| PRO0095 | WO 2004/070039 |

TABLE 2f
Examples of aleurone-specific promoters:

| Gene source | Reference |
|---|---|
| α-amylase (Amy32b) | Lanahan et al, Plant Cell 4:203-211, 1992; Skriver |
| | et al, Proc Natl Acad Sci USA 88:7266-7270, 1991 |
| cathepsin β-like gene | Cejudo et al, Plant Mol Biol 20:849-856, 1992 |
| Barley Ltp2 | Kalla et al., Plant J. 6:849-60, 1994 |
| Chi26 | Leah et al., Plant J. 4:579-89, 1994 |
| Maize B-Peru | Selinger et al., Genetics 149; 1125-38, 1998 |

A green tissue-specific promoter as defined herein is a promoter that is transcriptionally active predominantly in green tissue, substantially to the exclusion of any other parts of a plant, whilst still allowing for any leaky expression in these other plant parts.

Examples of green tissue-specific promoters which may be used to perform the methods of the invention are shown in Table 2g below.

TABLE 2g
Examples of green tissue-specific promoters

| Gene | Expression | Reference |
|---|---|---|
| Maize Orthophosphate | Leaf | Fukavama et al., Plant |
| dikinase | specific | Physiol. 2001 November; |
| | | 127(3):1136-46 |
| Maize Phosphoenolpyruvate | Leaf | Kausch et al., Plant Mol Biol. |
| carboxylase | specific | 2001 January; 45(1):1-15 |
| Rice Phosphoenolpyruvate | Leaf | Lin et al., 2004 DNA Seq. |
| carboxylase | specific | 2004 August; 15(4):269-76 |
| Rice small subunit Rubisco | Leaf | Nomura et al., Plant Mol |
| | specific | Biol. 2000 September; |
| | | 44(1):99-106 |
| rice beta expansin EXBP9 | Shoot | WO 2004/070039 |
| | specific | |
| Pigeonpea small subunit | Leaf | Panguluri et al., Indian J Exp |
| Rubisco | specific | Biol. 2005 April; 43(4):369-72 |
| Pea RBCS3A | Leaf | |
| | specific | |

Another example of a tissue-specific promoter is a meristem-specific promoter, which is transcriptionally active predominantly in meristematic tissue, substantially to the exclusion of any other parts of a plant, whilst still allowing for any leaky expression in these other plant parts. Examples of green meristem-specific promoters which may be used to perform the methods of the invention are shown in Table 2h below.

TABLE 2h
Examples of meristem-specific promoters

| Gene source | Expression pattern | Reference |
|---|---|---|
| rice OSH1 | Shoot apical meristem, | Sato et al. (1996) Proc. |
| | from embryo globular stage | Natl. Acad. Sci. USA, |
| | to seedling stage | 93: 8117-8122 |
| Rice | Meristem specific | BAD87835.1 |
| metallothionein | | |
| WAK1 & | Shoot and root apical | Wagner & Kohorn |
| WAK 2 | meristems, and in | (2001) Plant Cell |
| | expanding leaves and | 13(2): 303-318 |
| | sepals | |

### Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

### Selectable Marker (Gene)/Reporter Gene

“Selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luceferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

### Transgenic/Transgene/Recombinant

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not present in, or originating from, the genome of said plant, or are present in the genome of said plant but not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

It shall further be noted that in the context of the present invention, the term “isolated nucleic acid” or “isolated polypeptide” may in some instances be considered as a synonym for a “recombinant nucleic acid” or a “recombinant polypeptide”, respectively and refers to a nucleic acid or polypeptide that is not located in its natural genetic environment and/or that has been modified by recombinant methods.

### Modulation

The term “modulation” means in relation to expression or gene expression, a process in which the expression level is changed by said gene expression in comparison to the control plant, the expression level may be increased or decreased. The original, unmodulated expression may be of any kind of expression of a structural RNA (rRNA, tRNA) or mRNA with subsequent translation. For the purposes of this invention, the original unmodulated expression may also be absence of any expression. The term “modulating the activity” shall mean any change of the expression of the inventive nucleic acid sequences or encoded proteins, which leads to increased yield and/or increased growth of the plants. The expression can increase from zero (absence of, or immeasurable expression) to a certain amount, or can decrease from a certain amount to immeasurable small amounts or zero.

### Expression

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

### Increased Expression/Overexpression

The term “increased expression” or “overexpression” as used herein means any form of expression that is additional to the original wild-type expression level. For the purposes of this invention, the original wild-type expression level might also be zero, i.e. absence of expression or immeasurable expression.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

### Decreased Expression

Reference herein to “decreased expression” or “reduction or substantial elimination” of expression is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants.

For the reduction or substantial elimination of expression an endogenous gene in a plant, a sufficient length of substantially contiguous nucleotides of a nucleic acid sequence is required. In order to perform gene silencing, this may be as little as 20, 19, 18, 17, 16, 15, 14, 13, 12, 11, 10 or fewer nucleotides, alternatively this may be as much as the entire gene (including the 5′ and/or 3′ UTR, either in part or in whole). The stretch of substantially contiguous nucleotides may be derived from the nucleic acid encoding the protein of interest (target gene), or from any nucleic acid capable of encoding an orthologue, paralogue or homologue of the protein of interest. Preferably, the stretch of substantially contiguous nucleotides is capable of forming hydrogen bonds with the target gene (either sense or antisense strand), more preferably, the stretch of substantially contiguous nucleotides has, in increasing order of preference, 50%, 60%, 70%, 80%, 85%, 90%, 95%, 96%, 97%, 98%, 99%, 100% sequence identity to the target gene (either sense or antisense strand). A nucleic acid sequence encoding a (functional) polypeptide is not a requirement for the various methods discussed herein for the reduction or substantial elimination of expression of an endogenous gene.

This reduction or substantial elimination of expression may be achieved using routine tools and techniques. A preferred method for the reduction or substantial elimination of endogenous gene expression is by introducing and expressing in a plant a genetic construct into which the nucleic acid (in this case a stretch of substantially contiguous nucleotides derived from the gene of interest, or from any nucleic acid capable of encoding an orthologue, paralogue or homologue of any one of the protein of interest) is cloned as an inverted repeat (in part or completely), separated by a spacer (non-coding DNA).

In such a preferred method, expression of the endogenous gene is reduced or substantially eliminated through RNA-mediated silencing using an inverted repeat of a nucleic acid or a part thereof (in this case a stretch of substantially contiguous nucleotides derived from the gene of interest, or from any nucleic acid capable of encoding an orthologue, paralogue or homologue of the protein of interest), preferably capable of forming a hairpin structure. The inverted repeat is cloned in an expression vector comprising control sequences. A non-coding DNA nucleic acid sequence (a spacer, for example a matrix attachment region fragment (MAR), an intron, a polylinker, etc.) is located between the two inverted nucleic acids forming the inverted repeat. After transcription of the inverted repeat, a chimeric RNA with a self-complementary structure is formed (partial or complete). This double-stranded RNA structure is referred to as the hairpin RNA (hpRNA). The hpRNA is processed by the plant into siRNAs that are incorporated into an RNA-induced silencing complex (RISC). The RISC further cleaves the mRNA transcripts, thereby substantially reducing the number of mRNA transcripts to be translated into polypeptides. For further general details see for example, Grierson et al. (1998) WO 98/53083; Waterhouse et al. (1999) WO 99/53050).

Performance of the methods of the invention does not rely on introducing and expressing in a plant a genetic construct into which the nucleic acid is cloned as an inverted repeat, but any one or more of several well-known “gene silencing” methods may be used to achieve the same effects.

One such method for the reduction of endogenous gene expression is RNA-mediated silencing of gene expression (downregulation). Silencing in this case is triggered in a plant by a double stranded RNA sequence (dsRNA) that is substantially similar to the target endogenous gene. This dsRNA is further processed by the plant into about 20 to about 26 nucleotides called short interfering RNAs (siRNAs). The siRNAs are incorporated into an RNA-induced silencing complex (RISC) that cleaves the mRNA transcript of the endogenous target gene, thereby substantially reducing the number of mRNA transcripts to be translated into a polypeptide. Preferably, the double stranded RNA sequence corresponds to a target gene.

Another example of an RNA silencing method involves the introduction of nucleic acid sequences or parts thereof (in this case a stretch of substantially contiguous nucleotides derived from the gene of interest, or from any nucleic acid capable of encoding an orthologue, paralogue or homologue of the protein of interest) in a sense orientation into a plant. “Sense orientation” refers to a DNA sequence that is homologous to an mRNA transcript thereof. Introduced into a plant would therefore be at least one copy of the nucleic acid sequence. The additional nucleic acid sequence will reduce expression of the endogenous gene, giving rise to a phenomenon known as co-suppression. The reduction of gene expression will be more pronounced if several additional copies of a nucleic acid sequence are introduced into the plant, as there is a positive correlation between high transcript levels and the triggering of co-suppression.

Another example of an RNA silencing method involves the use of antisense nucleic acid sequences. An “antisense” nucleic acid sequence comprises a nucleotide sequence that is complementary to a “sense” nucleic acid sequence encoding a protein, i.e. complementary to the coding strand of a double-stranded cDNA molecule or complementary to an mRNA transcript sequence. The antisense nucleic acid sequence is preferably complementary to the endogenous gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire nucleic acid sequence (in this case a stretch of substantially contiguous nucleotides derived from the gene of interest, or from any nucleic acid capable of encoding an orthologue, paralogue or homologue of the protein of interest), but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine substituted nucleotides may be used. Examples of modified nucleotides that may be used to generate the antisense nucleic acid sequences are well known in the art. Known nucleotide modifications include methylation, cyclization and ‘caps’ and substitution of one or more of the naturally occurring nucleotides with an analogue such as inosine. Other modifications of nucleotides are well known in the art.

The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

The nucleic acid molecules used for silencing in the methods of the invention (whether introduced into a plant or generated in situ) hybridize with or bind to mRNA transcripts and/or genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using the vectors described herein.

According to a further aspect, the antisense nucleic acid sequence is an a-anomeric nucleic acid sequence. An a-anomeric nucleic acid sequence forms specific double-stranded hybrids with complementary RNA in which, contrary to the usual b-units, the strands run parallel to each other (Gaultier et al. (1987) Nucl Ac Res 15: 6625-6641). The antisense nucleic acid sequence may also comprise a 2′-o-methylribonucleotide (Inoue et al. (1987) Nucl Ac Res 15, 6131-6148) or a chimeric RNA-DNA analogue (Inoue et al. (1987) FEBS Lett. 215, 327-330).

The reduction or substantial elimination of endogenous gene expression may also be performed using ribozymes. Ribozymes are catalytic RNA molecules with ribonuclease activity that are capable of cleaving a single-stranded nucleic acid sequence, such as an mRNA, to which they have a complementary region. Thus, ribozymes (e.g., hammerhead ribozymes (described in Haselhoff and Gerlach (1988) Nature 334, 585-591) can be used to catalytically cleave mRNA transcripts encoding a polypeptide, thereby substantially reducing the number of mRNA transcripts to be translated into a polypeptide. A ribozyme having specificity for a nucleic acid sequence can be designed (see for example: Cech et al. U.S. Pat. No. 4,987,071; and Cech et al. U.S. Pat. No. 5,116,742). Alternatively, mRNA transcripts corresponding to a nucleic acid sequence can be used to select a catalytic RNA having a specific ribonuclease activity from a pool of RNA molecules (Bartel and Szostak (1993) Science 261, 1411-1418). The use of ribozymes for gene silencing in plants is known in the art (e.g., Atkins et al. (1994) WO 94/00012; Lenne et al. (1995) WO 95/03404; Lutziger et al. (2000) WO 00/00619; Prinsen et al. (1997) WO 97/13865 and Scott et al. (1997) WO 97/38116).

Gene silencing may also be achieved by insertion mutagenesis (for example, T-DNA insertion or transposon insertion) or by strategies as described by, among others, Angell and Baulcombe ((1999) Plant J 20(3): 357-62), (Amplicon VIGS WO 98/36083), or Baulcombe (WO 99/15682).

Gene silencing may also occur if there is a mutation on an endogenous gene and/or a mutation on an isolated gene/nucleic acid subsequently introduced into a plant. The reduction or substantial elimination may be caused by a non-functional polypeptide. For example, the polypeptide may bind to various interacting proteins; one or more mutation(s) and/or truncation(s) may therefore provide for a polypeptide that is still able to bind interacting proteins (such as receptor proteins) but that cannot exhibit its normal function (such as signalling ligand).

A further approach to gene silencing is by targeting nucleic acid sequences complementary to the regulatory region of the gene (e.g., the promoter and/or enhancers) to form triple helical structures that prevent transcription of the gene in target cells. See Helene, C., Anticancer Drug Res. 6, 569-84, 1991; Helene et al., Ann. N.Y. Acad. Sci. 660, 27-36 1992; and Maher, L. J. Bioassays 14, 807-15, 1992.

Other methods, such as the use of antibodies directed to an endogenous polypeptide for inhibiting its function in planta, or interference in the signalling pathway in which a polypeptide is involved, will be well known to the skilled man. In particular, it can be envisaged that manmade molecules may be useful for inhibiting the biological function of a target polypeptide, or for interfering with the signalling pathway in which the target polypeptide is involved.

Alternatively, a screening program may be set up to identify in a plant population natural variants of a gene, which variants encode polypeptides with reduced activity. Such natural variants may also be used for example, to perform homologous recombination.

Artificial and/or natural microRNAs (miRNAs) may be used to knock out gene expression and/or mRNA translation. Endogenous miRNAs are single stranded small RNAs of typically 19-24 nucleotides long. They function primarily to regulate gene expression and/or mRNA translation. Most plant microRNAs (miRNAs) have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. mRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes.

Artificial microRNAs (amiRNAs), which are typically 21 nucleotides in length, can be genetically engineered specifically to negatively regulate gene expression of single or multiple genes of interest. Determinants of plant microRNA target selection are well known in the art. Empirical parameters for target recognition have been defined and can be used to aid in the design of specific amiRNAs, (Schwab et al., Dev. Cell 8, 517-527, 2005). Convenient tools for design and generation of amiRNAs and their precursors are also available to the public (Schwab et al., Plant Cell 18, 1121-1133, 2006).

For optimal performance, the gene silencing techniques used for reducing expression in a plant of an endogenous gene requires the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid to be introduced.

Described above are examples of various methods for the reduction or substantial elimination of expression in a plant of an endogenous gene. A person skilled in the art would readily be able to adapt the aforementioned methods for silencing so as to achieve reduction of expression of an endogenous gene in a whole plant or in parts thereof through the use of an appropriate promoter, for example.

### Transformation

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet 208:1-9; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

The genetically modified plant cells can be regenerated via all methods with which the skilled worker is familiar. Suitable methods can be found in the above-mentioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

### T-DNA Activation Tagging

T-DNA activation tagging (Hayashi et al. Science (1992) 1350-1353), involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter.

### TILLING

The term “TILLING” is an abbreviation of “Targeted Induced Local Lesions In Genomes” and refers to a mutagenesis technology useful to generate and/or identify nucleic acids encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet 5(2): 145-50).

### Homologous Recombination

Homologous recombination allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offringa et al. (1990) EMBO J 9(10): 3077-84) but also for crop plants, for example rice (Terada et al. (2002) Nat Biotech 20(10): 1030-4; lida and Terada (2004) Curr Opin Biotech 15(2): 132-8), and approaches exist that are generally applicable regardless of the target organism (Miller et al, Nature Biotechnol. 25, 778-785, 2007).

### Yield Related Traits

Yield related traits are traits or features which are related to plant yield. Yield-related traits may comprise one or more of the following non-limitative list of features: early flowering time, yield, biomass, seed yield, early vigour, greenness index, increased growth rate, improved agronomic traits, such as e.g. improved Water Use Efficiency (WUE), improved Nitrogen Use Efficiency (NUE), etc.

### Yield

The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters.

The terms “yield” of a plant and “plant yield” are used interchangeably herein and are meant to refer to vegetative biomass such as root and/or shoot biomass, to reproductive organs, and/or to propagules such as seeds of that plant.

Taking corn as an example, male inflorescences (tassels) and female inflorescences (ears). The female inflorescence produces pairs of spikelets on the surface of a central axis (cob). Each of the female spikelets encloses two fertile florests, one of whose will usually mature into a maize kernel once fertilized. Hence a yield increase in maize may be manifested as one or more of the following: increase in the number of plants established per square meter, an increase in the number of ears per plant, an increase in the number of rows, number of kernels per row, kernel weight, thousand kernel weight, ear length/diameter, increase in the seed filling rate, which is the number of filled florets (i.e. florets containing seed) divided by the total number of florets and multiplied by 100), among others.

Inflorescences in rice plants are called panicles. The panicle bears spikelets. The spikelet is the basic unit of the panicles and consists of a pedicel and a floret. The floret is born on the pedicel. A floret includes a flower that is covered by two protective glumes: a larger glume (the lemma) and a shorter glume (the palea). Hence, taking rice as an example, a yield increase may manifest itself as an increase in one or more of the following: number of plants per square meter, number of panicles per plant, panicle length, number of spikelets per panicle, number of flowers (or florets) per panicle, increase in the seed filling rate which is the number of filled florets (i.e. florets containing seeds divided by the total number of florets and multiplied by 100), increase in thousand kernel weight, among others. In rice, submergence tolerance may also result in increased yield.

### Early Flowering Time

Plants having an “early flowering time” as used herein are plants which start to flower earlier than control plants. Hence this term refers to plants that show an earlier start of flowering. Flowering time of plants can be assessed by counting the number of days (“time to flower”) between sowing and the emergence of a first inflorescence. The “flowering time” of a plant can for instance be determined using the method as described in WO 2007/093444.

### Early Vigour

“Early vigour” refers to active healthy well-balanced growth especially during early stages of plant growth, and may result from increased plant fitness due to, for example, the plants being better adapted to their environment (i.e. optimizing the use of energy resources and partitioning between shoot and root). Plants having early vigour also show increased seedling survival and a better establishment of the crop, which often results in highly uniform fields (with the crop growing in uniform manner, i.e. with the majority of plants reaching the various stages of development at substantially the same time), and often better and higher yield. Therefore, early vigour may be determined by measuring various factors, such as thousand kernel weight, percentage germination, percentage emergence, seedling growth, seedling height, root length, root and shoot biomass and many more.

### Increased Growth Rate

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as speed of germination, early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per square meter (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

### Stress Resistance

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35%, 30% or 25%, more preferably less than 20% or 15% in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. “Mild stresses” are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures.

“Biotic stresses” are typically those stresses caused by pathogens, such as bacteria, viruses, fungi, nematodes and insects.

The “abiotic stress” may be an osmotic stress caused by a water stress, e.g. due to drought, salt stress, or freezing stress. Abiotic stress may also be an oxidative stress or a cold stress. “Freezing stress” is intended to refer to stress due to freezing temperatures, i.e. temperatures at which available water molecules freeze and turn into ice. “Cold stress”, also called “chilling stress”, is intended to refer to cold temperatures, e.g. temperatures below 10°, or preferably below 5° C., but at which water molecules do not freeze. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 97%, 95%, 92%, 90%, 87%, 85%, 83%, 80%, 77% or 75% of the average production of such plant in a given environment. Average production may be calculated on harvest and/or season basis. Persons skilled in the art are aware of average yield productions of a crop.

In particular, the methods of the present invention may be performed under non-stress conditions. In an example, the methods of the present invention may be performed under non-stress conditions such as mild drought to give plants having increased yield relative to control plants.

In another embodiment, the methods of the present invention may be performed under stress conditions.

In an example, the methods of the present invention may be performed under stress conditions such as drought to give plants having increased yield relative to control plants. In another example, the methods of the present invention may be performed under stress conditions such as nutrient deficiency to give plants having increased yield relative to control plants.

Nutrient deficiency may result from a lack of nutrients such as nitrogen, phosphates and other phosphorous-containing compounds, potassium, calcium, magnesium, manganese, iron and boron, amongst others.

In yet another example, the methods of the present invention may be performed under stress conditions such as salt stress to give plants having increased yield relative to control plants. The term salt stress is not restricted to common salt (NaCl), but may be any one or more of: NaCl, KCl, LiCl, MgCl₂, CaCl₂, amongst others.

In yet another example, the methods of the present invention may be performed under stress conditions such as cold stress or freezing stress to give plants having increased yield relative to control plants.

### Increase/Improve/Enhance

The terms “increase”, “improve” or “enhance” are interchangeable and shall mean in the sense of the application at least a 3%, 4%, 5%, 6%, 7%, 8%, 9%, or 10%, preferably at least 15% or 20%, more preferably 25%, 30%, 35% or 40% more yield and/or growth in comparison to control plants as defined herein.

### Seed Yield

Increased seed yield may manifest itself as one or more of the following:
a) an increase in seed biomass (total seed weight) which may be on an individual seed basis and/or per plant and/or per square meter;
b) increased number of flowers per plant;
c) increased number of seeds;
d) increased seed filling rate (which is expressed as the ratio between the number of filled florets divided by the total number of florets);
e) increased harvest index, which is expressed as a ratio of the yield of harvestable parts, such as seeds, divided by the biomass of aboveground plant parts; and
f) increased thousand kernel weight (TKW), which is extrapolated from the number of seeds counted and their total weight. An increased TKW may result from an increased seed size and/or seed weight, and may also result from an increase in embryo and/or endosperm size.

An increase in seed yield may also be manifested as an increase in seed size and/or seed volume. Furthermore, an increase in seed yield may also manifest itself as an increase in seed area and/or seed length and/or seed width and/or seed perimeter.

### Greenness Index

The “greenness index” as used herein is calculated from digital images of plants. For each pixel belonging to the plant object on the image, the ratio of the green value versus the red value (in the RGB model for encoding color) is calculated. The greenness index is expressed as the percentage of pixels for which the green-to-red ratio exceeds a given threshold. Under normal growth conditions, under salt stress growth conditions, and under reduced nutrient availability growth conditions, the greenness index of plants is measured in the last imaging before flowering. In contrast, under drought stress growth conditions, the greenness index of plants is measured in the first imaging after drought.

### Biomass

The term “biomass” as used herein is intended to refer to the total weight of a plant. Within the definition of biomass, a distinction may be made between the biomass of one or more parts of a plant, which may include any one or more of the following:
aboveground parts such as but not limited to shoot biomass, seed biomass, leaf biomass, etc.;
aboveground harvestable parts such as but not limited to shoot biomass, seed biomass, leaf biomass, etc.:
parts below ground, such as but not limited to root biomass, etc.;
harvestable parts below ground, such as but not limited to root biomass, etc.;
vegetative biomass such as root biomass, shoot biomass, etc.;
reproductive organs; and
propagules such as seed.

### Marker Assisted Breeding

Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

### Use as Probes in (Gene Mapping)

Use of nucleic acids encoding the protein of interest for genetically and physically mapping the genes requires only a nucleic acid sequence of at least 15 nucleotides in length. These nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the nucleic acids encoding the protein of interest. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the nucleic acid encoding the protein of interest in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

### Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

### Control Plant(s)

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. Nullizygotes are individuals missing the transgene by segregation. A “control plant” as used herein refers not only to whole plants, but also to plant parts, including seeds and seed parts.

### DETAILED DESCRIPTION OF THE INVENTION

Surprisingly, it has now been found that modulating expression in a plant of a nucleic acid encoding a GDH polypeptide gives plants having enhanced yield-related traits relative to control plants. According to a first embodiment, the present invention provides a method for enhancing yield-related traits in plants relative to control plants, comprising modulating expression in a plant of a nucleic acid encoding a GDH polypeptide and optionally selecting for plants having enhanced yield-related traits.

The invention also provides hitherto unknown GDH-encoding nucleic acids and GDH polypeptides. According to a further embodiment of the present invention, there is therefore provided an isolated nucleic acid molecule selected from:
(i) a nucleic acid represented by SEQ ID NO: 99;
(ii) the complement of a nucleic acid represented by SEQ ID NO: 99;
(iii) a nucleic acid encoding a GDH polypeptide having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 100, and having in increasing order of preference at least 50%, 55%, 60%, 65%, 70%, 75%, 80%, 85%, 90%, 95%, 96%, 97%, 98%, 99% or more sequence identity to any one of the motifs 1 to 6.

According to a further embodiment of the present invention, there is also provided an isolated polypeptide selected from:
(i) an amino acid sequence represented by SEQ ID NO: 176;
(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 176, and having in increasing order of preference at least 50%, 55%, 60%, 65%, 70%, 75%, 80%, 85%, 90%, 95%, 96%, 97%, 98%, 99% or more sequence identity to any one of the motifs 1 to 6;
(iii) derivatives of any of the amino acid sequences given in (i) or (ii) above.

Furthermore, it has now surprisingly been found that modulating expression in a plant of a nucleic acid encoding a FLA-like polypeptide gives plants having enhanced yield-related traits relative to control plants. According to a first embodiment, the present invention provides a method for enhancing yield-related traits in plants relative to control plants, comprising modulating expression in a plant of a nucleic acid encoding a FLA-like polypeptide and optionally selecting for plants having enhanced yield-related traits.

According to a further embodiment of the present invention, there is therefore provided an isolated nucleic acid molecule selected from:
(i) a nucleic acid represented by any one or more of the nucleic acids set forth in Table A2;
(ii) the complement of a nucleic acid of (i);
(iii) a nucleic acid encoding a FLA-like polypeptide having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence represented by any of the polypeptide sequences of Table A2, and additionally or alternatively comprising one or more motifs having in increasing order of preference at least 50%, 55%, 60%, 65%, 70%, 75%, 80%, 85%, 90%, 95%, 96%, 97%, 98%, 99% or 100% sequence identity to any one or more of the domains and motifs sequence given in SEQ ID NO: 487 to SEQ ID NO: 497 and further preferably conferring enhanced yield-related traits relative to control plants.
(iv) a nucleic acid molecule which hybridizes with a nucleic acid molecule of (i) to (iii) under high stringency hybridization conditions and preferably confers enhanced yield-related traits relative to control plants.

According to a further embodiment of the present invention, there is also provided an isolated polypeptide selected from:
(i) any of the amino acid sequence selected from the group of the polypeptides set forth in Table A2;
(ii) an amino acid sequence having, in increasing order of preference, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any one or more of the amino acid sequence of the polypeptides set forth in Table A2, and additionally or alternatively comprising one or more motifs having in increasing order of preference at least 50%, 55%, 60%, 65%, 70%, 75%, 80%, 85%, 90%, 95%, 96%, 97%, 98%, 99% or 100% sequence identity to any one or more of the motifs given in SEQ ID NO: 487 to SEQ ID NO: 497, and further preferably conferring enhanced yield-related traits relative to control plants;
(iii) derivatives of any of the amino acid sequences given in (i) or (ii) above.

Furthermore, it has now surprisingly been found that modulating expression in a plant of a nucleic acid encoding a SAUR polypeptide gives plants having enhanced yield-related traits relative to control plants. According to a first embodiment, the present invention provides a method for enhancing yield-related traits in plants relative to control plants, comprising modulating expression in a plant of a nucleic acid encoding a SAUR polypeptide and optionally selecting for plants having enhanced yield-related traits.

The invention also provides hitherto unknown SAUR-encoding nucleic acids and SAUR polypeptides useful for conferring enhanced yield-related traits in plants relative to control plants.

According to a further embodiment of the present invention, there is therefore provided an isolated nucleic acid molecule selected from:
(i) a nucleic acid represented by any one of the nucleic acids of Table A3;
(ii) the complement of a nucleic acid represented by (i);
(iii) a nucleic acid encoding a SAUR polypeptide having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of any of the polypeptides of Table A3 and additionally or alternatively comprising one or more motifs having in increasing order of preference at least 50%, 55%, 60%, 65%, 70%, 75%, 80%, 85%, 90%, 95%, 96%, 97%, 98%, 99% or more sequence identity to any one or more of the motifs given in SEQ ID NO: 1155 to SEQ ID NO: 1160, and further preferably conferring enhanced yield-related traits relative to control plants.
(iv) a nucleic acid molecule which hybridizes with a nucleic acid molecule of (i) to (iii) under high stringency hybridization conditions and preferably confers enhanced yield-related traits relative to control plants.

According to a further embodiment of the present invention, there is also provided an isolated polypeptide selected from:
(i) an amino acid sequence represented by any one of the polypeptides of Table A3;
(ii) an amino acid sequence having, in increasing order of preference, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of any one of the polypeptides of Table A3 and additionally or alternatively comprising one or more motifs having in increasing order of preference at least 50%, 55%, 60%, 65%, 70%, 75%, 80%, 85%, 90%, 95%, 96%, 97%, 98%, 99% or more sequence identity to any one or more of the motifs given in SEQ ID NO: 1155 to SEQ ID NO: 1160, and further preferably conferring enhanced yield-related traits relative to control plants;
(iii) derivatives of any of the amino acid sequences given in (i) or (ii) above.

Furthermore, it has now surprisingly been found that modulating expression in a plant of a nucleic acid encoding a SAUR polypeptide gives plants having enhanced yield-related traits relative to control plants. According to a first embodiment, the present invention provides a method for enhancing yield-related traits in plants relative to control plants, comprising modulating expression in a plant of a first nucleic acid encoding at least a SAUR polypeptide and a second nucleic acid encoding one or more SYNP polypeptides, or a nucleic acid encoding a protein fusion between at least a SAUR and one or more SYNP polypeptides wherein the first and second nucleic acids are comprised in a single nucleic acid molecule or in multiple, at least two, nucleic acid molecules and optionally selecting for plants having enhanced yield-related traits.

Furthermore, it has now surprisingly been found that modulating expression in a plant of a nucleic acid encoding a DHAR polypeptide gives plants having enhanced yield-related traits relative to control plants. According to a first embodiment, the present invention provides a method for enhancing yield-related traits in plants relative to control plants, comprising modulating expression in a plant of a nucleic acid encoding a DHAR polypeptide and optionally selecting for plants having enhanced yield-related traits.

The invention also provides hitherto unknown DHAR-encoding nucleic acids and DHAR polypeptides.

According to a further embodiment of the present invention, there is therefore provided an isolated nucleic acid molecule selected from:
(i) a nucleic acid represented by SEQ ID NO: 1997, SEQ ID NO: 2121 and SEQ ID NO: 2193;
(ii) the complement of a nucleic acid represented by SEQ ID NO: 1997, SEQ ID NO: 2121 and SEQ ID NO: 2193;
(iii) a nucleic acid encoding a DHAR polypeptide as represented by any one of SEQ ID NO: 1998, SEQ ID NO: 2122 and SEQ ID NO: 2194, preferably as a result of the degeneracy of the genetic code, said isolated nucleic acid can be derived from a polypeptide sequence as represented by any one of said SEQ IDs and further preferably confers enhanced yield related traits relative to control plants;
(iv) a nucleic acid having, in increasing order of preference, at least 50%, 55%, 60%, 65%, 70%, 75%, 80%, 85%, 90%, 95%, 96%, 97%, 98%, 99% or more sequence identity with any of the nucleic acid sequences of Table A5 and further preferably confers enhanced yield related traits relative to control plants;
(v) a nucleic acid molecule which hybridizes with a nucleic acid molecule of (i) to (iv) under stringent hybridization conditions and preferably confers enhanced yield related traits relative to control plants;
(vi) a nucleic acid encoding a DHAR polypeptide having in increasing order of preference at least 50%, 55%, 60%, 65%, 70%, 75%, 80%, 85%, 90%, 95%, 96%, 97%, 98%, 99% or more sequence identity to any one of SEQ ID NO: 1998, SEQ ID NO: 2122 and SEQ ID NO: 2194 and any of the other amino acid sequences in Table A5 and preferably confers enhanced yield related traits relative to control plants.

According to a further embodiment of the present invention, there is also provided an isolated polypeptide selected from:
(i) an amino acid sequence represented by of SEQ ID NO: 1998, SEQ ID NO: 2122 and SEQ ID NO: 2194;
(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 1998, SEQ ID NO: 2122 and SEQ ID NO: 2194, and having in increasing order of preference at least 50%, 55%, 60%, 65%, 70%, 75%, 80%, 85%, 90%, 95%, 96%, 97%, 98%, 99% or more sequence identity to of SEQ ID NO: 1998, SEQ ID NO: 2122 and SEQ ID NO: 2194;
(iii) derivatives of any of the amino acid sequences given in (i) or (ii) above.

A preferred method for modulating (preferably, increasing) expression of a nucleic acid encoding a GDH polypeptide is by introducing and expressing in a plant a nucleic acid encoding a GDH polypeptide. Another preferred method for modulating (preferably, increasing) expression of a nucleic acid encoding a FLA-like polypeptide is by introducing and expressing in a plant a nucleic acid encoding a FLA-like polypeptide. Yet another preferred method for modulating (preferably, increasing) expression of a nucleic acid encoding a SAUR polypeptide is by introducing and expressing in a plant a nucleic acid encoding a SAUR polypeptide. Another preferred method for modulating (preferably, increasing) expression of a nucleic acid encoding a SAUR and a SYNP polypeptide is by introducing and expressing in a plant a first nucleic acid encoding at least a SAUR polypeptide and a second nucleic acid encoding one or more SYNP polypeptides, or a nucleic acid encoding a protein fusion between at least a SAUR and one or more SYNP polypeptides, wherein the first and second nucleic acids are comprised in a single nucleic acid molecule or in multiple, at least two, nucleic acid molecules. Still another preferred method for modulating (preferably, increasing) expression of a nucleic acid encoding a DHAR polypeptide is by introducing and expressing in a plant a nucleic acid encoding a DHAR polypeptide.

In one embodiment a “protein useful in the methods of the invention” is taken to mean a GDH polypeptide as defined herein. Any reference hereinafter to a “nucleic acid useful in the methods of the invention” is taken to mean a nucleic acid capable of encoding such a GDH polypeptide. The nucleic acid to be introduced into a plant (and therefore useful in performing the methods of the invention) is any nucleic acid encoding the type of protein which will now be described, hereafter also named “GDH nucleic acid” or “GDH gene”.

A “GDH polypeptide” as defined herein refers to any polypeptide having glutamate dehydrogenase activity, preferably having NAD-dependent glutamate dehydrogenase activity (EC1.4.1.2). Typically, a GDH polypeptide useful in the methods of the present invention comprises a Glu/Phe/Leu/Val dehydrogenase, C-terminal domain (Pfam entry PF00208) and a Glu/Phe/Leu/Val dehydrogenase, dimerisation region (Pfam entry PF02812).

Preferably, the GDH polypeptide comprises one or more of the following motifs:

Motif 1, SEQ ID NO: 3:
L(L/M)IP(Y/F/L)RE(I/V)KVEC(T/S)(I/L)P(K/R)DDG
Motif 2, SEQ ID NO: 4:
EVNALAQLMTWK(T/S)AV
Motif 3, SEQ ID NO: 5:
PAPDMGT(N/G)(A/S/P)QTMAW(I/M)(L/F)DEYSKFHG(H/Y)
(T/S)PA(V/I)VTGKP
Motif 4, SEQ ID NO: 6:
CD(V/I)L(I/V/L)P(A/C)ALGGV
Motif 5, SEQ ID NO: 7:
LPD(I/V)(Y/F/L)AN(S/C/A)GGV(T/I/V)V(S/G)YFEWVQN
(I/T/K)QGF(M/P/R)W(D/E/N)E
Motif 6, SEQ ID NO: 8, in the C-terminal end:
RGW
Motif 7, SEQ ID NO: 9:
F(R/K)V(P/S/V)W(V/I/L/M/E)DD(R/K/S/N)G(E/V/Q/R/K)
(T/A/K/L/I/C)(H/C/R/Q)(V/I/F)N(R/K)G(F/W/Y)RV
(H/Q)(F/W)
Motif 8, SEQ ID NO: 10:
GP(C/Y/F)(R/K)GG(I/L/M)R(F/L)(H/R)
Motif 9, SEQ ID NO: 11:
GG(S/A)DF(D/N/E)PKG(K/R)(T/S)(D/E)
Motif 10, SEQ ID NO: 12:
VGX(R/K)E(M/I/L)GY(L/M)(L/F/Y)GQ(Y/W)(R/K)R(L/I)
wherein X in position 3 can be any amino acid,
preferably X is one of T, P, F, A, G
Motif 11, SEQ ID NO: 13:
P(A/G/S)(I/M/K)A(A/T/V/S)(G/A/N)AGGV
Motif 12, SEQ ID NO: 14:
YR(V/A)QH(N/D/S)(N/H)(A/V/S/H)(L/R)(G/L/M)P(F/Y/V)
KGG(I/L)
Motif 13, SEQ ID NO: 15:
LA(S/A)L(N/M)T(W/F)K
Motif 14, SEQ ID NO: 16:
(F/Y)GG(A/S)KGG(V/I)
Motif 15, SEQ ID NO: 17:
VTGKP(T/V/L)(W/Y/E)(L/E)(H/G)G(I/S)(V/L/D/H)GR
(D/E/T/Q)(K/S/A/E)A(G/T)G(R/L)G
Motif 16, SEQ ID NO: 18:
P(A/C)DV(F/L)IP(C/A)A(V/I/L)
Motif 17, SEQ ID NO: 19:
EAAN(G/S)(A/P)(L/T/V)TP
Motif 18, SEQ ID NO: 20:
LPD(L/I)(I/Y/L)(A/T/C/L)N(G/A)G(A/G)V(V/I/T)VS
(F/Y)FEW(V/L)(Q/K)N

More preferably, the GDH polypeptide comprises in increasing order of preference, at least 2, at least 3, at least 4, at least 5 of the motifs listed above. Most preferably, the GDH polypeptide has also one of the following motifs:

Motif 19, SEQ ID NO: 21:
MNAL(A/V)AT(N/S)R(N/S)F
or
Motif 20, SEQ ID NO: 22:
RIFCTVC(K/R)EYGRKHRRNPYGNEGSRNMQ

Alternatively or additionally, the homologue of a GDH protein has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 2, provided that the homologous protein comprises the conserved motifs as outlined above. The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered. Preferably the motifs in a GDH polypeptide have, in increasing order of preference, at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the motifs represented by SEQ ID NO: 3 to SEQ ID NO: 22 (Motifs 1 to 20).

Preferably, the polypeptide sequence which when used in the construction of a phylogenetic tree, such as the one depicted in FIG. 3, clusters with GDH polypeptides, preferably with class I GDH polypeptides or with moss GDH polypeptides related to class I, more preferably to group of beta subunit GDH polypeptides comprising the amino acid sequence represented by SEQ ID NO: 2 rather than with any other group. Several moss GDH sequences cluster with Class I GDH sequences (for example Physcomitrella patens 126976, SEQ ID NO: 77), and thus are equally useful in the methods of present invention.

In another embodiment a “protein useful in the methods of the invention” is taken to mean a FLA-like polypeptide as defined herein. Any reference hereinafter to a “nucleic acid useful in the methods of the invention” is taken to mean a nucleic acid capable of encoding such a FLA-like polypeptide. The nucleic acid to be introduced into a plant (and therefore useful in performing the methods of the invention) is any nucleic acid encoding the type of protein which will now be described, hereafter also named “FLA-like polypeptide nucleic acid” or “FLA-like polypeptide gene”.

A “FLA-Hike polypeptide” as defined herein refers to any Fasciclin-like arabinogalactan polypeptide which typically has the capability to be glycosylated in a cell.

A FLA-like polypeptide useful in the methods of the invention comprises in increasing order of preference one, two, three, four or more of the following:
(i) an AGP-like (arabinogalactan-like) glycosylation region; and
(ii) a fasciclin-like (FLA-like) domain

Fasciclin-like domain as refer herein means a protein domain present and conserved amongst FLA-like polypeptides originating from different organism represented by any one of the sequences as found in specialized databases for conserved proteins domains such as SMART (Schultz et al. (1998) Proc. Natl. Acad. Sci. USA 95, 5857-5864; Letunic et al. (2008) Nucleic Acids Res, doi:10.1093/nar/gkn808) having accession number Smart00554, or in pfam having the domain accession reference name “fasciclin” and accession number PF02469 (Finn et al. Nucleic Acids Research (2008) Database Issue 36:D281-D288; Pfam: the protein families database R. D. Finn (eds M. J. Dunn, L. B. Jorde, P. F. R. Little, S. Subramaniam) Genetics, Genomics, Proteomics and Bioinformatics, Section 6: Protein Families (2005) ISBN 978-0-470-84974-3). Other domain databases such as those integrated at Intepro (Hunter et al. 2009 Nucleic Acids Res. 37 (Database Issue):D224-228; Quevillon 2005 Nucleic Acids Res. 33 (Web Server issue):W116-W120) comprise further examples of amino acid sequences of Fasciclin-like domains. Methods to consult sequences of protein domain databases comprising the sequence of Fasciclin-like domains and methods to identify a FLA-like domain in a polypeptide are well known in the art. Further details on such methods are provided in the Examples section.

A preferred FLA-like polypeptide useful in the methods of the invention comprises at least one, two, three, or four fasciclin-like domains having in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid represented by:
(i) TITVCAVDNAGMSDLLSKQLSIYTIKNVLSFRVLLDYFDAKKLHQITNGTALAATM FQATGSATGSSGFVNITDLRGGKVGLSPADYNGPPPAKFVKSIAEIPYNISVIQIS TIL (SEQ ID NO: 487) which corresponds to the N-term FLA-like domain in SEQ ID NO: 172; or
(ii) VDGGVTIFCPRDDAMKKFLPKFKNLTAEGKQSLLEYHGIPIYQSISNLKSNNGD MNTLATDGAKKYAVVIQNDGEDVTIKTKIVTAKITATVVDKLPLAIYSLDKVL (SEQ ID NO: 488) which corresponds to the C-term FLA-like domain in SEQ ID NO: 2; or
(iii) to the amino acid sequence of any of the FLA-like domains as present in any of the polypeptides of Table A2.

Two highly conserved peptides have been identified in Fasciclin-like domains, the so called H1 and H2 regions (Johnson et al., Plant Physiol. (2003) 133 (4) 1911-1925).

A preferred FLA-like domain as present in a FLA-like polypeptide useful in the methods of the invention comprises any one or more of the following:
(i) An H1 region having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid ITVCAVDNAGM (SEQ ID NO: 491) or to the amino acid sequence GVTIFCPRDDAM (SEQ ID NO: 492) which represent the H1 region in the N-term and C-terminal FLA-like domains of SEQ ID NO: 172 respectively or to the consensus H1 sequence represented by [Ser/Thr]-[Val/Leu/Ile]-Phe-Ala-Pro-X-[Asp/Glu/Asn]-X-Ala (SEQ ID NO: 489) wherein X represents any amino acid;
(ii) An H2 region having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid: ISVIQISTIL (SEQ ID NO: 493) or to the amino acid sequence LAIYSLDK (SEQ ID NO: 494) which represent the H2 region in the N-term and C-terminal FLA-like domains of SEQ ID NO: 172 respectively, or to the consensus H2 sequence represented by [Val/Leu/Ile]-[Phe/Tyr/His/Gln]-X-[Val/Leu/Ile]-X-X-[Val/Leu/Ile]-[Val/Leu/Ile]-[Val/Leu/Ile]-Pro Ala (SEQ ID NO: 320) wherein X represents any amino acid.

The recent identification of a sos5 (salt overly sensitive) mutant in Arabidopsis with an amino acid substitution in the H2 region of FLA4 (Shi et al., 2003, Plant Cell. 2003 January; 15(1):19-32) indicates that this domain is important for FLA function. The junction region between the second fasciclin-like domain and the second AGP-like glycosylation region is very conserved in FLA-like polypeptides. This region encompasses a highly conserved Ser-348 in the proper function of SOS5. The sos5 mutant phenotypes clearly indicate a critical role for Ser-348.

The sugar residues typically found in glycosylated FLA-like polypeptides arabinooligosaccharides and large arabinoglactan polysaccharide chains. The presence of clustered, non-contiguous Proline residues, separated by Alanine or Serine residues in the proteins backbone in FLA-like polypeptide typically results in glycosylation with large arabinogalacta polysaccharide chains in a cellular environment. FLA1-like polypeptides contain N-glycosylation sites in the fasciclin domain and additional sites including O-glycosylation sites are present in other regions of the polypeptide. Typically glycosylation sites may be identified based on the presence of at least two non-contiguous Pro residues; for example, the sequence (A/S) P(A/S) P. In vivo, these regions are predicted to be hydroxyproline (HYP) glycosylated (Hyp-O-glycosylated) and are increasingly being referred to as “glycomodules”.

According to another embodiment FLA-like polypeptides useful in the methods of the invention typically have one or more AGP-like glycosylation regions comprising preferably a multiplicity of two, three or more of any one or more of the following motifs:

| (SEQ ID NO: 495) | |
|---|---|
| (i) | SPAD/E |
| (SEQ ID NO: 496) | |
| (ii) | SPPAPA |
| (SEQ ID NO: 497) | |
| (iii) | PAPAP |

According to another embodiment, in addition to any one or more of the domains, regions and motifs described above, a FLA-like polypeptide useful in the methods of the invention comprises Tyr-His dipeptides, which are usually fanked by [Leu/Val/Ile]-[Leu/Val/Ile] residues. These residues have been shown to play roles in integrin binding in animal cells.

According to another embodiment a preferred a FLA-like polypeptide useful in the methods of the invention comprises two FLA-like domains and any one of more of the following motifs:

| (SEQ ID NO: 495) | |
|---|---|
| (i) | SPAD/E |
| (SEQ ID NO: 496) | |
| (ii) | SPPAPA |
| (SEQ ID NO: 497) | |
| (iii) | PAPAP |

Additionally or alternatively, the homologue of a FLA-like protein useful in the methods of the invention has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 172, provided that the homologous protein comprises any one or more of the conserved domains, regions or motifs as outlined above. The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered. Preferably the motifs in a FLA-like polypeptide have, in increasing order of preference, at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any one or more of the domains and motifs represented by SEQ ID NO: 487 to SEQ ID NO: 497.

In another embodiment a “protein useful in the methods of the invention” is taken to mean a SAUR polypeptide as defined herein. Any reference hereinafter to a “nucleic acid useful in the methods of the invention” is taken to mean a nucleic acid capable of encoding such a SAUR polypeptide. The nucleic acid to be introduced into a plant (and therefore useful in performing the methods of the invention) is any nucleic acid encoding the type of protein which will now be described, hereafter also named “SAUR nucleic acid” or “SAUR gene”.

A “SAUR polypeptide” as defined herein refers to any polypeptide comprising an Auxin inducible domain. Auxin inducible domains are well known in the art as conserved protein domains present in auxin inducible proteins of plant origin. They are referred to as “Auxin inducible” or “Auxin responsive” in databases of conserved domains such as Pfam domains, where the domain is described under accession number PF02519 (Pfam 23.0 (10340 families; R. D. Finn eat al. Nucleic Acids Research (2008) Database Issue 36:D281-D288). The curation and HMM (hidden Markov model) building models for PF02519 as used in Pfam are described below:

Curation PF02519

| | |
|---|---|
| Seed source: | Pfam-B_1263 (release 5.4) |
| Author: | Bashton M, Bateman A |
| Number in seed: | 114 |
| Number in full: | 417 |
| Average length of the domain: | 62.9 aa |
| Average identity of full alignment: | 40% |
| Average coverage of the sequence | 44.16% |
| by the domain: | |

### HMM Information

### PF02519

| HMM build | ls model: hmmbuild -F HMM_Is SEED | | | | |
|---|---|---|---|---|---|
| commands: | hmmcalibrate --cpu 1 --seed 0 HMM_ls | | | | |
| | fs model: hmmbuild -f -F HMM_fs SEED | | | | |
| | hmmcalibrate --cpu 1 --seed 0 HMM_fs | | | | |
| | | ls | | fs | |
| Model details: | Parameter | Sequence | Domain | Sequence | Domain |
| | Gathering | 25.0 | 25.0 | 18.0 | 18.0 |
| | cut-off | | | | |
| | Trusted | 29.6 | 29.6 | 18.5 | 18.5 |
| | cut-off | | | | |
| | Noise cut-off | 23.8 | 23.8 | 15.0 | 15.0 |

| | |
|---|---|
| Model length: | 72 |
| Family (HMM) | 6 |
| version: | |

Proteins comprising Auxin inducible domains are involved in the response of plants and plant cells to the hormone auxin.

A preferred SAUR polypeptide useful for the methods of the invention comprises a conserved domain having in increasing order of preference at least 50%, 51%, 52%, 53%, 54%, 55%, 56%, 57%, 58%, 59%, 60%, 81%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity represented by amino acids 1 to 95 of SEQ ID NO: 502 (Auxin inducible domain in SEQ ID NO: 2) ore to any Auxin inducible domain as present in any one or more of the polypeptides of Table A3 or Table A3(i).

In another preferred embodiment the SAUR polypeptide useful for the methods of the invention comprises a motif having in increasing order of preference at least 1, 2, 3, 4, 5, 6, 7, 8, 8, 10, up to the maximum number of amino acid residues of the motif, amino acid sequence identity any one or more of the following conserved motifs:
(i) Motif 23: LAVYVGEMMQKRRFVVPVTYLSHPCFQKLLRKAEEEFGFDHPMGGL TIPC (SEQ ID NO: 1155) (corresponds to Motif 23 of SEQ ID NO: 502);
(ii) Motif 24: KHxxGVYTAEKxxYxxxIxxxxxxxxxAxxxxSxxxYxxxxPMPIx LxxC (SEQ ID NO: 1156) (corresponds to the conserved sequence of a motif equivalent to Motif 23 as found in SAUR polypeptides);
(iii) Motif 25: LQSSKQLLKSLSHSSNNVAIP (SEQ ID NO: 1157) (corresponds to Motif 2 of SEQ ID NO: 502);
(iv) Motif 26: VxxxKIAxKSQ (SEQ ID NO: 1158) (corresponds to the conserved sequence of a motif equivalent to Motif 25 as found in SAUR polypeptides);
(v) Motif 27: EQIFIDLASRL (SEQ ID NO: 1159) (corresponds to Motif 27 of SEQ ID NO: 502);
(vi) Motif 28: VExxxVxxxxL (SEQ ID NO: 1160) (corresponds to the conserved sequence of a motif equivalent to Motif 27 as found in SAUR polypeptides);
   Wherein X represents any amino acid.

Motifs 23 to 28 are relevant to the auxin response function of SAUR polypeptides.

Motifs 23 to 28 were identified using the MEME algorithm with a sub-set of polypeptide sequences of Table A3 or A3(i). Methods to identify conserved motifs are well known in the art, for example, The MEME algorithm (Bailey and Elkan, Proceedings of the Second International Conference on Intelligent Systems for Molecular Biology, pp. 28-36, AAAI Press, Menlo Park, Calif., 1994.)

More preferably, the SAUR polypeptide comprises in increasing order of preference, at least 2, at least 3, at least 4, at least 5, or all 6 motifs above described.

Further preferred SAUR polypeptides useful in the methods of the invention are encoded by Small Auxin Up RNAs of organisms of the viridiplantae kingdom. Small Auxin Up RNAs as well as methods to identify the same have been previously described and are well known in the art (Jain 2006; Hagen and Guilfoyle 2002).

Additionally or alternatively, SAUR polypeptides useful in the methods of the invention refer to a homologue of a SAUR protein. A preferred homologue of a SAUR protein has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by any one of the polypeptides of Table A3 or A3(i), more preferably by SEQ ID NO: 502, The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered. Preferably the motifs in a SAUR polypeptide have, in increasing order of preference, at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any one or more of the motifs represented by SEQ ID NO: 1155 to SEQ ID NO: 1160 (Motifs 23 to 28).

In a further embodiment of the present invention, there is provided a SAUR polypeptide useful in the methods of the invention having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the SAUR polypeptide represented by SEQ ID NO: X or to any of the SAUR polypeptide sequences listed in Table A3(i). The SAUR polypeptide represented by SEQ ID NO: X and the SAUR polypeptide sequences listed in Table A3(i) are examples of SAUR33-like polypeptides.

Further preferably, SAUR33-like polypeptides comprise the following Motifs I and II and optionally also one or both of Motifs III and IV or any sequence having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to Motifs I to IV.

Motif 47
(SEQ ID NO: 2284)
CEVVLFEHLLWMLENADPQ
Motif 48
(SEQ ID NO: 2285)
PESLDELVEYYAC
Motif 49
(SEQ ID NO: 2286)
GLSKLRCMIRRWHSSSRI
Motif 50
(SEQ ID NO: 2287)
SFHGADEVPKGLHPVYVGKSRRRYLIAEELVGHPLFQNLVDRT

The invention also provides hitherto unknown SAUR-encoding nucleic acids and SAUR polypeptides useful for conferring enhanced yield-related traits in plants relative to control plants.

According to a further embodiment of the present invention, there is therefore provided an isolated nucleic acid molecule selected from:
(i) a nucleic acid represented by any one of the nucleic acids of Table A3 or A3(i);
(ii) the complement of a nucleic acid represented by (i);
(iii) a nucleic acid encoding a SAUR polypeptide having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of any of the polypeptides of Table A3 or A3(i) and additionally or alternatively comprising one or more motifs having in increasing order of preference at least 50%, 55%, 60%, 65%, 70%, 75%, 80%, 85%, 90%, 95%, 96%, 97%, 98%, 99% or more sequence identity to any one or more of the motifs given in SEQ ID NO: 1155 to SEQ ID NO: 1160, and further preferably conferring enhanced yield-related traits relative to control plants.
(iv) a nucleic acid molecule which hybridizes with a nucleic acid molecule of (i) to (iii) under high stringency hybridization conditions and preferably confers enhanced yield-related traits relative to control plants.

According to a further embodiment of the present invention, there is also provided an isolated polypeptide selected from:
(i) an amino acid sequence represented by any one of the polypeptides of Table A3 or A3(i);
(ii) an amino acid sequence having, in increasing order of preference, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of any one of the polypeptides of Table A3 or A3(i) and additionally or alternatively comprising one or more motifs having in increasing order of preference at least 50%, 55%, 60%, 65%, 70%, 75%, 80%, 85%, 90%, 95%, 96%, 97%, 98%, 99% or more sequence identity to any one or more of the motifs given in SEQ ID NO: 1155 to SEQ ID NO: 1160, and further preferably conferring enhanced yield-related traits relative to control plants;
(iii) derivatives of any of the amino acid sequences given in (i) or (ii) above.

In another embodiment a “protein useful in the methods of the invention” is taken to mean any one or more polypeptides selected from the group of a SAUR polypeptide as defined herein, a SYNP polypeptide as defined herein and/or a protein fusion between at least one SAUR and one or more SYNP polypeptides. Any reference hereinafter to a “nucleic acid useful in the methods of the invention” is taken to mean any one or more nucleic acids selected from the group of a capable of encoding such a SAUR, a SYNP or a fusion thereof. The nucleic acid to be introduced into a plant (and therefore useful in performing the methods of the invention) is any nucleic acid encoding the type of protein which will now be described, hereafter also named “SAUR nucleic acid” or “SAUR gene”, or “SYNP nucleic acid” or “SYNP gene” or “SAUR-SYNP fusion nucleic acid” or “SAUR-SYNP fusion gene”.

### Concerning SAUR Polypeptides

A “SAUR polypeptide” as defined herein refers to any polypeptide comprising an Auxin inducible domain. Auxin inducible domains are well known in the art as conserved protein domains present in auxin inducible proteins of plant origin. They are referred to as “.Auxin inducible” or “Auxin responsive” in databases of conserved domains such as Pfam domains, where the domain is described under accession number PF02519 (Pfam 23.0 (10340 families; R. D. Finn eat al. Nucleic Acids Research (2008) Database Issue 36:D281-D288). The curation and HMM (hidden Markov model) building models for PF02519 as used in Pfam are described below:

Curation PF02519

| | |
|---|---|
| Seed source: | Pfam-B_1263 (release 5.4) |
| Author: | Bashton M, Bateman A |
| Number in seed: | 114 |
| Number in full: | 417 |
| Average length of the domain: | 62.9 aa |
| Average identity of full alignment: | 40% |
| Average coverage of the sequence by | 44.16% |
| the domain: | |

HMM information

### PF02519

| HMM build | ls model: hmmbuild -F HMM_ls SEED | | | | |
|---|---|---|---|---|---|
| commands: | hmmcalibrate --cpu 1 --seed 0 HMM_ls | | | | |
| | fs model: hmmbuild -f -F HMM_fs SEED | | | | |
| | hmmcalibrate --cpu 1 --seed 0 HMM_fs | | | | |
| | | ls | | fs | |
| Model details: | Parameter | Sequence | Domain | Sequence | Domain |
| | Gathering | 25.0 | 25.0 | 18.0 | 18.0 |
| | cut-off | | | | |
| | Trusted | 29.6 | 29.6 | 18.5 | 18.5 |
| | cut-off | | | | |
| | Noise cut-off | 23.8 | 23.8 | 15.0 | 15.0 |

| | |
|---|---|
| Model length: | 72 |
| Family (HMM) | 6 |
| version: | |

Proteins comprising Auxin inducible domains are involved in the response of plants and plant cells to the hormone auxin.

A preferred SAUR polypeptide useful for the methods of the invention comprises a conserved domain having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity represented by amino acids 1 to 95 of SEQ ID NO: 1164. (Auxin inducible domain in SEQ ID NO: 1164) or to any Auxin inducible domain as present in any one or more of the polypeptides of Table A3 and A3(i).

In another preferred embodiment the SAUR polypeptide useful for the methods of the invention comprises a motif having in increasing order of preference at least 1, 2, 3, 4, 5, 6, 7, 8, 9, 10, up to the maximum number of amino acid residues of the motif, amino acid sequence identity any one or more of the following conserved motifs:
(i) Motif 29: LAVYVGEMMQKRRFVVPVTYLSHPCFQKLLRKAEEEFGFDHPMGGLTI PC (SEQ ID NO: 1817) (corresponds to Motif 29 of SEQ ID NO: 1164);
(ii) Motif 30: KHxxGVYTAEKxxYxxxIxxxxxxxxxAxxxxS xxxYxxxxPMPIx LxxC (SEQ ID NO: 1818) (corresponds to the conserved sequence of a motif equivalent to Motif 29 as found in SAUR polypeptides);
(iii) Motif 31: LQSSKQLLKSLSHSSNNVAIP (SEQ ID NO: 1819) (corresponds to Motif 30 of SEQ ID NO: 1164);
(iv) Motif 32: VxxxKIAxKSQ (SEQ ID NO: 1820) (corresponds to the conserved sequence of a motif equivalent to Motif 31 as found in SAUR polypeptides);
(v) Motif 33: EQIFIDLASRL (SEQ ID NO: 1821) (corresponds to Motif 33 of SEQ ID NO: 1164);
(vi) Motif 34: VExxxVxxxxL (SEQ ID NO: 1822) (corresponds to the conserved sequence of a motif equivalent to Motif 34 as found in SAUR polypeptides);
   Wherein X represents any amino acid.

Motifs 29 to 34 are relevant to the auxin response function of SAUR polypeptides.

Motifs 29 to 34 were identified using the MEME algorithm with a sub-set of polypeptide sequences of Table A3. Methods to identify conserved motifs are well known in the art, for example, The MEME algorithm (Bailey and Elkan, Proceedings of the Second International Conference on Intelligent Systems for Molecular Biology, pp. 28-36, AAAI Press, Menlo Park, Calif., 1994.)

More preferably, the SAUR polypeptide comprises in increasing order of preference, at least 2, at least 3, at least 4, at least 5, or all 6 motifs above described.

Further preferred SAUR polypeptides useful in the methods of the invention are encoded by Small Auxin Up RNAs of organisms of the viridiplantae kingdom. Small Auxin Up RNAs as well as methods to identify the same have been previously described and are well known in the art (Jain 2006; Hagen and Guilfoyle 2002).

Additionally or alternatively, SAUR polypeptides useful in the methods of the invention refer to a homologue of a SAUR protein. A preferred homologue of a SAUR protein has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by any one of the polypeptides of Table A3 and A3(i), more preferably by SEQ ID NO: 1164, The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered. Preferably the motifs in a SAUR polypeptide have, in increasing order of preference, at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any one or more of the motifs represented by SEQ ID NO: 1817 to SEQ ID NO: 1822 (Motifs 29 to 34).

In a further embodiment of the present invention, there is provided a SAUR polypeptide useful in the methods of the invention having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the SAUR polypeptide represented by SEQ ID NO: X or to any of the SAUR polypeptide sequences listed in Table A3(i). The SAUR polypeptide represented by SEQ ID NO: X and the SAUR polypeptide sequences listed in Table A3(i) are examples of SAUR33-like polypeptides.

Further preferably, SAUR33-like polypeptides comprise the following Motifs I and II and optionally also one or both of Motifs III and IV or any sequence having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to Motifs 47 to 50.

Motif 47
(SEQ ID NO: 2284)
CEVVLFEHLLWMLENADPQ
Motif 48
(SEQ ID NO: 2285)
PESLDELVEYYAC
Motif 49
(SEQ ID NO: 2286)
GLSKLRCMIRRWHSSSRI
Motif 50
(SEQ ID NO: 2287)
SFHGADEVPKGLHPVYVGKSRRRYLIAEELVGHPLFQNLVDRT

### Concerning SYNP Polypeptides

A “SYNP (SAUR yield network protein) polypeptide” as defined herein refers to any polypeptide functioning in the same biological network as a SAUR protein modulating yield traits of a plant.

The “SYNP polypeptide” as defined herein preferably refers to a protein having one or more pfam domain having in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid of a pfam domain selected from the group of pfam domains present in any of the polypeptides of Table E and Table F.

Methods to identify pfam domain in a polypeptide are well known in the art and examples of the same are provided herein.

Further preferably the “SYNP polypeptide” as defined herein refers to a protein having in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% overall sequence identity to the amino acid represented by any one of the polypeptides of Table E and Table F.

In another embodiment a “protein useful in the methods of the invention” is taken to mean a DHAR polypeptide as defined herein. Any reference hereinafter to a “nucleic acid useful in the methods of the invention” is taken to mean a nucleic acid capable of encoding such a DHAR polypeptide. The nucleic acid to be introduced into a plant (and therefore useful in performing the methods of the invention) is any nucleic acid encoding the type of protein which will now be described, hereafter also named “DHAR nucleic acid” or “DHAR gene”.

A “DHAR polypeptide” as defined herein refers to any polypeptide comprising at least a dehydroascorbate reductase domain with an accession number PTHR11260:SF15 (HMMPanther Database). Proteins comprising DHAR polypeptide are involved in regeneration of ascorbic acid from oxidized ascorbate in the ascorbate-glutathione cycle. DHAR polypeptides typically belong to Enzyme Classification Number EC 1.8.5.1.

Preferably, the DHAR domain of an DHAR polypeptide has at least, in increasing order of preference, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the sequence located between amino acid 19 and 210 of SEQ ID NO 1958.

Additionally or alternatively, the DHAR polypeptide useful in the methods of the invention comprises one or more sequence motifs having at least, in increasing order of preference 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one or more of motifs 35 to 37:

The amino acids indicated herein in square brackets represent alternative amino acids for a particular position.

Motif 35:
(SEQ ID NO: 2239)
P[DN]KLGDCPF[SC]QRVLLTLEEK[KH][VL]PY[KD][ML][KH]
L[IV]
Motif 36:
(SEQ ID NO: 2240)
D[DEG]KW[VI][PAS]DSDVI[TV][QG][IL][LI]EEK[YF]PEP
[SP]L[VA]TPPE
Motif 37:
(SEQ ID NO: 2241)
P[FY][IV][NA]GE[KN][IV][ST]A[VA]DLSL[AG]PKLYHLE[V
I]ALGH[FY]K[KN]W[ST][VI]P

Motifs 35 to 37 are typically found in any DHAR polypeptide of any origin.

In another preferred embodiment of the present invention the DHAR polypeptide of the invention may comprise Motifs 38, 39 and 40 in addition to Motif 35, Motif 36 and Motif 37 as defined above, or may comprise a motif having, in increasing order of preference at least 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one or more of Motifs 38 to 40:

Motif 38:
(SEQ ID NO: 2242)
E[VI]CVKA[AS]V[GT]AP[DN][KV]LGDCPF[SC]QRVLLTLEE
Motif 39:
(SEQ ID NO: 2243)
PPE[FK]ASVGSKIF[PS][TS]F[VI][GT]FLKSKD[PA][NS]D
G[TS]EQ
Motif 40:
(SEQ ID NO: 2244)
[IV][ST]A[VA]DLSL[AG]PKLYHL[EQ][VI]ALGH[FY]K[KN]
W[ST][VI]P[ED]SL[TP]HV[HK][NS]Y[MT]K[ALS][LI]FS
[RL][ED]SF[EV]KT

Motifs 38, 39 and 40 correspond to a consensus sequences which represent conserved protein regions in a DHAR polypeptide of chloroplastic (CHL) and cytosolic (CYT) classes origin, to which S. lycopersicum and H. vulgare belong.

Most preferably, the DHAR polypeptide of the invention comprises Motifs 41, 42 and 43 in addition to any one or more of, preferably all of, Motif 35, Motif 36, Motif 37, Motif 38, Motif 39 and Motif 40 as defined above. Motifs 41, 42 and 43 may also, in increasing order of preference comprise motifs having at least 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of Motifs 41 to 43:

Motif 41:
(SEQ ID NO: 2245)
E[IV]CVKAA[VT]GAPD[VIT]LGDCPF[SC]QRVLLTLEE
Motif 42:
(SEQ ID NO: 2246)
PPE[FY]ASVGSKIF[PG][ST]FV[TK]FLKSKD[AP][NS]DG[TS]
E[QK]
Motif 43:
(SEQ ID NO: 2247)
[IV][TS]AVDLSLAPKLYHL[EQ]VAL[GE]HFK[KG]W[TSK][VI]
PE[SN]LTHVH[NA]Y[TM]K[LAS]LFSRESFEKT

Motifs 41, 42 and 43 correspond to a consensus sequences which represent conserved protein regions in a DHAR polypeptide of cytosolic class (CYT) to which S. lycopersicum and O. sativa belong.

In another most preferably embodiment of the present invention, the DHAR polypeptide of the invention comprises Motifs 10, 11 and 12 in addition to Motif 1, Motif 2, Motif 3, Motif 4, Motif 5 and Motif 6 as defined above. Motifs 10, 11 and 12 may also comprise a motif having in increasing order of preference at least 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one or more of Motifs 10 to 12:

Motif 44:
(SEQ ID NO: 2248)
PLE[VI]C[VA]KAS[ILV]T[TV]P[ND][KR]LGDCPF[TC]QRVLL
TLEEKHLPY[DE][ML]KLVDL[SG]NKP[ED]WF
Motif 45:
(SEQ ID NO: 2249)
W[VI][PA]DSDVITQ[AST]LEEK[YF]P[ED]P[PS]L[AV]TPPEK
ASVGSKIFSTF[IV]GFLKSKDP[SN]DG
Motif 46:
(SEQ ID NO: 2250)
QALL[ND]EL[ST][SA]FNDY[LI]KENGPFING[KE][KDE][IV]S
AADLSL[GA]PKLYH[LM]EIALGH[YF]K[NK]W

Motifs 44, 45 and 46 correspond to a consensus sequences which represent conserved protein regions in a DHAR polypeptide of chloroplast class (CHL) to which S. lycopersicum and O. sativa belong.

It is understood that Motif 35, 36, 37, 38, 39, 40, 41, 42, 43, 44, 45 and 46 as referred to herein represent the consensus sequence of the motifs as present in DHAR polypeptides represented in Table A5, especially in SEQ ID NO: 1958. However, it is to be understood that Motifs as defined herein are not limited to their respective sequence but they encompass the corresponding motifs as present in any DHAR polypeptide.

More preferably, the DHAR polypeptide useful in the methods of the invention comprises in increasing order of preference, at least 2, at least 3, at least 4, at least 5, at least 6, at least 7, at least 8, at least 9, at least 10, at least 11 or all 12 motifs.

Alternatively, the homologue of a DHAR protein has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 1958, provided that the DHAR polypeptide comprises any one or more of the 12 conserved motifs as outlined above. The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered. Preferably the motifs in a DHAR polypeptide have, in increasing order of preference, at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the motifs represented by SEQ ID NO: 2239 to SEQ ID NO: 2250 (Motifs 35 to 46).

Preferably, the polypeptide sequence which when used in the construction of a phylogenetic tree, such as the one depicted in FIG. 8, clusters with the group of DHAR polypeptides comprising the amino acid sequence represented by SEQ ID NO: 1958 rather than with any other group.

The terms “domain”, “signature” and “motif” are defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol. 147(1); 195-7).

Furthermore, GDH polypeptides (at least in their native form) typically have glutamate deaminating activity. Tools and techniques for measuring glutamate deaminating activity are well known in the art (Purnell et al., 2005; Skopelitis et al., 2007). Further details are provided in Example 6.

In addition, GDH polypeptides, when expressed in rice according to the methods of the present invention as outlined in Examples 7 and 8, give plants having increased yield related traits, in particular increased seed yield (such as number of (filled) seeds, seed weight and/or harvest index).

Furthermore, FLA-like polypeptides (at least in their native form) typically have glycosylation activity, that is, they are susceptible of glycosylation in a cellular environment. Tools and techniques for measuring glycosylation activity are well known in the art. For example detection of N-glycosylated FLA-like polypeptides expressed in Arabidopsis thaliana cells can be carried out by HPLC fractioning followed by colorimetric assays to detect sugars such as described by Johnson et al., Plant Physiol. (2003) 133 (4) 1911-1925.

In addition, FLA-like polypeptides, when expressed in rice according to the methods of the present invention as outlined in the Example section, give plants having increased yield related traits, in particular increase in any one or more of the following, seed yield, seed fill rate, root biomass and harvest index.

Furthermore, SAUR polypeptides have plant yield increasing activity, that is, when expressed in rice according to the methods of the present invention as outlined in the Examples section give plants having increased yield related traits, preferably selected from increased emergence vigour, increased number of seeds, increased number of filled seeds, increased number of first panicles, increase canopy and/or root biomass, increased emergence vigour and increased weight of seeds.

Furthermore, DHAR polypeptides (at least in their native form) typically have a dehydroascorbate reductase activity. Tools and techniques for measuring dehydroascorbate reductase activity are well known in the art (Kato, 1997—Plant Cell Physiol. 38(2): 173-178).

In addition, DHAR polypeptides, when expressed in rice according to the methods of the present invention as outlined in Examples 7 and 8, give plants having increased yield related traits, in particular increased number of filled seeds, increased number of florets, increased yield relative to control plants.

Concerning GDH polypeptides, the present invention is illustrated by transforming plants with the nucleic acid sequence represented by SEQ ID NO: 1, encoding the polypeptide sequence of SEQ ID NO: 2. However, performance of the invention is not restricted to these sequences; the methods of the invention may advantageously be performed using any GDH-encoding nucleic acid or GDH polypeptide as defined herein.

Examples of nucleic acids encoding GDH polypeptides are given in Table A1 of the Examples section herein. Such nucleic acids are useful in performing the methods of the invention. The amino acid sequences given in Table A1 of the Examples section are example sequences of orthologues and paralogues of the GDH polypeptide represented by SEQ ID NO: 2, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A1 of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 1 or SEQ ID NO: 2, the second BLAST would therefore be against Zea mays sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning FLA-like polypeptides, the present invention is illustrated by transforming plants with the nucleic acid sequence represented by SEQ ID NO: 171, encoding the polypeptide sequence of SEQ ID NO: 172. However, performance of the invention is not restricted to these sequences; the methods of the invention may advantageously be performed using any FLA-like-encoding nucleic acid or FLA-like polypeptide as defined herein.

Examples of nucleic acids encoding FLA-like polypeptides are given in Table A2 of the Examples section herein. Such nucleic acids are useful in performing the methods of the invention. The amino acid sequences given in Table A2 of the Examples section are example sequences of orthologues and paralogues of the FLA-like polypeptide represented by SEQ ID NO: 172, the terms “orthologues” and “paralogues” being as defined herein. Further orthologues and paralogues may readily be identified by performing a so-called reciprocal blast search as described in the definitions section; where the query sequence is SEQ ID NO: 171 or SEQ ID NO: 172, the second BLAST (back-BLAST) would be against Lycopersicum esculentum sequences.

Concerning SAUR polypeptides, the present invention is illustrated by transforming plants with the nucleic acid sequence represented by SEQ ID NO: 501, encoding the polypeptide sequence of SEQ ID NO: 502. However, performance of the invention is not restricted to these sequences; the methods of the invention may advantageously be performed using any SAUR-encoding nucleic acid or SAUR polypeptide as defined herein.

Examples of nucleic acids encoding SAUR polypeptides are given in Table A3 or A3(i) of the Examples section herein. Such nucleic acids are useful in performing the methods of the invention. The amino acid sequences given in Table A3 or A3(i) of the Examples section are example sequences of orthologues and paralogues of the SAUR polypeptide represented by SEQ ID NO: 502, the terms “orthologues” and “paralogues” being as defined herein. Further orthologues and paralogues may readily be identified by performing a so-called reciprocal blast search as described in the definitions section; where the query sequence is SEQ ID NO: 501 or SEQ ID NO: 502, the second BLAST (back-BLAST) would be against Arabidopsis sequences.

Concerning SAUR polypeptides, the present invention may be illustrated by transforming plants with the nucleic acid sequence represented by SEQ ID NO: 1163, encoding the polypeptide sequence of SEQ ID NO: 1164 and a nucleic acid encoding any of the polypeptides of Table E and Table F. However, performance of the invention is not restricted to these sequences; the methods of the invention may advantageously be performed using any SAUR-encoding, any SYNP-encoding nucleic acid or SAUR, SYNP polypeptide as defined herein.

Examples of nucleic acids encoding SAUR polypeptides are given in Table A4 of the Examples section herein. Such nucleic acids are useful in performing the methods of the invention. The amino acid sequences given in Table A4 of the Examples section are example sequences of orthologues and paralogues of the SAUR polypeptide represented by SEQ ID NO: 1164, the terms “orthologues” and “paralogues” being as defined herein. Further orthologues and paralogues may readily be identified by performing a so-called reciprocal blast search as described in the definitions section; where the query sequence is SEQ ID NO: 1163 or SEQ ID NO: 1164, the second BLAST (back-BLAST) would be against Arabidopsis sequences.

Examples of nucleic acids encoding SYNP polypeptides are given in Table E of the Examples section herein. Such nucleic acids are useful in performing the methods of the invention. The amino acid sequences given in Table F of the Examples section are example sequences of orthologues and paralogues of the SYNP polypeptide represented by the polypeptides of Table E, the terms “orthologues” and “paralogues” being as defined herein. Further orthologues and paralogues may readily be identified by performing a so-called reciprocal blast search as described in the definitions section.

Concerning DHAR polypeptides, the present invention is illustrated by transforming plants with the nucleic acid sequence represented by SEQ ID NO: 1957, encoding the polypeptide sequence of SEQ ID NO: 1958. However, performance of the invention is not restricted to these sequences; the methods of the invention may advantageously be performed using any DHAR-encoding nucleic acid or DHAR polypeptide as defined herein.

Examples of nucleic acids encoding DHAR polypeptides are given in Table A5 of the Examples section herein. Such nucleic acids are useful in performing the methods of the invention. The amino acid sequences given in Table A5 of the Examples section are example sequences of orthologues and paralogues of the DHAR polypeptide represented by SEQ ID NO: 1958, the terms “orthologues” and “paralogues” being as defined herein. Further orthologues and paralogues may readily be identified by performing a so-called reciprocal blast search as described in the definitions section; where the query sequence is SEQ ID NO: 1957 or SEQ ID NO: 1958, the second BLAST (back-BLAST) would be against tomato sequences.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants may also be useful in practising the methods of the invention. Examples of such variants include nucleic acids encoding homologues and derivatives of any one of the amino acid sequences given in Table A1 to A5, and Table E, and Table F of the Examples section, the terms “homologue” and “derivative” being as defined herein. Also useful in the methods of the invention are nucleic acids encoding homologues and derivatives of orthologues or paralogues of any one of the amino acid sequences given in Table A1 to A5, and Table E, and Table F of the Examples section. Homologues and derivatives useful in the methods of the present invention have substantially the same biological and functional activity as the unmodified protein from which they are derived. Further variants useful in practising the methods of the invention are variants in which codon usage is optimised or in which miRNA target sites are removed.

Further nucleic acid variants useful in practising the methods of the invention include portions of nucleic acids encoding GDH polypeptides, or FLA polypeptides, or SAUR polypeptides, or DHAR polypeptides, nucleic acids hybridising to nucleic acids encoding GDH polypeptides, or FLA polypeptides, or SAUR polypeptides, or DHAR polypeptides, splice variants of nucleic acids encoding GDH polypeptides, allelic variants of nucleic acids encoding GDH polypeptides, or FLA polypeptides, or SAUR polypeptides, or DHAR polypeptides, and variants of nucleic acids encoding GDH polypeptides, or FLA polypeptides, or SAUR polypeptides, or DHAR polypeptides, obtained by gene shuffling. The terms hybridising sequence, splice variant, allelic variant and gene shuffling are as described herein.

Further nucleic acid variants useful in practising the methods of the invention include portions of nucleic acids encoding SAUR polypeptides, SYNP polypeptides of protein fusions thereof. Also useful are nucleic acids hybridising to nucleic acids encoding SAUR polypeptides, SYNP polypeptides of protein fusions thereof; splice variants of nucleic acids encoding SAUR polypeptides, SYNP polypeptides of protein fusions thereof; allelic variants of nucleic acids encoding SAUR polypeptides, SYNP polypeptides of protein fusions thereof and variants of nucleic acids encoding SAUR polypeptides, SYNP polypeptides of protein fusions thereof obtained by gene shuffling. The terms hybridising sequence, splice variant, allelic variant and gene shuffling are as described herein.

Nucleic acids encoding GDH polypeptides, or FLA polypeptides, or SAUR polypeptides, or SYNP polypeptides, or fusion proteins of SAUR polypeptides and SYNP polypeptides, or DHAR polypeptides, need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table A1 to A5, and Table E, and Table F of the Examples section, or a portion of a nucleic acid encoding an orthologue, paralogue or homologue of any of the amino acid sequences given in Table A1 to A5, and Table E, and Table F of the Examples section.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Concerning GDH polypeptides, portions useful in the methods of the invention, encode a GDH polypeptide as defined herein, and have substantially the same biological activity as the amino acid sequences given in Table A1 of the Examples section. Preferably, the portion is a portion of any one of the nucleic acids given in Table A1 of the Examples section, or is a portion of a nucleic acid encoding an orthologue or paralogue of any one of the amino acid sequences given in Table A1 of the Examples section. Preferably the portion is at least 500, 550, 600, 650, 700, 750, 800, 850, 900, 950, 1000, 1050, 1100, 1150, 1200, 1250, 1300, 1350, 1400 consecutive nucleotides in length, the consecutive nucleotides being of any one of the nucleic acid sequences given in Table A1 of the Examples section, or of a nucleic acid encoding an orthologue or paralogue of any one of the amino acid sequences given in Table A1 of the Examples section. Most preferably the portion is a portion of the nucleic acid of SEQ ID NO: 1. Preferably, the portion encodes a fragment of an amino acid sequence which comprises a Glu/Phe/Leu/Val dehydrogenase, C-terminal domain (Pfam entry PF00208) and a Glu/Phe/Leu/Val dehydrogenase, dimerisation region (Pfam entry PF02812), which has glutamate deaminating activity and which, when used in the construction of a phylogenetic tree, such as the one depicted in FIG. 3, clusters with GDH polypeptides, preferably with class I GDH polypeptides or with moss GDH polypeptides related to class I, more preferably to group of beta subunit GDH polypeptides comprising the amino acid sequence represented by SEQ ID NO: 2 rather than with any other group.

Concerning FLA-like polypeptides, portions useful in the methods of the invention, encode a FLA-like polypeptide as defined herein, and have substantially the same biological activity as the amino acid sequences given in Table A2 of the Examples section. Preferably, the portion is a portion of any one of the nucleic acids given in Table A2 of the Examples section, or is a portion of a nucleic acid encoding an orthologue or paralogue of any one of the amino acid sequences given in Table A2 of the Examples section. Preferably the portion is at least 100, 200, 300, 400, 500, 550, 600, 650, 700, 750, 800, 850, 900, 950, 1000 consecutive nucleotides in length, the consecutive nucleotides being of any one of the nucleic acid sequences given in Table A2 of the Examples section, or of a nucleic acid encoding an orthologue or paralogue of any one of the amino acid sequences given in Table A2 of the Examples section. Most preferably the portion is a portion of the nucleic acid of SEQ ID NO: 171. Preferably, the portion encodes a fragment of an amino acid sequence which comprises at least one fasciclin domain.

Concerning SAUR polypeptides, portions useful in the methods of the invention, encode a SAUR polypeptide as defined herein, and have substantially the same biological activity as the amino acid sequences given in Table A3 or A3(i) of the Examples section. Preferably, the portion is a portion of any one of the nucleic acids given in Table A3 or A3(i) of the Examples section, or is a portion of a nucleic acid encoding an orthologue or paralogue of any one of the amino acid sequences given in Table A3 or A3(i) of the Examples section. Preferably the portion is at least 50, 100, 200, 300, 400, 500, 550, 600, 650, 700, 750, 800, 850, 900, 950, 1000 consecutive nucleotides in length, the consecutive nucleotides being of any one of the nucleic acid sequences given in Table A3 or A3(i) of the Examples section, or of a nucleic acid encoding an orthologue or paralogue of any one of the amino acid sequences given in Table A3 or A3(i) of the Examples section. Most preferably the portion is a portion of the nucleic acid of SEQ ID NO: 501. Preferably, the portion encodes a fragment of an amino acid sequence comprising an Auxin inducible domain.

Concerning SAUR polypeptides, portions useful in the methods of the invention, encode a SAUR polypeptide, a SYNP polypeptide or protein fusions thereof as defined herein, and have substantially the same biological activity as the amino acid sequences given in Table A4, Table E and Table F of the Examples section. Preferably, the portion is a portion of any one of the nucleic acids given in Table A, Table E or Table F of the Examples section, or is a portion of a nucleic acid encoding an orthologue or paralogue of any one of the amino acid sequences given in Table A, Table E or Table F of the Examples section. Preferably the portion is at least 50, 100, 200, 300, 400, 500, 550, 600, 650, 700, 750, 800, 850, 900, 950, 1000 consecutive nucleotides in length, the consecutive nucleotides being of any one of the nucleic acid sequences given in Table A, Table E or Table F of the Examples section, or of a nucleic acid encoding an orthologue or paralogue of any one of the amino acid sequences given in Table A, Table E or Table F of the Examples section.

Concerning DHAR polypeptides, portions useful in the methods of the invention, encode a DHAR polypeptide as defined herein, and have substantially the same biological activity as the amino acid sequences given in Table A5 of the Examples section. Preferably, the portion is a portion of any one of the nucleic acids given in Table A5 of the Examples section, or is a portion of a nucleic acid encoding an orthologue or paralogue of any one of the amino acid sequences given in Table A5 of the Examples section. Preferably the portion is at least 500, 550, 600, 650, 700, 750, 800, 850, 900, 950, 1000 consecutive nucleotides in length, the consecutive nucleotides being of any one of the nucleic acid sequences given in Table A5 of the Examples section, or of a nucleic acid encoding an orthologue or paralogue of any one of the amino acid sequences given in Table A5 of the Examples section. Most preferably the portion is a portion of the nucleic acid of SEQ ID NO: 1957. Preferably, the portion encodes a fragment of an amino acid sequence which, when used in the construction of a phylogenetic tree, such as the one depicted in FIG. 8, clusters with the group of DHAR polypeptides comprising the amino acid sequence represented by SEQ ID NO: 1958 rather than with any other group. Clustering sequences preferably comprise any one or more of motifs 35 to 46; and/or comprises dehydroascorbate reductase biological activity; and/or have at least 49% sequence identity to SEQ ID NO: 1958 or to any of the polypeptides depicted in Table A5.

Another nucleic acid variant useful in the methods of the invention is a nucleic acid capable of hybridising, under reduced stringency conditions, preferably under stringent conditions, with a nucleic acid encoding a GDH polypeptide, or a FLA polypeptide, or a SAUR polypeptide, or an SYNP polypeptide, or a fusion protein of SAUR polypeptides and SYNP polypeptide, or a DHAR polypeptide, as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table A1 to A5, and Table E, and Table F of the Examples section, or comprising introducing and expressing in a plant a nucleic acid capable of hybridising to a nucleic acid encoding an orthologue, paralogue or homologue of any of the nucleic acid sequences given in Table A1 to A5, and Table E, and Table F of the Examples section.

Concerning GDH polypeptides, hybridising sequences useful in the methods of the invention encode a GDH polypeptide as defined herein, having substantially the same biological activity as the amino acid sequences given in Table A1 of the Examples section.

Preferably, the hybridising sequence is capable of hybridising to the complement of any one of the nucleic acids given in Table A1 of the Examples section, or to a portion of any of these sequences, a portion being as defined above, or the hybridising sequence is capable of hybridising to the complement of a nucleic acid encoding an orthologue or paralogue of any one of the amino acid sequences given in Table A1 of the Examples section. Most preferably, the hybridising sequence is capable of hybridising to the complement of a nucleic acid as represented by SEQ ID NO: 1 or to a portion thereof.

Preferably, the hybridising sequence encodes a polypeptide with an amino acid sequence which comprises a Glu/Phe/Leu/Val dehydrogenase, C-terminal domain (Pfam entry PF00208) and a Glu/Phe/Leu/Val dehydrogenase, dimerisation region (Pfam entry PF02812), which has glutamate deaminating activity and which, when full-length and used in the construction of a phylogenetic tree, such as the one depicted in FIG. 3, clusters with GDH polypeptides, preferably with class I GDH polypeptides or with moss GDH polypeptides related to class I, more preferably to group of beta subunit GDH polypeptides comprising the amino acid sequence represented by SEQ ID NO: 2 rather than with any other group.

Concerning FLA-like polypeptides, hybridising sequences useful in the methods of the invention encode a FLA-like polypeptide as defined herein, having substantially the same biological activity as the amino acid sequences given in Table A2 of the Examples section. Preferably, the hybridising sequence is capable of hybridising to the complement of any one of the nucleic acids given in Table A2 of the Examples section, or to a portion of any of these sequences, a portion being as defined above, or the hybridising sequence is capable of hybridising to the complement of a nucleic acid encoding an orthologue or paralogue of any one of the amino acid sequences given in Table A2 of the Examples section. Most preferably, the hybridising sequence is capable of hybridising to the complement of a nucleic acid as represented by SEQ ID NO: 171 or to a portion thereof.

Preferably, the hybridising sequence encodes a polypeptide comprising at least one fasciclin domain.

Concerning SAUR polypeptides, hybridising sequences useful in the methods of the invention encode a SAUR polypeptide as defined herein, having substantially the same biological activity as the amino acid sequences given in Table A3 or A3(i) of the Examples section. Preferably, the hybridising sequence is capable of hybridising to the complement of any one of the nucleic acids given in Table A3 or A3(i) of the Examples section, or to a portion of any of these sequences, a portion being as defined above, or the hybridising sequence is capable of hybridising to the complement of a nucleic acid encoding an orthologue or paralogue of any one of the amino acid sequences given in Table A3 or A3(i) of the Examples section. Most preferably, the hybridising sequence is capable of hybridising to the complement of a nucleic acid as represented by SEQ ID NO: 501 or to a portion thereof.

Preferably, the hybridising sequence encodes a polypeptide with an amino acid sequence comprising an Auxin inducible domain.

Concerning SAUR polypeptides, or SYNP polypeptides, or fusion proteins of SAUR polypeptides and SYNP polypeptides, hybridising sequences useful in the methods of the invention encode a SAUR polypeptide as defined herein, having substantially the same biological activity as the amino acid sequences given in Table A4, E, F of the Examples section. Preferably, the hybridising sequence is capable of hybridising to the complement of any one of the nucleic acids given in Table A4, E, F of the Examples section, or to a portion of any of these sequences, a portion being as defined above, or the hybridising sequence is capable of hybridising to the complement of a nucleic acid encoding an orthologue or paralogue of any one of the amino acid sequences given in Table A4, E, F of the Examples section.

Concerning DHAR polypeptides, hybridising sequences useful in the methods of the invention encode a DHAR polypeptide as defined herein, having substantially the same biological activity as the amino acid sequences given in Table A5 of the Examples section. Preferably, the hybridising sequence is capable of hybridising to the complement of any one of the nucleic acids given in Table A5 of the Examples section, or to a portion of any of these sequences, a portion being as defined above, or the hybridising sequence is capable of hybridising to the complement of a nucleic acid encoding an orthologue or paralogue of any one of the amino acid sequences given in Table A5 of the Examples section. Most preferably, the hybridising sequence is capable of hybridising to the complement of a nucleic acid as represented by SEQ ID NO: 1957 or to a portion thereof.

Preferably, the hybridising sequence encodes a polypeptide with an amino acid sequence which, when full-length and used in the construction of a phylogenetic tree, such as the one depicted in FIG. 8, clusters with the group of DHAR polypeptides (EC 1.8.5.1.) comprising the amino acid sequence represented by SEQ ID NO: 1958 rather than with any other group. Hybridising sequences preferably encode a polypeptide comprising any one or more of motifs 35 to 46 and/or have dehydroascorbate reductase biological activity and/or at least 49% sequence identity to SEQ ID NO: 1958 or to any of the polypeptides depicted in Table A5.

Another nucleic acid variant useful in the methods of the invention is a splice variant encoding a GDH polypeptide, or a FLA polypeptide, or a SAUR polypeptide, or a DHAR polypeptide, as defined hereinabove, a splice variant being as defined herein. Another nucleic acid variant useful in the methods of the invention is a splice variant encoding a SAUR polypeptide, a SYNP polypeptide as defined hereinabove or a fusion of both polypeptides, a splice variant being as defined herein.

According to the present invention, there is provided a method for enhancing yield-related traits in plants, comprising introducing and expressing in a plant a splice variant of any one of the nucleic acid sequences given in Table A1 to A5, and Table E, and Table F of the Examples section, or a splice variant of a nucleic acid encoding an orthologue, paralogue or homologue of any of the amino acid sequences given in Table A1 to A5, and Table E, and Table F of the Examples section.

Concerning GDH polypeptides, preferred splice variants are splice variants of a nucleic acid represented by SEQ ID NO: 1, or a splice variant of a nucleic acid encoding an orthologue or paralogue of SEQ ID NO: 2. Preferably, the amino acid sequence encoded by the splice variant comprises a Glu/Phe/Leu/Val dehydrogenase, C-terminal domain (Pfam entry PF00208) and a Glu/Phe/Leu/Val dehydrogenase, dimerisation region (Pfam entry PF02812), has glutamate deaminating activity and, when used in the construction of a phylogenetic tree, such as the one depicted in FIG. 3, clusters with GDH polypeptides, preferably with class I GDH polypeptides or with moss GDH polypeptides related to class I, more preferably to group of beta subunit GDH polypeptides comprising the amino acid sequence represented by SEQ ID NO: 2 rather than with any other group.

Concerning FLA-like polypeptides, preferred splice variants are splice variants of a nucleic acid represented by SEQ ID NO: 171, or a splice variant of a nucleic acid encoding an orthologue or paralogue of SEQ ID NO: 172. Preferably, the amino acid sequence encoded by the splice variant comprises at least one fasciclin domain.

Concerning SAUR polypeptides, preferred splice variants are splice variants of a nucleic acid represented by SEQ ID NO: 501, or a splice variant of a nucleic acid encoding an orthologue or paralogue of SEQ ID NO: 502. Preferably, the amino acid sequence encoded by the splice variant comprises an Auxin inducible domain.

Concerning DHAR polypeptides, preferred splice variants are splice variants of a nucleic acid represented by SEQ ID NO: 1957, or a splice variant of a nucleic acid encoding an orthologue or paralogue of SEQ ID NO: 1958. Preferably, the amino acid sequence encoded by the splice variant, when used in the construction of a phylogenetic tree, such as the one depicted in FIG. 8, clusters with the group of DHAR polypeptides comprising the amino acid sequence represented by SEQ ID NO: 1958 rather than with any other group. The splice variants preferably comprise any one or more of motifs 35 to 46 and/or have dehydroascorbate reductase biological activity and/or encode a polypeptide having at least 49% sequence identity to SEQ ID NO: 1958 or to any of the DHAR polypeptides depicted in Table A5.

Another nucleic acid variant useful in performing the methods of the invention is an allelic variant of a nucleic acid encoding a GDH polypeptide, or a FLA polypeptide, or a SAUR polypeptide, or a DHAR polypeptide, as defined hereinabove, an allelic variant being as defined herein.

Concerning SAUR polypeptides, or SYNP polypeptides, or fusion proteins of SAUR polypeptides and SYNP polypeptides, another nucleic acid variant useful in performing the methods of the invention is an allelic variant of a nucleic acid encoding a SAUR polypeptide, a SYNP polypeptide as defined hereinabove or a fusion of both polypeptides, an allelic variant being as defined herein.

According to the present invention, there is provided a method for enhancing yield-related traits in plants, comprising introducing and expressing in a plant an allelic variant of any one of the nucleic acids given in Table A1 to A5, and Table E, and Table F of the Examples section, or comprising introducing and expressing in a plant an allelic variant of a nucleic acid encoding an orthologue, paralogue or homologue of any of the amino acid sequences given in Table A1 to A5, and Table E, and Table F of the Examples section.

Concerning GDH polypeptides, the polypeptides encoded by allelic variants useful in the methods of the present invention have substantially the same biological activity as the GDH polypeptide of SEQ ID NO: 2 and any of the amino acids depicted in Table A1 of the Examples section. Allelic variants exist in nature, and encompassed within the methods of the present invention is the use of these natural alleles. Preferably, the allelic variant is an allelic variant of SEQ ID NO: 1 or an allelic variant of a nucleic acid encoding an orthologue or paralogue of SEQ ID NO: 2. Preferably, the amino acid sequence encoded by the allelic variant comprises a Glu/Phe/Leu/Val dehydrogenase, C-terminal domain (Pfam entry PF00208) and a Glu/Phe/Leu/Val dehydrogenase, dimerisation region (Pfam entry PF02812), has glutamate deaminating activity and, when used in the construction of a phylogenetic tree, such as the one depicted in FIG. 3, clusters with GDH polypeptides, preferably with class I GDH polypeptides or with moss GDH polypeptides related to class I, more preferably to group of beta subunit GDH polypeptides comprising the amino acid sequence represented by SEQ ID NO: 2 rather than with any other group.

Concerning FLA-like polypeptides, the polypeptides encoded by allelic variants useful in the methods of the present invention have substantially the same biological activity as the FLA-like polypeptide of SEQ ID NO: 172 and any of the amino acids depicted in Table A2 of the Examples section. Allelic variants exist in nature, and encompassed within the methods of the present invention is the use of these natural alleles. Preferably, the allelic variant is an allelic variant of SEQ ID NO: 171 or an allelic variant of a nucleic acid encoding an orthologue or paralogue of SEQ ID NO: 172. Preferably, the amino acid sequence encoded by the allelic variant comprises at least one fasciclin domain.

Concerning SAUR polypeptides, the polypeptides encoded by allelic variants useful in the methods of the present invention have substantially the same biological activity as the SAUR polypeptide of SEQ ID NO: 502 and any of the amino acids depicted in Table A3 or A3(i) of the Examples section. Allelic variants exist in nature, and encompassed within the methods of the present invention is the use of these natural alleles. Preferably, the allelic variant is an allelic variant of SEQ ID NO: 501 or an allelic variant of a nucleic acid encoding an orthologue or paralogue of SEQ ID NO: 502. Preferably, the amino acid sequence encoded by the allelic variant comprises an Auxin inducible domain.

Concerning SAUR polypeptides, or SYNP polypeptides, or fusion proteins of SAUR polypeptides and SYNP polypeptides, the polypeptides encoded by allelic variants useful in the methods of the present invention have substantially the same biological activity as the SAUR polypeptide and the SYNP or a protein fusion of the same and any of the amino acids depicted in Table A4, and Table E, and Table F of the Examples section. Allelic variants exist in nature, and encompassed within the methods of the present invention is the use of these natural alleles.

Concerning DHAR polypeptides, the polypeptides encoded by allelic variants useful in the methods of the present invention have substantially the same biological activity as the DHAR polypeptide of SEQ ID NO: 1958 and any of the amino acids depicted in Table A5 of the Examples section. Allelic variants exist in nature, and encompassed within the methods of the present invention is the use of these natural alleles. Preferably, the allelic variant is an allelic variant of SEQ ID NO: 1957 or an allelic variant of a nucleic acid encoding an orthologue or paralogue of SEQ ID NO: 1958. Preferably, the amino acid sequence encoded by the allelic variant, when used in the construction of a phylogenetic tree, such as the one depicted in FIG. 8, clusters with the group of DHAR polypeptides comprising the amino acid sequence represented by SEQ ID NO: 1958 rather than with any other group. Clustering sequences preferably comprise any one or more of motifs 35 to 46 and/or dehydroascorbate reductase biological activity and/or at least 49% sequence identity to SEQ ID NO: 1958 or to any of the polypeptides depicted in Table A5.

Gene shuffling or directed evolution may also be used to generate variants of nucleic acids encoding GDH polypeptides, or FLA polypeptides, or SAUR polypeptides, or SYNP polypeptides, or fusion proteins of SAUR polypeptides and SYNP polypeptides, or DHAR polypeptides, as defined above; the term “gene shuffling” being as defined herein.

According to the present invention, there is provided a method for enhancing yield-related traits in plants, comprising introducing and expressing in a plant a variant of any one of the nucleic acid sequences given in Table A1 to A5, and Table E, and Table F of the Examples section, or comprising introducing and expressing in a plant a variant of a nucleic acid encoding an orthologue, paralogue or homologue of any of the amino acid sequences given in Table A1 to A5, and Table E, and Table F of the Examples section, which variant nucleic acid is obtained by gene shuffling.

Concerning GDH polypeptides, preferably, the amino acid sequence encoded by the variant nucleic acid obtained by gene shuffling comprises a Glu/Phe/Leu/Val dehydrogenase, C-terminal domain (Pfam entry PF00208) and a Glu/Phe/Leu/Val dehydrogenase, dimerisation region (Pfam entry PF02812), has glutamate deaminating activity and, when used in the construction of a phylogenetic tree such as the one depicted in FIG. 3, clusters with GDH polypeptides, preferably with class I GDH polypeptides or with moss GDH polypeptides related to class I, more preferably to group of beta subunit GDH polypeptides comprising the amino acid sequence represented by SEQ ID NO: 2 rather than with any other group.

Concerning FLA-like polypeptides, preferably, the amino acid sequence encoded by the variant nucleic acid obtained by gene shuffling comprises at least one fasciclin domain.

Concerning SAUR polypeptides, preferably, the amino acid sequence encoded by the variant nucleic acid obtained by gene shuffling, comprises an Auxin inducible domain.

Concerning DHAR polypeptides, preferably, the amino acid sequence encoded by the variant nucleic acid obtained by gene shuffling, when used in the construction of a phylogenetic tree such as the one depicted FIG. 8, clusters with the group of DHAR polypeptides comprising the amino acid sequence represented by SEQ ID NO: 1958 rather than with any other group and/or comprises any one or more of motifs 35 to 46 and/or has dehydroascorbate reductase biological activity and/or has at least 49% sequence identity to SEQ ID NO: 1958 or to any one or more of the polypeptides depicted in Table A5.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding GDH polypeptides may be derived from any natural or artificial source. The nucleic acid may be modified from its native form in composition and/or genomic environment through deliberate human manipulation. Preferably the GDH polypeptide-encoding nucleic acid is from a plant, further preferably from a monocotyledonous plant, more preferably from the family Poaceae, most preferably the nucleic acid is from Zea mays.

Nucleic acids encoding FLA-like polypeptides may be derived from any natural or artificial source. The nucleic acid may be modified from its native form in composition and/or genomic environment through deliberate human manipulation. Preferably the FLA-like polypeptide-encoding nucleic acid is from a plant, further preferably from a dicotyledonous plant, more preferably from the family brassicaceae or from the populus genus, most preferably the nucleic acid is from Lycopersicum esculentum or from Populus trichocarpa.

Nucleic acids encoding SAUR polypeptides may be derived from any natural or artificial source. The nucleic acid may be modified from its native form in composition and/or genomic environment through deliberate human manipulation. Preferably the SAUR polypeptide-encoding nucleic acid is from a plant, further preferably from a dicotyledonous plant, more preferably from the family brasicaceae, most preferably the nucleic acid is from Arabidopsis thaliana.

Nucleic acids encoding the polypeptides useful in the methods of the invention may be derived from any natural or artificial source. The nucleic acid may be modified from its native form in composition and/or genomic environment through deliberate human manipulation. Preferably the SAUR, SYNP or fusion thereof polypeptide-encoding nucleic acid is from a plant, further preferably from a dicotyledonous plant, more preferably from the family brasicaceae, most preferably the nucleic acid is from Arabidopsis thaliana.

Nucleic acids encoding DHAR polypeptides may be derived from any natural or artificial source. The nucleic acid may be modified from its native form in composition and/or genomic environment through deliberate human manipulation. Preferably the DHAR polypeptide-encoding nucleic acid is from a plant, further preferably from a dicotyledonous plant, more preferably from the family Solanaceae, most preferably the nucleic acid is from Solanum lycopersicum.

Concerning GDH polypeptides, performance of the methods of the invention gives plants having enhanced yield-related traits. In particular performance of the methods of the invention gives plants having increased yield, especially increased seed yield and/or enhanced root growth and/or increased early vigour, relative to control plants under non-stress conditions or under stress conditions, provided that the stress conditions do not encompass nitrogen deficiency. The terms “yield”, “seed yield” and “early vigour” are described in more detail in the “definitions” section herein.

Concerning FLA-like polypeptides, performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Concerning SAUR polypeptides, performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Concerning SAUR polypeptides, or SYNP polypeptides, or fusion proteins of SAUR polypeptides and SYNP polypeptides, performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Concerning DHAR polypeptides, performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds and/or roots, and performance of the methods of the invention results in plants having increased seed yield relative to the seed yield of control plants and/or enhanced root growth, compared to control plants.

Reference herein to enhanced yield-related traits is taken to mean an increase early vigour and/or in biomass (weight) of one or more parts of a plant, which may include aboveground (harvestable) parts and/or (harvestable) parts below ground. In particular, such harvestable parts are seeds, and performance of the methods of the invention results in plants having increased seed yield relative to the seed yield of control plants.

Taking corn as an example, a yield increase may be manifested as one or more of the following: increase in the number of plants established per square meter, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others.

Taking rice as an example, a yield increase may manifest itself as an increase in one or more of the following: number of plants per square meter, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

Concerning GDH polypeptides, the present invention provides a method for increasing yield, especially seed yield and/or root yield of plants, relative to control plants, which method comprises modulating expression in a plant of a nucleic acid encoding a GDH polypeptide as defined herein.

Concerning FLA-like polypeptides, the present invention provides a method for increasing yield-related traits, especially seed yield of plants, relative to control plants, which method comprises modulating expression in a plant of a nucleic acid encoding a FLA-like polypeptide as defined herein.

According to a preferred feature of the present invention, performance of the methods of the invention gives plants having an increased growth rate relative to control plants. Therefore, according to the present invention, there is provided a method for increasing the growth rate of plants, which method comprises modulating expression in a plant of a nucleic acid encoding a FLA-like polypeptide as defined herein.

Concerning SAUR polypeptides, the present invention provides a method for increasing yield-related traits, especially seed yield of plants, relative to control plants, which method comprises modulating expression in a plant of a nucleic acid encoding a SAUR polypeptide as defined herein.

According to a preferred feature of the present invention, performance of the methods of the invention gives plants having an increased growth rate relative to control plants. Therefore, according to the present invention, there is provided a method for increasing the growth rate of plants, which method comprises modulating expression in a plant of a nucleic acid encoding a SAUR polypeptide as defined herein.

Concerning SAUR polypeptides, or SYNP polypeptides, or fusion proteins of SAUR polypeptides and SYNP polypeptides, the present invention provides a method for increasing yield-related traits, especially seed yield of plants, relative to control plants, which method comprises modulating expression in a plant of a nucleic acid encoding a SAUR and a SYNP polypeptide or a fusion thereof as defined herein.

According to a preferred feature of the present invention, performance of the methods of the invention gives plants having an increased growth rate relative to control plants. Therefore, according to the present invention, there is provided a method for increasing the growth rate of plants, which method comprises modulating expression in a plant of a nucleic acid encoding a polypeptide useful in the method of the as defined herein.

Concerning DHAR polypeptides, the present invention provides a method for increasing yield, especially seed yield of plants, relative to control plants, which method comprises modulating expression in a plant of a nucleic acid encoding a DHAR polypeptide as defined herein.

According to a preferred feature of the present invention, performance of the methods of the invention gives plants having an increased growth rate relative to control plants. Therefore, according to the present invention, there is provided a method for increasing the growth rate of plants, which method comprises modulating expression in a plant of a nucleic acid encoding a DHAR polypeptide as defined herein.

Since the transgenic plants according to the present invention have increased yield and/or yield-related traits, it is likely that these plants exhibit an increased growth rate (during at least part of their life cycle), relative to the growth rate of control plants at a corresponding stage in their life cycle.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises modulating expression in a plant of a nucleic acid encoding a GDH polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises modulating expression in a plant of a nucleic acid encoding a GDH polypeptide, a FLA polypeptide, or a SAUR polypeptide or an SYNP polypeptide, or a fusion protein of SAUR polypeptides and SYNP polypeptide, or a DHAR polypeptide. Nutrient deficiency may result from a lack of nutrients such as phosphates and other phosphorous-containing compounds, potassium, calcium, magnesium, manganese, iron and boron, amongst others.

Performance of the methods of the invention gives plants grown under conditions of salt stress, increased yield relative to control plants grown under comparable conditions. Therefore, according to the present invention, there is provided a method for increasing yield in plants grown under conditions of salt stress, which method comprises modulating expression in a plant of a nucleic acid encoding a GDH polypeptide, or a FLA polypeptide, or a SAUR polypeptide, or an SYNP polypeptide, or a fusion protein of SAUR polypeptides and SYNP polypeptide, or a DHAR polypeptide. The term salt stress is not restricted to common salt (NaCl), but may be any one or more of: NaCl, KCl, LiCl, MgCl₂, CaCl₂, amongst others.

The present invention encompasses plants or parts thereof (including seeds) obtainable by the methods according to the present invention. The plants or parts thereof comprise a nucleic acid transgene encoding a GDH polypeptide as defined above.

The invention also provides genetic constructs and vectors to facilitate introduction and/or expression in plants of nucleic acids encoding GDH polypeptides, or FLA polypeptides, or SAUR polypeptides, or DHAR polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding a GDH polypeptide as defined above;
(b) one or more control sequences capable of driving expression of the nucleic acid sequence of (a); and optionally
(c) a transcription termination sequence.

Preferably, the nucleic acid encoding a GDH polypeptide, or a FLA polypeptide, or a SAUR polypeptide, or a DHAR polypeptide, is as defined above. The term “control sequence” and “termination sequence” are as defined herein.

More specifically, the present invention provides a construct comprising:
(i) a first nucleic acid encoding at least a SAUR polypeptide and a second nucleic acid encoding one or more SYNP polypeptides, wherein the first and second nucleic acids are comprised in a single nucleic acid molecule or in multiple, at least two, nucleic acid molecules;
(ii) one or more control sequences capable of driving expression of the nucleic acid sequence of (i), preferably a plant promoter, more preferably a constitutive promoter, even more preferably a GOS2 promoter, most preferably a GOS2 promoter from rice; and optionally
(iii) a transcription termination sequence.

Preferably, the nucleic acid encoding a polypeptide useful in the methods of the invention is as defined above. The term “control sequence” and “termination sequence” are as defined herein.

The present invention also provides for a mixture of constructs useful for example, for simultaneous introduction and expression in plants of two or three nucleic acid sequence encoding a SAUR and a SYNP polypeptide as defined herein; wherein at least one construct comprises:
(a) a nucleic acid sequence encoding a SAUR polypeptide;
(b) one or more control sequences capable of driving expression of the nucleic acid sequence of (a); and optionally
(c) a transcription termination sequence,
   and wherein at least one other construct comprises:
(d) a nucleic acid sequence nucleic acid sequence encoding a SYNP polypeptide as defined herein;
(e) one or more control sequences capable of driving expression of the nucleic acid sequence of (d); and optionally
(f) a transcription termination sequence.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence, but preferably the promoter is of plant origin. A constitutive promoter is particularly useful in the methods. Preferably the constitutive promoter is a ubiquitous constitutive promoter of medium strength. See the “Definitions” section herein for definitions of the various promoter types.

Concerning GDH polypeptides, also useful in the methods of the invention is a root-specific promoter.

Concerning SAUR polypeptides, also useful in the methods of the invention is a leaf-specific promoter. Further the promoter useful in the methods of the invention is an Auxin inducible promoter. Preferably said Auxin inducible promoter comprises the well known Auxin response elements TGTCTC and GGTCCCAT as represented by SEQ ID NO: 1151 and 1152, respectively. Examples of promoters inducible by the hormone Auxin are well known in the art, for example promoters of naturally occurring SAUR genes.

Concerning DHAR polypeptides, also useful in the methods of the invention is a root-specific promoter.

Concerning GDH polypeptides, it should be clear that the applicability of the present invention is not restricted to the GDH polypeptide-encoding nucleic acid represented by SEQ ID NO: 1, nor is the applicability of the invention restricted to expression of a GDH polypeptide-encoding nucleic acid when driven by a constitutive promoter, or when driven by a root-specific promoter.

The constitutive promoter is preferably a medium strength promoter, more preferably selected from a plant derived promoter, such as a GOS2 promoter, more preferably the GOS2 promoter is from rice. Further preferably the constitutive promoter is represented by a nucleic acid sequence substantially similar to SEQ ID NO: 23, most preferably the constitutive promoter is as represented by SEQ ID NO: 23. See the “Definitions” section herein for further examples of constitutive promoters.

According to another preferred feature of the invention, the nucleic acid encoding a GDH polypeptide is operably linked to a root-specific promoter. The root-specific promoter is preferably an RCc3 promoter (Plant Mol Biol. 1995 January; 27(2):237-48), more preferably the RCc3 promoter is from rice, further preferably the RCc3 promoter is represented by a nucleic acid sequence substantially similar to SEQ ID NO: 24, most preferably the promoter is as represented by SEQ ID NO: 24. Examples of other root-specific promoters which may also be used to perform the methods of the invention are shown in Table 3 in the “Definitions” section above.

Optionally, one or more terminator sequences may be used in the construct introduced into a plant. Preferably, the construct comprises an expression cassette comprising a GOS2 promoter or comprising the RCc3, and the nucleic acid encoding the GDH polypeptide.

Concerning FLA-like polypeptides, it should be clear that the applicability of the present invention is not restricted to the FLA-like polypeptide-encoding nucleic acid represented by SEQ ID NO: 171, nor is the applicability of the invention restricted to expression of a FLA-like polypeptide-encoding nucleic acid when driven by a constitutive promoter.

The constitutive promoter is preferably a medium strength promoter, more preferably selected from a plant derived promoter, such as a GOS2 promoter, more preferably the GOS2 promoter is from rice. Further preferably the constitutive promoter is represented by a nucleic acid sequence substantially similar to SEQ ID NO: 500, most preferably the constitutive promoter is as represented by SEQ ID NO: 500. See the “Definitions” section herein for further examples of constitutive promoters.

According to a preferred feature of the invention, the modulated expression is increased expression. Methods for increasing expression of nucleic acids or genes, or gene products, are well documented in the art and examples are provided in the definitions section.

It should be clear that the applicability of the present invention is not restricted to the SAUR polypeptide-encoding nucleic acid represented by SEQ ID NO: 501, nor is the applicability of the invention restricted to expression of a SAUR polypeptide-encoding nucleic acid when driven by a constitutive promoter, or when driven by a leaf-specific promoter.

Further preferably the leaf promoter is represented by a nucleic acid sequence substantially similar to SEQ ID NO: 1163, most preferably the constitutive promoter is as represented by SEQ ID NO: 1163. See the “Definitions” section herein for further examples of leaf promoters.

The constitutive promoter is preferably a medium strength promoter, more preferably selected from a plant derived promoter, such as a GOS2 promoter, more preferably the GOS2 promoter is from rice.

According to a preferred feature of the invention, the modulated expression is increased expression. Methods for increasing expression of nucleic acids or genes, or gene products, are well documented in the art and examples are provided in the definitions section.

Concerning SAUR polypeptides, or SYNP polypeptides, or fusion proteins of SAUR polypeptides and SYNP polypeptides, it should be clear that the applicability of the present invention is not restricted to the SAUR, SYNP polypeptide-, fusions thereof-encoding nucleic acid represented by those of Tables A4, E, and F, nor is the applicability of the invention restricted to expression of a SAUR, SYNP polypeptide-fusions thereof-encoding nucleic acid when driven by a constitutive promoter, or when driven by a leaf-specific promoter.

The constitutive promoter is preferably a medium strength promoter, more preferably selected from a plant derived promoter, such as a GOS2 promoter, more preferably the GOS2 promoter is from rice.

Further preferably the leaf promoter is represented by a nucleic acid sequence substantially similar to SEQ ID NO: 1825, most preferably the constitutive promoter is as represented by SEQ ID NO: 1825. See the “Definitions” section herein for further examples of leaf promoters.

According to a preferred feature of the invention, the modulated expression is increased expression. Methods for increasing expression of nucleic acids or genes, or gene products, are well documented in the art and examples are provided in the definitions section.

It should be clear that the applicability of the present invention is not restricted to the DHAR polypeptide-encoding nucleic acid represented by SEQ ID NO: 1957, nor is the applicability of the invention restricted to expression of a DHAR polypeptide-encoding nucleic acid when driven by a constitutive promoter.

The constitutive promoter is preferably a medium strength promoter, more preferably selected from a plant derived promoter, such as a GOS2 promoter, more preferably the GOS2 promoter is from rice. Further preferably the constitutive promoter is represented by a nucleic acid sequence substantially similar to SEQ ID NO: 2251, most preferably the constitutive promoter is as represented by SEQ ID NO: 2251. See the “Definitions” section herein for further examples of constitutive promoters.

Optionally, one or more terminator sequences may be used in the construct introduced into a plant. Preferably, the construct comprises an expression cassette comprising a GOS2 promoter, substantially similar to SEQ ID NO: 2251, and the nucleic acid encoding the DHAR polypeptide.

According to a preferred feature of the invention, the modulated expression is increased expression. Methods for increasing expression of nucleic acids or genes, or gene products, are well documented in the art and examples are provided in the definitions section.

Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

As mentioned above, a preferred method for modulating expression of a nucleic acid encoding a a GDH polypeptide, or a FLA polypeptide, or a SAUR polypeptide, or a DHAR polypeptide, is by introducing and expressing in a plant a nucleic acid encoding a a GDH polypeptide, or a FLA polypeptide, or a SAUR polypeptide, or a DHAR polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

In order to modulate the expression of the nucleic acid encoding a SAUR polypeptide, said introduced nucleic acid may for example comprise one or more DST elements, said elements preferably comprising the conserved DST motif ATAGAT and GAT (SEQ ID NO: 653 and 654). Preferably the DST elements are located downstream, in the 3′ UTR, of the coding region of nucleic acid encoding a SAUR polypeptide. The DST elements are well known in the art and play an important role in the stability of the transcripts encoding SAUR polypeptides. DST elements refer to approximately 40 nucleotide long elements typically present in the 3′ UTR (untranslated region) of mRNAs and involved in regulating mRNA decay M. A. Perez-Amador, et al., New molecular phenotypes in the dst mutants of Arabidopsis revealed by DNA microarray analysis, Plant Cell 13 (2001) 2703-2717. In a further example the expression of the nucleic acid encoding a SAUR polypeptide may be modulated by modification of DST elements in an endogenous SAUR nucleic acid by for example TILLING.

As mentioned above, a preferred method for modulating expression of a nucleic acid encoding the polypeptide useful in the methods of the invention is by introducing and expressing in a plant a nucleic acid encoding a SAUR, a SYNP polypeptide or a fusion thereof; however the effects of performing the method, i.e. enhancing yield-related traits may also be achieved using other well known techniques, including but not limited to T-DNA activation tagging, TILLING, homologous recombination. A description of these techniques is provided in the definitions section.

The invention also provides a method for the production of transgenic plants having enhanced yield-related traits relative to control plants, comprising introduction and expression in a plant of any nucleic acid encoding a GDH polypeptide, or a FLA polypeptide, or a SAUR polypeptide, or a DHAR polypeptide, as defined hereinabove.

More specifically, the present invention provides a method for the production of transgenic plants having enhanced yield-related traits, particularly increased yield and/or increased early vigour, which method comprises:
(i) introducing and expressing in a plant or plant cell nucleic acid encoding a GDH polypeptide, or a FLA polypeptide, or a SAUR polypeptide, or a DHAR polypeptide; and
(ii) cultivating the plant cell under conditions promoting plant growth and development.

The nucleic acid of (i) may be any of the nucleic acids capable of encoding a GDH polypeptide, or a FLA polypeptide, or a SAUR polypeptide, or a DHAR polypeptide, as defined herein.

The invention also provides a method for the production of transgenic plants having enhanced yield-related traits relative to control plants, comprising introduction and expression in a plant of any nucleic acid encoding a polypeptide useful in the methods of the invention as defined hereinabove.

Methods for introducing and expressing two or more trans-genes (also called gene stacking) in transgenic plants are well known in the art (see for example, a review by Halpin (2005) Plant Biotech J (3): 141-155. Gene stacking can proceed by interative steps, where two or more transgenes can be sequentially introduced into a plant by crossing a plant containing one transgene with individuals harbouring other transgenes or, alternatively, by re-transforming (or super-transforming) a plant containing one transgene with new genes. The two or more transgenes may be introduced simultaneously by transformation with for example a culture of mix Agroacterium tumefaciens strains harbouring each of the transgenes of to be introduced in the plant.

Concerning SAUR polypeptides, or SYNP polypeptides, or fusion proteins of SAUR polypeptides and SYNP polypeptides, according to the present invention, there is also provided a method for enhancing yield-related traits in plants, which method comprises simultaneously introducing and expressing in a plant: (i) a first nucleic acid sequence encoding at least one SAUR polypeptides; and (ii) a second nucleic acid sequence encoding a SYNP polypeptide or (iii) a introducing and expressing a nucleic acid encoding a fusion of a SAUR and a SYNP polypeptide, which plants have enhanced yield-related traits relative to plants having increased expression of one of:
(i) a first nucleic acid encoding at least a SAUR polypeptide; or
(ii) a second nucleic acid encoding one or more SYNP polypeptides; or
(iii) a nucleic acid according to (i) and a nucleic according to (ii), wherein preferably said SAUR polypeptide is selected from the group consisting of any of the polypeptides of Table A and homologues thereof; wherein preferably said SYNP polypeptide is selected from the group consisting of any of the polypeptides of Table E and F.

The nucleic acid sequences that are simultaneously introduced and expressed, are comprised in one or more nucleic acid molecules. Therefore, according to the present invention is provided increasing yield-related traits in plants, which method comprises simultaneously introducing and expressing in a plant:
(i) a nucleic acid molecule encoding a SAUR and a SYNP polypeptide; or
(ii) any two or three nucleic acids, each encoding at least a SAUR or a SYNP polypeptide; or
(iii) a nucleic acid encoding a fusion of a SAUR and a SYNP polypeptide
   wherein preferably said SAUR polypeptide is selected from the group consisting of any of the polypeptides of Table A and homologues thereof and said SYNP polypeptide is selected from the group consisting of any of the polypeptides of Tables E and F and homologues thereof.

More specifically, the present invention provides a method for the production of transgenic plants having enhanced yield-related traits, which method comprises:
(i) introducing and expressing in a plant a first nucleic acid encoding at least a SAUR polypeptide and a second nucleic acid encoding one or more SYNP polypeptides, or a nucleic acid encoding a protein fusion between at least a SAUR and one or more SYNP polypeptides wherein the first and second nucleic acids are comprised in a single nucleic acid molecule or in multiple, at least two, nucleic acid molecules; and
(ii) cultivating the plant cell under conditions promoting plant growth and development.

The nucleic acid sequence introduced in the plant is preferably a nucleic acid molecule comprising a nucleic acid sequence encoding fusions of at least one SAUR or a portion thereof and at least one SYNP polypeptide or a portion thereof. Methods to make nucleic acids encoding protein fusions are well known in the art, and include but are not limited to PCR, DNA restriction and ligation. The nucleic acid sequences encoding the SAUR and the SYNP polypeptides may be fused to each other or separated by coding or non-coding DNA, such as promoters, introns, subcellular targeting signal, or stuffed DNA such as the MARs (Matrix attachment Regions) regions. The SAUR encoding part may be at the N-terminus of the fusion protein or vice versa.

Preferably, the nucleic acid sequences of (i) are sequentially introduced and expressed by crossing. A cross is performed between a female parent plant comprising an introduced and expressed isolated nucleic acid sequence encoding at least one SAUR polypeptide, and a male parent plant also comprising an introduced and expressed isolated nucleic acid sequence encoding one or two SYNP polypeptides, and preferably selecting in the progeny for the presence and expression of both transgenes. Therefore, according to the present invention, there is provided a method for enhancing yield-related traits in plants, by crossing a female or male parent plant comprising an introduced and expressed isolated nucleic acid sequence encoding at least a SAUR polypeptide, and a male or female parent plant comprising an introduced and expressed isolated nucleic acid sequence encoding one or more SYNP polypeptides, and preferably selecting in the progeny for the presence and expression of at least two of the introduced transgenes encoding the corresponding SAUR and SYNP polypeptides, wherein said plants have enhanced yield-related traits relative to the parent plants, or to any other control plants as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the above-mentioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

Concerning SAUR polypeptides, or SYNP polypeptides, or fusion proteins of SAUR polypeptides and SYNP polypeptides, alternatively the nucleic acid sequences encoding the polypeptides useful in the methods of the invention are sequentially introduced and expressed by re-transformation. Re-transformation is performed by introducing and expressing a first nucleic acid sequence encoding at least a SAUR polypeptide in a plant part, or plant cell comprising a introduced and expressed nucleic acid sequence encoding one or more SYNP polypeptides, and preferably by selecting in the progeny for the presence and expression of both transgenes; or vice versa introducing the nucleic acid encoding the SYNP polypeptide in the plant already comprising the isolated nucleic acid encoding the SAUR polypeptide. Therefore, according to the present invention, there is provided a method for enhancing yield-related traits in plants, by re-transformation performed by introducing and expressing a nucleic acid sequence encoding at least a SAUR polypeptide into a plant, plant part, or plant cell comprising an introduced and expressed nucleic acid sequence encoding one or more SYNP polypeptides, and by preferably selecting in the progeny for the presence and expression of both transgenes, wherein said plants have enhanced yield-related traits relative to the plants having increased expression of one of:
(i) a first nucleic acid encoding at least a SAUR polypeptide; or
(ii) a second nucleic acid encoding one or more SYNP polypeptides; or
(iii) a nucleic acid according to (i) and a nucleic according to (ii), wherein preferably said SAUR polypeptide is selected from the group consisting of any of the polypeptides of Table A4 and homologues thereof; wherein preferably said SYNP polypeptide is selected from the group consisting of any of the polypeptides of Table E and F.

Alternatively, gene stacking can occur via simultaneous transformation, or co-transformation, which is faster and can be used in a whole range of transformation techniques, as described in the definitions section herein.

When direct genetic transformation is considered, using physical or chemical delivery systems (e.g., microprojectile bombardment, PEG, electroporation, liposome, glass needles, etc.), the transgenes (at least two) can also be present in a number of conformations, but essentially do not need to be comprised in a vector capable of being replicated in Agrobacteria or viruses, intermediates of the genetic transformation. The two transgenes can be comprised in one or more nucleic acid molecules, but simultaneously used for the genetic transformation process.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention encompasses plants or parts thereof (including seeds) obtainable by the methods according to the present invention. The plants or parts thereof comprise a nucleic acid transgene encoding a GDH polypeptide, or a FLA polypeptide, or a SAUR polypeptide, or a DHAR polypeptide, as defined above. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

Concerning SAUR polypeptides, or SYNP polypeptides, or fusion proteins of SAUR polypeptides and SYNP polypeptides, the present invention clearly extends to any plant cell or plant produced by any of the methods described herein, and to all plant parts and propagules thereof. The present invention encompasses plants or parts thereof (including seeds) obtainable by the methods according to the present invention. The plants or parts thereof comprise a nucleic acid transgene encoding an (isolated) SAUR polypeptide and an (isolated) SYNP polypeptide as defined above. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a GDH polypeptide, or a FLA polypeptide, or a SAUR polypeptide, or a DHAR polypeptide, as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, linseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum, emmer, spelt, secale, einkorn, teff, milo and oats.

The invention also extends to harvestable parts of a plant such as, but not limited to seeds, leaves, fruits, flowers, stems, roots, rhizomes, tubers and bulbs, which harvestable parts comprise a recombinant nucleic acid encoding a GDH polypeptide, or a FLA polypeptide, or a SAUR polypeptide, or an SYNP polypeptide, or a fusion protein of SAUR polypeptides and SYNP polypeptides, or a DHAR polypeptide. The invention furthermore relates to products derived, preferably directly derived, from a harvestable part of such a plant, such as dry pellets or powders, oil, fat and fatty acids, starch or proteins.

Concerning SAUR polypeptides, or SYNP polypeptides, or fusion proteins of SAUR polypeptides and SYNP polypeptides, the invention also provides for the use of a construct comprising:
(i) a first nucleic acid encoding at least a SAUR polypeptide and a second nucleic acid encoding one or more SYNP polypeptides, wherein the first and second nucleic acids are comprised in a single nucleic acid molecule or in multiple, at least two, nucleic acid molecules;
(ii) one or more control sequences capable of driving expression of the nucleic acid sequence of (i), preferably a plant promoter, more preferably a constitutive promoter, even more preferably a GOS2 promoter, most preferably a GOS2 promoter from rice; and optionally
(iii) a transcription termination sequence.
   or of a mixture of constructs as described above, in a method for making plants having enhanced yield-related traits relative to plants having increased expression of one of the nucleic acids encoding the corresponding SAUR and SYNP polypeptides which increased yield-related traits are one or more of: (i) increased early vigour; (ii) increased aboveground biomass or root biomass; (iii) increased total seed yield per plant; (iv) increased seed filling rate; (v) increased number of (filled) seeds; (vi) increased harvest index; or (vii) increased thousand kernel weight (TKW).

The present invention also encompasses use of nucleic acids encoding GDH polypeptides, or FLA polypeptides, or SAUR polypeptides, or SYNP polypeptides, or fusion proteins of SAUR polypeptides and SYNP polypeptides, or DHAR polypeptides, as described herein and use of these GDH polypeptides, or FLA polypeptides, or SAUR polypeptides, or SYNP polypeptides, or fusion proteins of SAUR polypeptides and SYNP polypeptides, or DHAR polypeptides, in enhancing any of the aforementioned yield-related traits in plants. For example, nucleic acids encoding GDH polypeptides, or FLA polypeptides, or SAUR polypeptides, or SYNP polypeptides, or fusion proteins of SAUR polypeptides and SYNP polypeptides, or DHAR polypeptides, described herein, or the GDH polypeptides, or FLA polypeptides, or SAUR polypeptides, or SYNP polypeptides, or fusion proteins of SAUR polypeptides and SYNP polypeptides, or DHAR polypeptides, themselves, may find use in breeding programmes in which a DNA marker is identified which may be genetically linked to gene encoding GDH polypeptides, or FLA polypeptides, or SAUR polypeptides, or SYNP polypeptides, or fusion proteins of SAUR polypeptides and SYNP polypeptides, or DHAR polypeptides. The nucleic acids/genes, or the GDH polypeptides, or FLA polypeptides, or SAUR polypeptides, or SYNP polypeptides, or fusion proteins of SAUR polypeptides and SYNP polypeptides, or DHAR polypeptides, themselves may be used to define a molecular marker. This DNA or protein marker may then be used in breeding programmes to select plants having enhanced yield-related traits as defined hereinabove in the methods of the invention. Furthermore, allelic variants of a nucleic acid/gene encoding GDH polypeptides, or FLA polypeptides, or SAUR polypeptides, or SYNP polypeptides, or fusion proteins of SAUR polypeptides and SYNP polypeptides, or DHAR polypeptides, may find use in marker-assisted breeding programmes. Nucleic acids encoding GDH polypeptides, or FLA polypeptides, or SAUR polypeptides, or SYNP polypeptides, or fusion proteins of SAUR polypeptides and SYNP polypeptides, or DHAR polypeptides, may also be used as probes for genetically and physically mapping the genes that they are a part of, and as markers for traits linked to those genes. Such information may be useful in plant breeding in order to develop lines with desired phenotypes.

Concerning SAUR polypeptides, or SYNP polypeptides, or fusion proteins of SAUR polypeptides and SYNP polypeptides, encompassed within the invention are the following described embodiments:
Item 1. An isolated SAUR-based protein complex, comprising at least a SAUR polypeptide selected from the group of polypeptides of Table A and homologues and portions thereof and one or more SYNP polypeptides selected from the group of Tables E and F and homologues and portions thereof.
Item 2. A nucleic acid comprising a sequence encoding the proteins of the complex of item 1.
Item 3. The use of a protein complex according to item 1 to enhance yield-related traits.
Item 4. The use of a nucleic acid according to item 2 to enhance yield-related traits.
Item 5. A method to promote SAUR-based protein complex formation by simultaneous overexpression of at least two proteins of the complex.
Item 6. A method to enhance yield related traits modulating levels and or activity of a SAUR-base protein complex, said complex comprising at least one SAUR polypeptide and one SYNP polypeptide, by modulating one or more nucleic acids comprising the coding region of a SAUR polypeptide, homologue or portion thereof and the coding region of a SYNP polypeptide, homologue or portion thereof.

### Items

### 1. Glutamate Dehydrogenase (GDH) Polypeptides

1. A method for enhancing yield-related traits in plants relative to control plants, comprising modulating expression in a plant of a nucleic acid encoding a glutamate dehydrogenase (GDH) polypeptide, wherein said GDH polypeptide is a NAD-dependent GDH having glutamate dehydrogenase activity.
2. Method according to item 1, wherein said GDH polypeptide comprises one or more of the motifs 1 to 20 (SEQ ID NO: 3 to SEQ ID NO: 22).
3. Method according to item 1 or 2, wherein said modulated expression is effected by introducing and expressing in a plant a nucleic acid encoding a GDH polypeptide.
4. Method according to any one of items 1 to 3, wherein said nucleic acid encoding a GDH polypeptide encodes any one of the proteins listed in Table A1 or is a portion of such a nucleic acid, or a nucleic acid capable of hybridising with such a nucleic acid.
5. Method according to any one of items 1 to 4, wherein said nucleic acid sequence encodes an orthologue or paralogue of any of the proteins given in Table A1.
6. Method according to any preceding item, wherein said enhanced yield-related traits comprise increased yield, preferably increased biomass, increased early vigour and/or increased seed yield relative to control plants.
7. Method according to any one of items 1 to 6, wherein said enhanced yield-related traits are obtained under non-stress conditions.
8. Method according to any one of items 1 to 6, wherein said enhanced yield-related traits are obtained under conditions of nitrogen deficiency.
9. Method according to any one of items 3 to 8, wherein said nucleic acid is operably linked to one of
   (i) a constitutive promoter, preferably to a GOS2 promoter, most preferably to a GOS2 promoter from rice;
   (ii) a root specific promoter, preferably a RCc3 promoter, most preferably a RCc3 promoter from rice.
10. Method according to any one of items 1 to 9, wherein said nucleic acid encoding a GDH polypeptide is of plant origin, preferably from a dicotyledonous plant, further preferably from the family Poaceae, more preferably from the genus Zea or Oryza, most preferably from maize or rice.
11. Plant or part thereof, including seeds, obtainable by a method according to any one of items 1 to 10, wherein said plant or part thereof comprises a recombinant nucleic acid encoding a GDH polypeptide.
12. Construct comprising:
   (i) nucleic acid encoding a GDH polypeptide as defined in items 1 or 2;
   (ii) one or more control sequences capable of driving expression of the nucleic acid sequence of (a); and optionally
   (iii) a transcription termination sequence.
13. Construct according to item 12, wherein one of said control sequences is one of
   (i) a constitutive promoter, preferably a GOS2 promoter, most preferably a GOS2 promoter from rice;
   (ii) a root specific promoter, preferably a RCc3 promoter, most preferably a RCc3 promoter from rice.
14. Use of a construct according to item 12 or 13 in a method for making plants having increased yield, particularly increased biomass, increased early vigour and/or increased seed yield relative to control plants.
15. Plant, plant part or plant cell transformed with a construct according to item 12 or 13.
16. Method for the production of a transgenic plant having increased yield, particularly increased biomass, increased early vigour and/or increased seed yield relative to control plants, comprising:
   1. introducing and expressing in a plant a nucleic acid encoding a GDH polypeptide as defined in item 1 or 2; and
   2. cultivating the plant cell under conditions promoting plant growth and development.
17. Transgenic plant having increased yield, particularly increased biomass, increased early vigour and/or increased seed yield, relative to control plants, resulting from modulated expression of a nucleic acid encoding a GDH polypeptide as defined in item 1 or 2, or a transgenic plant cell derived from said transgenic plant.
18. Transgenic plant according to item 11, 15 or 17, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
19. Harvestable parts of a plant according to item 18, wherein said harvestable parts are preferably shoot biomass, root biomass and/or seeds.
20. Products derived from a plant according to item 18 and/or from harvestable parts of a plant according to item 19.
21. Use of a nucleic acid encoding a GDH polypeptide in increasing yield, particularly in increasing seed yield, early vigour and/or biomass in plants, relative to control plants.
22. An isolated nucleic acid molecule comprising:
   (a) a nucleic acid represented by SEQ ID NO: 99;
   (b) the complement of a nucleic acid represented by SEQ ID NO: 99;
   (c) at least 50%, 55%, 60%, 65%, 70%, 75%, 80%, 85%, 90%, 95%, 96%, 97%, 98%, 99% or more sequence identity to the amino acid sequence represented by SEQ ID NO: 176, and having in increasing order of preference at least 50%, 55%, 60%, 65%, 70%, 75%, 80%, 85%, 90%, 95%, 96%, 97%, 98%, 99% or more sequence identity to any one of the motifs 4 to 6.
23. An isolated polypeptide comprising:
   (i) an amino acid sequence represented by SEQ ID NO: 176;
   (ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 176, and having in increasing order of preference at least 50%, 55%, 60%, 65%, 70%, 75%, 80%, 85%, 90%, 95%, 96%, 97%, 98%, 99% or more sequence identity to any one of the motifs 4 to 6;
   3. derivatives of any of the amino acid sequences given in (i) or (ii) above.

### 2. Fasciclin-Like AGP (FLAB) Polypeptides

1. A method for enhancing yield-related traits in plants relative to control plants, comprising modulating expression in a plant of a nucleic acid encoding a polypeptide encoding a Fasciclin-like (FLA-like) polypeptide.
2. Method according to item 1, wherein said FLA-like polypeptide comprises at least one, two, three, or four fasciclin-like domains having in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid represented by:
   (i) TITVCAVDNAGMSDLLSKQLSIYTIKNVLSFRVLLDYFDAKKLHQITNGTALAATM FQATGSATGSSGFVNITDLRGGKVGLSPADYNGPPPAKFVKSIAEIPYNISVIQIS TIL (SEQ ID NO: 487) which corresponds to the N-term FLA_like domain in SEQ ID NO: 172; or
   (ii) VDGGVTIFCPRDDAMKKFLPKFKNLTAEGKQSLLEYHGIPIYQSISNLKSNNGD MNTLATDGAKKYAVVIQNDGEDVTIKTKIVTAKITATVVDKLPLAIYSLDKVL (SEQ ID NO: 488) which corresponds to the C-term FLA_like domain in SEQ ID NO: 172.
3. Method according to item 1 or 2, wherein said modulated expression is effected by introducing and expressing in a plant a nucleic acid encoding a FLA-like polypeptide.
4. Method according to any one of items 1 to 3, wherein said nucleic acid encoding a FLA-like polypeptide encodes any one of the proteins listed in Table A2 or is a portion of such a nucleic acid, or a nucleic acid capable of hybridising with such a nucleic acid.
5. Method according to any one of items 1 to 4, wherein said nucleic acid sequence encodes an orthologue or paralogue of any of the proteins given in Table A2.
6. Method according to any preceding item, wherein said enhanced yield-related traits comprise increased yield preferably increased biomass and/or increased seed yield relative to control plants.
7. Method according to any one of items 1 to 6, wherein said enhanced yield-related traits are obtained under drought conditions.
8. Method according to any one of items 1 to 6, wherein said enhanced yield-related traits are obtained under non-stress or under nitrogen deficiency.
9. Method according to any one of items 3 to 8, wherein said nucleic acid is operably linked to a constitutive promoter, preferably to a GOS2 promoter, most preferably to a GOS2 promoter from rice.
10. Method according to any one of items 1 to 9, wherein said nucleic acid encoding a FLA-like polypeptide is of plant origin, preferably from a dicotyledonous plant, further preferably from the family solanaceae, more preferably from the genus Lycopersicum, most preferably from Lycopersicum esculentum.
11. Plant or part thereof, including seeds, obtainable by a method according to any one of items 1 to 10, wherein said plant or part thereof comprises a recombinant nucleic acid encoding a FLA-like polypeptide.
12. Construct comprising:
   (i) nucleic acid encoding a FLA-like polypeptide as defined in items 1 or 2;
   (ii) one or more control sequences capable of driving expression of the nucleic acid sequence of (a); and optionally
   (iii) a transcription termination sequence.
13. Construct according to item 12, wherein one of said control sequences is a constitutive promoter, preferably a GOS2 promoter, most preferably a GOS2 promoter from rice.
14. Use of a construct according to item 12 or 13 in a method for making plants having increased yield, particularly increased biomass and/or increased seed yield relative to control plants.
15. Plant, plant part or plant cell transformed with a construct according to item 12 or 13.
16. Method for the production of a transgenic plant having increased yield, particularly increased biomass and/or increased seed yield relative to control plants, comprising:
   (i) introducing and expressing in a plant a nucleic acid encoding a FLA-like polypeptide as defined in item 1 or 2; and
   (ii) cultivating the plant cell under conditions promoting plant growth and development.
17. Transgenic plant having increased yield, particularly increased biomass and/or increased seed yield, relative to control plants, resulting from modulated expression of a nucleic acid encoding a FLA-like polypeptide as defined in item 1 or 2, or a transgenic plant cell derived from said transgenic plant.
18. Transgenic plant according to item 11, 15 or 17, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
19. Harvestable parts of a plant according to item 18, wherein said harvestable parts are preferably shoot biomass and/or seeds.
20. Products derived from a plant according to item 18 and/or from harvestable parts of a plant according to item 19.
21. Use of a nucleic acid encoding a FLA-like polypeptide in increasing yield, particularly in increasing seed yield and/or shoot biomass in plants, relative to control plants.

### 3. Small Auxin-Up RNA (SAUR) Polypeptides

1. A method for enhancing yield-related traits in plants relative to control plants, comprising modulating expression in a plant of a nucleic acid encoding a SAUR polypeptide, wherein said SAUR polypeptide comprises an Auxin inducible domain.
2. Method according to item 1, wherein said SAUR polypeptide comprises one or more of the following motifs:

(i)
Motif 23:
(SEQ ID NO: 1155)
LAVYVGEMMQKRRFVVPVTYLSHPCFQKLLRKAEEEFGFDHPMGGLTI
PC;
(ii)
Motif 24:
(SEQ ID NO: 1156)
KHxxGVYTAEKxxYxxxIxxxxxxxxxAxxxxS xxxYxxxxPMPIx
LxxC;
(iii)
Motif 25:
(SEQ ID NO: 1157)
LQSSKQLLKSLSHSSNNVAIP;
(iv)
Motif 26:
(SEQ ID NO: 1158)
VxxxKIAxKSQ;
(v)
Motif 27:
(SEQ ID NO: 1159)
EQIFIDLASRL;
(vi)
Motif 28:
(SEQ ID NO: 1159)
VExxxVxxxxL;

Wherein X represents any amino acid.
3. Method according to item 1, wherein said SAUR polypeptide is a SAUR33-like polypeptide and comprises the following Motifs 47 and 48 and optionally also one or both of Motifs 49 and 50 or any sequence having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to Motifs 47 to 50

Motif 47
(SEQ ID NO: 2284)
CEVVLFEHLLWMLENADPQ
Motif 48
(SEQ ID NO: 2285)
PESLDELVEYYAC
Motif 49
(SEQ ID NO: 2286)
GLSKLRCMIRRWHSSSRI
Motif 50
(SEQ ID NO: 2287)
SFHGADEVPKGLHPVYVGKSRRRYLIAEELVGHPLFQNLVDRT

4. Method according to any one of items 1 to 3, wherein said modulated expression is effected by introducing and expressing in a plant a nucleic acid encoding a SAUR polypeptide.
5. Method according to any one of items 1 to 4, wherein said nucleic acid encoding a SAUR polypeptide encodes any one of the proteins listed in Table A3 or A3(i) or is a portion of such a nucleic acid, or a nucleic acid capable of hybridising with such a nucleic acid.
6. Method according to any one of items 1 to 5, wherein said nucleic acid sequence encodes an orthologue or paralogue of any of the proteins given in Table A3 or A3(i).
7. Method according to any preceding item, wherein said enhanced yield-related traits comprise any one or more of increased yield, increased early vigour, increased biomass and increased seed yield relative to control plants.
8. Method according to any one of items 1 to 7, wherein said enhanced yield-related traits are obtained under non-stress conditions.
9. Method according to any one of items 1 to 8, wherein said enhanced yield-related traits are obtained under conditions of drought stress, salt stress or nitrogen deficiency.
10. Method according to any one of items 4 to 9, wherein said nucleic acid is operably linked to a constitutive promoter, preferably to a leaf specific promoter, most preferably to the promoter represented by SEQ ID NO: 1163.
11. Method according to any one of items 1 to 9, wherein said nucleic acid encoding a SAUR polypeptide is of plant origin, preferably from a dicotyledonous plant, further preferably from the family Brassicaceae, more preferably from the genus Arabidopsis, most preferably from Arabidopsis thaliana.
12. Plant or part thereof, including seeds, obtainable by a method according to any one of items 1 to 11, wherein said plant or part thereof comprises a recombinant nucleic acid encoding a SAUR polypeptide.
13. Construct comprising:
   (i) nucleic acid encoding a SAUR polypeptide as defined in items 1 to 3;
   (ii) one or more control sequences capable of driving expression of the nucleic acid sequence of (a); and optionally
   (iii) a transcription termination sequence.
14. Construct according to item 13, wherein one of said control sequences is a constitutive promoter, preferably a leaf specific promoter, most preferably a promoter represented by SEQ ID NO: 1163.
15. Use of a construct according to item 13 or 14 in a method for making plants having increased yield, particularly increased biomass and/or increased seed yield relative to control plants.
16. Plant, plant part or plant cell transformed with a construct according to item 13 or 14.
17. Method for the production of a transgenic plant having increased yield, particularly increased biomass and/or increased seed yield relative to control plants, comprising:
   (i) introducing and expressing in a plant a nucleic acid encoding a SAUR polypeptide as defined in item 1 to 3; and
   (ii) cultivating the plant cell under conditions promoting plant growth and development.
18. Transgenic plant having increased yield, particularly increased biomass and/or increased seed yield, relative to control plants, resulting from modulated expression of a nucleic acid encoding a SAUR polypeptide as defined in item 1 to 3, or a transgenic plant cell derived from said transgenic plant.
19. Transgenic plant according to item 12, 16 or 18, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
20. Harvestable parts of a plant according to item 18, wherein said harvestable parts are preferably shoot biomass and/or seeds.
21. Products derived from a plant according to item 19 and/or from harvestable parts of a plant according to item 20.
22. Use of a nucleic acid encoding a SAUR polypeptide in increasing yield, particularly in increasing seed yield and/or shoot biomass in plants, relative to control plants.

### 4. SAUR Polypeptides, or SYNP Polypeptides, or Fusion Proteins of SAUR Polypeptides and SYNP Polypeptides

1. A method for enhancing yield-related traits in plants relative to control plants, comprising modulating expression in a plant of a first nucleic acid encoding at least a SAUR polypeptide and a second nucleic acid encoding one or more SYNP polypeptides, or a nucleic acid encoding a protein fusion between at least a SAUR and one or more SYNP polypeptides wherein the first and second nucleic acids are comprised in a single nucleic acid molecule or in multiple, at least two, nucleic acid molecules.
2. A method according to item 1 wherein
   (i) the SAUR polypeptide useful in the method in the invention has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% overall sequence identity to the amino acid represented by any one of the polypeptide sequences of Table A4, more preferably by SEQ ID NO: 1164 or a variant thereof;
   (ii) the SYNP polypeptide useful in the method in the invention has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% overall sequence identity to the amino acid represented by any one of the polypeptide sequences of Table E or Table F.
3. Method according to items 1 or 2, wherein said modulated expression is effected by introducing and expressing in a plant a first nucleic acid encoding at least a SAUR polypeptide and a second nucleic acid encoding one or more SYNP polypeptides, or a nucleic acid encoding a protein fusion between at least a SAUR and one or more SYNP polypeptides wherein the first and second nucleic acids are comprised in a single nucleic acid molecule or in multiple, at least two, nucleic acid molecules.
4. Method according to any preceding item, wherein said enhanced yield-related traits comprise increased yield, preferably increased biomass and/or increased seed yield relative to control plants.
5. Method according to any preceding item, wherein said enhanced yield-related traits are obtained under non-stress conditions.
6. Method according to any preceding item, wherein said enhanced yield-related traits are obtained under conditions of drought stress, salt stress or nitrogen deficiency.
7. Method according to any one of items 3 to 6, wherein said one or more said nucleic acids are operably linked to a plant promoter, preferably to a constitutive promoter, more preferably to a GOS2 promoter, most preferably to a GOS2 promoter from rice.
8. Method according to any preceding item, wherein said one or more said nucleic acids is of plant origin, preferably from a dicotyledonous plant, further preferably from the family Brassicaceae, more preferably from the genus Arabidopsis, most preferably from Arabidopsis thaliana.
9. Plant or part thereof, including seeds, obtainable by a method according to any preceding item, wherein said plant or part thereof comprises:
   (i) a first nucleic acid encoding at least a SAUR polypeptide, preferably selected from the polypeptides of Table A4 or a homologue or derivative thereof; and
   (ii) a second nucleic acid encoding one or more SYNP polypeptides, preferably selected from the polypeptides of Tables E and F or a homologue or derivative thereof; or
   (iii) a nucleic acid encoding a protein fusion between any two polypeptides of (i) and (ii);
      wherein the first and second nucleic acids are comprised in a single nucleic acid molecule or in multiple, at least two, nucleic acid molecules.
10. Construct comprising:
   (i) a first nucleic acid encoding at least a SAUR polypeptide and a second nucleic acid encoding one or more SYNP polypeptides, wherein the first and second nucleic acids are comprised in a single nucleic acid molecule or in multiple, at least two, nucleic acid molecules;
   (ii) one or more control sequences capable of driving expression of the nucleic acid sequence of (i), preferably a plant promoter, more preferably a constitutive promoter, even more preferably a GOS2 promoter, most preferably a GOS2 promoter from rice; and optionally
   (iii) a transcription termination sequence.
11. Use of a construct according to item 10 in a method for making plants having increased yield, particularly increased biomass and/or increased seed yield relative to control plants.
12. Plant, plant part or plant cell transformed with a construct according to item 11.
13. Method for the production of a transgenic plant having increased yield, particularly increased biomass and/or increased seed yield relative to control plants, comprising:
   (i) introducing and expressing in a plant a first nucleic acid encoding at least a SAUR polypeptide and a second nucleic acid encoding one or more SYNP polypeptides, or a nucleic acid encoding a protein fusion between at least a SAUR and one or more SYNP polypeptides wherein the first and second nucleic acids are comprised in a single nucleic acid molecule or in multiple, at least two, nucleic acid molecules; and
   (ii) cultivating the plant cell under conditions promoting plant growth and development.
14. Transgenic plant having increased yield, particularly increased biomass and/or increased seed yield, relative to control plants, resulting from modulated expression of a first nucleic acid encoding at least a SAUR polypeptide and a second nucleic acid encoding one or more SYNP polypeptides, or a nucleic acid encoding a protein fusion between at least a SAUR and one or more SYNP polypeptides wherein the first and second nucleic acids are comprised in a single nucleic acid molecule or in multiple, at least two, nucleic acid molecules or a transgenic plant cell derived from said transgenic plant.
15. Transgenic plant according to item 9, 12 or 14, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
16. Harvestable parts of a plant according to item 15, wherein said harvestable parts are preferably shoot biomass and/or seeds.
17. Products derived from a plant according to item 14 or 15 and/or from harvestable parts of a plant according to item 16.
18. Use of any one, two or more nucleic acids according to item 1 in increasing yield, particularly in increasing seed yield and/or shoot biomass in plants, relative to control plants.

### 5. Dehydroascorbate Reductase (DHAR) Polypeptides

1. A method for enhancing yield-related traits in plants relative to control plants, comprising modulating expression in a plant of a nucleic acid encoding a DHAR polypeptide, wherein said polypeptide comprising at least a dehydroascorbate reductase domain with an accession number PTHR11260:SF15.
2. Method according to item 1, wherein the DHAR domain the DHAR polypeptide has at least, in increasing order of preference, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the sequence located between amino acid 19 and 210 of SEQ ID NO 1958.
3. Method according to item 1, wherein said DHAR polypeptide comprises motifs having at least, in increasing order of preference 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to anyone of the following motifs:

(i)
Motif 35:
(SEQ ID NO: 2239)
P[DN]KLGDCPF[SC]QRVLLTLEEK[KH][VL]PY[KD][ML][KH]
L[IV];
(ii)
Motif 36:
(SEQ ID NO: 2240)
D[DEG]KW[VI][PAS]DSDVI[TV][QG][IL][LI]EEK[YF]PEP
[SP]L[VA]TPPE,
(iii)
Motif 37:
(SEQ ID NO: 2241)
P[FY][IV][NA]GE[KN][IV][ST]A[VA]DLSL[AG]PKLYHLE
[VI]ALGH[FY]K[KN]W[ST][VI]P

4. Method, according to any of the items 1 to 3, wherein said DHAR polypeptide comprises any one or more sequence motifs having at least, in increasing order of preference 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to anyone of the following motifs:

(i)
Motif 38:
(SEQ ID NO: 2242)
E[VI]CVKA[AS]V[GT]AP[DN][KV]LGDCPF[SC]QRVLLTLEE,
(ii)
Motif 39:
(SEQ ID NO: 2243)
PPE[FK]ASVGSKIF[PS][TS]F[VI][GT]FLKSKD[PA][NS]D
G[TS]EQ,
(iii)
Motif 40:
(SEQ ID NO: 2243)
[IV][ST]A[VA]DLSL[AG]PKLYHL[EQ][VI]ALGH[FY]K[KN]
W[ST][VI]P[ED]SL[TP]HV[HK][NS]Y[MT]K[ALS][LI]FS
[RL][ED]SF[EV]KT

5. Method, according to any of the items 1 to 4, wherein said DHAR polypeptide comprises any one or more of the following sequence motifs having at least, in increasing order of preference 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to anyone of the following motifs:

(i)
Motif 41:
(SEQ ID NO: 2244)
E[IV]CVKAA[VT]GAPD[VIT]LGDCPF[SC]QRVLLTLEE,
(ii)
Motif 42:
(SEQ ID NO: 2245)
PPE[FY]ASVGSKIF[PG][ST]FV[TK]FLKSKD[AP][NS]DG
[TS]E[QK]
(iii)
Motif 43:
(SEQ ID NO: 2246)
[IV][TS]AVDLSLAPKLYHL[EQ]VAL[GE]HFK[KG]W[TSK]
[VI]PE[SN]LTHVH[NA]Y[TM]K[LAS]LFSRESFEKT.

6. Method, according to any of the items 1 to 4, wherein said DHAR polypeptide comprises any one or more of the following sequence motifs having at least, in increasing order of preference 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to anyone of the following motifs:

(i)
Motif 44:
(SEQ ID NO: 2247)
PLE[VI]C[VA]KAS[ILV]T[TV]P[ND][KR]LGDCPF[TC]QRVLL
TLEEKHLPY[DE][ML]KLVDL[SG]NKP[ED]WF,
(ii)
Motif 45:
(SEQ ID NO: 2248)
PPE[VI][PA]DSDVITQ[AST]LEEK[YF]P[ED]P[PS]L[AV]TPP
EKASVGSKIFSTF[IV]GFLKSKDP[SN]DG,
(iii)
Motif 46:
(SEQ ID NO: 2249)
QALL[ND]EL[ST][SA]FNDY[LI]KENGPFING[KE][KDE][IV]
SAADLSL[GA]PKLYH[LM]EIALGH[YF]K[NK]W.

7. Method, according to any of the items 1 to 6, wherein the DHAR polypeptide has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to any of the amino acid sequences depicted in of Table A, preferably by the SEQ ID NO: 1958.
8. Method, according to any of the items 1 to 7, wherein said modulated expression is effected by introducing and expressing in a plant a nucleic acid encoding a DHAR polypeptide as defined in any of the previous items.
9. Method according to any one of items 1 to 8, wherein said nucleic acid encoding a DHAR polypeptide encodes any one of the proteins listed in Table A5 or is a portion of such a nucleic acid, or a nucleic acid capable of hybridising with such a nucleic acid.
10. Method according to any one of items 1 to 9, wherein said nucleic acid sequence encodes an orthologue or paralogue of any of the proteins given in Table A5.
11. Method according to any preceding item, wherein said enhanced yield-related traits comprise increased yield, preferably increased seed yield relative to control plants.
12. Method according to any one of items 1 to 11, wherein said enhanced yield-related traits are obtained under non-stress conditions.
13. Method according to any one of items 1 to 12, wherein said enhanced yield-related traits are obtained under conditions of drought stress, salt stress or nitrogen deficiency.
14. Method according to any one of items 8 to 10, wherein said nucleic acid is operably linked to a constitutive promoter, preferably to a GOS2 promoter, most preferably to a GOS2 promoter from rice.
15. Method according to any one of items 1 to 14, wherein said nucleic acid encoding a DHAR polypeptide is of plant origin.
16. Method according to item 15 wherein said nucleic acid encoding a DHAR polypeptide is from a dicotyledonous plant, further preferably from the family Solanaceae, more preferably from the genus Solanum, most preferably from Solanum lycopersicum.
17. Method according to item 15 wherein said nucleic acid encoding a DHAR polypeptide is from a monocotyledonous plant, further preferably from the family Poaceae, more preferably from the genus Oryza, most preferably from Oryza sativa.
18. Method according to item 15 wherein said nucleic acid encoding a DHAR polypeptide is from a monocotyledonous plant, further preferably from the family Poaceae, more preferably from the genus Hordeum, most preferably from Hordeum vulgare.
19. Plant or part thereof, including seeds, obtainable by a method according to any one of items 1 to 18, wherein said plant or part thereof comprises a recombinant nucleic acid encoding a DHAR polypeptide.
20. Construct comprising:
   (i) nucleic acid encoding a DHAR polypeptide as defined in items 1 to 7;
   (ii) one or more control sequences capable of driving expression of the nucleic acid sequence of (a); and optionally
   (iii) a transcription termination sequence.
21. Construct according to item 20, wherein one of said control sequences is a constitutive promoter, preferably a GOS2 promoter, most preferably a GOS2 promoter from rice.
22. Use of a construct according to items 20 or 21 in a method for making plants having increased yield, particularly increased seed yield relative to control plants.
23. Plant, plant part or plant cell transformed with a construct according to items 20 or 21.
24. Method for the production of a transgenic plant having increased yield, particularly increased biomass and/or increased seed yield relative to control plants, comprising:
   1. introducing and expressing in a plant a nucleic acid encoding a DHAR polypeptide as defined in items 1 to 7; and
   2. cultivating the plant cell under conditions promoting plant growth and development.
25. Transgenic plant having increased yield, particularly increased biomass and/or increased seed yield, relative to control plants, resulting from modulated expression of a nucleic acid encoding a DHAR polypeptide as defined in items 1 to 7, or a transgenic plant cell derived from said transgenic plant.
26. Transgenic plant according to item 19, 23 or 25, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
27. Harvestable parts of a plant according to item 26, wherein said harvestable parts are preferably seeds.
28. Products derived from a plant according to item 26 and/or from harvestable parts of a plant according to item 27.
29. Use of a nucleic acid encoding a DHAR polypeptide in enhancing yield characteristics, particularly in increasing seed yield in plants, relative to control plants.
29. An isolated nucleic acid molecule selected from:
   (i) a nucleic acid represented by SEQ ID NO: 1997, SEQ ID NO: 2121 and SEQ ID NO: 2193;
   (ii) the complement of a nucleic acid represented by SEQ ID NO: 1997, SEQ ID NO: 2121 and SEQ ID NO: 2193;
   (iii) a nucleic acid encoding a DHAR polypeptide as represented by any one of SEQ ID NO: 1998, SEQ ID NO: 2122 and SEQ ID NO: 2194, preferably as a result of the degeneracy of the genetic code, said isolated nucleic acid can be derived from a polypeptide sequence as represented by any one of said SEQ IDs and further preferably confers enhanced yield related traits relative to control plants;
   (iv) a nucleic acid having, in increasing order of preference, at least 50%, 55%, 60%, 65%, 70%, 75%, 80%, 85%, 90%, 95%, 96%, 97%, 98%, 99% or more sequence identity with any of the nucleic acid sequences of Table A5 and further preferably confers enhanced yield related traits relative to control plants;
   (v) a nucleic acid molecule which hybridizes with a nucleic acid molecule of (i) to (iv) under stringent hybridization conditions and preferably confers enhanced yield related traits relative to control plants;
   (vi) a nucleic acid encoding a DHAR polypeptide having in increasing order of preference at least 50%, 55%, 60%, 65%, 70%, 75%, 80%, 85%, 90%, 95%, 96%, 97%, 98%, 99% or more sequence identity to any one of SEQ ID NO: 1998, SEQ ID NO: 2122 and SEQ ID NO: 2194 and any of the other amino acid sequences in Table A5 and preferably confers enhanced yield related traits relative to control plants.
30. According to a further embodiment of the present invention, there is also provided an isolated polypeptide selected from:
   (i) an amino acid sequence represented by SEQ ID NO: 1998, SEQ ID NO: 2122 and SEQ ID NO: 2194;
   (ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: Y, and having in increasing order of preference at least 50%, 55%, 60%, 65%, 70%, 75%, 80%, 85%, 90%, 95%, 96%, 97%, 98%, 99% or more sequence identity to SEQ ID NO: 1998, SEQ ID NO: 2122 and SEQ ID NO: 2194;
   (iii) derivatives of any of the amino acid sequences given in (i) or (ii) above.

### DESCRIPTION OF FIGURES

The present invention will now be described with reference to the following figures in which:

FIG. 1 represents the domain structure of SEQ ID NO: 2 with the conserved domains PF02812 and PF00208.

FIG. 2 represents a multiple alignment of various GDH polypeptides. FIG. 2a shows an alignment of Class I GDH polypeptides, FIG. 2b shows an alignment of Class II GDH polypeptides, FIG. 2c shows an alignment of algal GDH polypeptides, FIG. 2d shows an alignment of moss GDH polypeptides. The asterisks indicate identical amino acids among the various protein sequences, colons represent highly conserved amino acid substitutions, and the dots represent less conserved amino acid substitution; on other positions there is no sequence conservation. These alignments can be used for defining further motifs, when using conserved amino acids.

FIG. 3 shows phylogenetic tree of GDH polypeptides. The proteins were aligned using MUSCLE (Edgar (2004), Nucleic Acids Research 32(5): 1792-97). A Neighbour-Joining tree was calculated using Quick-Tree (Howe et al. (2002), Bioinformatics 18(11): 1546-7). Support of the major branching after 100 bootstrap repetitions is indicated. A circular phylogram was drawn using Dendroscope (Huson et al. (2007), BMC Bioinformatics 8(1):460). Class I is the regular EC1.4.1.2 Glade, which is divided in alpha and beta subunits. Several moss GDH sequences cluster with Class I GDH sequences, and are equally useful in the methods of present invention.

FIG. 4 represents the binary vector used for increased expression in Oryza sativa of a GDH-encoding nucleic acid under the control of a rice GOS2 promoter (pGOS2). The vector for GDH expression under control of a rice RCc3 promoter has, except for the promoter sequence, an identical architecture.

FIG. 5 represents the binary vector used for increased expression in Oryza sativa of a FLA-like-encoding nucleic acid under the control of a rice GOS2 promoter (pGOS2).

FIG. 6 represents the binary vector used for increased expression in Oryza sativa of a SAUR-encoding nucleic acid under the control of a rice leaf-specific promoter comprising the sequence represented by SEQ ID NO: 1163.

FIG. 7 represents a part of the L-ascorbic acid biosynthesis and oxidation pathway in tomato showing the role of DHAR.

FIG. 8 shows phylogenetic tree of DHAR polypeptides. H. vulgare_c62776255 (CHL), H. vulgare_gi_{—}21150952 (CYT), S. lycopersicum_TC196877 (CYT) and S. lycopersicum_AY971874 (CHL); CHL=chloroplasmatic; CYT=Cytosolic.

FIG. 9 represents the binary vector used for increased expression in Oryza sativa of a DHAR-encoding nucleic acid under the control of a rice GOS2 promoter (pGOS2).

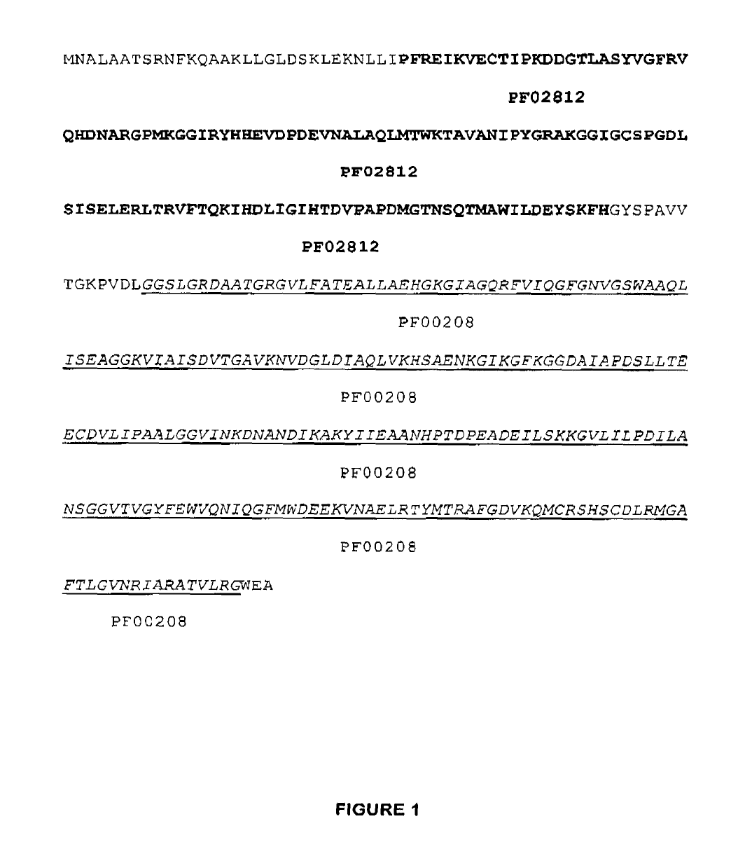

### EXAMPLES

The present invention will now be described with reference to the following examples, which are by way of illustration alone. The following examples are not intended to completely define or otherwise limit the scope of the invention.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### Example 1

### Identification of Sequences Related to the Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

### 1. Glutamate Dehydrogenase (GDH) Polypeptides

Table A1 provides a list of nucleic acid sequences related to the nucleic acid sequence used in the methods of the present invention.

TABLE A1
Examples of GDH polypeptides:

| | Nucleic acid | Polypeptide |
|---|---|---|
| Name | SEQ ID NO | SEQ ID NO |
| Z. mays AY106054#1 (beta) | 1 | 2 |
| A. thaliana AT5G07440.1#1 | 29 | 100 |
| B. napus AB066298#1 | 30 | 101 |
| C. solstitialis TA305 347529#1 | 31 | 102 |
| G. hirsutum TA29927 3635#1 | 32 | 103 |
| G. max TA64336 3847#1 | 33 | 104 |
| G. raimondii TA10049 29730#1 | 34 | 105 |
| G. raimondii TA10097 29730#1 | 35 | 106 |
| H. vulgare TA34363 4513#1 | 36 | 107 |
| M. truncatula CR931735 5.4#1 | 37 | 108 |
| O. sativa LOC Os04g45970.1#1 | 38 | 109 |
| O. sativa Os02g0650900 | 39 | 110 |
| P. canadensis TA199 3690#1 | 40 | 111 |
| P. taeda TA12661 3352#1 | 41 | 112 |
| P. tremula TA7375 113636#1 | 42 | 113 |
| P. trichocarpa 575509#1 | 43 | 114 |
| P. trichocarpa 828764#1 | 44 | 115 |
| S. bicolor 5286803#1 | 45 | 116 |
| S. lycopersicum TA48180 4081#1 | 46 | 117 |
| T. aestivum TA70276 4565#1 | 47 | 118 |
| V. vinifera TA36948 29760#1 | 48 | 119 |
| Z. mays TA160461 4577#1 | 49 | 120 |
| A. formosa x pubescens TA8521 338618#1 | 50 | 121 |
| A. officinalis TA1966 4686#1 | 51 | 122 |
| A. thaliana AT3G03910.1#1 | 52 | 123 |
| A. thaliana AT5G18170.1#1 | 53 | 124 |
| C. solstitialis TA153 347529#1 | 54 | 125 |
| G. max Gm0155x00045.1#1 | 55 | 126 |
| G. raimondii TA9810 29730#1 | 56 | 127 |
| H. annuus TA8643 4232#1 | 57 | 128 |
| H. argophyllus TA1274 73275#1 | 58 | 129 |
| H. vulgare TA35352 4513#1 | 59 | 130 |
| I. nil TA11677 35883#1 | 60 | 131 |
| M. crystallinum TA3379 3544#1 | 61 | 132 |
| M. truncatula AC174375 7.5#1 | 62 | 133 |
| M. truncatula TA21862 3880#1 | 63 | 134 |
| N. tabacum TA18464 4097#1 | 64 | 135 |
| O. basilicum TA2019 39350#1 | 65 | 136 |
| O. sativa Os03g0794500#1 | 66 | 137 |
| P. glauca TA14146 3330#1 | 67 | 138 |
| P. sitchensis TA13960 3332#1 | 68 | 139 |
| P. trichocarpa 571209#1 | 69 | 140 |
| P. trichocarpa 826140#1 | 70 | 141 |
| S. lycopersicum TA35879 4081#1 | 71 | 142 |
| S. tuberosum TA29537 4113#1 | 72 | 143 |
| T .aestivum TA69991 4565#1 | 73 | 144 |
| V. vinifera GSVIVT00025474001#1 | 74 | 145 |
| V. vinifera TA43933 29760#1 | 75 | 146 |
| M. polymorpha TA1057 3197#1 | 76 | 147 |
| P. patens 126976#1 | 77 | 148 |
| S. moellendorffii 78170#1 | 78 | 149 |
| A. thaliana AT1G51720.1#1 | 79 | 150 |
| Chlorella 25065#1 | 80 | 151 |
| Chlorella 52469#1 | 81 | 152 |
| E. huxleyi 69206#1 | 82 | 153 |
| G. max Gm0146x00171#1 | 83 | 154 |
| O. sativa AK107677#1 | 84 | 155 |
| O. sativa LOC Os01g37760.1#1 | 85 | 156 |
| P. patens 70453#1 | 86 | 157 |
| P. trichocarpa scaff 97.38#1 | 87 | 158 |
| P. tricornutum 13951#1 | 88 | 159 |
| S. bicolor 5282378#1 | 89 | 160 |
| S. moellendorffii 90033#1 | 90 | 161 |
| V. vinifera GSVIVT00034207001#1 | 91 | 162 |
| C. reinhardtii 82916#1 | 92 | 163 |
| Chlorella 31314#1 | 93 | 164 |
| Chlorella 34336#1 | 94 | 165 |
| V. carteri 63307#1 | 95 | 166 |
| V. carteri 65188#1 | 96 | 167 |
| P. patens 190253#1 | 97 | 168 |
| S. moellendorffii 402894#1 | 98 | 169 |
| B. napus BN06MC06056 | 99 | 170 |

In some instances, related sequences have tentatively been assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. On other instances, special nucleic acid sequence databases have been created for particular organisms, such as by the Joint Genome Institute. Further, access to proprietary databases, has allowed the identification of novel nucleic acid and polypeptide sequences.

### 2. Fasciclin-Like AGP (FLAB) Polypeptides

Table A2 provides a list of nucleic acid sequences related to SEQ ID NO: 171 and SEQ ID NO: 172.

TABLE A2
Examples of FLA-like nucleic acids and polypeptides:

| | | Poly- |
|---|---|---|
| | Nucleic | nucleo- |
| | acid | tide |
| | SEQ ID | SEQ ID |
| Name | NO: | NO: |
| L. esculemtum_25487; 6372; 12; 1247; 4081; 39#1 | 171 | 172 |
| A. cepa_TC4580#1 | 173 | 174 |
| A. thaliana_AT2G45470.1#1 | 175 | 176 |
| A. thaliana_AT2G04780.2#1 | 177 | 178 |
| A. thaliana_AT3G52370.1#1 | 179 | 180 |
| A. thaliana_AT1G03870.1#1 | 181 | 182 |
| A. thaliana_AT3G46550.1#1 | 183 | 184 |
| A. thaliana_AT4G31370.1#2 | 185 | 186 |
| A. thaliana_AT5G44130.1#1 | 187 | 188 |
| A. thaliana_AT3G11700.1#1 | 189 | 190 |
| A. thaliana_AT5G03170.1#1 | 191 | 192 |
| A. thaliana_AT5G06390.1#1 | 193 | 194 |
| A. thaliana_AT4G31370.1#1 | 195 | 196 |
| A. thaliana_AT4G12730.1#1 | 197 | 198 |
| A. thaliana_AT2G48140.1#1 | 199 | 200 |
| A. thaliana_AT1G28410.1#1 | 201 | 202 |
| A. thaliana_AT3G60900.1#1 | 203 | 204 |
| A. thaliana_AT3G12660.1#1 | 205 | 206 |
| A. thaliana_AT2G04780.1#1 | 207 | 208 |
| A. thaliana_AT5G60490.1#1 | 209 | 210 |
| A. thaliana_AT2G20520.1#1 | 211 | 212 |
| A. thaliana_AT5G55730.1#1 | 213 | 214 |
| A. thaliana_AT2G35860.1#1 | 215 | 216 |
| A. thaliana_AT2G24450.1#1 | 217 | 218 |
| B. napus_BN06MC20798_46793445@20729#1 | 219 | 220 |
| G. max_GM06MC34339_so40f04@33543#1 | 221 | 222 |
| H. vulgare_gi_24275592#1 | 223 | 224 |
| H. vulgare_gi_21973830#1 | 225 | 226 |
| H. vulgare_c62592154hv270303@7445#1 | 227 | 228 |
| M. truncatula_AC139344_22.4#1 | 229 | 230 |
| M. truncatula_AC141108_13.4#1 | 231 | 232 |
| M. truncatula_AC148446_20.4#1 | 233 | 234 |
| M. truncatula_AC183923_29.4#1 | 235 | 236 |
| M. truncatula_AC141110_2.5#1 | 237 | 238 |
| M. truncatula_AC183305_19.5#1 | 239 | 240 |
| M. truncatula_AC152407_24.4#1 | 241 | 242 |
| M. truncatula_CU062643_4.3#1 | 243 | 244 |
| M. truncatula_AC152407_10.4#1 | 245 | 246 |
| M. truncatula_AC128638_8.4#1 | 247 | 248 |
| M. truncatula_AC139344_27.4#1 | 249 | 250 |
| M. truncatula_AC152407_34.4#1 | 251 | 252 |
| M. truncatula_AC183923_12.4#1 | 253 | 254 |
| M. truncatula_AC150890_10.4#1 | 255 | 256 |
| M. truncatula_AC150890_2.4#1 | 257 | 258 |
| M. truncatula_AC141108_46.4#1 | 259 | 260 |
| M. truncatula_AC202334_12.4#1 | 261 | 262 |
| M. truncatula_AC141108_11.4#1 | 263 | 264 |
| M. truncatula_AC148655_16.4#1 | 265 | 266 |
| M. truncatula_AC140026_25.4#1 | 267 | 268 |
| M. truncatula_AC139344_13.4#1 | 269 | 270 |
| M. truncatula_AC150890_21.4#1 | 271 | 272 |
| M. truncatula_AC202325_14.4#1 | 273 | 274 |
| M. truncatula_AC144515_29.5#1 | 275 | 276 |
| M. truncatula_AC139344_21.4#1 | 277 | 278 |
| O. sativa_LOC_Os08g39270.1#1 | 279 | 280 |
| O. sativa_LOC_Os07g06680.1#1 | 281 | 282 |
| O. sativa_LOC_Os08g38270.1#1 | 283 | 284 |
| O. sativa_LOC_Os04g48490.1#1 | 285 | 286 |
| O. sativa_LOC_Os09g30010.1#1 | 287 | 288 |
| O. sativa_LOC_Os01g06580.1#1 | 289 | 290 |
| O. sativa_LOC_Os04g39590.1#1 | 291 | 292 |
| O. sativa_LOC_Os05g38500.1#1 | 293 | 294 |
| O. sativa_LOC_Os02g20540.1#1 | 295 | 296 |
| O. sativa_LOC_Os04g39600.1#1 | 297 | 298 |
| O. sativa_LOC_Os03g03600.1#1 | 299 | 300 |
| O. sativa_LOC_Os01g62380.2#1 | 301 | 302 |
| O. sativa_LOC_Os01g62380.1#1 | 303 | 304 |
| O. sativa_LOC_Os09g07350.1#1 | 305 | 306 |
| O. sativa_LOC_Os02g20560.1#1 | 307 | 308 |
| O. sativa_LOC_Os06g44660.1#1 | 309 | 310 |
| O. sativa_LOC_Os05g48890.1#1 | 311 | 312 |
| O. sativa_LOC_Os08g23180.1#1 | 313 | 314 |
| O. sativa_LOC_Os01g47780.1#1 | 315 | 316 |
| O. sativa_LOC_Os02g26290.1#1 | 317 | 318 |
| O. sativa_LOC_Os03g57460.1#1 | 319 | 320 |
| O. sativa_LOC_Os02g26320.1#1 | 321 | 322 |
| O. sativa_LOC_Os04g21570.1#1 | 323 | 324 |
| O. sativa_LOC_Os06g17460.1#1 | 325 | 326 |
| O. sativa_LOC_Os05g07060.1#1 | 327 | 328 |
| O. sativa_LOC_Os05g48900.1#1 | 329 | 330 |
| O. sativa_LOC_Os02g49420.1#1 | 331 | 332 |
| O. sativa_LOC_Os09g30486.1#1 | 333 | 334 |
| P. trichocarpa_scaff_XI.599#1 | 335 | 336 |
| P. trichocarpa_scaff_I.2651#1 | 337 | 338 |
| P. trichocarpa_scaff_XIV.1154#1 | 339 | 340 |
| P. trichocarpa_scaff_IX.1517#1 | 341 | 342 |
| P. trichocarpa_scaff_VI.1374#1 | 343 | 344 |
| P. trichocarpa_scaff_II.2021#1 | 345 | 346 |
| P. trichocarpa_scaff_I.170#1 | 347 | 348 |
| P. trichocarpa_scaff_147.53#1 | 349 | 350 |
| P. trichocarpa_scaff_XVI.813#1 | 351 | 352 |
| P. trichocarpa_scaff_XIV.1105#1 | 353 | 354 |
| P. trichocarpa_scaff_193.12#1 | 355 | 356 |
| S. lycopersicum_TC198578#1 | 357 | 358 |
| S. lycopersicum_TC199817#1 | 359 | 360 |
| S. lycopersicum_TC192348#1 | 361 | 362 |
| S. lycopersicum_TC204739#1 | 363 | 364 |
| S. lycopersicum_TC192457#1 | 365 | 366 |
| S. lycopersicum_TC215657#1 | 367 | 368 |
| 25227; 3269; 66; 1526; 4530; 39#1 | 369 | 370 |
| 958; 1671; 46; 1308; 3702; 39#1 | 371 | 372 |
| 951; 1678; 25; 1293; 3702; 39#1 | 373 | 374 |
| 955; 1674; 25; 1287; 3702; 39#1 | 375 | 376 |
| 2644; 0067; 85; 849; 3702; 32#1 | 377 | 378 |
| 24310; 6373; 108; 1313; 4530; 39#1 | 379 | 380 |
| 3260; 3252; 71; 1345; 3702; 39#1 | 381 | 382 |
| T. aestivum_NP9351276#1 | 383 | 384 |
| T. aestivum_TC305123#1 | 385 | 386 |
| T. aestivum_TC325646#1 | 387 | 388 |
| T. aestivum_TC344445#1 | 389 | 390 |
| T. aestivum_TC305090#1 | 391 | 392 |
| T. aestivum_TC299845#1 | 393 | 394 |
| T. aestivum_CK216481#1 | 395 | 396 |
| T. aestivum_CA627200#1 | 397 | 398 |
| T. aestivum_TC291284#1 | 399 | 400 |
| T. aestivum_TC312495#1 | 401 | 402 |
| T. aestivum_TC304072#1 | 403 | 404 |
| T. aestivum_TC277533#1 | 405 | 406 |
| T. aestivum_BQ245887#1 | 407 | 408 |
| T. aestivum_TC318484#1 | 409 | 410 |
| T. aestivum_DR740621#1 | 411 | 412 |
| T. aestivum_TC304082#1 | 413 | 414 |
| T. aestivum_TC293788#1 | 415 | 416 |
| T. aestivum_CV769415#1 | 417 | 418 |
| T. aestivum_NP9351269#1 | 419 | 420 |
| T. aestivum_CV762263#1 | 421 | 422 |
| T. aestivum_TC311105#1 | 423 | 424 |
| T. aestivum_TC292041#1 | 425 | 426 |
| T. aestivum_TC288810#1 | 427 | 428 |
| T. aestivum_TC296661#1 | 429 | 430 |
| T. aestivum_TC306434#1 | 431 | 432 |
| T. aestivum_TC278029#1 | 433 | 434 |
| T. aestivum_TC328684#1 | 435 | 436 |
| T. aestivum_TC296079#1 | 437 | 438 |
| T. aestivum_TC277708#1 | 439 | 440 |
| T. aestivum_TC298398#1 | 441 | 442 |
| T. aestivum_CV780845#1 | 443 | 444 |
| T. aestivum_TC278190#1 | 445 | 446 |
| T. aestivum_NP9351275#1 | 447 | 448 |
| T. aestivum_TC294615#1 | 449 | 450 |
| T. aestivum_CK212201#1 | 451 | 452 |
| T. aestivum_TC287761#1 | 453 | 454 |
| T. aestivum_NP9351277#1 | 455 | 456 |
| T. aestivum_TC321709#1 | 457 | 458 |
| T. aestivum_TC279125#1 | 459 | 460 |
| T. aestivum_TC277909#1 | 461 | 462 |
| T. aestivum_TC278001#1 | 463 | 464 |
| T. aestivum_NP9351274#1 | 465 | 466 |
| T. aestivum_TC330335#1 | 467 | 468 |
| Z. mays_ZM07MC23583_BFb0033M16@23517#1 | 469 | 470 |
| Z. mays_ZM07MC29916_BFb0139D12@29826#1 | 471 | 472 |
| Z. mays_ZM07MC14325_59158483@14291#1 | 473 | 474 |
| Z. mays_ZM07MC23082_BFb0380F07@23017#1 | 475 | 476 |
| Z. mays_ZM07MC29207_BFb0067H14@29117#1 | 477 | 478 |
| Z. mays_ZM07MC33226_BFb0305O24@33126#1 | 479 | 480 |
| Z. mays_ZM07MC33725_BFb0316M24@33623#1 | 481 | 482 |
| Z. mays_ZM07MC26383_BFb0121A09@26305#1 | 483 | 484 |
| Z. mays_ZM07MC32279_BFb0284P13@32182#1 | 485 | 486 |

Sequences have been tentatively assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. Special nucleic acid sequence databases have been created for particular organisms, such as by the Joint Genome Institute. Furthermore, access to proprietary databases, has allowed the identification of novel nucleic acid and polypeptide sequences.

### 3. Small Auxin-Up RNA (SAUR) Polypeptides

Table A3 provides a list of nucleic acid sequences related to SEQ ID NO: 501 and SEQ ID NO: 502.

TABLE A3
Examples of SAUR polypeptides:

| | Nucleic acid | Polypeptide |
|---|---|---|
| Name (AGI code or gene model) | SEQ ID NO: | SEQ ID NO: |
| AT2G21210 | 501 | 502 |
| AT1G75590.1 | 503 | 504 |
| AT1G75580.1 | 505 | 506 |
| AT1G29430.1 | 507 | 508 |
| AT1G29440.1 | 509 | 510 |
| AT1G29450.1 | 511 | 512 |
| AT1G29460.1 | 513 | 514 |
| AT1G29490.1 | 515 | 516 |
| AT1G29500.1 | 517 | 518 |
| AT1G29510.1 | 519 | 520 |
| AT1G29420.1 | 521 | 522 |
| AT1G43040.1 | 523 | 524 |
| AT1G16510.1 | 525 | 526 |
| AT1G20470.1 | 527 | 528 |
| AT1G19840.1 | 529 | 530 |
| AT2G16580.1 | 531 | 532 |
| AT2G21200.1 | 533 | 534 |
| AT2G36210.1 | 535 | 536 |
| AT2G45210.1 | 537 | 538 |
| AT2G42870.1 | 539 | 540 |
| AT2G37030.1 | 541 | 542 |
| AT2G18010.1 | 543 | 544 |
| AT2G24400.1 | 545 | 546 |
| AT2G46690.1 | 547 | 548 |
| AT3G03850.1 | 549 | 550 |
| AT3G03840.1 | 551 | 552 |
| AT3G03830.1 | 553 | 554 |
| AT3G03820.1 | 555 | 556 |
| AT3G61900.1 | 557 | 558 |
| AT3G51200.1 | 559 | 560 |
| AT3G43120.1 | 561 | 562 |
| AT3G09870.1 | 563 | 564 |
| AT3G20220.1 | 565 | 566 |
| AT3G12830.1 | 567 | 568 |
| AT3G58850.1 | 569 | 570 |
| AT3G60690.1 | 571 | 572 |
| AT3G53250.1 | 573 | 574 |
| AT4G34770.1 | 575 | 576 |
| AT4G34780.1 | 577 | 578 |
| AT4G34790.1 | 579 | 580 |
| AT4G34800.1 | 581 | 582 |
| AT4G34810.1 | 583 | 584 |
| AT4G13790.1 | 585 | 586 |
| AT4G31320.1 | 587 | 588 |
| AT4G09530.1 | 589 | 590 |
| AT4G00880.1 | 591 | 592 |
| AT4G36110.1 | 593 | 594 |
| AT5G03310.1 | 595 | 596 |
| AT5G18060.1 | 597 | 598 |
| AT5G18010.1 | 599 | 600 |
| AT5G18020.1 | 601 | 602 |
| AT5G18030.1 | 603 | 604 |
| AT5G18050.1 | 605 | 606 |
| AT5G42410.1 | 607 | 608 |
| AT5G50760.1 | 609 | 610 |
| AT5G53590.1 | 611 | 612 |
| AT5G27780.1 | 613 | 614 |
| AT5G20810.2 | 615 | 616 |
| AT5G20820.1 | 617 | 618 |
| AT1G72430.1 | 619 | 620 |
| AT1G76190.1 | 621 | 622 |
| AT1G79130.1 | 623 | 624 |
| AT1G19830.1 | 625 | 626 |
| AT1G56150.1 | 627 | 628 |
| AT2G21220.1 | 629 | 630 |
| AT4G22620.1 | 631 | 632 |
| AT4G12410.1 | 633 | 634 |
| AT4G34750.1 | 635 | 636 |
| AT4G34760.1 | 637 | 638 |
| AT4G38840.1 | 639 | 640 |
| AT4G38850.1 | 641 | 642 |
| AT4G38860.1 | 643 | 644 |
| AT5G66260.1 | 645 | 646 |
| AT5G18080.1 | 647 | 648 |
| AT5G10990.1 | 649 | 650 |
| AT2G28085.1 | 651 | 652 |
| AT3G12955.1 | 653 | 654 |
| AT3G03847.1 | 655 | 656 |
| AT1G17345.1 | 657 | 658 |
| AT5G20810.2 | 659 | 660 |
| AT4G38825.1 | 661 | 662 |
| A. formosa_TA10626_338618 | 663 | 664 |
| A. formosa_TA19524_338618 | 665 | 666 |
| A. hypogaea_EE125424 | 667 | 668 |
| A. hypogaea_EG029475 | 669 | 670 |
| A. majus_AJ787925 | 671 | 672 |
| A. majus_AJ789609 | 673 | 674 |
| A. majus_AJ792146 | 675 | 676 |
| A. majus_AJ793092 | 677 | 678 |
| A. stenosperma_EH042215 | 679 | 680 |
| A. stenosperma_EH042858 | 681 | 682 |
| A. stenosperma_EH044560 | 683 | 684 |
| B. vulgaris_EG549451 | 685 | 686 |
| B. vulgaris_EG551164 | 687 | 688 |
| C. annuum_AF492625 | 689 | 690 |
| C. annuum_AF492626 | 691 | 692 |
| C. annuum_AF492628 | 693 | 694 |
| C. endivia_EL370469 | 695 | 696 |
| C. endivia_EL370631 | 697 | 698 |
| C. endivia_TA1177_114280 | 699 | 700 |
| C. endivia_TA539_114280 | 701 | 702 |
| C. intybus_EH692435 | 703 | 704 |
| C. intybus_EH703830 | 705 | 706 |
| C. intybus_EH706838 | 707 | 708 |
| C. intybus_TA1426_13427 | 709 | 710 |
| C. maculosa_EH742386 | 711 | 712 |
| C. maculosa_EH742856 | 713 | 714 |
| C. maculosa_EH749319 | 715 | 716 |
| C. maculosa_TA4833_215693 | 717 | 718 |
| C. reticulata_TA893_85571 | 719 | 720 |
| C. tinctorius_EL383021 | 721 | 722 |
| C. tinctorius_EL402472 | 723 | 724 |
| C. tinctorius_EL404623 | 725 | 726 |
| C. tinctorius_EL409357 | 727 | 728 |
| C. tinctorius_EL410673 | 729 | 730 |
| C. tinctorius_EL511071 | 731 | 732 |
| F. vesca_DV439835 | 733 | 734 |
| F. vesca_DY671180 | 735 | 736 |
| F. vesca_EX674842 | 737 | 738 |
| F. vesca_TA11405_57918 | 739 | 740 |
| G. hirsutum_DW503615 | 741 | 742 |
| G. hirsutum_DW509807 | 743 | 744 |
| G. hirsutum_DW512758 | 745 | 746 |
| G. hirsutum_DW519665 | 747 | 748 |
| G. hirsutum_TA37257_3635 | 749 | 750 |
| G. hirsutum_TA38692_3635 | 751 | 752 |
| G. hirsutum_TA41018_3635 | 753 | 754 |
| G. hybrid_TA4305_18101 | 755 | 756 |
| G. max_AW597359 | 757 | 758 |
| G. max_BE347326 | 759 | 760 |
| G. max_BQ298199 | 761 | 762 |
| G. max_CD391803 | 763 | 764 |
| G. max_TA64893_3847 | 765 | 766 |
| G. max_TA67504_3847 | 767 | 768 |
| G. max_TA70411_3847 | 769 | 770 |
| G. raimondii_CO102462 | 771 | 772 |
| G. raimondii_CO116635 | 773 | 774 |
| H. annuus_DY910229 | 775 | 776 |
| H. annuus_TA11757_4232 | 777 | 778 |
| H. centranthoides_CB088060 | 779 | 780 |
| H. exilis_TA2077_400408 | 781 | 782 |
| H. exilis_TA3412_400408 | 783 | 784 |
| H. exilis_TA3635_400408 | 785 | 786 |
| H. paradoxus_EL483081 | 787 | 788 |
| H. paradoxus_EL491346 | 789 | 790 |
| H. paradoxus_TA5129_73304 | 791 | 792 |
| H. petiolaris_DY943735 | 793 | 794 |
| H. petiolaris_DY952438 | 795 | 796 |
| H. petiolaris_DY954041 | 797 | 798 |
| H. terminalis_CB076591 | 799 | 800 |
| H. tuberosus_EL457421 | 801 | 802 |
| H. tuberosus_EL463700 | 803 | 804 |
| H. tuberosus_EL465041 | 805 | 806 |
| H. tuberosus_EL468225 | 807 | 808 |
| H. tuberosus_TA4474_4233 | 809 | 810 |
| L. japonicus_AW719590 | 811 | 812 |
| L. japonicus_TA9854_34305 | 813 | 814 |
| L. perennis_DW080686 | 815 | 816 |
| L. perennis_DW081245 | 817 | 818 |
| L. perennis_DW083509 | 819 | 820 |
| L. perennis_TA3114_43195 | 821 | 822 |
| L. saligna_DW047772 | 823 | 824 |
| L. saligna_DW053071 | 825 | 826 |
| L. saligna_DW067950 | 827 | 828 |
| L. saligna_DW070649 | 829 | 830 |
| L. saligna_TA3313_75948 | 831 | 832 |
| L. sativa_DW127263 | 833 | 834 |
| L. sativa_DY980480 | 835 | 836 |
| L. sativa_TA10685_4236 | 837 | 838 |
| L. sativa_TA10743_4236 | 839 | 840 |
| L. sativa_TA4959_4236 | 841 | 842 |
| L. serriola_DW112451 | 843 | 844 |
| L. serriola_DW116661 | 845 | 846 |
| L. serriola_TA3320_75943 | 847 | 848 |
| L. serriola_TA4842_75943 | 849 | 850 |
| L. usitatissimus_8480 | 851 | 852 |
| L. virosa_DW153036 | 853 | 854 |
| L. virosa_DW155923 | 855 | 856 |
| L. virosa_DW174061 | 857 | 858 |
| L. virosa_TA2170_75947 | 859 | 860 |
| L. virosa_TA3812_75947 | 861 | 862 |
| M. domestica_CO067974 | 863 | 864 |
| M. domestica_CO756438 | 865 | 866 |
| M. domestica_CX023091 | 867 | 868 |
| M. domestica_TA37946_3750 | 869 | 870 |
| M. domestica_TA38953_3750 | 871 | 872 |
| M. domestica_TA39022_3750 | 873 | 874 |
| M. domestica_TA40409_3750 | 875 | 876 |
| M. domestica_TA44601_3750 | 877 | 878 |
| M. sativa_CO513303 | 879 | 880 |
| M. sativa_CO515084 | 881 | 882 |
| M. sativa_CO515320 | 883 | 884 |
| M. sativa_CO515889 | 885 | 886 |
| M. sativa_CO517303 | 887 | 888 |
| M. truncatula_BQ150674 | 889 | 890 |
| M. truncatula_BQ152587 | 891 | 892 |
| M. truncatula_BQ153482 | 893 | 894 |
| M. truncatula_BQ157435 | 895 | 896 |
| M. truncatula_TA25290_3880 | 897 | 898 |
| M. truncatula_TA35769_3880 | 899 | 900 |
| N. benthamiana_EH366121 | 901 | 902 |
| N. benthamiana_EH368884 | 903 | 904 |
| N. benthamiana_EH370704 | 905 | 906 |
| N. tabacum_BP137194 | 907 | 908 |
| N. tabacum_BP533711 | 909 | 910 |
| N. tabacum_DV158716 | 911 | 912 |
| N. tabacum_EB427417 | 913 | 914 |
| N. tabacum_EB439329 | 915 | 916 |
| N. tabacum_EB439347 | 917 | 918 |
| O. sativa_OsSAUR1 | 919 | 920 |
| O. sativa_OsSAUR10 | 921 | 922 |
| O. sativa_OsSAUR11 | 923 | 924 |
| O. sativa_OsSAUR12 | 925 | 926 |
| O. sativa_OsSAUR13 | 927 | 928 |
| O. sativa_OsSAUR14 | 929 | 930 |
| O. sativa_OsSAUR15 | 931 | 932 |
| O. sativa_OsSAUR16 | 933 | 934 |
| O. sativa_OsSAUR17 | 935 | 936 |
| O. sativa_OsSAUR18 | 937 | 938 |
| O. sativa_OsSAUR19 | 939 | 940 |
| O. sativa_OsSAUR2 | 941 | 942 |
| O. sativa_OsSAUR20 | 943 | 944 |
| O. sativa_OsSAUR21 | 945 | 946 |
| O. sativa_OsSAUR22 | 947 | 948 |
| O. sativa_OsSAUR23 | 949 | 950 |
| O. sativa_OsSAUR24 | 951 | 952 |
| O. sativa_OsSAUR25 | 953 | 954 |
| O. sativa_OsSAUR26 | 955 | 956 |
| O. sativa_OsSAUR27 | 957 | 958 |
| O. sativa_OsSAUR28 | 959 | 960 |
| O. sativa_OsSAUR29 | 961 | 962 |
| O. sativa_OsSAUR3 | 963 | 964 |
| O. sativa_OsSAUR30 | 965 | 966 |
| O. sativa_OsSAUR31 | 967 | 968 |
| O. sativa_OsSAUR32 | 969 | 970 |
| O. sativa_OsSAUR33 | 971 | 972 |
| O. sativa_OsSAUR34 | 973 | 974 |
| O. sativa_OsSAUR35 | 975 | 976 |
| O. sativa_OsSAUR36 | 977 | 978 |
| O. sativa_OsSAUR37 | 979 | 980 |
| O. sativa_OsSAUR38 | 981 | 982 |
| O. sativa_OsSAUR39 | 983 | 984 |
| O. sativa_OsSAUR4 | 985 | 986 |
| O. sativa_OsSAUR40 | 987 | 988 |
| O. sativa_OsSAUR41 | 989 | 990 |
| O. sativa_OsSAUR42 | 991 | 992 |
| O. sativa_OsSAUR44 | 993 | 994 |
| O. sativa_OsSAUR45 | 995 | 996 |
| O. sativa_OsSAUR46 | 997 | 998 |
| O. sativa_OsSAUR47 | 999 | 1000 |
| O. sativa_OsSAUR48 | 1001 | 1002 |
| O. sativa_OsSAUR49 | 1003 | 1004 |
| O. sativa_OsSAUR5 | 1005 | 1006 |
| O. sativa_OsSAUR51 | 1007 | 1008 |
| O. sativa_OsSAUR52 | 1009 | 1010 |
| O. sativa_OsSAUR53 | 1011 | 1012 |
| O. sativa_OsSAUR54 | 1013 | 1014 |
| O. sativa_OsSAUR55 | 1015 | 1016 |
| O. sativa_OsSAUR56 | 1017 | 1018 |
| O. sativa_OsSAUR57 | 1019 | 1020 |
| O. sativa_OsSAUR58 | 1021 | 1022 |
| O. sativa_OsSAUR6 | 1023 | 1024 |
| O. sativa_OsSAUR7 | 1025 | 1026 |
| O. sativa_OsSAUR8 | 1027 | 1028 |
| O. sativa_OsSAUR9 | 1029 | 1030 |
| P. armeniaca_CV047410 | 1031 | 1032 |
| P. armeniaca_TA4482_36596 | 1033 | 1034 |
| P. ginseng_DV554701 | 1035 | 1036 |
| P. persica_DY646333 | 1037 | 1038 |
| P. persica_TA3244_3760 | 1039 | 1040 |
| P. persica_TA3246_3760 | 1041 | 1042 |
| P. persica_TA8343_3760 | 1043 | 1044 |
| P. sativum_CD860263 | 1045 | 1046 |
| P. trichocarpa_scaff_IV.1273 | 1047 | 1048 |
| P. trichocarpa_scaff_IV.1274 | 1049 | 1050 |
| P. trichocarpa_scaff_IV.1275 | 1051 | 1052 |
| P. trichocarpa_scaff_IV.1277 | 1053 | 1054 |
| P. trichocarpa_scaff_IV.1278 | 1055 | 1056 |
| P. trichocarpa_scaff_IV.1283 | 1057 | 1058 |
| P. trichocarpa_scaff_IX.408 | 1059 | 1060 |
| P. trichocarpa_scaff_IX.410 | 1061 | 1062 |
| P. trichocarpa_scaff_IX.412 | 1063 | 1064 |
| P. trichocarpa_scaff_IX.414 | 1065 | 1066 |
| P. vulgaris_CV540653 | 1067 | 1068 |
| P. vulgaris_TA5174_3885 | 1069 | 1070 |
| P. vulgaris_TA5755_3885 | 1071 | 1072 |
| R. communis_EE260652 | 1073 | 1074 |
| R. communis_TA1427_3988 | 1075 | 1076 |
| R. communis_TA1793_3988 | 1077 | 1078 |
| R. communis_TA4032_3988 | 1079 | 1080 |
| R. communis_TA4537_3988 | 1081 | 1082 |
| S. lycopersicum_AI772038 | 1083 | 1084 |
| S. lycopersicum_BG133762 | 1085 | 1086 |
| S. lycopersicum_BI925420 | 1087 | 1088 |
| S. lycopersicum_BP891299 | 1089 | 1090 |
| S. lycopersicum_TA47646_4081 | 1091 | 1092 |
| S. lycopersicum_TA51451_4081 | 1093 | 1094 |
| S. lycopersicum_TA55498_4081 | 1095 | 1096 |
| S. rebaudiana_BG524629 | 1097 | 1098 |
| S. tuberosum_BQ506350 | 1099 | 1100 |
| S. tuberosum_CV469819 | 1101 | 1102 |
| S. tuberosum_CV497917 | 1103 | 1104 |
| S. tuberosum_CV500881 | 1105 | 1106 |
| S. tuberosum_CV506321 | 1107 | 1108 |
| S. tuberosum_DN921670 | 1109 | 1110 |
| S. tuberosum_TA40200_4113 | 1111 | 1112 |
| T. erecta_1962 | 1113 | 1114 |
| T. erecta_3566 | 1115 | 1116 |
| T. erecta_6288 | 1117 | 1118 |
| T. erecta_684 | 1119 | 1120 |
| T. halophila_EC599448 | 1121 | 1122 |
| T. halophila_EC599895 | 1123 | 1124 |
| T. kok-saghyz_DR398853 | 1125 | 1126 |
| T. kok-saghyz_DR399601 | 1127 | 1128 |
| T. kok-saghyz_DR400973 | 1129 | 1130 |
| T. kok-saghyz_DR402050 | 1131 | 1132 |
| T. kok-saghyz_TA924_333970 | 1133 | 1134 |
| V. vinifera_EC943632 | 1135 | 1136 |
| V. vinifera_EC944862 | 1137 | 1138 |
| V. vinifera_EC955742 | 1139 | 1140 |
| V. vinifera_EC990676 | 1141 | 1142 |
| V. vinifera_EE085076 | 1143 | 1144 |
| V. vinifera_TA50539_29760 | 1145 | 1146 |
| V. vinifera_TA51081_29760 | 1147 | 1148 |
| V. vinifera_TA53202_29760 | 1149 | 1150 |

(i) Examples of SAUR polypeptides:

| | Nucleic | Poly- |
|---|---|---|
| | acid | peptide |
| | SEQ | SEQ |
| Name | ID NO: | ID NO: |
| O. sativa_TC318156#1 | 2210 | 2211 |
| A. thaliana_AT1G17345.1#1 | 2212 | 2213 |
| A. thaliana_AT1G72430.1#1 | 2214 | 2215 |
| A. thaliana_AT3G12955.1#1 | 2216 | 2217 |
| A. thaliana_AT5G20820.1#1 | 2218 | 2219 |
| G. max_Glyma07g04850.1#1 | 2220 | 2221 |
| G. max_Glyma07g08130.1#1 | 2222 | 2223 |
| G. max_Glyma08g25650.1#1 | 2224 | 2225 |
| G. max_Glyma09g08490.1#1 | 2226 | 2227 |
| G. max_Glyma13g17400.1#1 | 2228 | 2229 |
| G. max_Glyma16g01430.1#1 | 2230 | 2231 |
| G. max_Glyma17g05090.1#1 | 2232 | 2233 |
| M. truncatula_AC124967_17.5#1 | 2234 | 2235 |
| M. truncatula_AC152424_21.4#1 | 2236 | 2237 |
| M. truncatula_AC152424_22.4#1 | 2238 | 2239 |
| M. truncatula_AC186199_6.5#1 | 2240 | 2241 |
| O. sativa_TC322544#1 | 2242 | 2243 |
| O. sativa_TC333315#1 | 2244 | 2245 |
| P. patens_TC33761#1 | 2246 | 2247 |
| P. patens_TC42583#1 | 2248 | 2249 |
| P. patens_TC44119#1 | 2250 | 2251 |
| P. patens_TC51099#1 | 2252 | 2253 |
| P. trichocarpa_scaff_158.41#1 | 2254 | 2255 |
| P. trichocarpa_scaff_28.75#1 | 2256 | 2257 |
| P. trichocarpa_scaff_I.2982#1 | 2258 | 2259 |
| P. trichocarpa_scaff_III.441#1 | 2260 | 2261 |
| P. trichocarpa_scaff_XI.1199#1 | 2262 | 2263 |
| S. lycopersicum_ES895607#1 | 2264 | 2265 |
| S. lycopersicum_TC204920#1 | 2266 | 2267 |
| TM-C_23062; C_; #1 | 2268 | 2269 |
| TM-C_24207; C_; PL; O.#1 | 2270 | 2271 |
| TM-C_25713; C_; #1 | 2272 | 2273 |
| TM-C_26635; C_; GL; A.#1 | 2274 | 2275 |
| TM-C_49318; C_; GL; A.#1 | 2276 | 2277 |
| Z. mays_ZM07MC00881_57442342@880#1 | 2278 | 2279 |
| Z. mays_ZM07MSbpsHQ_59325429.r01@42013#1 | 2280 | 2281 |
| Z. mays_ZM07MSbpsHQ_65311827.f01@48806#1 | 2282 | 2283 |

Sequences have been tentatively assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. Special nucleic acid sequence databases have been created for particular organisms, such as by the Joint Genome Institute. Furthermore, access to proprietary databases, has allowed the identification of novel nucleic acid and polypeptide sequences.

### 4. SAUR Polypeptides, or SYNP Polypeptides, or Fusion Proteins of SAUR Polypeptides and SYNP Polypeptides

Table A4 provides a list of nucleic acid sequences related to SEQ ID NO: 1163 and SEQ ID NO: 1164.

TABLE A4
Examples of SAUR polypeptides:

| | Nucleic acid | Polypeptide |
|---|---|---|
| Name (AGI code or gene model) | SEQ ID NO: | SEQ ID NO: |
| AT2G21210 | 1163 | 1164 |
| AT1G75590.1 | 1165 | 1166 |
| AT1G75580.1 | 1167 | 1168 |
| AT1G29430.1 | 1169 | 1170 |
| AT1G29440.1 | 1171 | 1172 |
| AT1G29450.1 | 1173 | 1174 |
| AT1G29460.1 | 1175 | 1176 |
| AT1G29490.1 | 1177 | 1178 |
| AT1G29500.1 | 1179 | 1180 |
| AT1G29510.1 | 1181 | 1182 |
| AT1G29420.1 | 1183 | 1184 |
| AT1G43040.1 | 1185 | 1186 |
| AT1G16510.1 | 1187 | 1188 |
| AT1G20470.1 | 1189 | 1190 |
| AT1G19840.1 | 1191 | 1192 |
| AT2G16580.1 | 1193 | 1194 |
| AT2G21200.1 | 1195 | 1196 |
| AT2G36210.1 | 1197 | 1198 |
| AT2G45210.1 | 1199 | 1200 |
| AT2G42870.1 | 1201 | 1202 |
| AT2G37030.1 | 1203 | 1204 |
| AT2G18010.1 | 1205 | 1206 |
| AT2G24400.1 | 1207 | 1208 |
| AT2G46690.1 | 1209 | 1210 |
| AT3G03850.1 | 1211 | 1212 |
| AT3G03840.1 | 1213 | 1214 |
| AT3G03830.1 | 1215 | 1216 |
| AT3G03820.1 | 1217 | 1218 |
| AT3G61900.1 | 1219 | 1220 |
| AT3G51200.1 | 1221 | 1222 |
| AT3G43120.1 | 1223 | 1224 |
| AT3G09870.1 | 1225 | 1226 |
| AT3G20220.1 | 1227 | 1228 |
| AT3G12830.1 | 1229 | 1230 |
| AT3G58850.1 | 1231 | 1232 |
| AT3G60690.1 | 1233 | 1234 |
| AT3G53250.1 | 1235 | 1236 |
| AT4G34770.1 | 1237 | 1238 |
| AT4G34780.1 | 1239 | 1240 |
| AT4G34790.1 | 1241 | 1242 |
| AT4G34800.1 | 1243 | 1244 |
| AT4G34810.1 | 1245 | 1246 |
| AT4G13790.1 | 1247 | 1248 |
| AT4G31320.1 | 1249 | 1250 |
| AT4G09530.1 | 1251 | 1252 |
| AT4G00880.1 | 1253 | 1254 |
| AT4G36110.1 | 1255 | 1256 |
| AT5G03310.1 | 1257 | 1258 |
| AT5G18060.1 | 1259 | 1260 |
| AT5G18010.1 | 1261 | 1262 |
| AT5G18020.1 | 1263 | 1264 |
| AT5G18030.1 | 1265 | 1266 |
| AT5G18050.1 | 1267 | 1268 |
| AT5G42410.1 | 1269 | 1270 |
| AT5G50760.1 | 1271 | 1272 |
| AT5G53590.1 | 1273 | 1274 |
| AT5G27780.1 | 1275 | 1276 |
| AT5G20810.2 | 1277 | 1278 |
| AT5G20820.1 | 1279 | 1280 |
| AT1G72430.1 | 1281 | 1282 |
| AT1G76190.1 | 1283 | 1284 |
| AT1G79130.1 | 1285 | 1286 |
| AT1G19830.1 | 1287 | 1288 |
| AT1G56150.1 | 1289 | 1290 |
| AT2G21220.1 | 1291 | 1292 |
| AT4G22620.1 | 1293 | 1294 |
| AT4G12410.1 | 1295 | 1296 |
| AT4G34750.1 | 1297 | 1298 |
| AT4G34760.1 | 1299 | 1300 |
| AT4G38840.1 | 1301 | 1302 |
| AT4G38850.1 | 1303 | 1304 |
| AT4G38860.1 | 1305 | 1306 |
| AT5G66260.1 | 1307 | 1308 |
| AT5G18080.1 | 1309 | 1310 |
| AT5G10990.1 | 1311 | 1312 |
| AT2G28085.1 | 1313 | 1314 |
| AT3G12955.1 | 1315 | 1316 |
| AT3G03847.1 | 1317 | 1318 |
| AT1G17345.1 | 1319 | 1320 |
| AT5G20810.2 | 1321 | 1322 |
| AT4G38825.1 | 1323 | 1324 |
| A. formosa_TA10626_338618 | 1325 | 1326 |
| A. formosa_TA19524_338618 | 1327 | 1328 |
| A. hypogaea_EE125424 | 1329 | 1330 |
| A. hypogaea_EG029475 | 1331 | 1332 |
| A. majus_AJ787925 | 1333 | 1334 |
| A. majus_AJ789609 | 1335 | 1336 |
| A. majus_AJ792146 | 1337 | 1338 |
| A. majus_AJ793092 | 1339 | 1340 |
| A. stenosperma_EH042215 | 1341 | 1342 |
| A. stenosperma_EH042858 | 1343 | 1344 |
| A. stenosperma_EH044560 | 1345 | 1346 |
| B. vulgaris_EG549451 | 1347 | 1348 |
| B. vulgaris_EG551164 | 1349 | 1350 |
| C. annuum_AF492625 | 1351 | 1352 |
| C. annuum_AF492626 | 1353 | 1354 |
| C. annuum_AF492628 | 1355 | 1356 |
| C. endivia_EL370469 | 1357 | 1358 |
| C. endivia_EL370631 | 1359 | 1360 |
| C. endivia_TA1177_114280 | 1361 | 1362 |
| C. endivia_TA539_114280 | 1363 | 1364 |
| C. intybus_EH692435 | 1365 | 1366 |
| C. intybus_EH703830 | 1367 | 1368 |
| C. intybus_EH706838 | 1369 | 1370 |
| C. intybus_TA1426_13427 | 1371 | 1372 |
| C. maculosa_EH742386 | 1373 | 1374 |
| C. maculosa_EH742856 | 1375 | 1376 |
| C. maculosa_EH749319 | 1377 | 1378 |
| C. maculosa_TA4833_215693 | 1379 | 1380 |
| C. reticulata_TA893_85571 | 1381 | 1382 |
| C. tinctorius_EL383021 | 1383 | 1384 |
| C. tinctorius_EL402472 | 1385 | 1386 |
| C. tinctorius_EL404623 | 1387 | 1388 |
| C. tinctorius_EL409357 | 1389 | 1390 |
| C. tinctorius_EL410673 | 1391 | 1392 |
| C. tinctorius_EL511071 | 1393 | 1394 |
| F. vesca_DV439835 | 1395 | 1396 |
| F. vesca_DY671180 | 1397 | 1398 |
| F. vesca_EX674842 | 1399 | 1400 |
| F. vesca_TA11405_57918 | 1401 | 1402 |
| G. hirsutum_DW503615 | 1403 | 1404 |
| G. hirsutum_DW509807 | 1405 | 1406 |
| G. hirsutum_DW512758 | 1407 | 1408 |
| G. hirsutum_DW519665 | 1409 | 1410 |
| G. hirsutum_TA37257_3635 | 1411 | 1412 |
| G. hirsutum_TA38692_3635 | 1413 | 1414 |
| G. hirsutum_TA41018_3635 | 1415 | 1416 |
| G. hybrid_TA4305_18101 | 1417 | 1418 |
| G. max_AW597359 | 1419 | 1420 |
| G. max_BE347326 | 1421 | 1422 |
| G. max_BQ298199 | 1423 | 1424 |
| G. max_CD391803 | 1425 | 1426 |
| G. max_TA64893_3847 | 1427 | 1428 |
| G. max_TA67504_3847 | 1429 | 1430 |
| G. max_TA70411_3847 | 1431 | 1432 |
| G. raimondii_CO102462 | 1433 | 1434 |
| G. raimondii_CO116635 | 1435 | 1436 |
| H. annuus_DY910229 | 1437 | 1438 |
| H. annuus_TA11757_4232 | 1439 | 1440 |
| H. centranthoides_CB088060 | 1441 | 1442 |
| H. exilis_TA2077_400408 | 1443 | 1444 |
| H. exilis_TA3412_400408 | 1445 | 1446 |
| H. exilis_TA3635_400408 | 1447 | 1448 |
| H. paradoxus_EL483081 | 1449 | 1450 |
| H. paradoxus_EL491346 | 1451 | 1452 |
| H. paradoxus_TA5129_73304 | 1453 | 1454 |
| H. petiolaris_DY943735 | 1455 | 1456 |
| H. petiolaris_DY952438 | 1457 | 1458 |
| H. petiolaris_DY954041 | 1459 | 1460 |
| H. terminalis_CB076591 | 1461 | 1462 |
| H. tuberosus_EL457421 | 1463 | 1464 |
| H. tuberosus_EL463700 | 1465 | 1466 |
| H. tuberosus_EL465041 | 1467 | 1468 |
| H. tuberosus_EL468225 | 1469 | 1470 |
| H. tuberosus_TA4474_4233 | 1471 | 1472 |
| L. japonicus_AW719590 | 1473 | 1474 |
| L. japonicus_TA9854_34305 | 1475 | 1476 |
| L. perennis_DW080686 | 1477 | 1478 |
| L. perennis_DW081245 | 1479 | 1480 |
| L. perennis_DW083509 | 1481 | 1482 |
| L. perennis_TA3114_43195 | 1483 | 1484 |
| L. saligna_DW047772 | 1485 | 1486 |
| L. saligna_DW053071 | 1487 | 1488 |
| L. saligna_DW067950 | 1489 | 1490 |
| L. saligna_DW070649 | 1491 | 1492 |
| L. saligna_TA3313_75948 | 1493 | 1494 |
| L. sativa_DW127263 | 1495 | 1496 |
| L. sativa_DY980480 | 1497 | 1498 |
| L. sativa_TA10685_4236 | 1499 | 1500 |
| L. sativa_TA10743_4236 | 1501 | 1502 |
| L. sativa_TA4959_4236 | 1503 | 1504 |
| L. serriola_DW112451 | 1505 | 1506 |
| L. serriola_DW116661 | 1507 | 1508 |
| L. serriola_TA3320_75943 | 1509 | 1510 |
| L. serriola_TA4842_75943 | 1511 | 1512 |
| L. usitatissimus_8480 | 1513 | 1514 |
| L. virosa_DW153036 | 1515 | 1516 |
| L. virosa_DW155923 | 1517 | 1518 |
| L. virosa_DW174061 | 1519 | 1520 |
| L. virosa_TA2170_75947 | 1521 | 1522 |
| L. virosa_TA3812_75947 | 1523 | 1524 |
| M. domestica_CO067974 | 1525 | 1526 |
| M. domestica_CO756438 | 1527 | 1528 |
| M. domestica_CX023091 | 1529 | 1530 |
| M. domestica_TA37946_3750 | 1531 | 1532 |
| M. domestica_TA38953_3750 | 1533 | 1534 |
| M. domestica_TA39022_3750 | 1535 | 1536 |
| M. domestica_TA40409_3750 | 1537 | 1538 |
| M. domestica_TA44601_3750 | 1539 | 1540 |
| M. sativa_CO513303 | 1541 | 1542 |
| M. sativa_CO515084 | 1543 | 1544 |
| M. sativa_CO515320 | 1545 | 1546 |
| M. sativa_CO515889 | 1547 | 1548 |
| M. sativa_CO517303 | 1549 | 1550 |
| M. truncatula_BQ150674 | 1551 | 1552 |
| M. truncatula_BQ152587 | 1553 | 1554 |
| M. truncatula_BQ153482 | 1555 | 1556 |
| M. truncatula_BQ157435 | 1557 | 1558 |
| M. truncatula_TA25290_3880 | 1559 | 1560 |
| M. truncatula_TA35769_3880 | 1561 | 1562 |
| N. benthamiana_EH366121 | 1563 | 1564 |
| N. benthamiana_EH368884 | 1565 | 1566 |
| N. benthamiana_EH370704 | 1567 | 1568 |
| N. tabacum_BP137194 | 1569 | 1570 |
| N. tabacum_BP533711 | 1571 | 1572 |
| N. tabacum_DV158716 | 1573 | 1574 |
| N. tabacum_EB427417 | 1575 | 1576 |
| N. tabacum_EB439329 | 1577 | 1578 |
| N. tabacum_EB439347 | 1579 | 1580 |
| O. sativa_OsSAUR1 | 1581 | 1582 |
| O. sativa_OsSAUR10 | 1583 | 1584 |
| O. sativa_OsSAUR11 | 1585 | 1586 |
| O. sativa_OsSAUR12 | 1587 | 1588 |
| O. sativa_OsSAUR13 | 1589 | 1590 |
| O. sativa_OsSAUR14 | 1591 | 1592 |
| O. sativa_OsSAUR15 | 1593 | 1594 |
| O. sativa_OsSAUR16 | 1595 | 1596 |
| O. sativa_OsSAUR17 | 1597 | 1598 |
| O. sativa_OsSAUR18 | 1599 | 1600 |
| O. sativa_OsSAUR19 | 1601 | 1602 |
| O. sativa_OsSAUR2 | 1603 | 1604 |
| O. sativa_OsSAUR20 | 1605 | 1606 |
| O. sativa_OsSAUR21 | 1607 | 1608 |
| O. sativa_OsSAUR22 | 1609 | 1610 |
| O. sativa_OsSAUR23 | 1611 | 1612 |
| O. sativa_OsSAUR24 | 1613 | 1614 |
| O. sativa_OsSAUR25 | 1615 | 1616 |
| O. sativa_OsSAUR26 | 1617 | 1618 |
| O. sativa_OsSAUR27 | 1619 | 1620 |
| O. sativa_OsSAUR28 | 1621 | 1622 |
| O. sativa_OsSAUR29 | 1623 | 1624 |
| O. sativa_OsSAUR3 | 1625 | 1626 |
| O. sativa_OsSAUR30 | 1627 | 1628 |
| O. sativa_OsSAUR31 | 1629 | 1630 |
| O. sativa_OsSAUR32 | 1631 | 1632 |
| O. sativa_OsSAUR33 | 1633 | 1634 |
| O. sativa_OsSAUR34 | 1635 | 1636 |
| O. sativa_OsSAUR35 | 1637 | 1638 |
| O. sativa_OsSAUR36 | 1639 | 1640 |
| O. sativa_OsSAUR37 | 1641 | 1642 |
| O. sativa_OsSAUR38 | 1643 | 1644 |
| O. sativa_OsSAUR39 | 1645 | 1646 |
| O. sativa_OsSAUR4 | 1647 | 1648 |
| O. sativa_OsSAUR40 | 1649 | 1650 |
| O. sativa_OsSAUR41 | 1651 | 1652 |
| O. sativa_OsSAUR42 | 1653 | 1654 |
| O. sativa_OsSAUR44 | 1655 | 1656 |
| O. sativa_OsSAUR45 | 1657 | 1658 |
| O. sativa_OsSAUR46 | 1659 | 1660 |
| O. sativa_OsSAUR47 | 1661 | 1662 |
| O. sativa_OsSAUR48 | 1663 | 1664 |
| O. sativa_OsSAUR49 | 1665 | 1666 |
| O. sativa_OsSAUR5 | 1667 | 1668 |
| O. sativa_OsSAUR51 | 1669 | 1670 |
| O. sativa_OsSAUR52 | 1671 | 1672 |
| O. sativa_OsSAUR53 | 1673 | 1674 |
| O. sativa_OsSAUR54 | 1675 | 1676 |
| O. sativa_OsSAUR55 | 1677 | 1678 |
| O. sativa_OsSAUR56 | 1679 | 1680 |
| O. sativa_OsSAUR57 | 1681 | 1682 |
| O. sativa_OsSAUR58 | 1683 | 1684 |
| O. sativa_OsSAUR6 | 1685 | 1686 |
| O. sativa_OsSAUR7 | 1687 | 1688 |
| O. sativa_OsSAUR8 | 1689 | 1690 |
| O. sativa_OsSAUR9 | 1691 | 1692 |
| P. armeniaca_CV047410 | 1693 | 1694 |
| P. armeniaca_TA4482_36596 | 1695 | 1696 |
| P. ginseng_DV554701 | 1697 | 1698 |
| P. persica_DY646333 | 1699 | 1700 |
| P. persica_TA3244_3760 | 1701 | 1702 |
| P. persica_TA3246_3760 | 1703 | 1704 |
| P. persica_TA8343_3760 | 1705 | 1706 |
| P. sativum_CD860263 | 1707 | 1708 |
| P. trichocarpa_scaff_IV.1273 | 1709 | 1710 |
| P. trichocarpa_scaff_IV.1274 | 1711 | 1712 |
| P. trichocarpa_scaff_IV.1275 | 1713 | 1714 |
| P. trichocarpa_scaff_IV.1277 | 1715 | 1716 |
| P. trichocarpa_scaff_IV.1278 | 1717 | 1718 |
| P. trichocarpa_scaff_IV.1283 | 1719 | 1720 |
| P. trichocarpa_scaff_IX.408 | 1721 | 1722 |
| P. trichocarpa_scaff_IX.410 | 1723 | 1724 |
| P. trichocarpa_scaff_IX.412 | 1725 | 1726 |
| P. trichocarpa_scaff_IX.414 | 1727 | 1728 |
| P. vulgaris_CV540653 | 1729 | 1730 |
| P. vulgaris_TA5174_3885 | 1731 | 1732 |
| P. vulgaris_TA5755_3885 | 1733 | 1734 |
| R. communis_EE260652 | 1735 | 1736 |
| R. communis_TA1427_3988 | 1737 | 1738 |
| R. communis_TA1793_3988 | 1739 | 1740 |
| R. communis_TA4032_3988 | 1741 | 1742 |
| R. communis_TA4537_3988 | 1743 | 1744 |
| S. lycopersicum_AI772038 | 1745 | 1746 |
| S. lycopersicum_BG133762 | 1747 | 1748 |
| S. lycopersicum_BI925420 | 1749 | 1750 |
| S. lycopersicum_BP891299 | 1751 | 1752 |
| S. lycopersicum_TA47646_4081 | 1753 | 1754 |
| S. lycopersicum_TA51451_4081 | 1755 | 1756 |
| S. lycopersicum_TA55498_4081 | 1757 | 1758 |
| S. rebaudiana_BG524629 | 1759 | 1760 |
| S. tuberosum_BQ506350 | 1761 | 1762 |
| S. tuberosum_CV469819 | 1763 | 1764 |
| S. tuberosum_CV497917 | 1765 | 1766 |
| S. tuberosum_CV500881 | 1767 | 1768 |
| S. tuberosum_CV506321 | 1769 | 1770 |
| S. tuberosum_DN921670 | 1771 | 1772 |
| S. tuberosum_TA40200_4113 | 1773 | 1774 |
| T. erecta_1962 | 1775 | 1776 |
| T. erecta_3566 | 1777 | 1778 |
| T. erecta_6288 | 1779 | 1780 |
| T. erecta_684 | 1781 | 1782 |
| T. halophila_EC599448 | 1783 | 1784 |
| T. halophila_EC599895 | 1785 | 1786 |
| T. kok-saghyz_DR398853 | 1787 | 1788 |
| T. kok-saghyz_DR399601 | 1789 | 1790 |
| T. kok-saghyz_DR400973 | 1791 | 1792 |
| T. kok-saghyz_DR402050 | 1793 | 1794 |
| T. kok-saghyz_TA924_333970 | 1795 | 1796 |
| V. vinifera_EC943632 | 1797 | 1798 |
| V. vinifera_EC944862 | 1799 | 1800 |
| V. vinifera_EC955742 | 1801 | 1802 |
| V. vinifera_EC990676 | 1803 | 1804 |
| V. vinifera_EE085076 | 1805 | 1806 |
| V. vinifera_TA50539_29760 | 1807 | 1808 |
| V. vinifera_TA51081_29760 | 1809 | 1810 |
| V. vinifera_TA53202_29760 | 1811 | 1812 |

Sequences have been tentatively assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. Special nucleic acid sequence databases have been created for particular organisms, such as by the Joint Genome Institute. Furthermore, access to proprietary databases, has allowed the identification of novel nucleic acid and polypeptide sequences.

### 5. Dehydroascorbate Reductase (DHAR) Polypeptides

Table A5 provides a list of nucleic acid sequences related to SEQ ID NO 1957 and SEQ ID NO 1958.

TABLE A5
Examples of DHAR nucleic acids and polypeptides:

| | Nucleic acid | Polypeptide |
|---|---|---|
| Name | SEQ ID NO: | SEQ ID NO: |
| A. majus_TA5689_4151 | 1957 | 1958 |
| A. thaliana_AT5G16710.1 | 1959 | 1960 |
| Aquilegia_sp_TC22354 | 1961 | 1962 |
| B. juncea_AF536329 | 1963 | 1964 |
| B. napus_TC90979 | 1965 | 1966 |
| B. oleracea_AB125638 | 1967 | 1968 |
| B. vulgaris_TA8095_161934 | 1969 | 1970 |
| C. canephora_TC4074 | 1971 | 1972 |
| C. maculosa_TA559_215693 | 1973 | 1974 |
| C. sinensis_TC723 | 1975 | 1976 |
| C. solstitialis_TA2280_347529 | 1977 | 1978 |
| C. tinctorius_TA1280_4222 | 1979 | 1980 |
| G. hirsutum_TC83927 | 1981 | 1982 |
| G. max_Glyma11g33700.1 | 1983 | 1984 |
| G. max_Glyma18g04510.1 | 1985 | 1986 |
| G. raimondii_TC4866 | 1987 | 1988 |
| H. annuus_TC33319 | 1989 | 1990 |
| H. ciliaris_TA2767_73280 | 1991 | 1992 |
| H. tuberosus_TA3283_4233 | 1993 | 1994 |
| H. vulgare_c62776255 | 1995 | 1996 |
| I. nil_TC1722 | 1997 | 1998 |
| L. japonicus_TC31996 | 1999 | 2000 |
| L. virosa_TA4153_75947 | 2001 | 2002 |
| M. domestica_TC557 | 2003 | 2004 |
| M. truncatula_DQ006811 | 2005 | 2006 |
| N. tabacum_TC17223 | 2007 | 2008 |
| O. sativa_LOC_Os06g12630.1 | 2009 | 2010 |
| P. trichocarpa_EF146363 | 2011 | 2012 |
| P. trifoliata_CV707148 | 2013 | 2014 |
| R. communis_TA3568_3988 | 2015 | 2016 |
| S. bicolor_Sb10g008310.1 | 2017 | 2018 |
| S. lycopersicum_AY971874 | 2019 | 2020 |
| S. oleracea_AF195783 | 2021 | 2022 |
| S. tuberosum_TC165600 | 2023 | 2024 |
| T. aestivum_TC328219 | 2025 | 2026 |
| Triphysaria_sp_TC13209 | 2027 | 2028 |
| Triphysaria_sp_TC4796 | 2029 | 2030 |
| V. vinifera_GSVIVT00032988001 | 2031 | 2032 |
| Z. mays_TC441527 | 2033 | 2034 |
| A. capillaris_DV853556 | 2035 | 2036 |
| A. thaliana_AT1G19570.1 | 2037 | 2038 |
| A. thaliana_AT1G75270.1 | 2039 | 2040 |
| A. thaliana_AT5G36270.1 | 2041 | 2042 |
| Aquilegia_sp_TC25677 | 2043 | 2044 |
| B. napus_TC63930 | 2045 | 2046 |
| B. napus_TC66670 | 2047 | 2048 |
| B. oleracea_TA5797_3712 | 2049 | 2050 |
| B. rapa_TA5347_3711 | 2051 | 2052 |
| C. annuum_TC8154 | 2053 | 2054 |
| C. aurantium_TA900_43166 | 2055 | 2056 |
| C. canephora_DV706668 | 2057 | 2058 |
| C. clementina_DY270995 | 2059 | 2060 |
| C. intybus_TA3336_13427 | 2061 | 2062 |
| C. longa_TA1900_136217 | 2063 | 2064 |
| C. maculosa_TA1265_215693 | 2065 | 2066 |
| C. reticulata_TA1023_85571 | 2067 | 2068 |
| C. sinensis_EY698722 | 2069 | 2070 |
| C. solstitialis_EH753758 | 2071 | 2072 |
| C. tetragonoloba_TA307_3832 | 2073 | 2074 |
| C. tinctorius_TA1301_4222 | 2075 | 2076 |
| D. sophia_BU238196 | 2077 | 2078 |
| E. coracana_TA104_4511 | 2079 | 2080 |
| E. esula_TC2212 | 2081 | 2082 |
| F. arundinacea_TC6317 | 2083 | 2084 |
| F. vesca_TA9444_57918 | 2085 | 2086 |
| G. arboreum_TA6227_29729 | 2087 | 2088 |
| G. hirsutum_TC94056 | 2089 | 2090 |
| G. max_Glyma10g43730.1 | 2091 | 2092 |
| G. max_Glyma20g38440.1 | 2093 | 2094 |
| G. raimondii_TC7963 | 2095 | 2096 |
| H. annuus_TC29862 | 2097 | 2098 |
| H. ciliaris_TA629_73280 | 2099 | 2100 |
| H. exilis_TA561_400408 | 2101 | 2102 |
| H. paradoxus_EL479335 | 2103 | 2104 |
| H. tuberosus_TA3353_4233 | 2105 | 2106 |
| H. vulgare_gi_21150952 | 2107 | 2108 |
| I. nil_TC10530 | 2109 | 2110 |
| L. japonicus_TC24095 | 2111 | 2112 |
| L. perennis_TA1555_43195 | 2113 | 2114 |
| L. saligna_TA1997_75948 | 2115 | 2116 |
| L. serriola_TC1476 | 2117 | 2118 |
| L. usitatissimum_c61921689 | 2119 | 2120 |
| L. virosa_DW147931 | 2121 | 2122 |
| M. crystallinum_TC8343 | 2123 | 2124 |
| M. domestica_TC224 | 2125 | 2126 |
| M. esculenta_TA5449_3983 | 2127 | 2128 |
| M. truncatula_BT051804 | 2129 | 2130 |
| N. tabacum_EB428961 | 2131 | 2132 |
| O. minuta_TA741_63629 | 2133 | 2134 |
| O. rufipogon_CT841597 | 2135 | 2136 |
| O. sativa_AB037970 | 2137 | 2138 |
| P. euphratica_TA2242_75702 | 2139 | 2140 |
| P. hybrida_TC2952 | 2141 | 2142 |
| P. persica_TC1266 | 2143 | 2144 |
| P. tremula_TA7101_113636 | 2145 | 2146 |
| P. trichocarpa_563837 | 2147 | 2148 |
| P. trichocarpa_833836 | 2149 | 2150 |
| P. trifoliata_TA5514_37690 | 2151 | 2152 |
| P. virgatum_TA3280_38727 | 2153 | 2154 |
| R. communis_EE257079 | 2155 | 2156 |
| S. aethnensis_TA30_121540 | 2157 | 2158 |
| S. bicolor_Sb09g001690.1 | 2159 | 2160 |
| S. bicolor_Sb09g001700.1 | 2161 | 2162 |
| S. indicum_DQ287974 | 2163 | 2164 |
| S. lycopersicum_TC196877 | 2165 | 2166 |
| S. tuberosum_TC167795 | 2167 | 2168 |
| T. aestivum_CA484858 | 2169 | 2170 |
| T. aestivum_TC278165 | 2171 | 2172 |
| T. hispida_TA863_189793 | 2173 | 2174 |
| T. kok-saghyz_TA971_333970 | 2175 | 2176 |
| T. officinale_TA193_50225 | 2177 | 2178 |
| T. salsuginea_TA1203_72664 | 2179 | 2180 |
| T. turgidum_AJ614920 | 2181 | 2182 |
| V. aestivalis_CF355345 | 2183 | 2184 |
| V. vinifera_GSVIVT00029528001 | 2185 | 2186 |
| Z. elegans_AB158512 | 2187 | 2188 |
| Z. mays_EU975087 | 2189 | 2190 |
| Z. mays_TC373023 | 2191 | 2192 |
| Z. mays_ZM07MC18926 | 2193 | 2194 |
| Z. violacea_AB158512 | 2195 | 2196 |
| A. cepa_CF447692 | 2197 | 2198 |
| A. cepa_CF449839 | 2199 | 2200 |
| C. japonica_TA1618_3369 | 2201 | 2202 |
| C. reinhardtii_143082 | 2203 | 2204 |
| C. richardii_CV735137 | 2205 | 2206 |
| C. vulgaris_44999 | 2207 | 2208 |
| C. vulgaris_77112 | 2209 | 2210 |
| Chlorella_143262 | 2211 | 2212 |
| P. bungeana_EU665680 | 2213 | 2214 |
| P. glauca_TA17594_3330 | 2215 | 2216 |
| P. glauca_TA19404_3330 | 2217 | 2218 |
| P. patens_223859 | 2219 | 2220 |
| P. patens_55477 | 2221 | 2222 |
| P. patens_93368 | 2223 | 2224 |
| P. sitchensis_TA10352_3332 | 2225 | 2226 |
| P. taeda_TA10755_3352 | 2227 | 2228 |
| P. taeda_TA6062_3352 | 2229 | 2230 |
| S. moellendorffii_139875 | 2231 | 2232 |
| S. moellendorffii_271409 | 2233 | 2234 |
| T. ruralis_CN201906 | 2235 | 2236 |
| W. mirabilis_TA1159_3377 | 2237 | 2238 |

Sequences have been tentatively assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. Special nucleic acid sequence databases have been created for particular organisms, such as by the Joint Genome Institute. Furthermore, access to proprietary databases, has allowed the identification of novel nucleic acid and polypeptide sequences.

### Example 2

### Alignment of Sequences Related to the Polypeptide Sequences Used in the Methods of the Invention

### 1. Glutamate Dehydrogenase (GDH) Polypeptides

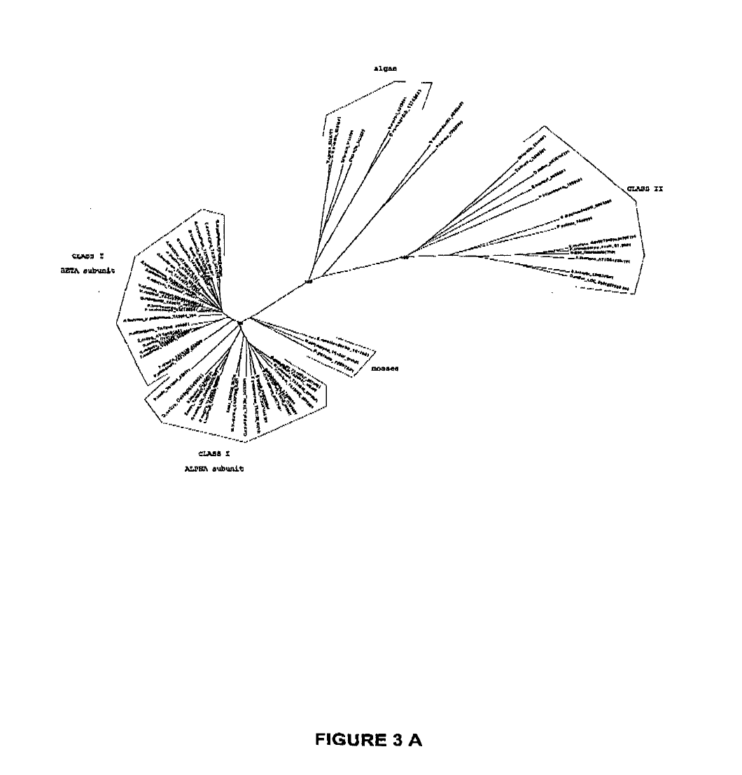

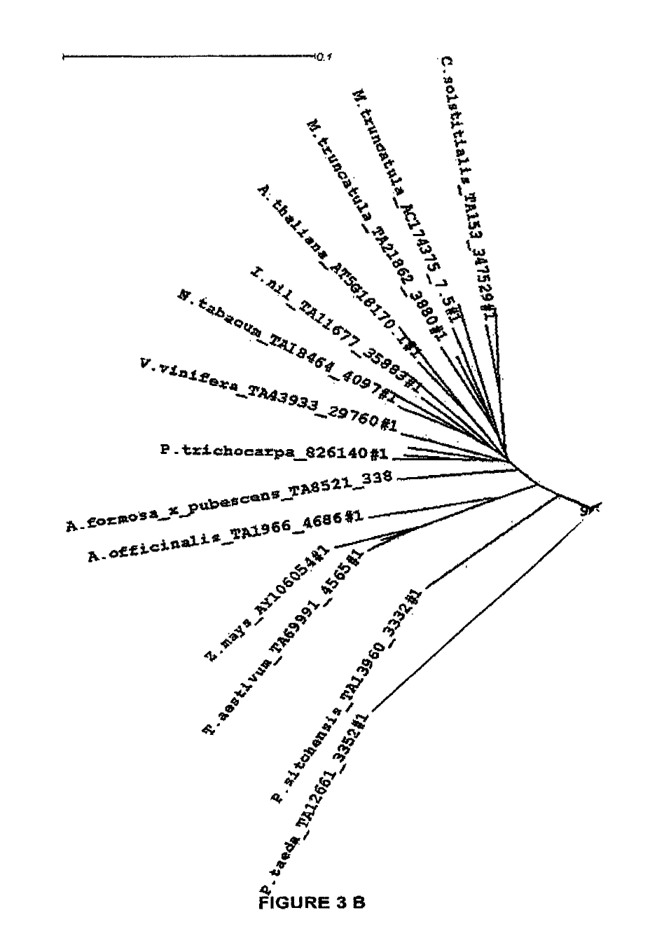

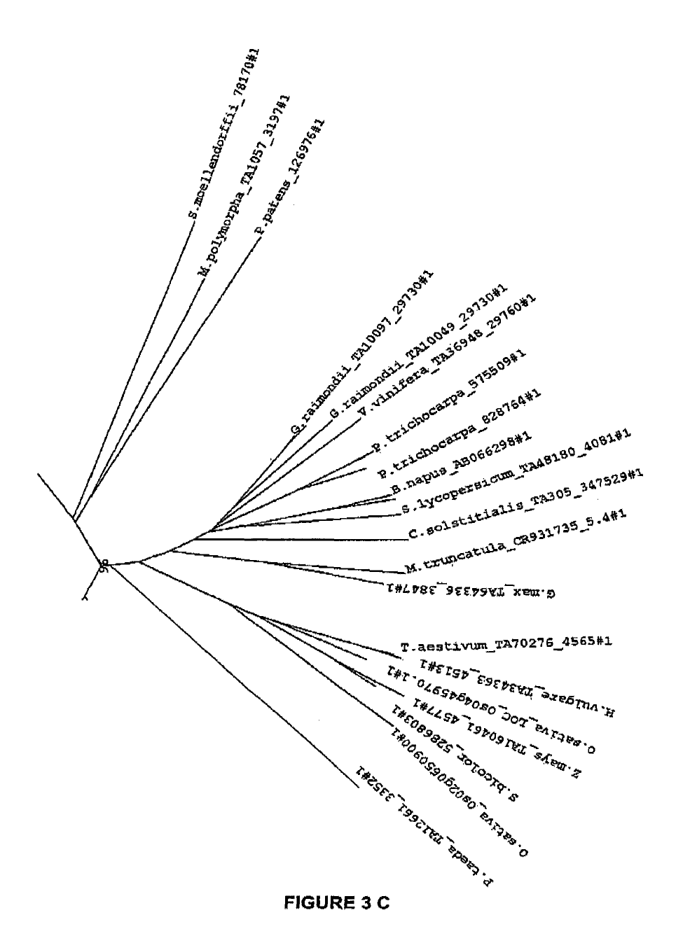

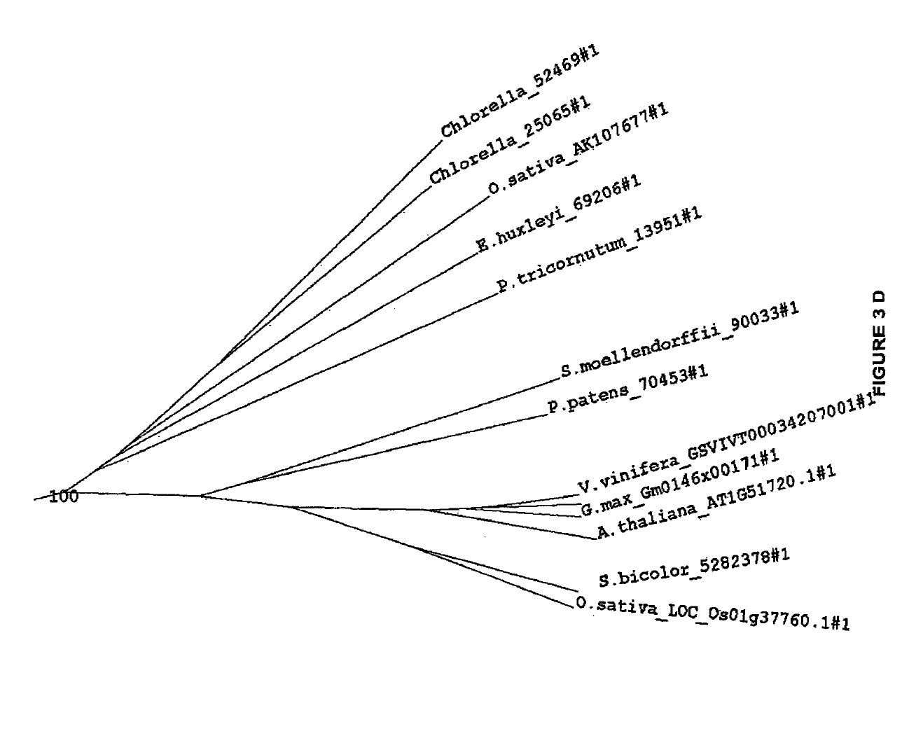

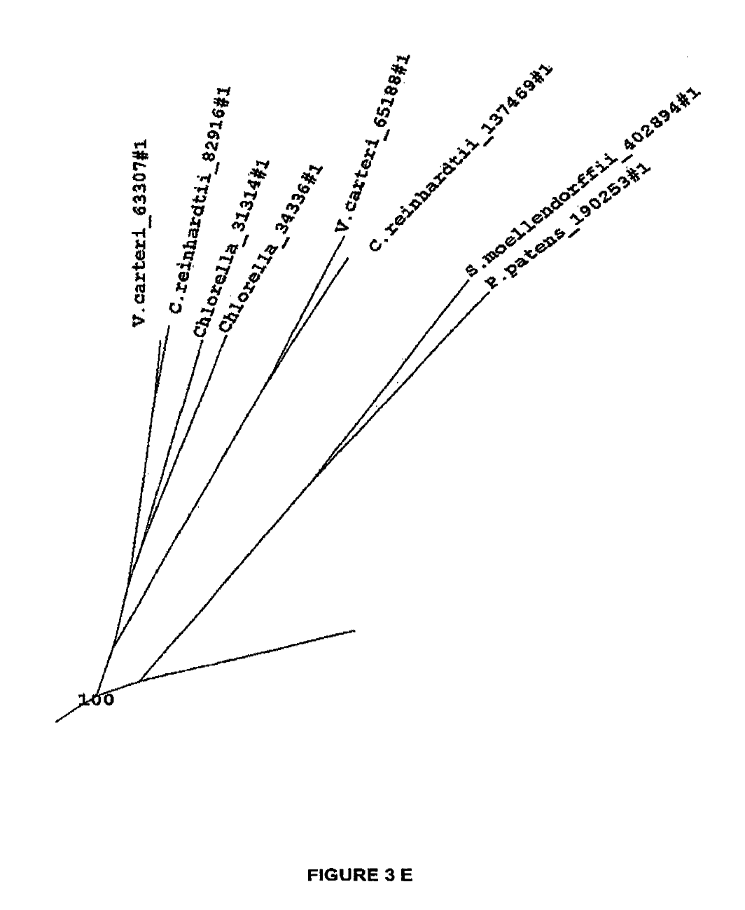

A phylogenetic tree of GDH polypeptides (FIG. 3) was constructed after aligning GDH polypeptides with MUSCLE (Edgar (2004), Nucleic Acids Research 32(5): 1792-97). The Neighbour-Joining tree was calculated using Quick-Tree (Howe et al. (2002), Bioinformatics 18(11): 1546-7). A circular phylogram was drawn using Dendroscope (Huson et al. (2007), BMC Bioinformatics 8(1):460). The tree shows a clear delineation of subgroups within the GDH polypeptides: Class I, Class II, algal and moss GDH proteins. Within Class I, the alpha and beta subunit polypeptides cluster together. Table A above lists the subgroup to which each of the sequences belongs to.

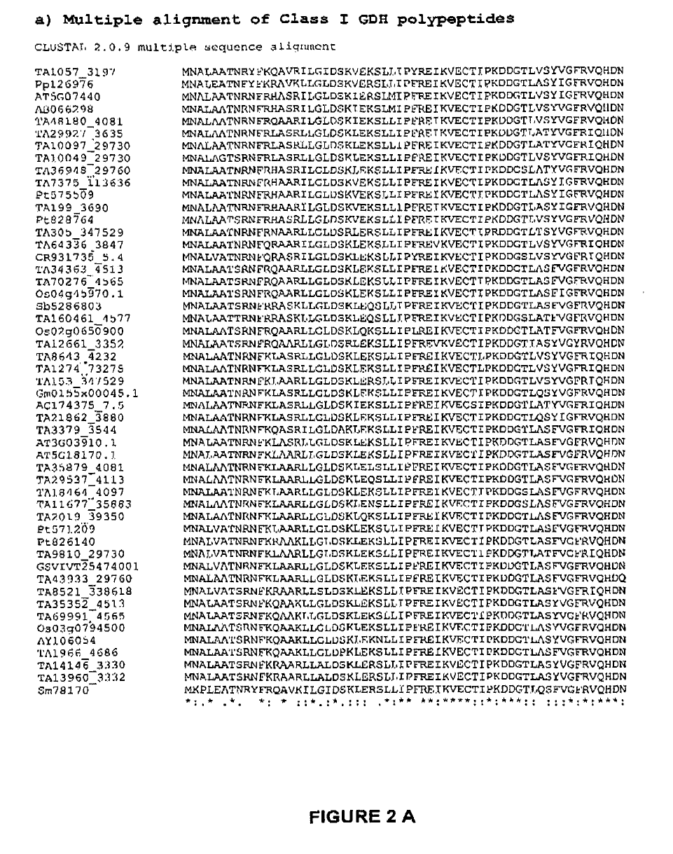

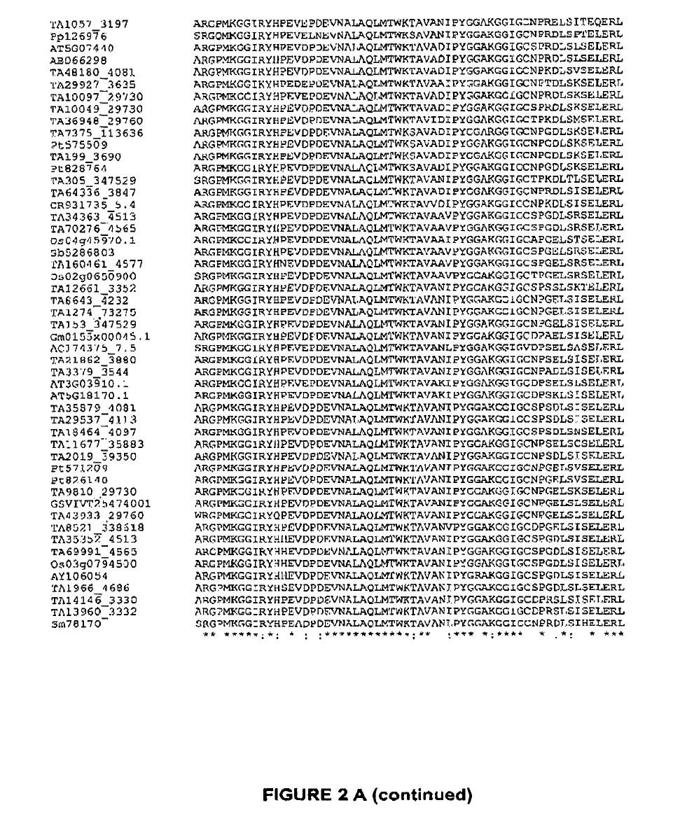

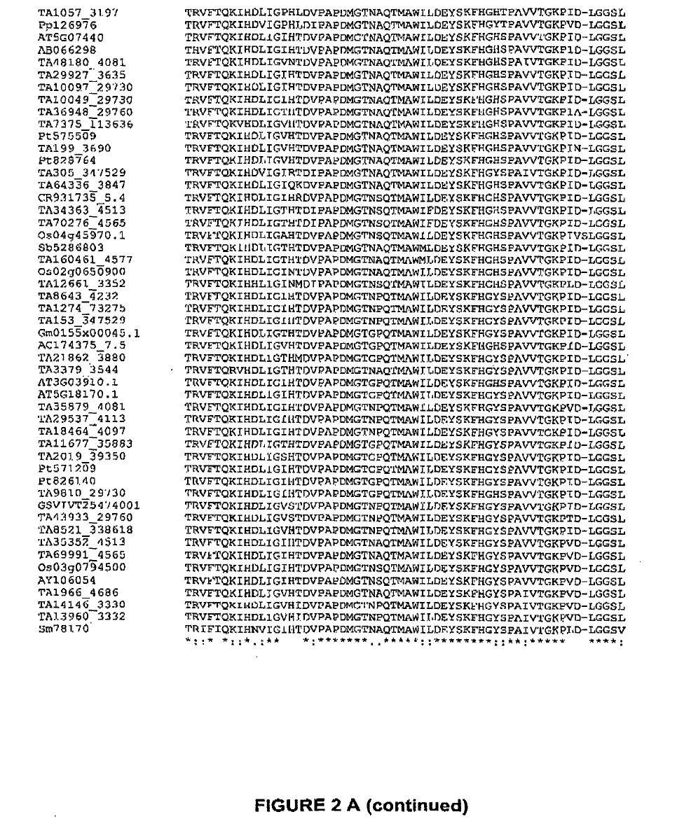

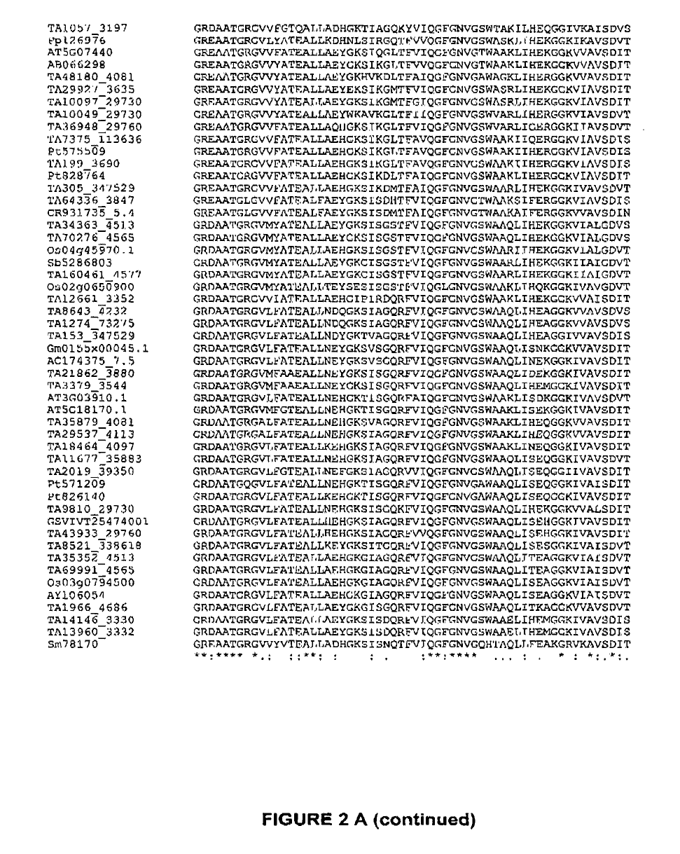

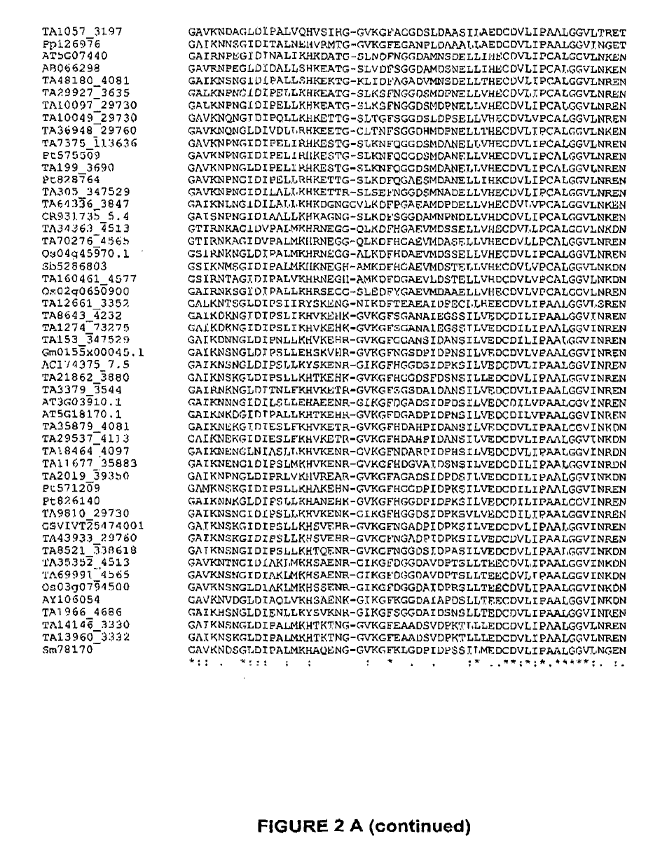

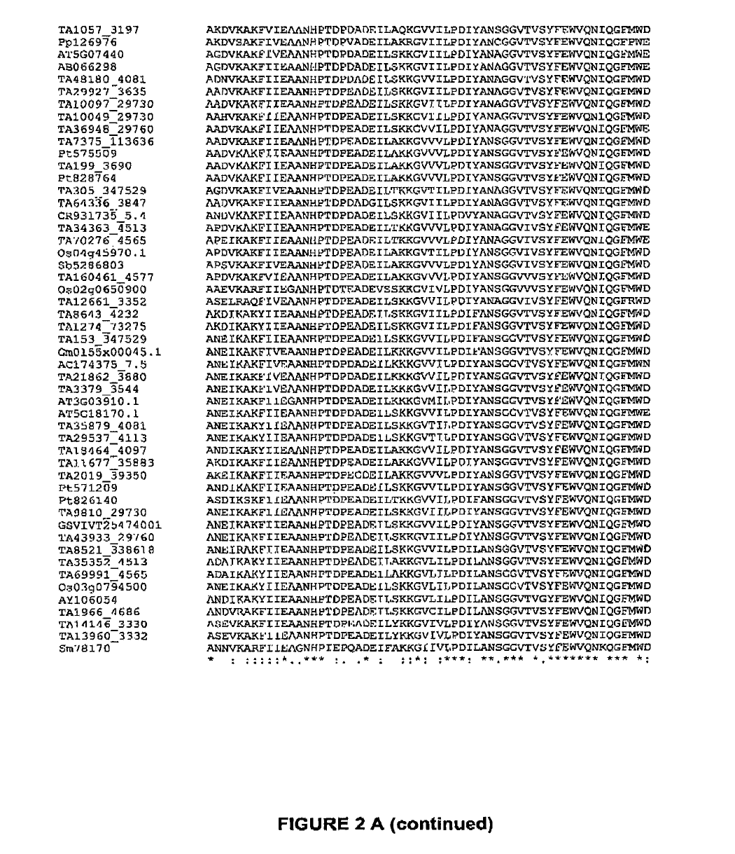

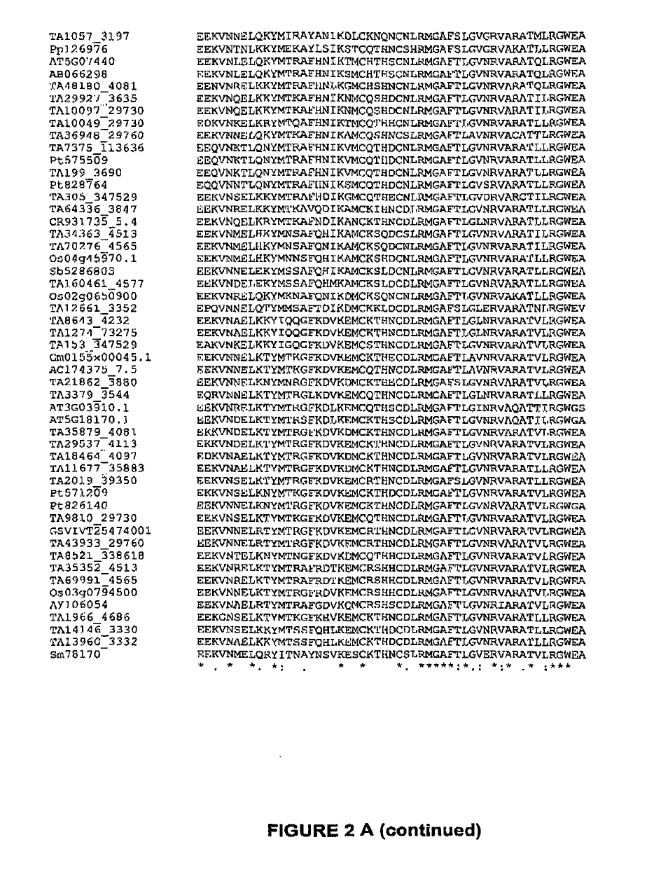

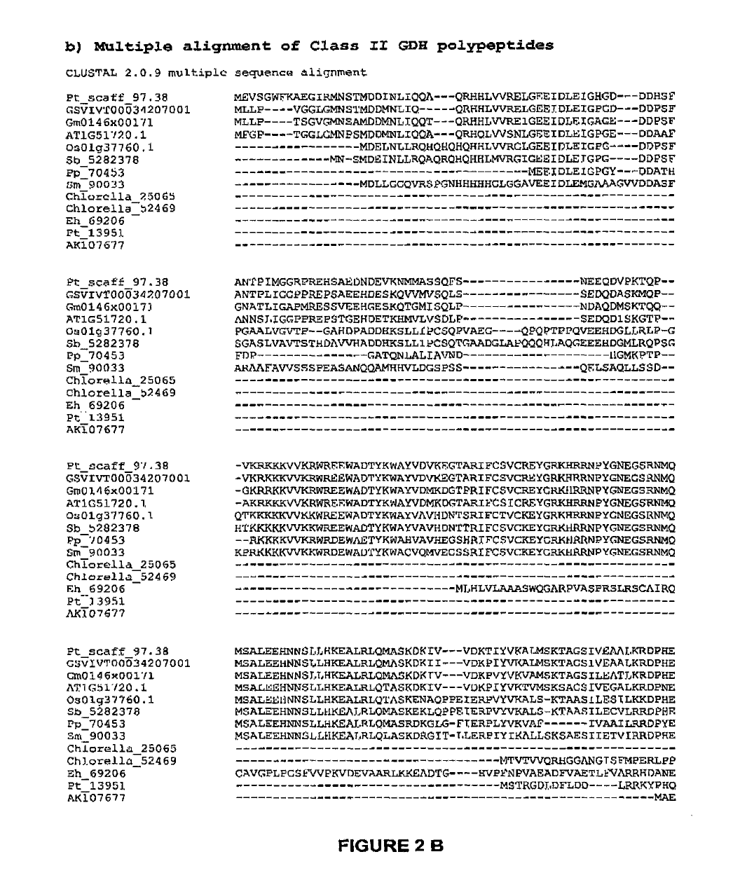

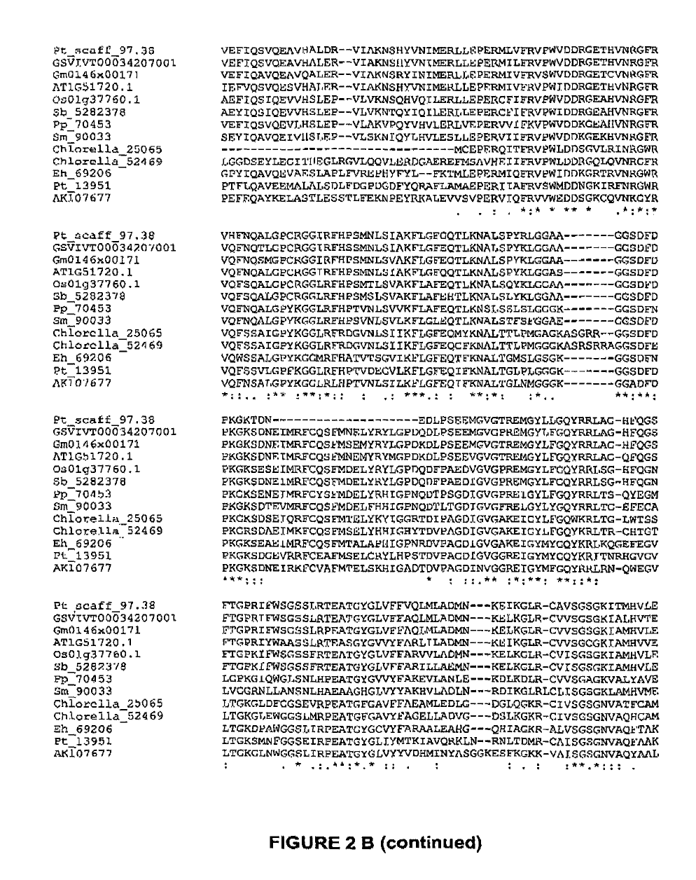

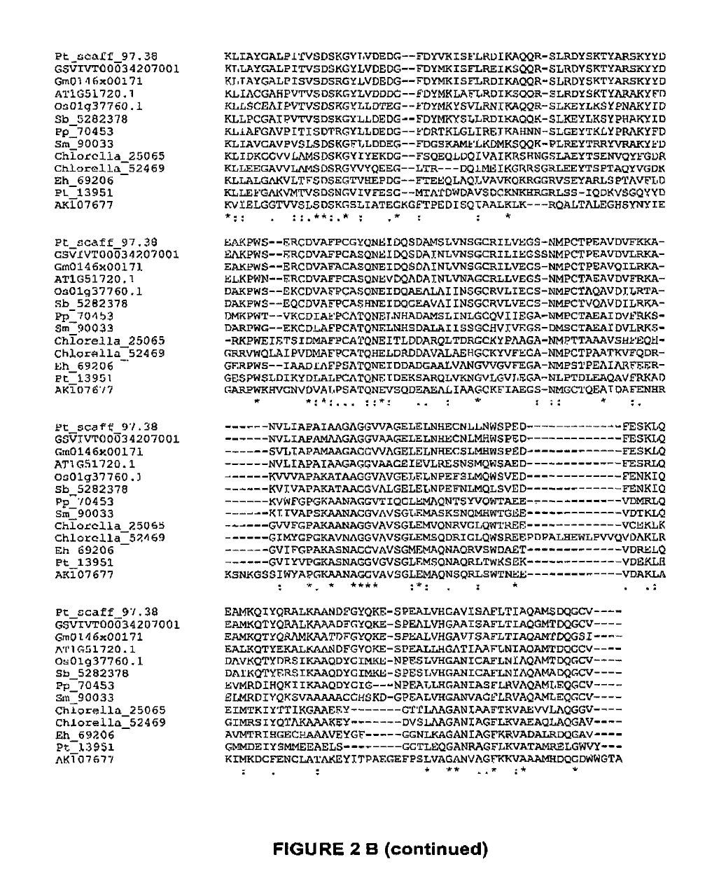

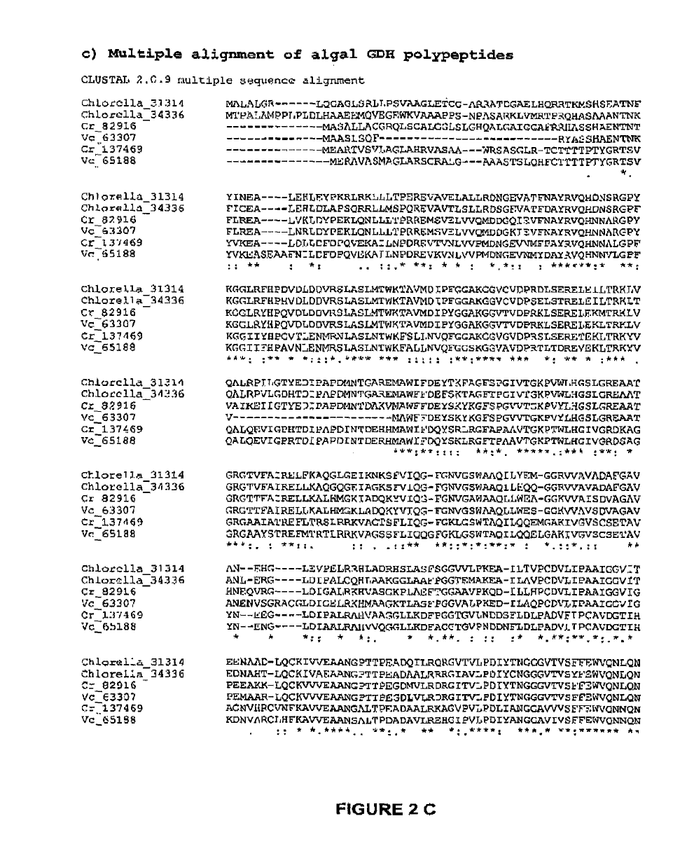

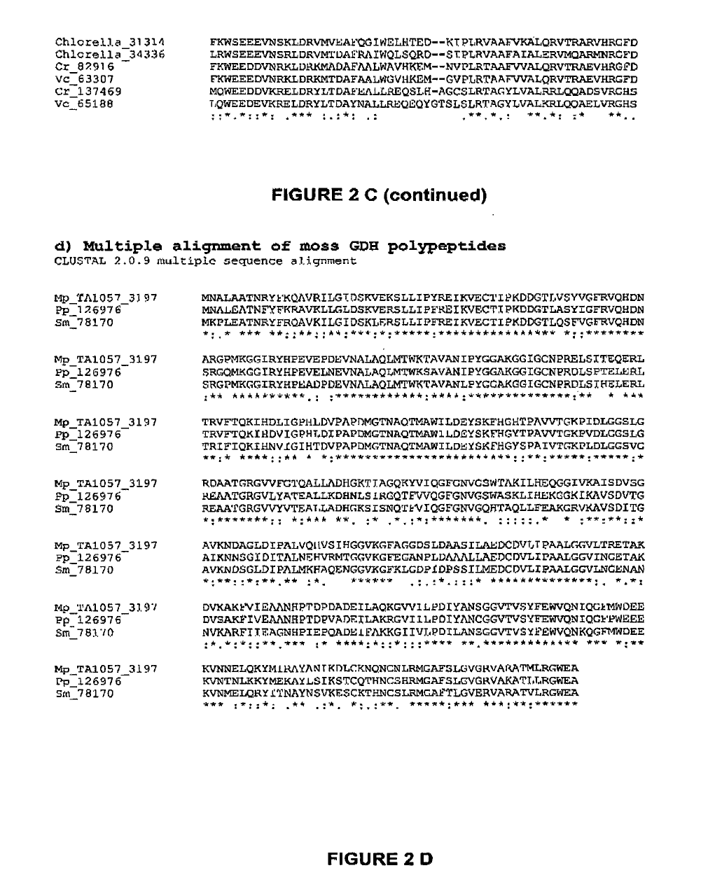

The sequence conservation is high when proteins of a subgroup are aligned, as shown in FIG. 2; alignments were made using the ClustalW 2.0 algorithm of progressive alignment (Thompson et al. (1997) Nucleic Acids Res 25:4876-4882; Chema et al. (2003). Nucleic Acids Res 31:3497-3500) with standard setting (slow alignment, similarity matrix: Gonnet, gap opening penalty 10, gap extension penalty: 0.2). Minor manual editing was done to further optimise the alignment. From these alignments a person skilled in the art can easily derive motifs for identifying other GDH proteins for use in the methods of this invention, in particular amino acid stretches comprising identical amino acids (labelled with an asterisk), or with conserved substitutions (labelled with a colon or a dot) are useful.

### 2. Fasciclin-Like AGP (FLAB) Polypeptides

Alignment of polypeptide sequences is performed using the ClustalW 2.0 algorithm of progressive alignment (Thompson et al. (1997) Nucleic Acids Res 25:4876-4882; Chema et al. (2003). Nucleic Acids Res 31:3497-3500) with standard setting (slow alignment, similarity matrix: Gonnet (or Blosum 62 (if polypeptides are aligned), gap opening penalty 10, gap extension penalty: 0.2).

A phylogenetic tree of FLA-like polypeptides is constructed using a neighbour-joining clustering algorithm as provided in the AlignX programme from the Vector NTI (Invitrogen).

### 3. Small Auxin-Up RNA (SAUR) Polypeptides

Alignment of polypeptide sequences is performed using the ClustalW 2.0 algorithm of progressive alignment (Thompson et al. (1997) Nucleic Acids Res 25:4876-4882; Chema et al. (2003). Nucleic Acids Res 31:3497-3500) with standard setting (slow alignment, similarity matrix: Gonnet (or Blosum 62 (if polypeptides are aligned), gap opening penalty 10, gap extension penalty: 0.2). Minor manual editing is done to further optimise the alignment.

### 4. SAUR Polypeptides, or SYNP Polypeptides, or Fusion Proteins of SAUR Polypeptides and SYNP Polypeptides

Alignment of polypeptide sequences is performed using the ClustalW 2.0 algorithm of progressive alignment (Thompson et al. (1997) Nucleic Acids Res 25:4876-4882; Chema et al. (2003). Nucleic Acids Res 31:3497-3500) with standard setting (slow alignment, similarity matrix: Gonnet (or Blosum 62 (if polypeptides are aligned), gap opening penalty 10, gap extension penalty: 0.2). Minor manual editing is done to further optimise the alignment.

### 5. Dehydroascorbate Reductase (DHAR) Polypeptides

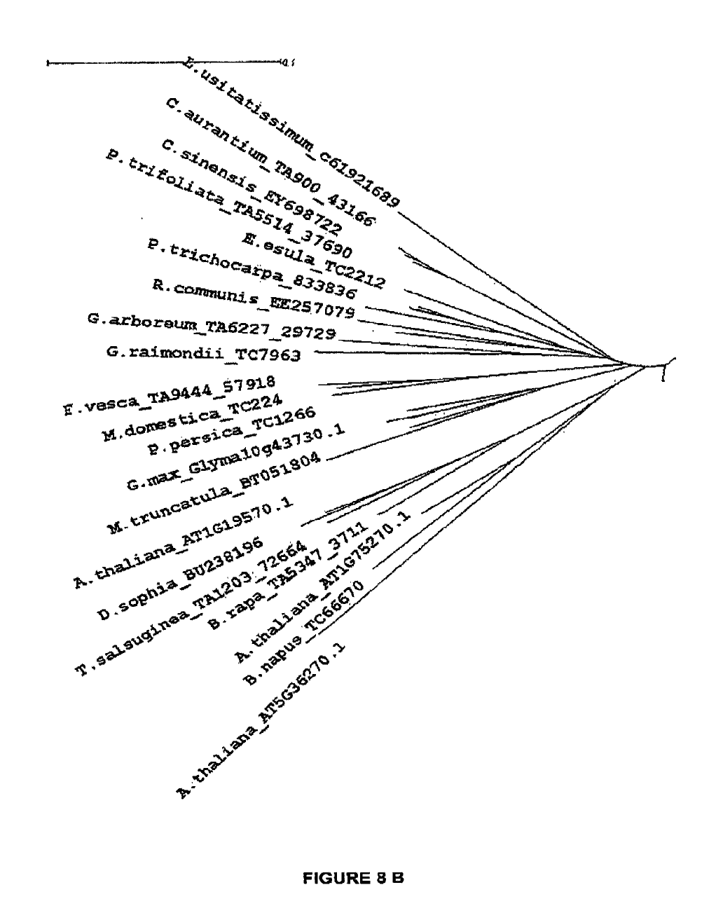

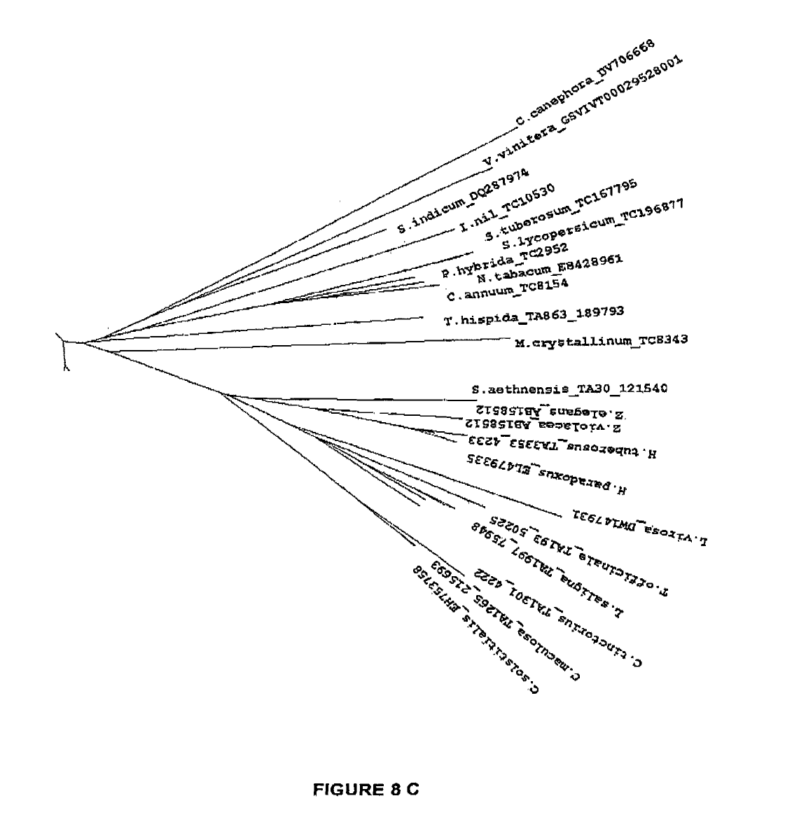

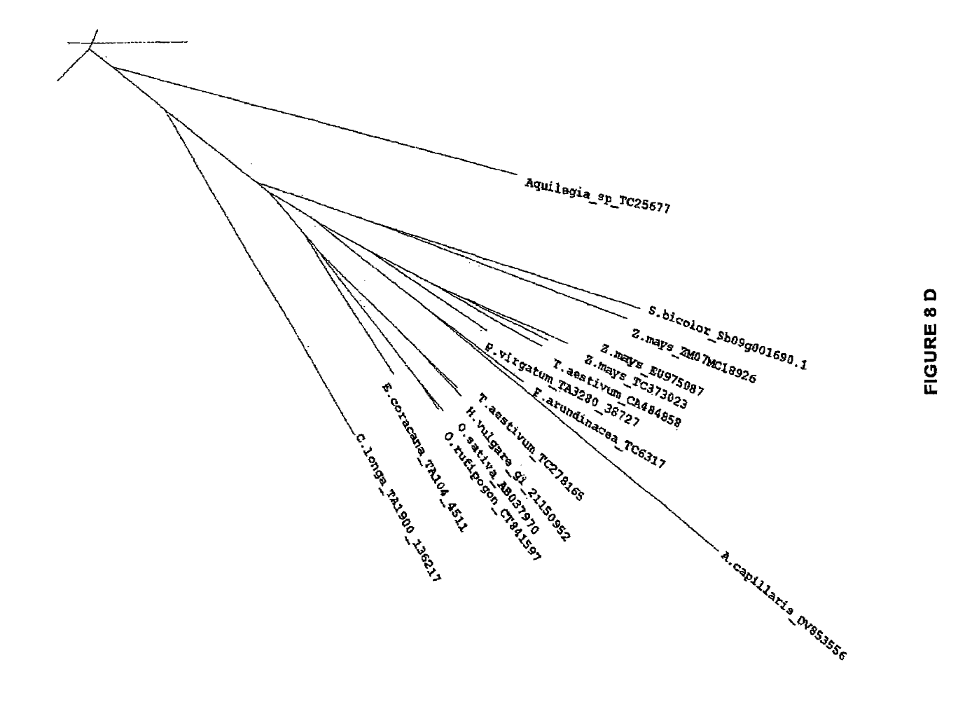

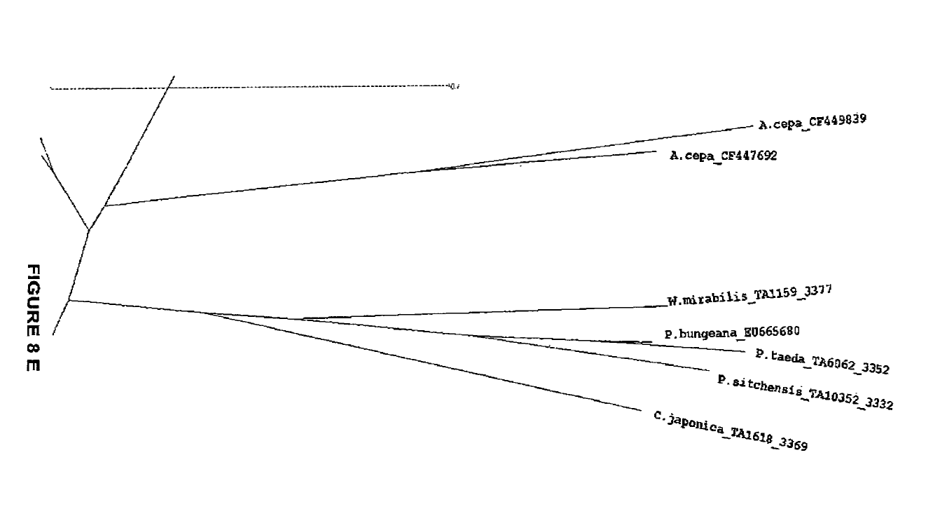

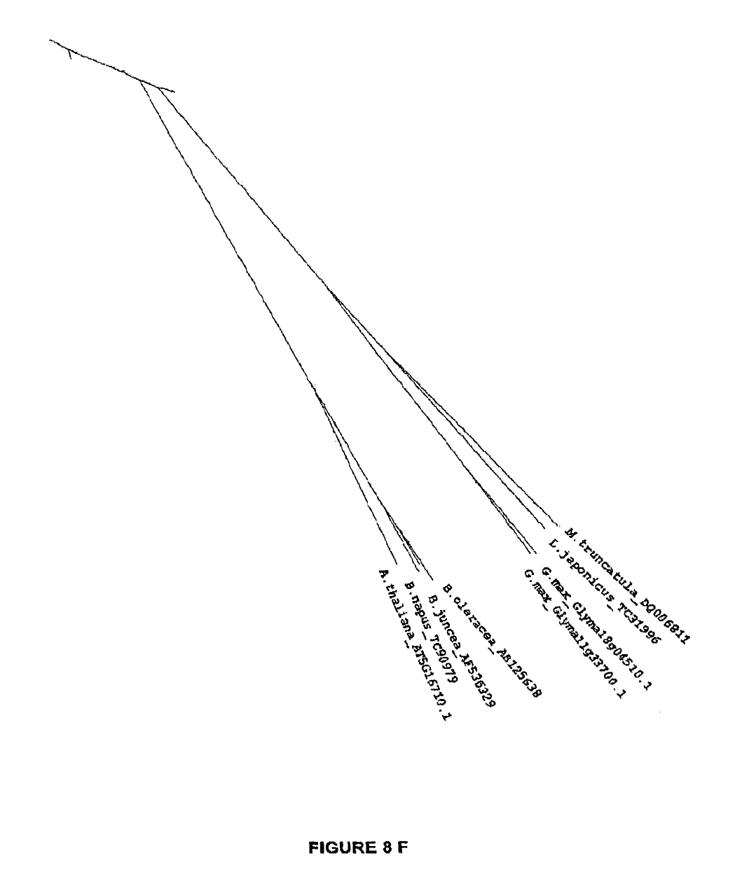

The alignment was generated using MAFFT (Katoh and Toh (2008)—Briefings in Bioinformatics 9:286-298). A neighbour-joining tree was calculated using Quick-Tree (Howe et al. (2002), Bioinformatics 18(11): 1546-7), 100 bootstrap repetitions. The circular phylogram was drawn using Dendroscope (Huson et al. (2007), BMC Bioinformatics 8(1):460)—FIG. 8. Confidence for 100 bootstrap repetitions is indicated for major branching.

### Example 3

### Calculation of Global Percentage Identity Between Polypeptide Sequences Useful in Performing the Methods of the Invention

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

### 1. Glutamate Dehydrogenase (GDH) Polypeptides

Parameters used in the comparison were:
Scoring matrix: Blosum62
First Gap: 12
Extending gap: 2

Results of the analysis are shown in Table B1 for the global similarity and identity over the full length of the polypeptide sequences from the Class I and mosses subgroups. Percentage identity is given above the diagonal and percentage similarity is given below the diagonal.

TABLE B1
MatGAT results for global similarity and identity over the full length of the
polypeptide sequences within the Class I and mosses subgroups.

| | 1 | 2 | 3 | 4 | 5 | 6 | 7 | 8 | 9 | 10 | 11 | 12 |
|---|---|---|---|---|---|---|---|---|---|---|---|---|
| 1. AT5G07440 | | 95.4 | 86.4 | 87.6 | 86.4 | 86.6 | 88.6 | 82.7 | 86.1 | 82.5 | 81.0 | 86.9 |
| 2. AB066298 | 99.3 | | 85.2 | 88.8 | 85.7 | 87.6 | 89.8 | 82.5 | 86.1 | 82.5 | 81.3 | 88.3 |
| 3. TA305_347529 | 96.1 | 95.1 | | 86.9 | 82.3 | 85.4 | 87.6 | 80.0 | 82.2 | 80.8 | 79.6 | 84.9 |
| 4. TA29927_3635 | 95.4 | 95.1 | 94.2 | | 85.9 | 90.8 | 98.5 | 83.7 | 85.4 | 84.5 | 83.0 | 88.6 |
| 5. TA64336_3847 | 92.7 | 92.7 | 92.2 | 92.5 | | 85.2 | 86.9 | 82.8 | 90.5 | 82.6 | 81.3 | 84.0 |
| 6. TA10049_29730 | 95.1 | 94.9 | 94.2 | 96.6 | 92.2 | | 91.7 | 82.0 | 84.2 | 82.8 | 81.8 | 87.3 |
| 7. TA10097_29730 | 96.1 | 95.9 | 95.1 | 98.8 | 93.2 | 97.1 | | 83.9 | 86.4 | 84.7 | 83.0 | 89.5 |
| 8. TA34363_4513 | 92.2 | 91.7 | 90.5 | 92.7 | 90.8 | 91.2 | 92.7 | | 79.6 | 92.0 | 86.4 | 81.8 |
| 9. CR931735_5.4 | 93.7 | 93.4 | 92.9 | 93.2 | 95.4 | 92.9 | 94.2 | 90.0 | | 79.9 | 77.9 | 83.2 |
| 10. Os04g45970.1 | 92.2 | 91.5 | 90.8 | 92.5 | 91.0 | 91.3 | 92.5 | 97.3 | 90.0 | | 86.4 | 84.5 |
| 11. Os02g0650900 | 91.2 | 90.5 | 90.0 | 92.7 | 90.5 | 92.0 | 92.5 | 96.1 | 89.8 | 95.1 | | 80.3 |
| 12. TA199_3690 | 95.9 | 95.4 | 94.9 | 97.3 | 92.5 | 95.9 | 98.1 | 92.5 | 93.7 | 92.7 | 92.5 | |
| 13. TA12661_3352 | 88.8 | 88.6 | 89.1 | 90.3 | 88.8 | 89.1 | 90.5 | 88.8 | 88.3 | 88.8 | 89.3 | 90.0 |
| 14. TA7375_113636 | 95.6 | 95.1 | 94.6 | 97.1 | 92.5 | 95.6 | 97.8 | 92.2 | 93.7 | 92.2 | 92.2 | 99.8 |
| 15. Pt575509 | 95.9 | 95.4 | 94.9 | 97.3 | 92.5 | 95.9 | 98.1 | 92.5 | 93.7 | 92.5 | 92.5 | 100.0 |
| 16. Pt828764 | 95.6 | 95.1 | 95.1 | 96.1 | 93.2 | 95.4 | 96.8 | 92.5 | 93.4 | 92.5 | 92.0 | 98.8 |
| 17. Sb5286803 | 92.5 | 91.7 | 91.5 | 93.2 | 91.7 | 92.0 | 93.2 | 97.6 | 90.5 | 96.8 | 95.6 | 93.2 |
| 18. TA48180_4081 | 94.9 | 95.4 | 93.7 | 93.2 | 92.2 | 93.7 | 94.2 | 91.5 | 92.7 | 91.0 | 90.5 | 94.2 |
| 19. TA70276_4565 | 92.2 | 91.7 | 90.8 | 92.7 | 91.3 | 91.2 | 92.7 | 100.0 | 90.5 | 97.3 | 96.1 | 92.5 |
| 20. TA36948_29760 | 94.4 | 93.9 | 93.4 | 94.2 | 91.3 | 94.6 | 94.9 | 91.2 | 91.7 | 91.3 | 90.0 | 93.9 |
| 21. TA160461_4577 | 92.2 | 91.5 | 90.8 | 92.7 | 91.0 | 91.5 | 92.7 | 97.1 | 90.0 | 96.4 | 95.4 | 92.7 |
| 22. TA8521_338618 | 90.3 | 90.3 | 90.3 | 91.5 | 90.3 | 91.0 | 92.0 | 89.8 | 90.8 | 90.0 | 90.3 | 91.5 |
| 23. TA1966_4686 | 90.8 | 90.5 | 90.3 | 90.8 | 89.6 | 90.3 | 91.2 | 89.3 | 89.5 | 89.8 | 89.1 | 91.2 |
| 24. AT3G03910.1 | 90.5 | 90.0 | 90.3 | 91.5 | 90.8 | 89.5 | 91.7 | 88.1 | 90.0 | 88.3 | 89.3 | 90.8 |
| 25. AT5G18170.1 | 90.8 | 90.5 | 90.0 | 92.0 | 90.5 | 90.5 | 92.0 | 89.5 | 89.8 | 90.0 | 90.0 | 91.0 |
| 26. TA153_347529 | 91.5 | 91.2 | 90.3 | 91.7 | 90.5 | 91.5 | 92.2 | 89.8 | 90.3 | 89.8 | 90.0 | 92.0 |
| 27. Gm0155x00045 | 90.0 | 90.0 | 90.3 | 91.2 | 90.0 | 90.5 | 91.7 | 89.3 | 89.5 | 89.3 | 88.6 | 90.5 |
| 28. TA9810_29730 | 91.5 | 91.0 | 91.0 | 92.9 | 90.5 | 92.2 | 93.4 | 90.5 | 90.8 | 90.3 | 90.8 | 92.7 |
| 29. TA8643_4232 | 91.5 | 91.7 | 90.8 | 92.2 | 90.8 | 92.5 | 92.7 | 90.5 | 90.5 | 90.5 | 91.0 | 92.0 |
| 30. TA1274_73275 | 91.5 | 91.7 | 90.8 | 92.2 | 90.8 | 92.5 | 92.7 | 90.5 | 90.5 | 90.5 | 91.0 | 92.0 |
| 31. TA35352_4513 | 90.5 | 89.8 | 89.8 | 91.5 | 90.5 | 91.5 | 92.0 | 88.8 | 90.8 | 89.3 | 89.8 | 91.5 |
| 32. TA11677_35883 | 91.0 | 90.5 | 90.3 | 92.2 | 89.8 | 91.2 | 92.5 | 90.0 | 89.3 | 90.5 | 89.8 | 91.7 |
| 33. TA3379_3544 | 91.2 | 91.0 | 90.8 | 91.5 | 90.5 | 91.0 | 92.0 | 90.3 | 89.5 | 90.0 | 89.8 | 91.7 |
| 34. AC174375_7.5 | 90.5 | 90.3 | 90.5 | 92.5 | 90.0 | 91.2 | 92.7 | 89.1 | 89.8 | 89.6 | 89.8 | 91.7 |
| 35. TA21862_3880 | 90.8 | 90.3 | 90.3 | 91.2 | 89.3 | 90.0 | 91.5 | 89.8 | 89.1 | 89.3 | 88.6 | 91.0 |
| 36. TA18464_4097 | 91.0 | 91.0 | 90.5 | 92.7 | 90.5 | 91.7 | 92.9 | 90.0 | 90.5 | 91.0 | 90.5 | 91.7 |
| 37. TA2019_39350 | 90.3 | 90.0 | 89.1 | 91.5 | 89.8 | 90.8 | 91.7 | 88.8 | 89.3 | 88.8 | 88.8 | 90.8 |
| 38. Os03g0794500 | 90.8 | 90.0 | 90.5 | 91.5 | 90.5 | 91.0 | 92.0 | 89.3 | 90.8 | 89.6 | 89.8 | 91.5 |
| 39. TA14146_3330 | 90.5 | 90.5 | 90.5 | 91.5 | 91.7 | 91.0 | 92.2 | 90.0 | 91.0 | 90.3 | 90.3 | 92.0 |
| 40. TA13960_3332 | 90.8 | 90.8 | 90.3 | 91.2 | 91.5 | 90.8 | 92.0 | 90.0 | 90.8 | 90.0 | 90.0 | 91.7 |
| 41. Pt571209 | 91.0 | 90.5 | 90.5 | 92.0 | 90.0 | 90.5 | 92.5 | 90.0 | 90.3 | 89.8 | 90.0 | 92.0 |
| 42. Pt826140 | 91.2 | 90.3 | 90.0 | 91.5 | 90.5 | 90.5 | 92.0 | 90.3 | 90.3 | 89.8 | 90.5 | 91.5 |
| 43. TA35879_4081 | 91.2 | 91.0 | 90.8 | 91.5 | 89.3 | 90.3 | 91.7 | 89.8 | 88.6 | 90.8 | 89.8 | 91.0 |
| 44. TA29537_4113 | 91.5 | 91.2 | 91.0 | 91.5 | 89.6 | 90.5 | 91.7 | 90.0 | 88.8 | 91.0 | 90.0 | 91.2 |
| 45. TA69991_4565 | 90.5 | 89.8 | 89.8 | 91.5 | 90.5 | 91.5 | 92.0 | 88.8 | 90.8 | 89.3 | 89.8 | 91.5 |
| 46. GSVIVT25474001 | 91.0 | 90.5 | 90.0 | 91.2 | 90.5 | 90.8 | 91.7 | 90.0 | 90.3 | 89.8 | 90.0 | 91.0 |
| 47. TA43933_29760 | 90.3 | 89.8 | 89.3 | 90.5 | 89.8 | 90.0 | 91.0 | 89.3 | 89.1 | 89.1 | 89.3 | 90.3 |
| 48. AY106054 | 90.8 | 90.3 | 90.0 | 91.0 | 90.3 | 90.3 | 91.5 | 88.3 | 90.5 | 88.6 | 88.6 | 91.5 |
| 49. TA1057_3197 | 90.0 | 89.8 | 88.1 | 89.5 | 89.1 | 89.8 | 90.3 | 89.5 | 89.3 | 88.6 | 88.8 | 89.3 |
| 50. Pp126976 | 87.8 | 87.3 | 85.9 | 87.8 | 85.7 | 87.8 | 88.3 | 86.4 | 85.6 | 86.2 | 86.4 | 87.8 |
| 51. Sm78170 | 87.1 | 87.1 | 86.4 | 87.8 | 86.4 | 88.1 | 88.3 | 86.1 | 87.6 | 86.2 | 85.6 | 87.6 |
| | 13 | 14 | 15 | 16 | 17 | 18 | 19 | 20 | 21 | 22 | 23 | 24 |
| 1. AT5G07440 | 76.9 | 86.6 | 87.3 | 86.6 | 82.2 | 88.6 | 82.5 | 85.9 | 80.3 | 78.8 | 79.8 | 78.6 |
| 2. AB066298 | 76.6 | 87.6 | 88.3 | 88.3 | 81.3 | 89.3 | 82.2 | 87.8 | 79.8 | 78.3 | 80.3 | 78.6 |
| 3. TA305_347529 | 76.2 | 84.7 | 85.4 | 85.6 | 80.0 | 85.6 | 80.5 | 84.2 | 77.6 | 79.8 | 79.8 | 80.0 |
| 4. TA29927_3635 | 77.6 | 88.3 | 89.1 | 88.6 | 83.5 | 86.9 | 84.2 | 88.1 | 82.0 | 81.5 | 80.8 | 80.8 |
| 5. TA64336_3847 | 77.2 | 84.0 | 84.5 | 84.5 | 82.0 | 85.9 | 82.3 | 84.0 | 80.6 | 80.3 | 80.1 | 79.4 |
| 6. TA10049_29730 | 76.6 | 87.1 | 87.8 | 88.6 | 82.7 | 86.6 | 82.7 | 87.6 | 80.8 | 81.5 | 81.0 | 80.5 |
| 7. TA10097_29730 | 78.1 | 89.3 | 90.0 | 89.5 | 83.7 | 87.8 | 84.4 | 88.8 | 82.2 | 82.0 | 81.3 | 81.5 |
| 8. TA34363_4513 | 78.1 | 81.5 | 82.2 | 82.2 | 91.2 | 81.8 | 98.3 | 82.0 | 89.8 | 80.3 | 79.6 | 76.9 |
| 9. CR931735_5.4 | 75.4 | 83.2 | 83.7 | 83.7 | 78.6 | 84.7 | 79.1 | 83.7 | 78.3 | 80.5 | 80.3 | 78.8 |
| 10. Os04g45970.1 | 78.4 | 83.5 | 84.2 | 83.7 | 90.3 | 82.3 | 91.7 | 82.3 | 88.3 | 80.3 | 81.3 | 78.9 |
| 11. Os02g0650900 | 76.4 | 80.0 | 80.8 | 81.5 | 86.4 | 81.5 | 87.6 | 80.3 | 85.4 | 79.3 | 77.9 | 78.6 |
| 12. TA199_3690 | 77.4 | 98.8 | 99.5 | 94.6 | 81.5 | 86.6 | 82.5 | 87.8 | 79.6 | 79.8 | 80.0 | 79.1 |
| 13. TA12661_3352 | | 76.6 | 77.4 | 79.1 | 78.1 | 77.1 | 78.8 | 75.9 | 75.7 | 77.6 | 78.1 | 76.9 |
| 14. TA7375_113636 | 89.8 | | 99.3 | 94.4 | 81.3 | 86.4 | 82.2 | 87.1 | 79.3 | 79.8 | 79.6 | 79.1 |
| 15. Pt575509 | 90.0 | 99.8 | | 95.1 | 82.0 | 87.1 | 83.0 | 87.6 | 80.0 | 80.3 | 80.0 | 79.6 |
| 16. Pt828764 | 90.5 | 98.5 | 98.8 | | 83.2 | 87.6 | 83.0 | 86.9 | 81.0 | 80.0 | 80.0 | 79.8 |
| 17. Sb5286803 | 89.8 | 92.9 | 93.2 | 93.4 | | 81.3 | 90.3 | 81.8 | 95.4 | 81.3 | 79.8 | 78.6 |
| 18. TA48180_4081 | 89.3 | 93.9 | 94.2 | 94.6 | 92.2 | | 82.2 | 85.4 | 79.3 | 78.8 | 80.8 | 79.1 |
| 19. TA70276_4565 | 89.3 | 92.2 | 92.5 | 92.5 | 97.6 | 91.2 | | 81.5 | 88.3 | 80.3 | 79.1 | 77.9 |
| 20. TA36948_29760 | 88.3 | 93.9 | 93.9 | 93.7 | 91.5 | 92.9 | 91.2 | | 80.8 | 79.6 | 80.0 | 79.1 |
| 21. TA160461_4577 | 90.0 | 92.5 | 92.7 | 92.9 | 99.0 | 91.7 | 97.3 | 90.8 | | 80.5 | 79.6 | 78.3 |
| 22. TA8521_338618 | 90.0 | 91.5 | 91.5 | 90.5 | 90.5 | 89.8 | 90.3 | 89.5 | 90.5 | | 87.6 | 87.1 |
| 23. TA1966_4686 | 90.0 | 91.2 | 91.2 | 90.5 | 90.5 | 90.5 | 89.5 | 90.3 | 90.8 | 95.6 | | 84.2 |
| 24. AT3G03910.1 | 89.3 | 90.8 | 90.8 | 90.0 | 89.1 | 90.3 | 88.6 | 89.3 | 88.8 | 94.6 | 93.2 | |
| 25. AT5G18170.1 | 90.0 | 91.0 | 91.0 | 90.8 | 90.3 | 90.0 | 90.0 | 90.0 | 90.3 | 95.1 | 92.9 | 97.3 |
| 26. TA153_347529 | 89.5 | 91.7 | 92.0 | 91.5 | 90.3 | 91.2 | 90.3 | 89.1 | 89.8 | 95.1 | 93.9 | 95.1 |
| 27. Gm0155x00045.1 | 89.5 | 90.5 | 90.5 | 90.5 | 90.3 | 89.5 | 89.8 | 89.8 | 90.0 | 94.9 | 93.4 | 96.8 |
| 28. TA9810_29730 | 91.5 | 92.5 | 92.7 | 92.0 | 91.7 | 90.3 | 91.0 | 90.0 | 91.0 | 97.1 | 94.6 | 96.1 |
| 29. TA8643_4232 | 89.8 | 91.7 | 92.0 | 91.7 | 91.0 | 90.8 | 91.0 | 89.8 | 90.5 | 95.6 | 94.2 | 94.6 |
| 30. TA1274_73275 | 89.8 | 91.7 | 92.0 | 91.7 | 91.0 | 90.8 | 91.0 | 89.8 | 90.5 | 95.6 | 94.2 | 94.6 |
| 31. TA35352_4513 | 89.8 | 91.5 | 91.5 | 90.5 | 90.8 | 90.5 | 89.3 | 90.0 | 91.0 | 95.4 | 96.4 | 93.4 |
| 32. TA11677_35883 | 89.8 | 91.7 | 91.7 | 90.8 | 90.8 | 89.5 | 90.5 | 89.8 | 90.0 | 94.6 | 93.9 | 95.4 |
| 33. TA3379_3544 | 90.3 | 91.5 | 91.7 | 91.5 | 91.0 | 90.5 | 90.8 | 90.8 | 90.5 | 94.4 | 94.4 | 94.9 |
| 34. AC174375_7.5 | 91.0 | 91.5 | 91.7 | 91.0 | 90.5 | 89.8 | 89.5 | 89.8 | 90.0 | 96.1 | 93.9 | 96.8 |
| 35. TA21862_3880 | 89.8 | 91.0 | 91.0 | 90.5 | 90.3 | 89.1 | 90.3 | 88.8 | 90.0 | 94.6 | 93.2 | 94.9 |
| 36. TA18464_4097 | 91.0 | 91.5 | 91.7 | 91.0 | 91.0 | 90.3 | 90.5 | 90.5 | 90.5 | 95.1 | 94.2 | 95.6 |
| 37. TA2019_39350 | 89.3 | 90.8 | 90.8 | 90.5 | 90.3 | 89.1 | 89.3 | 89.5 | 89.5 | 94.2 | 93.2 | 94.9 |
| 38. Os03g0794500 | 90.5 | 91.5 | 91.5 | 91.0 | 90.8 | 90.3 | 89.8 | 90.5 | 91.5 | 96.1 | 97.3 | 94.2 |
| 39. TA14146_3330 | 92.5 | 91.7 | 92.0 | 92.2 | 90.8 | 90.8 | 90.3 | 89.5 | 90.8 | 95.1 | 92.7 | 93.2 |
| 40. TA13960_3332 | 92.0 | 91.5 | 91.7 | 91.7 | 90.3 | 90.5 | 90.3 | 89.1 | 90.8 | 94.6 | 92.5 | 92.9 |
| 41. Pt571209 | 90.5 | 92.0 | 92.0 | 91.2 | 91.2 | 90.3 | 90.5 | 90.0 | 90.8 | 96.8 | 94.4 | 95.6 |
| 42. Pt826140 | 90.0 | 91.5 | 91.5 | 90.8 | 91.0 | 89.8 | 90.8 | 89.8 | 90.5 | 97.1 | 94.6 | 95.6 |
| 43. TA35879_4081 | 89.5 | 90.8 | 91.0 | 90.8 | 90.3 | 89.8 | 90.3 | 89.5 | 89.8 | 93.2 | 93.2 | 94.2 |
| 44. TA29537_4113 | 89.8 | 91.0 | 91.2 | 91.0 | 90.5 | 90.0 | 90.5 | 89.8 | 90.0 | 93.4 | 93.4 | 94.4 |
| 45. TA69991_4565 | 89.8 | 91.5 | 91.5 | 90.5 | 90.8 | 90.5 | 89.3 | 90.0 | 91.0 | 95.4 | 96.4 | 93.7 |
| 46. GSVIVT25474001 | 90.8 | 91.0 | 91.0 | 90.8 | 90.8 | 90.0 | 90.5 | 89.5 | 90.8 | 96.4 | 95.1 | 95.9 |
| 47. TA43933_29760 | 90.0 | 90.3 | 90.3 | 90.0 | 90.0 | 89.3 | 89.8 | 88.8 | 90.0 | 95.1 | 94.4 | 95.1 |
| 48. AY106054 | 89.3 | 91.5 | 91.5 | 90.8 | 89.3 | 90.0 | 88.8 | 90.0 | 89.5 | 93.9 | 96.1 | 92.9 |
| 49. TA1057_3197 | 89.3 | 89.1 | 89.3 | 89.1 | 89.5 | 89.1 | 89.5 | 88.3 | 89.3 | 90.3 | 90.5 | 89.3 |
| 50. Pp126976 | 85.4 | 87.6 | 87.8 | 87.3 | 86.4 | 87.1 | 86.6 | 85.9 | 86.1 | 87.8 | 86.6 | 86.4 |
| 51. Sm78170 | 86.1 | 87.6 | 87.6 | 87.3 | 86.6 | 86.9 | 86.1 | 86.4 | 85.9 | 89.8 | 88.3 | 87.1 |
| | 25 | 26 | 27 | 28 | 29 | 30 | 31 | 32 | 33 | 34 | 5 | 36 |
| 1. AT5G07440 | 80.5 | 78.8 | 79.8 | 80.5 | 79.8 | 79.8 | 80.3 | 79.6 | 80.5 | 79.3 | 81.0 | 79.8 |
| 2. AB066298 | 80.5 | 79.1 | 79.8 | 80.5 | 79.3 | 79.3 | 80.0 | 79.3 | 80.8 | 79.1 | 80.8 | 79.6 |
| 3. TA305_347529 | 79.8 | 79.1 | 79.8 | 80.8 | 78.6 | 78.6 | 80.5 | 80.5 | 79.3 | 78.8 | 79.8 | 80.5 |
| 4. TA29927_3635 | 81.3 | 81.3 | 82.0 | 85.2 | 81.3 | 81.3 | 81.8 | 81.0 | 80.8 | 82.2 | 81.3 | 81.3 |
| 5. TA64336_3847 | 81.3 | 80.8 | 80.6 | 82.0 | 81.3 | 81.3 | 81.1 | 80.3 | 80.6 | 79.1 | 79.9 | 81.1 |
| 6. TA10049_29730 | 80.3 | 81.5 | 81.3 | 83.0 | 81.3 | 81.3 | 82.2 | 80.0 | 80.0 | 80.3 | 79.8 | 80.5 |
| 7. TA10097_29730 | 81.8 | 81.8 | 82.5 | 85.6 | 81.8 | 81.8 | 82.2 | 81.5 | 81.3 | 82.7 | 81.8 | 81.8 |
| 8. TA34363_4513 | 80.0 | 78.1 | 78.3 | 80.3 | 79.1 | 79.1 | 81.0 | 80.5 | 78.3 | 77.1 | 79.1 | 79.3 |
| 9. CR931735_5.4 | 80.3 | 79.6 | 80.3 | 82.5 | 81.0 | 81.0 | 79.6 | 80.3 | 81.3 | 79.6 | 80.3 | 80.8 |
| 10. Os04g45970.1 | 80.8 | 79.4 | 79.6 | 82.3 | 80.3 | 80.3 | 81.6 | 82.5 | 79.6 | 78.4 | 80.8 | 81.3 |
| 11. Os02g0650900 | 79.8 | 77.4 | 77.4 | 80.0 | 77.6 | 77.6 | 77.4 | 78.3 | 77.6 | 77.1 | 77.6 | 78.1 |
| 12. TA199_3690 | 79.6 | 80.3 | 79.8 | 81.8 | 79.1 | 79.1 | 81.0 | 81.0 | 80.8 | 79.1 | 80.5 | 80.8 |
| 13. TA12661_3352 | 78.1 | 76.9 | 78.3 | 79.3 | 76.9 | 76.9 | 79.3 | 78.1 | 77.1 | 77.9 | 78.8 | 79.6 |
| 14. TA7375_113636 | 79.6 | 79.6 | 79.3 | 81.5 | 78.8 | 78.8 | 81.0 | 81.0 | 80.5 | 78.6 | 80.0 | 80.3 |
| 15. Pt575509 | 80.0 | 80.3 | 79.8 | 82.2 | 79.6 | 79.6 | 81.5 | 81.5 | 80.8 | 79.1 | 80.5 | 80.8 |
| 16. Pt828764 | 79.8 | 80.3 | 80.3 | 81.5 | 79.6 | 79.6 | 81.0 | 80.8 | 80.8 | 79.1 | 80.5 | 81.0 |
| 17. Sb5286803 | 80.0 | 78.1 | 79.8 | 80.0 | 78.3 | 78.3 | 82.0 | 81.5 | 79.1 | 78.6 | 80.5 | 79.6 |
| 18. TA48180_4081 | 80.3 | 80.0 | 79.8 | 80.5 | 78.8 | 78.8 | 80.3 | 79.3 | 79.8 | 79.3 | 79.8 | 79.6 |
| 19. TA70276_4565 | 81.0 | 79.3 | 79.3 | 81.3 | 79.6 | 79.6 | 80.8 | 80.3 | 79.6 | 78.1 | 79.8 | 79.3 |
| 20. TA36948_29760 | 79.6 | 78.1 | 80.3 | 80.8 | 78.3 | 78.3 | 81.3 | 80.5 | 80.0 | 80.3 | 79.6 | 80.5 |
| 21. TA160461_4577 | 80.0 | 77.6 | 78.6 | 79.8 | 78.1 | 78.1 | 81.3 | 81.3 | 78.3 | 77.6 | 79.1 | 79.8 |
| 22. TA8521_338618 | 88.1 | 88.3 | 89.5 | 91.0 | 87.3 | 87.3 | 87.6 | 90.0 | 86.9 | 89.1 | 88.8 | 89.8 |
| 23. TA1966_4686 | 84.7 | 85.9 | 87.1 | 87.6 | 85.2 | 85.2 | 88.8 | 87.1 | 86.4 | 85.9 | 84.9 | 86.6 |
| 24. AT3G03910.1 | 92.0 | 85.9 | 89.1 | 89.8 | 85.4 | 85.4 | 84.2 | 88.6 | 85.4 | 88.6 | 86.6 | 87.8 |
| 25. AT5G18170.1 | | 88.3 | 90.5 | 89.8 | 86.9 | 86.9 | 84.7 | 90.0 | 87.8 | 88.1 | 89.8 | 89.8 |
| 26. TA153_347529 | 94.9 | | 90.3 | 90.3 | 93.4 | 93.4 | 84.4 | 89.1 | 88.1 | 87.8 | 88.6 | 88.8 |
| 27. Gm0155x00045.1 | 97.1 | 94.9 | | 90.5 | 88.6 | 88.6 | 84.9 | 89.8 | 89.5 | 92.5 | 92.7 | 89.5 |
| 28. TA9810_29730 | 96.4 | 96.4 | 96.4 | | 90.0 | 90.0 | 86.4 | 91.2 | 88.1 | 92.2 | 89.8 | 89.8 |
| 29. TA8643_4232 | 95.6 | 97.6 | 95.1 | 96.6 | | 100.0 | 85.2 | 90.0 | 87.1 | 85.9 | 87.6 | 88.6 |
| 30. TA1274_73275 | 95.6 | 97.6 | 95.1 | 96.6 | 100.0 | | 85.2 | 90.0 | 87.1 | 85.9 | 87.6 | 88.6 |
| 31. TA35352_4513 | 93.7 | 93.7 | 93.2 | 94.6 | 93.9 | 93.9 | | 86.9 | 83.7 | 83.5 | 83.5 | 87.3 |
| 32. TA11677_35883 | 96.1 | 95.1 | 95.9 | 97.1 | 96.1 | 96.1 | 93.2 | | 88.6 | 88.6 | 90.3 | 93.9 |
| 33. TA3379_3544 | 94.6 | 95.1 | 95.4 | 95.4 | 95.6 | 95.6 | 93.4 | 95.4 | | 88.3 | 89.8 | 88.1 |
| 34. AC174375_7.5 | 96.6 | 95.4 | 97.6 | 98.3 | 95.4 | 95.4 | 93.9 | 96.6 | 95.4 | | 90.8 | 88.3 |
| 35. TA21862_3880 | 95.1 | 94.9 | 96.8 | 95.9 | 94.6 | 94.6 | 92.5 | 95.6 | 95.1 | 96.6 | | 88.6 |
| 36. TA18464_4097 | 96.6 | 95.6 | 95.9 | 97.1 | 96.4 | 96.4 | 94.2 | 97.3 | 95.6 | 96.1 | 94.6 | |
| 37. TA2019_39350 | 95.9 | 95.1 | 95.9 | 95.9 | 95.6 | 95.6 | 92.9 | 96.4 | 95.4 | 95.9 | 94.6 | 95.4 |
| 38. Os03g0794500 | 94.2 | 94.4 | 94.2 | 95.9 | 94.4 | 94.4 | 98.8 | 93.9 | 94.4 | 95.4 | 93.4 | 95.1 |
| 39. TA14146_3330 | 93.9 | 93.7 | 93.9 | 94.6 | 94.2 | 94.2 | 92.7 | 92.9 | 93.4 | 94.6 | 93.4 | 93.9 |
| 40. TA13960_3332 | 93.7 | 93.4 | 93.4 | 94.4 | 93.9 | 93.9 | 92.5 | 92.7 | 92.9 | 94.2 | 93.4 | 93.7 |
| 41. Pt571209 | 96.1 | 95.4 | 96.8 | 98.3 | 95.6 | 95.6 | 94.2 | 96.1 | 94.2 | 97.6 | 96.4 | 96.1 |
| 42. Pt826140 | 96.4 | 94.6 | 95.9 | 97.6 | 94.6 | 94.6 | 93.9 | 94.9 | 93.9 | 96.8 | 95.4 | 95.6 |
| 43. TA35879_4081 | 95.1 | 95.1 | 94.6 | 95.4 | 95.4 | 95.4 | 92.2 | 96.1 | 96.1 | 94.9 | 94.4 | 96.8 |
| 44. TA29537_4113 | 95.4 | 95.4 | 94.9 | 95.6 | 95.6 | 95.6 | 92.5 | 96.1 | 96.4 | 95.1 | 94.6 | 97.1 |
| 45. TA69991_4565 | 93.7 | 93.9 | 93.2 | 94.6 | 93.9 | 93.9 | 100.0 | 93.2 | 93.4 | 93.9 | 92.5 | 94.2 |
| 46. GSVIVT25474001 | 96.8 | 95.6 | 96.4 | 97.6 | 95.9 | 95.9 | 94.9 | 95.1 | 94.9 | 96.8 | 95.1 | 96.4 |
| 47. TA43933_29760 | 96.1 | 94.9 | 95.6 | 96.4 | 95.1 | 95.1 | 94.2 | 94.4 | 94.2 | 96.1 | 94.4 | 95.6 |
| 48. AY106054 | 93.4 | 93.2 | 92.5 | 94.2 | 93.4 | 93.4 | 97.3 | 92.9 | 93.2 | 93.4 | 91.7 | 93.7 |
| 49. TA1057_3197 | 90.8 | 91.7 | 90.0 | 91.2 | 92.2 | 92.2 | 90.8 | 91.0 | 90.8 | 90.5 | 90.8 | 90.5 |
| 50. Pp126976 | 87.1 | 87.6 | 87.1 | 88.3 | 88.6 | 88.6 | 86.6 | 87.3 | 88.3 | 86.9 | 86.6 | 88.3 |
| 51. Sm78170 | 87.8 | 88.1 | 87.8 | 88.3 | 88.6 | 88.6 | 89.1 | 87.8 | 86.9 | 87.6 | 86.6 | 88.6 |
| | 37 | 38 | 39 | 40 | 41 | 42 | 43 | 44 | 45 | 46 | 47 | 48 |
| 1. AT5G07440 | 78.6 | 79.8 | 80.8 | 80.8 | 79.1 | 78.8 | 80.5 | 81.3 | 80.3 | 80.5 | 79.8 | 79.8 |
| 2. AB066298 | 78.6 | 80.0 | 80.3 | 80.3 | 79.3 | 79.3 | 79.8 | 80.5 | 80.0 | 80.3 | 80.0 | 79.6 |
| 3. TA305_347529 | 79.3 | 79.8 | 81.5 | 81.0 | 79.8 | 79.6 | 80.0 | 80.3 | 80.5 | 81.3 | 80.5 | 78.8 |
| 4. TA29927_3635 | 80.3 | 81.3 | 82.0 | 81.8 | 82.0 | 80.5 | 80.8 | 81.0 | 81.8 | 82.2 | 81.5 | 80.0 |
| 5. TA64336_3847 | 79.9 | 80.3 | 83.3 | 83.0 | 80.6 | 80.1 | 80.6 | 81.3 | 81.1 | 81.1 | 80.3 | 79.4 |
| 6. TA10049_29730 | 79.1 | 81.8 | 81.8 | 81.5 | 80.5 | 79.3 | 80.5 | 80.3 | 82.2 | 80.8 | 80.0 | 80.3 |
| 7. TA10097_29730 | 80.8 | 81.8 | 82.7 | 82.5 | 82.5 | 81.0 | 81.3 | 81.5 | 82.2 | 82.7 | 82.0 | 80.5 |
| 8. TA34363_4513 | 78.8 | 80.8 | 81.5 | 81.5 | 79.3 | 79.1 | 79.6 | 79.8 | 81.0 | 79.8 | 78.6 | 79.8 |
| 9. CR931735_5.4 | 78.8 | 79.6 | 81.3 | 81.0 | 80.8 | 80.0 | 79.8 | 80.5 | 79.6 | 80.5 | 79.3 | 78.8 |
| 10. Os04g45970.1 | 79.4 | 81.8 | 83.3 | 83.0 | 80.8 | 80.8 | 80.8 | 80.8 | 81.6 | 81.3 | 80.3 | 80.3 |
| 11. Os02g0650900 | 77.4 | 77.1 | 79.6 | 79.6 | 77.9 | 77.6 | 78.1 | 78.1 | 77.4 | 79.8 | 79.3 | 75.7 |
| 12. TA199_3690 | 80.8 | 80.5 | 82.0 | 81.8 | 80.5 | 80.5 | 80.0 | 80.3 | 81.0 | 81.5 | 80.8 | 79.6 |
| 13. TA12661_3352 | 76.6 | 80.3 | 79.8 | 79.8 | 78.3 | 77.4 | 79.1 | 78.8 | 79.1 | 79.3 | 78.3 | 78.8 |
| 14. TA7375_113636 | 80.3 | 80.0 | 81.3 | 81.0 | 80.5 | 80.0 | 79.8 | 80.0 | 81.0 | 81.5 | 80.8 | 79.1 |
| 15. Pt575509 | 80.8 | 80.5 | 82.0 | 81.8 | 81.0 | 80.5 | 80.5 | 80.8 | 81.5 | 82.0 | 81.3 | 79.6 |
| 16. Pt828764 | 80.3 | 80.3 | 82.5 | 82.2 | 80.5 | 80.0 | 80.5 | 80.8 | 81.0 | 82.0 | 80.8 | 79.1 |
| 17. Sb5286803 | 79.3 | 81.0 | 82.0 | 82.0 | 80.0 | 80.3 | 79.6 | 80.0 | 82.0 | 80.5 | 79.3 | 79.6 |
| 18. TA48180_4081 | 79.1 | 79.6 | 81.8 | 81.5 | 79.8 | 79.6 | 79.8 | 80.0 | 80.5 | 81.5 | 80.3 | 78.3 |
| 19. TA70276_4565 | 78.8 | 80.8 | 81.8 | 81.8 | 79.8 | 79.6 | 79.8 | 80.0 | 80.8 | 80.8 | 79.6 | 79.3 |
| 20. TA36948_29760 | 78.8 | 81.5 | 79.3 | 79.1 | 79.6 | 79.6 | 78.8 | 79.1 | 81.3 | 80.8 | 79.6 | 80.3 |
| 21. TA160461_4577 | 79.1 | 79.8 | 81.0 | 81.0 | 79.6 | 79.6 | 79.1 | 79.6 | 81.0 | 79.8 | 78.6 | 79.1 |
| 22. TA8521_338618 | 88.8 | 89.3 | 86.6 | 86.4 | 90.8 | 90.0 | 88.6 | 88.6 | 87.8 | 92.2 | 90.5 | 86.4 |
| 23. TA1966_4686 | 85.2 | 90.0 | 86.4 | 85.9 | 86.6 | 86.6 | 86.1 | 86.1 | 89.1 | 88.1 | 87.3 | 87.6 |
| 24. AT3G03910.1 | 87.6 | 84.9 | 83.2 | 83.0 | 88.6 | 88.6 | 86.6 | 87.1 | 84.2 | 89.1 | 88.1 | 82.5 |
| 25. AT5G18170.1 | 89.8 | 85.6 | 86.1 | 86.1 | 91.2 | 90.5 | 89.8 | 90.8 | 84.7 | 91.2 | 90.0 | 83.7 |
| 26. TA153_347529 | 89.1 | 86.1 | 85.6 | 85.4 | 89.5 | 89.3 | 89.8 | 89.8 | 84.4 | 90.3 | 89.1 | 83.5 |
| 27. Gm0155x00045.1 | 88.8 | 87.1 | 86.1 | 85.9 | 90.8 | 90.3 | 89.1 | 89.1 | 85.2 | 92.0 | 90.8 | 83.9 |
| 28. TA9810_29730 | 89.8 | 87.8 | 86.4 | 85.9 | 93.7 | 91.7 | 89.8 | 90.3 | 86.6 | 92.7 | 91.5 | 85.4 |
| 29. TA8643_4232 | 87.3 | 85.9 | 84.4 | 84.4 | 88.8 | 88.6 | 88.6 | 89.1 | 85.2 | 89.3 | 88.6 | 84.7 |
| 30. TA1274_73275 | 87.3 | 85.9 | 84.4 | 84.4 | 88.8 | 88.6 | 88.6 | 89.1 | 85.2 | 89.3 | 88.6 | 84.7 |
| 31. TA35352_4513 | 85.9 | 96.8 | 84.4 | 84.2 | 85.6 | 85.6 | 87.1 | 87.1 | 99.8 | 87.1 | 86.4 | 93.9 |
| 32. TA11677_35883 | 92.2 | 87.3 | 85.6 | 85.6 | 91.7 | 91.2 | 92.2 | 92.2 | 86.9 | 91.7 | 90.5 | 85.4 |
| 33. TA3379_3544 | 88.1 | 85.2 | 84.2 | 83.9 | 86.9 | 86.9 | 88.3 | 89.3 | 83.7 | 88.8 | 87.6 | 82.2 |
| 34. AC174375_7.5 | 87.6 | 85.4 | 84.9 | 84.7 | 89.1 | 88.3 | 86.9 | 87.3 | 83.7 | 90.3 | 89.3 | 82.2 |
| 35. TA21862_3880 | 88.6 | 85.2 | 86.1 | 86.4 | 90.5 | 90.3 | 88.3 | 88.8 | 83.7 | 90.5 | 89.3 | 83.0 |
| 36. TA18464_4097 | 90.8 | 88.3 | 85.6 | 85.6 | 90.5 | 90.5 | 93.9 | 93.9 | 87.3 | 92.2 | 91.0 | 86.6 |
| 37. TA2019_39350 | | 87.1 | 85.9 | 85.4 | 89.5 | 89.5 | 89.8 | 90.3 | 85.9 | 91.2 | 90.0 | 84.9 |
| 38. Os03g0794500 | 93.9 | | 84.9 | 84.7 | 87.1 | 87.1 | 88.3 | 88.3 | 97.1 | 89.1 | 88.3 | 95.1 |
| 39. TA14146_3330 | 92.7 | 93.7 | | 99.5 | 85.6 | 85.4 | 85.2 | 85.2 | 84.7 | 86.6 | 86.1 | 82.2 |
| 40. TA13960_3332 | 92.5 | 93.2 | 99.8 | | 85.6 | 85.6 | 85.4 | 85.4 | 84.4 | 86.9 | 86.4 | 82.5 |
| 41. Pt571209 | 95.1 | 95.4 | 94.2 | 94.4 | | 95.4 | 90.0 | 90.5 | 85.9 | 93.7 | 92.0 | 85.2 |
| 42. Pt826140 | 94.2 | 96.1 | 93.9 | 93.9 | 98.8 | | 88.6 | 89.1 | 85.6 | 93.2 | 91.5 | 85.4 |
| 43. TA35879_4081 | 94.2 | 93.2 | 92.2 | 92.5 | 95.1 | 94.6 | | 98.8 | 87.1 | 91.2 | 90.5 | 86.1 |
| 44. TA29537_4113 | 94.4 | 93.4 | 92.5 | 92.7 | 95.4 | 94.9 | 99.8 | | 87.1 | 91.7 | 90.8 | 85.9 |
| 45. TA69991_4565 | 92.9 | 98.8 | 92.7 | 92.5 | 94.2 | 94.2 | 92.2 | 92.5 | | 87.3 | 86.6 | 93.9 |
| 46. GSVIVT25474001 | 94.9 | 96.1 | 94.4 | 94.4 | 98.3 | 97.8 | 95.6 | 95.9 | 94.9 | | 98.3 | 86.6 |
| 47. TA43933_29760 | 94.2 | 95.4 | 93.7 | 93.7 | 97.1 | 96.6 | 94.9 | 95.1 | 94.2 | 98.8 | | 85.9 |
| 48. AY106054 | 92.9 | 97.6 | 92.5 | 92.2 | 93.7 | 93.9 | 91.7 | 92.2 | 97.3 | 93.9 | 93.2 | |
| 49. TA1057_3197 | 91.0 | 91.2 | 91.5 | 91.2 | 90.8 | 91.0 | 90.0 | 90.3 | 90.8 | 90.3 | 89.5 | 90.3 |
| 50. Pp126976 | 86.9 | 87.1 | 88.3 | 87.8 | 87.8 | 87.3 | 87.8 | 88.1 | 86.9 | 87.3 | 86.4 | 86.1 |
| 51. Sm78170 | 86.4 | 88.8 | 89.5 | 89.3 | 89.1 | 88.8 | 87.1 | 87.1 | 89.1 | 88.3 | 87.3 | 88.6 |

| | 49 | 50 | 51 |
|---|---|---|---|
| 1. AT5G07440 | 77.6 | 76.4 | 74.5 |
| 2. AB066298 | 78.6 | 76.2 | 75.2 |
| 3. TA305_347529 | 75.2 | 76.2 | 74.0 |
| 4. TA29927_3635 | 77.4 | 76.6 | 74.5 |
| 5. TA64336_3847 | 77.4 | 75.0 | 74.8 |
| 6. TA10049_29730 | 77.1 | 74.7 | 74.5 |
| 7. TA10097_29730 | 78.1 | 77.1 | 74.9 |
| 8. TA34363_4513 | 75.2 | 73.2 | 72.7 |
| 9. CR931735_5.4 | 76.2 | 73.2 | 74.7 |
| 10. Os04g45970.1 | 76.9 | 74.0 | 73.8 |
| 11. Os02g0650900 | 74.7 | 73.2 | 72.0 |
| 12. TA199_3690 | 80.0 | 76.2 | 74.5 |
| 13. TA12661_3352 | 74.7 | 71.5 | 70.8 |
| 14. TA7375_113636 | 79.3 | 75.9 | 74.0 |
| 15. Pt575509 | 80.0 | 76.6 | 74.5 |
| 16. Pt828764 | 79.1 | 76.4 | 74.2 |
| 17. Sb5286803 | 75.4 | 74.2 | 72.7 |
| 18. TA48180_4081 | 77.6 | 74.0 | 74.0 |
| 19. TA70276_4565 | 75.9 | 73.0 | 72.5 |
| 20. TA36948_29760 | 77.4 | 74.9 | 74.0 |
| 21. TA160461_4577 | 75.4 | 73.0 | 71.5 |
| 22. TA8521_338618 | 77.4 | 75.7 | 76.4 |
| 23. TA1966_4686 | 76.4 | 75.4 | 76.9 |
| 24. AT3G03910.1 | 76.2 | 74.7 | 72.7 |
| 25. AT5G18170.1 | 78.3 | 75.7 | 75.9 |
| 26. TA153_347529 | 80.0 | 75.7 | 76.6 |
| 27. Gm0155x00045.1 | 77.9 | 75.2 | 76.4 |
| 28. TA9810_29730 | 78.3 | 76.2 | 77.1 |
| 29. TA8643_4232 | 79.3 | 76.2 | 76.2 |
| 30. TA1274_73275 | 79.3 | 76.2 | 76.2 |
| 31. TA35352_4513 | 78.1 | 75.4 | 77.1 |
| 32. TA11677_35883 | 79.6 | 76.9 | 76.4 |
| 33. TA3379_3544 | 78.1 | 75.9 | 75.9 |
| 34. AC174375_7.5 | 76.9 | 74.9 | 74.9 |
| 35. TA21862_3880 | 80.0 | 76.2 | 75.9 |
| 36. TA18464_4097 | 78.6 | 76.6 | 76.9 |
| 37. TA2019_39350 | 79.8 | 75.9 | 76.4 |
| 38. Os03g0794500 | 78.1 | 74.5 | 77.4 |
| 39. TA14146_3330 | 78.3 | 77.4 | 77.6 |
| 40. TA13960_3332 | 78.3 | 77.4 | 77.6 |
| 41. Pt571209 | 78.6 | 75.2 | 77.4 |
| 42. Pt826140 | 78.8 | 76.2 | 78.1 |
| 43. TA35879_4081 | 77.9 | 76.9 | 76.4 |
| 44. TA29537_4113 | 78.3 | 76.9 | 76.9 |
| 45. TA69991_4565 | 78.1 | 75.4 | 77.1 |
| 46. GSVIVT25474001 | 79.1 | 76.6 | 77.6 |
| 47. TA43933_29760 | 77.9 | 76.6 | 77.1 |
| 48. AY106054 | 77.1 | 73.5 | 76.2 |
| 49. TA1057_3197 | | 78.3 | 76.6 |
| 50. Pp126976 | 91.0 | | 74.0 |
| 51. Sm78170 | 89.1 | 87.3 | |

The percentage identity between SEQ ID NO: 2 (AY106054 on line 48) and other GDH polypeptide sequences within the subgroups of Class I and mosses does not fall below 73%, whereas the percentage identity between SEQ ID NO: 110 (Os02g0650900 on line 11) and other GDH polypeptide sequences within the subgroups of Class I and mosses does not fall below 72%, which illustrates the high sequence conservation. Minimal identity among the sequences within alpha subunit subgroup of GDH proteins is 75%. Minimal identity within beta subunit subgroup of GDH is 82%. Identity between alpha and beta subunit subgroups ranges between 75-85%. Most of the alpha and beta subunit sequences have 80% or more identity with AY106054 and Os02g0650900. Minimal identity within class II GDH proteins is 26%.

### 2. Small Auxin-Up RNA (SAUR) Polypeptides

Parameters used in the comparison were:
Scoring matrix: Blosum62,
First Gap: 12,
Extending Gap: 2.

Results of the software analysis are shown in Table B2 for the global similarity and identity over the full length of the polypeptide sequences. The sequence identity (in %) between a selection of SAUR polypeptide sequences from Table A3. A SAUR polypeptide useful in performing the methods of the invention is generally higher than 22.8% compared to SEQ ID NO: 502 (A. thaliana_AT2G21210).

TABLE B2
MatGAT results for global similarity and identity over the full length of the
polypeptide sequences.

| | 1. | 2. | 77. | 111. | 112. | 113. | 114. | 128. |
|---|---|---|---|---|---|---|---|---|
| 1. O.sativa_OsSAUR1 | | 31.7 | 23.9 | 26 | 22.8 | 26.5 | 24.8 | 23.4 |
| 2. O.sativa_OsSAUR2 | 38.5 | | 30.1 | 22.8 | 25.8 | 23.6 | 24.8 | 22.1 |
| 77. A.thaliana_AT2G21210 | 44.9 | 41.8 | | 49 | 68.9 | 49.5 | 43.6 | 39.4 |
| 112. A.thaliana_AT4G38840 | 42.4 | 41 | 82.8 | 69.7 | | 53.5 | 46.2 | 41.9 |
| 113. A.thaliana_AT4G38850 | 47.3 | 35.2 | 68.4 | 77.5 | 68.7 | | 43.5 | 39.8 |
| 114. A.thaliana_AT4G38860 | 40 | 41.8 | 65.7 | 52.4 | 62.9 | 58.1 | | 57.5 |
| 128. A.thaliana_AT5G66260 | 38.4 | 36.9 | 57.6 | 55.6 | 56.6 | 55.6 | 67.6 | |

### 3. SAUR Polypeptides, or SYNP Polypeptides, or Fusion Proteins of SAUR Polypeptides and SYNP Polypeptides

Results of the software analysis are shown in Table B3 for the global similarity and identity over the full length of the polypeptide sequences. The sequence identity (in %) between a selection of SAUR polypeptide sequences from Table A4. A SAUR polypeptide useful in performing the methods of the invention is generally higher than 22.8% compared to SEQ ID NO: 1164 (A. thaliana_AT2G21210).

TABLE B3
MatGAT results for global similarity and identity over the full length of the
polypeptide sequences.

| | 1. | 2. | 77. | 111. | 112. | 113. | 114. | 128. |
|---|---|---|---|---|---|---|---|---|
| 1. O.sativa_OsSAUR1 | | 31.7 | 23.9 | 26 | 22.8 | 26.5 | 24.8 | 23.4 |
| 2. O.sativa_OsSAUR2 | 38.5 | | 30.1 | 22.8 | 25.8 | 23.6 | 24.8 | 22.1 |
| 77. A.thaliana_AT2G21210 | 44.9 | 41.8 | | 49 | 68.9 | 49.5 | 43.6 | 39.4 |
| 112. A.thaliana_AT4G38840 | 42.4 | 41 | 82.8 | 69.7 | | 53.5 | 46.2 | 41.9 |
| 113. A.thaliana_AT4G38850 | 47.3 | 35.2 | 68.4 | 77.5 | 68.7 | | 43.5 | 39.8 |
| 114. A.thaliana_AT4G38860 | 40 | 41.8 | 65.7 | 52.4 | 62.9 | 58.1 | | 57.5 |
| 128. A.thaliana_AT5G66260 | 38.4 | 36.9 | 57.6 | 55.6 | 56.6 | 55.6 | 67.6 | |

### 4. Dehydroascorbate Reductase (DHAR) Polypeptides

Parameters used in the comparison were:
Scoring matrix: Blosum62
First Gap: 12
Extending gap: 2

Results of the software analysis are shown in Table B4 for the global and identity over the full length of the polypeptide sequences.

The percentage identity between the DHAR polypeptide sequences useful in performing the methods of the invention can be as low as 49% amino acid identity compared to SEQ ID NO: 1958.

TABLE B4
MatGAT results for global similarity and identity over the full length of the
polypeptide sequences.

| Name | 101 | 102 | 103 | 104 | 105 | 106 | 107 | 108 | 109 | 110 |
|---|---|---|---|---|---|---|---|---|---|---|
| 101. S.aethnensis_TA30_121540 | | 52.6 | 70.4 | 78.5 | 75.2 | 74.8 | 66.7 | 69.8 | 75.7 | 83.7 |
| 102. S.bicolor_Sb09g001690.1 | | | 64 | 55.4 | 55.8 | 55.4 | 62.2 | 65.2 | 55.1 | 51.3 |
| 103. S.bicolor_Sb09g001700.1 | | | | 74.3 | 69.6 | 69.2 | 95.8 | 82.7 | 71 | 69.8 |
| 104. S.indicum_DQ287974 | | | | | 78.3 | 78.3 | 70.6 | 72.8 | 79.7 | 78.4 |
| 105. S.lycopersicum_TC196877 | | | | | | 97.6 | 67.5 | 72.3 | 75.9 | 72.3 |
| 106. S.tuberosum_TC167795 | | | | | | | 67 | 71.4 | 75.9 | 72.3 |
| 107. T.aestivum_CA484858 | | | | | | | | 79 | 67.3 | 66 |
| 108. T.aestivum_TC278165 | | | | | | | | | 73.2 | 67.8 |
| 109. T.hispida_TA863_189793 | | | | | | | | | | 76.1 |
| 110. T.kok-saghyz_TA971_333970 | | | | | | | | | | |

### Example 4

### Identification of Domains Comprised in Polypeptide Sequences Useful in Performing the Methods of the Invention

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

### 1. Glutamate Dehydrogenase (GDH) Polypeptides

The results of the InterPro scan of the polypeptide sequence as represented by SEQ ID NO: 2 are presented in Table C1.

TABLE C1
InterPro scan results (major accession numbers) of the polypeptide
sequence as represented by SEQ ID NO: 2.

| | | | Amino acid |
|---|---|---|---|
| | | | coordinates |
| | | | (SEQ ID |
| Database | Accession number | Accession name | NO 2) |
| Interpro | IPR006095 | Glutamate/phenylalanine/leucine/ | |
| | | valine dehydrogenase | |
| PRINTS | PR00082 | GLFDHDRGNASE | [88-102]T |
| | | | [167-189]T |
| | | | [209-229]T |
| | | | [336-347]T |
| PANTHER | PTHR11606:SF2 | GLUTAMATE DEHYDROGENASE | [1-410]T |
| Interpro | IPR006096 | Glutamate/phenylalanine/leucine/valine | |
| | | dehydrogenase, C-terminal | |
| PFAM | PF00208 | ELFV_dehydrog | [176-408]T |
| Interpro | IPR006097 | Glutamate/phenylalanine/leucine/valine | |
| | | dehydrogenase, dimerisation region | |
| PFAM | PF02812 | ELFV_dehydrog_N | [31-161]T |
| Interpro | IPR014362 | Glutamate dehydrogenase | |
| PIR | PIRSF000185 | Glutamate dehydrogenase | [1-411]T |
| Interpro | IPR016040 | NAD(P)-binding | |
| GENE3D | G3DSA:3.40.50.720 | no description | [176-409]T |
| SUPERFAMILY | SSF51735 | NAD(P)-binding Rossmann-fold | [175-409]T |
| | | domains | |
| | noIPR | unintegrated | |
| GENE3D | G3DSA:3.40.192.10 | no description | [33-175]T |
| PANTHER | PTHR11606 | GLUTAMATE DEHYDROGENASE | [1-410]T |
| SUPERFAMILY | SSF53223 | Aminoacid dehydrogenase-like, N- | [1-170]T |
| | | terminal domain | |

### 2. Fasciclin-Like AGP (FLAs) Polypeptides

The results of the Pfam search of the polypeptide sequence as represented by SEQ ID NO: 172 are presented in Table C2.

TABLE C2
InterPro scan results (major accession numbers) of the polypeptide
sequence as represented by SEQ ID NO: 172.

| | | Accesion | Amino acid coordinates |
|---|---|---|---|
| Database | Accession name | number | on SEQ ID NO 172 |
| Pfam | fasciclin | (PF02469) | 37-64 and 198-327 |

Alternatively, conserved domains may be found by searching or scanning InterPro database. The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs.

### 3. Small Auxin-Up RNA (SAUR) Polypeptides

The results of the InterPro scan of the polypeptide sequence as represented by SEQ ID NO: 502 are presented in Table C3.

TABLE C3
InterPro scan results (major accession numbers) of the polypeptide
sequence as represented by SEQ ID NO: 502.

| | | | Amino acid coordinates |
|---|---|---|---|
| Database | Accession number | Accession name | on SEQ ID NO 502 |
| InterPro | IPR003676 | Auxin responsive | |
| | | SAUR protein | |
| PFAM | PF02519 | Auxin_inducible | 1-95 |

An auxin inducible domain is also referred to as Auxin responsive.

### 4. SAUR Polypeptides, or SYNP Polypeptides, or Fusion Proteins of SAUR Polypeptides and SYNP Polypeptides

The results of the InterPro scan of the polypeptide sequence as represented by SEQ ID NO: 1164 are presented in Table C4.

TABLE C4
InterPro scan results (major accession numbers) of the polypeptide
sequence as represented by SEQ ID NO: 1164.

| | | | Amino acid coordinates |
|---|---|---|---|
| Database | Accession number | Accession name | on SEQ ID NO 1164 |
| InterPro | IPR003676 | Auxin responsive | |
| | | SAUR protein | |
| PFAM | PF02519 | Auxin_inducible | 1-95 |

An auxin inducible domain is also referred to as Auxin responsive.

### 5. Dehydroascorbate Reductase (DHAR) Polypeptides

The results of the InterPro scan of the polypeptide sequence as represented by SEQ ID NO: 1958 are presented in Table C5.

TABLE C5
InterPro scan results (major accession numbers) of the polypeptide
sequence as represented by SEQ ID NO: 1958.

| | | | Amino acid |
|---|---|---|---|
| | | | coordinates on |
| | | | SEQ ID NO: |
| Database | Accession number | Accession name | 1958 |
| ProfileScan | PS50404 | GST_NTER | 10-88 |
| ProfileScan | PS50405 | GST_CTER | 66-210 |
| Gene3D | G3DSA:3.40.30.10 | no description | 20-90 |
| Gene3D | G3DSA:1.20.1050.10 | no description | 90-210 |
| HMMPanther | PTHR11260:SF15 | DEHYDROASCORBATE | 19-210 |
| | | REDUCTASE | |
| HMMPanther | PTHR11260 | GLUTATHIONE S-TRANSFERASE, | 19-210 |
| | | GST, SUPERFAMILY, | |
| | | GST DOMAIN CONTAINING | |
| HMMPfam | PF02798 | GST_N | 20-82 |
| superfamily | SSF47616 | Glutathione S-transferase | 83-209 |
| | | (GST), C-terminal domain | |
| superfamily | SSF52833 | Thioredoxin-like | 20-79 |

### Example 5

### Topology Prediction of the Polypeptide Sequences Useful in Performing the Methods of the Invention

### 1. Glutamate Dehydrogenase (GDH) Polypeptides

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The results of TargetP 1.1 analysis of the polypeptide sequence as represented by SEQ ID NO: 2 are presented Table D1. The “plant” organism group has been selected, no cutoffs defined, and the predicted length of the transit peptide requested. No particular subcellular localization of the polypeptide sequence was predicted.

TABLE D1
TargetP 1.1 analysis of the polypeptide sequence as represented by
SEQ ID NO: 2.

| Name | Len | cTP | mTP | SP | other | Loc | RC | TPlen |
|---|---|---|---|---|---|---|---|---|
| SEQ ID NO: 2 | 411 | 0.086 | 0.295 | 0.203 | 0.378 | — | 5 | — |
| cutoff | | 0.000 | 0.000 | 0.000 | 0.000 | | | |

Abbreviations:
Len, Length;
cTP, Chloroplastic transit peptide;
mTP, Mitochondrial transit peptide,
SP, Secretory pathway signal peptide,
other, Other subcellular targeting,
Loc, Predicted Location;
RC, Reliability class;
TPlen, Predicted transit peptide length.

When using other algorithms, a mitochondrial location is predicted (e.g. psort: mitochondrial: 0.508, cytoplasm 0.450; MitoP2: 0.6568), which is in agreement with the literature data.

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).

### 2. Fasciclin-Like AGP (FLAB) Polypeptides

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

FLA-like polypeptides are typically found anchored at a membrane, more typically the plasmatic membrane.

### 3. Small Auxin-Up RNA (SAUR) Polypeptides

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

Alternatively, many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).

### 4. SAUR Polypeptides, or SYNP Polypeptides, or Fusion Proteins of SAUR Polypeptides and SYNP Polypeptides

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

Alternatively, many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).

### 5. Dehydroascorbate Reductase (DHAR) Polypeptides

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).

### Example 6

### Assay Related to the Polypeptide Sequences Useful in Performing the Methods of the Invention

### 1. Glutamate Dehydrogenase (GDH) Polypeptides

Tissues are ground under liquid N₂, extracted in five volumes of GDH extraction buffer (100 mM Tris [pH 8.0], 2 mM EDTA, 5% insoluble PVPP, 5% soluble PVP-40, 1 mM DTT, 1 mM reduced glutathione, 0.1% v/v Triton X-100), and the extracts are clarified by centrifugation (13,000 g, 15 min, 4° C.).

GDH activity may be determined in both aminating and deaminating directions. The standard amination reaction mixture contains 100 mM Tris-HCl, pH 8.0, 20 mM α-ketoglutarate, 200 mM NH₄Cl, 1 mM CaCl₂, 0.2 mM NAD(P)H, enzyme solution, and deionized water to a final volume of 1 cm³. The standard deamination reaction mixture contains 100 mM Tris-HCl, pH 9.3, 100 mM L-Glu, 1 mM NAD(P)⁺, 0.5 mM CaCl₂, enzyme solution, and deionized water to a final volume of 1 cm³. All assays are performed at 30° C. The absorption change at 340 nm is measured using a Perkin-Elmer UV/VIS spectrophotometer. One unit of GDH activity is defined as the reduction or oxidation of 1 mmol of coenzyme [NAD(P)⁺, NAD(P)H, respectively] min⁻¹ at 30° C.

### 2. Fasciclin-Like AGP (FLAs) Polypeptides

The N-glycosylation activity of FLA-like polypeptides may be detected as described by Johnson et al., Plant Physiol. (2003) 133 (4) 1911-1925.

### 3. Dehydroascorbate Reductase (DHAR) Polypeptides

The functional assay for the DAHR polypeptide is described in: Kato, Y. (1997)—Plant Cell Physiol. 38(2): 173-178.

### Example 7

### Cloning of the Nucleic Acid Sequence Used in the Methods of the Invention

### 1. Glutamate Dehydrogenase (GDH) Polypeptides

a) cloning of Zm_GDH (SEQ ID NO: 1/2)

The nucleic acid sequence used in the methods of the invention was amplified by PCR using as template a custom-made Zea mays seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm7065 (SEQ ID NO: 27; sense, start codon in bold): 5′-ggggacaagtttgtacaaaaaagcaggcttaaa caatgaatgcattggcagca-3′ and prm7066 (SEQ ID NO: 28; reverse, complementary): 5′-ggggaccactttgta caagaaagctgggtggaggtcatgcttcccatc-3′, which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, pZmGDH. Plasmid pDONR201 was purchased from Invitrogen, as part of the Gateway® technology.

The entry clone comprising SEQ ID NO: 1 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 23) for constitutive specific expression (or a rice RCc3 promoter (SEQ ID NO: 24) for root specific expression) was located upstream of this Gateway cassette.

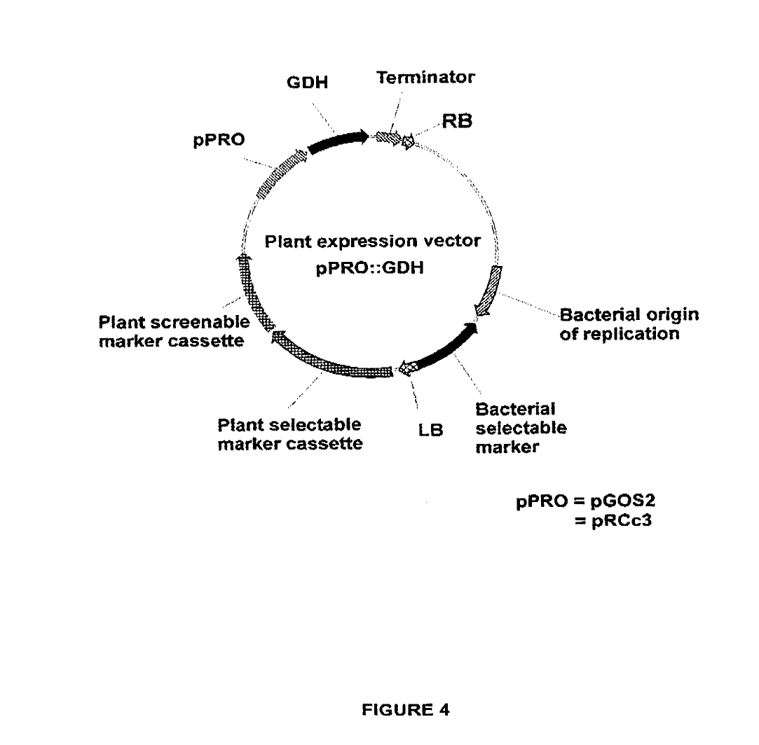

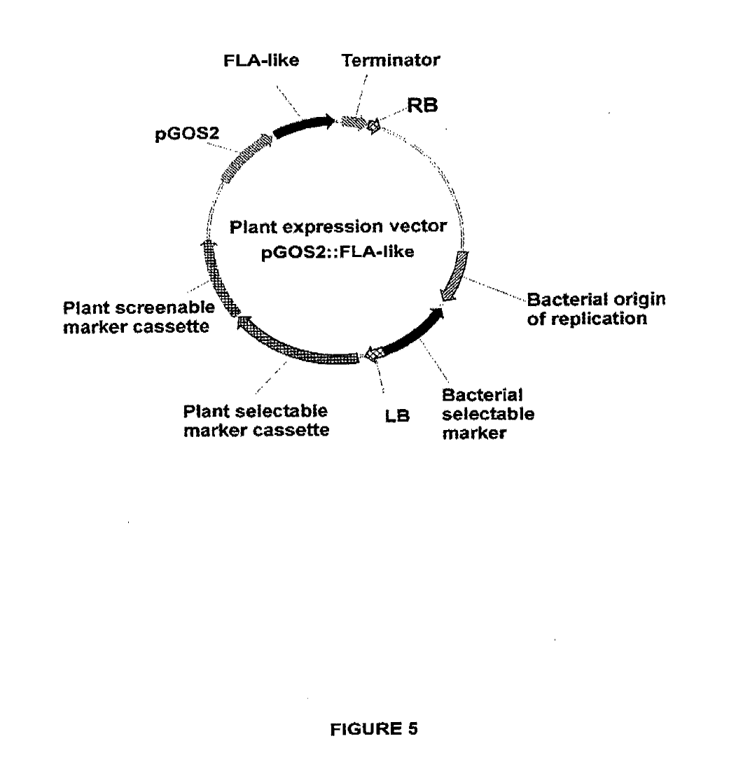

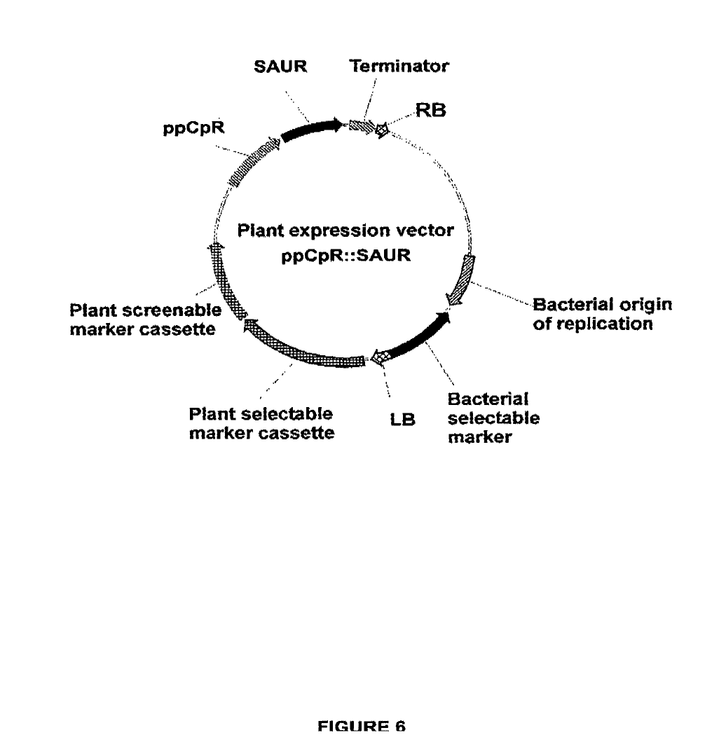

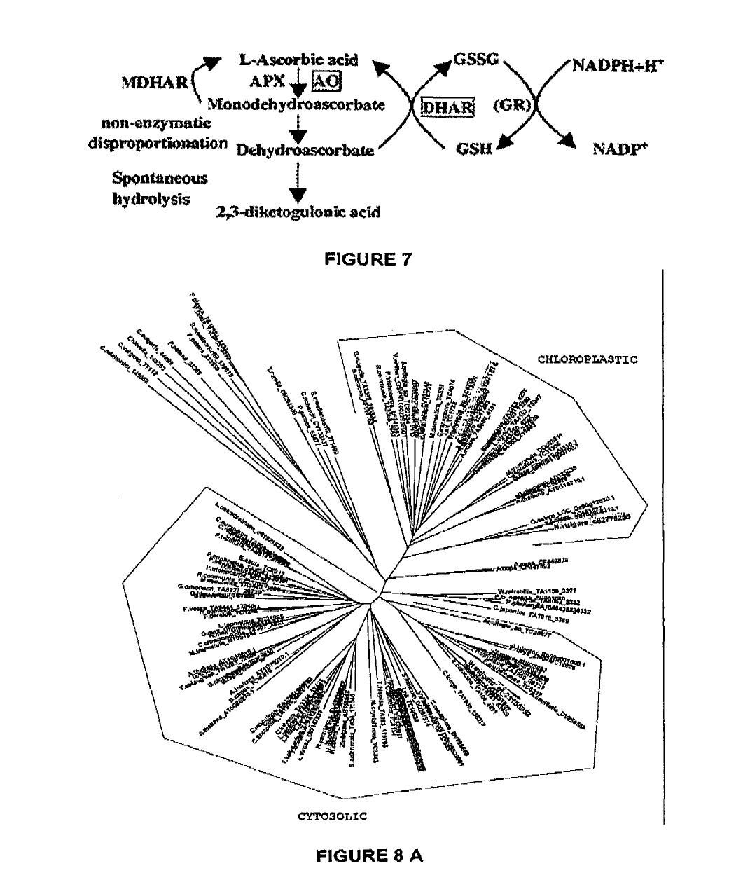

After the LR recombination step, the resulting expression vector pGOS2::ZmGDH (FIG. 4) or the pRCc3::ZmGDH was transformed into Agrobacterium strain LBA4044 according to methods well known in the art.

b) cloning of Os_GDH (SEQ ID NO: 39/116)

The nucleic acid sequence used in the methods of the invention was amplified by PCR using as template a custom-made Oryza sativa seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm7063 (SEQ ID NO: 25; sense, start codon in bold): 5′-ggggacaagtttgtacaaaaaagc aggcttaaacaatgaacgcgctagccg-3′ and prm7064 (SEQ ID NO: 26; reverse, complementary): 5′-ggggaccactttgtacaagaaagctgggtcctcaacagattctcatgcc t-3′, which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, pOsGDH. Plasmid pDONR201 was purchased from Invitrogen, as part of the Gateway® technology.

The entry clone comprising SEQ ID NO: 39 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 23) for constitutive specific expression (or a rice RCc3 promoter (SEQ ID NO: 24) for root specific expression) was located upstream of this Gateway cassette.

After the LR recombination step, the resulting expression vector pGOS2::OsGDH or the pRCc3::OsGDH was transformed into Agrobacterium strain LBA4044 according to methods well known in the art.

### 2. Fasciclin-Like AGP (FLAB) Polypeptides

The nucleic acid sequence was amplified by PCR using as template a custom-made Lycopersicum esculentum seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were as in SEQ ID NO: 498; sense): 5′-ggggacaagtttgtacaaaaaagcaggcttaaacaatgcagcttccgtcgtc-3′ and as in SEQ ID NO: 499; reverse, complementary: 5′-ggggaccactttgtacaagaaagctgggtttctttttcaaacttccatcaa-3′, which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, pFLA-like. Plasmid pDONR201 was purchased from Invitrogen, as part of the Gateway® technology.

The entry clone comprising SEQ ID NO: 171 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 500) for constitutive specific expression was located upstream of this Gateway cassette.

After the LR recombination step, the resulting expression vector pGOS2::FLA-like polypeptide was transformed into Agrobacterium strain LBA4044 according to methods well known in the art.

### 3. Small Auxin-Up RNA (SAUR) Polypeptides

The nucleic acid sequence was amplified by PCR using as template a custom-made Arabidopsis thaliana seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were a represented by SEQ ID NO: 1161 and 1162; which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, pSAUR. Plasmid pDONR201 was purchased from Invitrogen, as part of the Gateway® technology.

The entry clone comprising the coding region of SEQ ID NO: 501 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A leaf-specific promoter (SEQ ID NO: 1163) for leaf-specific expression was located upstream of this Gateway cassette.

After the LR recombination step, the resulting expression vector ppCpR::SAUR was transformed into Agrobacterium strain LBA4044 according to methods well known in the art.

The above identical procedure was carried out for SAUR-33 represented by SEQ ID NO: 2210 under the control of an gos2 promoter as represented by SEQ ID NO: 2288.

### 4. SAUR Polypeptides, or SYNP Polypeptides, or Fusion Proteins of SAUR Polypeptides and SYNP Polypeptides

The nucleic acid sequence was amplified by PCR using as template a custom-made Arabidopsis thaliana seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were a represented by SEQ ID NO: 1823 and 1824; which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, pSAUR. Plasmid pDONR201 was purchased from Invitrogen, as part of the Gateway® technology.

The entry clone comprising the coding region of SEQ ID NO: 1163 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice leaf-specific promoter (SEQ ID NO: 1825) for leaf-specific expression was located upstream of this Gateway cassette.

After the LR recombination step, the resulting expression vector pGOS2::SAUR was transformed into Agrobacterium strain LBA4044 according to methods well known in the art.

### 5. Dehydroascorbate Reductase (DHAR) Polypeptides

The nucleic acid sequence was amplified by PCR using as template a custom-made Solanum lycopersicum seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm12191 (SEQ ID NO: 2252; sense, start codon in bold): 5′-ggggacaagtttgtacaaaaaagcaggcttaaacaatggttgttgaagtttgtgtc-3′ and prm12192 (SEQ ID NO: 2253; reverse, complementary): 5′-ggggaccactttgtacaagaa agctgggttcatacgttaaacctttg gag-3′, which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, pDHAR. Plasmid pDONR201 was purchased from Invitrogen, as part of the Gateway® technology.

The entry clone comprising SEQ ID NO: 1957 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 2251) for constitutive specific expression was located upstream of this Gateway cassette.

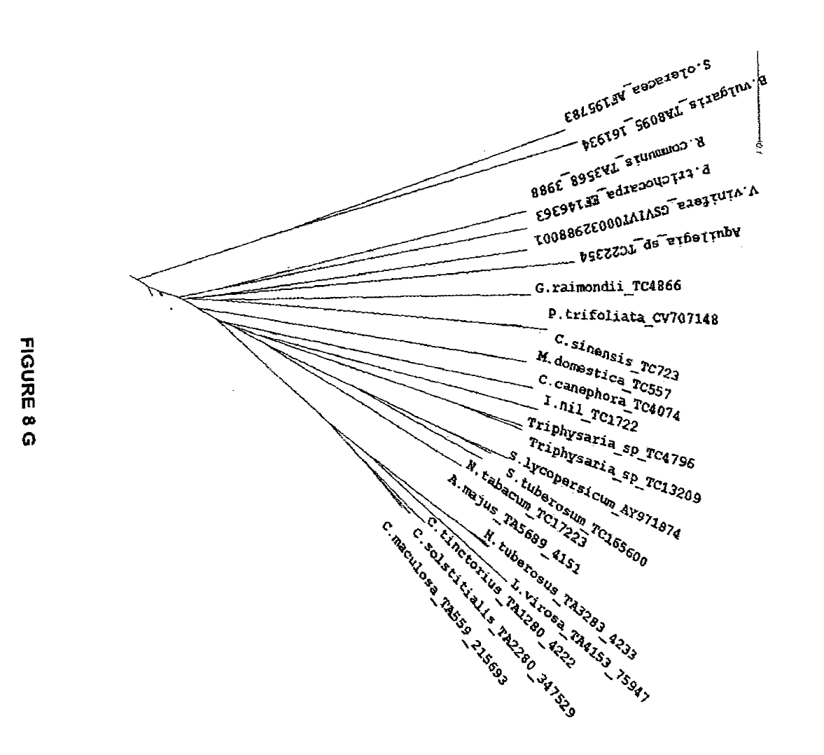

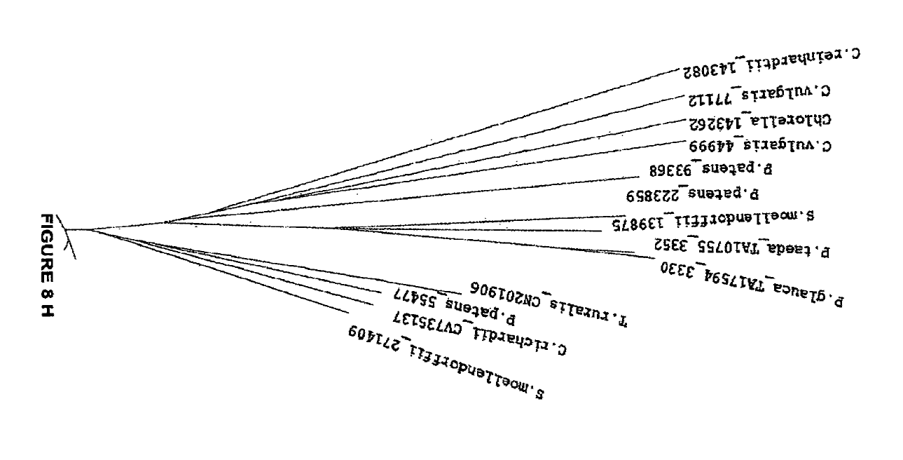

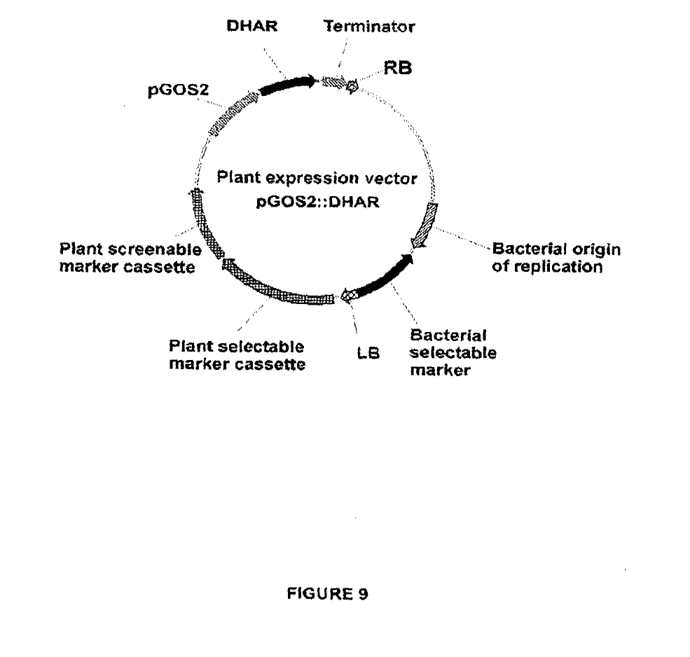

After the LR recombination step, the resulting expression vector pGOS2::DHAR (FIG. 9) was transformed into Agrobacterium strain LBA4044 according to methods well known in the art.

### Example 8

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges 1996, Chan et al. 1993, Hiei et al. 1994).

### Example 9

### Transformation of Other Crops

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7 Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Cotton Transformation

Cotton is transformed using Agrobacterium tumefaciens according to the method described in U.S. Pat. No. 5,159,135. Cotton seeds are surface sterilised in 3% sodium hypochlorite solution during 20 minutes and washed in distilled water with 500 μg/ml cefotaxime. The seeds are then transferred to SH-medium with 50 μg/ml benomyl for germination. Hypocotyls of 4 to 6 days old seedlings are removed, cut into 0.5 cm pieces and are placed on 0.8% agar. An Agrobacterium suspension (approx. 108 cells per ml, diluted from an overnight culture transformed with the gene of interest and suitable selection markers) is used for inoculation of the hypocotyl explants. After 3 days at room temperature and lighting, the tissues are transferred to a solid medium (1.6 g/l Gelrite) with Murashige and Skoog salts with B5 vitamins (Gamborg et al., Exp. Cell Res. 50:151-158 (1968)), 0.1 mg/l 2,4-D, 0.1 mg/l 6-furfurylaminopurine and 750 μg/ml MgCL2, and with 50 to 100 μg/ml cefotaxime and 400-500 μg/ml carbenicillin to kill residual bacteria. Individual cell lines are isolated after two to three months (with subcultures every four to six weeks) and are further cultivated on selective medium for tissue amplification (30° C., 16 hr photoperiod). Transformed tissues are subsequently further cultivated on non-selective medium during 2 to 3 months to give rise to somatic embryos. Healthy looking embryos of at least 4 mm length are transferred to tubes with SH medium in fine vermiculite, supplemented with 0.1 mg/l indole acetic acid, 6 furfurylaminopurine and gibberellic acid. The embryos are cultivated at 30° C. with a photoperiod of 16 hrs, and plantlets at the 2 to 3 leaf stage are transferred to pots with vermiculite and nutrients. The plants are hardened and subsequently moved to the greenhouse for further cultivation.

### Example 10

### Phenotypic Evaluation Procedure

### 10.1 Evaluation Setup

Approximately 35 independent T0 rice transformants were generated. The primary transformants were transferred from a tissue culture chamber to a greenhouse for growing and harvest of T1 seed. Six or eight events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. Plants grown under non-stress conditions were watered at regular intervals to ensure that water and nutrients were not limiting and to satisfy plant needs to complete growth and development.

For some T1 experiments, four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

### Drought Screen (FLA-Like Polypeptides—DHAR Polypeptides)

Plants from T2 seeds were grown in potting soil under normal conditions until they approached the heading stage. They were then transferred to a “dry” section where irrigation was withheld. Humidity probes were inserted in randomly chosen pots to monitor the soil water content (SWC). When SWC goes below certain thresholds, the plants were automatically re-watered continuously until a normal level was reached again. The plants were then re-transferred again to normal conditions. The rest of the cultivation (plant maturation, seed harvest) was the same as for plants not grown under abiotic stress conditions. Growth and yield parameters were recorded as detailed for growth under normal conditions.

### Nitrogen Use Efficiency Screen (GDH Polypeptides—SAUR Polypeptides)

Rice plants from T2 seeds were grown in potting soil under normal conditions except for the nutrient solution. The pots were watered from transplantation to maturation with a specific nutrient solution containing reduced N nitrogen (N) content, usually between 7 to 8 times less. The rest of the cultivation (plant maturation, seed harvest) was the same as for plants not grown under abiotic stress. Growth and yield parameters were recorded as detailed for growth under normal conditions.

### Salt Stress Screen (SAUR Polypeptides)

Plants were grown on a substrate made of coco fibers and argex (3 to 1 ratio). A normal nutrient solution was used during the first two weeks after transplanting the plantlets in the greenhouse. After the first two weeks, 25 mM of salt (NaCl) was added to the nutrient solution, until the plants were harvested. Seed-related parameters were then measured.

10.2 Statistical Analysis: F test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Where two experiments with overlapping events were carried out (T1 and T2 events), a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment—event—segregants). P values were obtained by comparing likelihood ratio test to chi square distributions.

### 10.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass, areamax) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant, rootmax); or as an increase in the root/shoot index (measured as the ratio between root mass and shoot mass in the period of active growth of root and shoot).

Early vigour (EmerVigor) was determined by counting the total number of pixels from aboveground plant parts discriminated from the background. This value was averaged for the pictures taken on the same time point from different angles and was converted to a physical surface value expressed in square mm by calibration. The results described below are for plants three weeks post-germination.

### Seed-Related Parameter Measurements

The mature primary panicles were harvested, counted, bagged, barcode-labelled and then dried for three days in an oven at 37° C. The panicles were then threshed and all the seeds were collected and counted (firstpan). The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield (totalwgseeds) was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Examples 11

### Results of the Phenotypic Evaluation of the Transgenic Plants

### 1. Glutamate Dehydrogenase (GDH) Polypeptides

a) Oryza sativa Transformed with pGOS2::ZmGDH

Plants were evaluated in both T1 and T2 generation. When grown under non-stress conditions, the transgenic plants showed an increase in above ground area (areamax), early vigour, root growth and seed yield. Details are given in Tables G1 and G2 below:

TABLE G1
Data summary of the evaluation of T1 pGOS2::ZmGDH transgenics
under non-stress conditions. For each parameter, the percentage overall
increase is shown, for each the p-value was ≦0:05.

| Parameter | Overall |
|---|---|
| EmerVigor | 10.9 |
| firstpan | 14.8 |
| nrtotalseed | 11.8 |

TABLE G2
Data summary of the evaluation of T2 pGOS2::ZmGDH transgenics
under non-stress conditions. For each parameter, the percentage overall
increase is shown, for each the p-value was ≦ 0:05.

| Parameter | Overall |
|---|---|
| EmerVigor | 21.4 |
| RootMax | 9.5 |
| totalwgseeds | 11.9 |
| nrfilledseed | 13.0 |
| harvestindex | 9.1 |
| nrtotalseed | 11.9 |
| RootThickMax | 10.1 |

When pGOS2::ZmGDH transgenics was grown under conditions of nitrogen limitation (nutrient stress), there was a tendency for increased TKW and early vigour.

b) Oryza sativa Transformed with pRCc3::ZmGDH

Plants were evaluated in T1 generation. When grown under non-stress conditions, the transgenic plants showed an increase in above ground area (areamax), early vigour, root growth and seed yield. Details are given in Table G3:

TABLE G3
Data summary of the evaluation of T1 pRCc3::ZmGDH transgenics
under non-stress conditions. For each parameter, the percentage
overall increase is shown, for each the p-value was ≦0:05.

| Parameter | Overall |
|---|---|
| totalwgseeds | 9.2 |
| nrfilledseed | 7.9 |
| harvestindex | 7.9 |
| nrtotalseed | 6.7 |

When grown under conditions of nitrogen limitation, an increase was observed for early vigour, root growth (root/shoot index), and seed yield (higher total weight of seeds, number of filled seeds, fill rate, harvest index and number of flowers per panicle).

c) Oryza sativa Transformed with pGOS2::OsGDH

Plants were evaluated in T1 generation. When grown under non-stress conditions, the transgenic plants showed an increase in above ground area (areamax) and seed yield (total weight of seeds, number of filled seeds, fill rate, number of flowers per panicle, harvest index, TKW, number of first panicles). Details are given in Table G4:

TABLE G4
Data summary of the evaluation of T1 pGOS2::OsGDH transgenics
under non-stress conditions. For each parameter, the percentage overall
increase is shown, for each the p-value was ≦0:05.

| Parameter | Overall |
|---|---|
| AreaMax | 6.5 |
| totalwgseeds | 11.5 |
| nrfilledseed | 12.1 |
| flowerperpan | 6.3 |
| nrtotalseed | 7.6 |

When grown under conditions of nitrogen limitation, an increase was observed for above ground area, early vigour, root growth (rootmax & root/shoot index), and seed yield (higher total weight of seeds, number of filled seeds, fill rate, and number of flowers per panicle).

d) Oryza sativa Transformed with pRCc3::OsGDH

Plants were evaluated in T1 generation. When grown under non-stress conditions, the transgenic plants showed an increase in above ground area (areamax) and had an increased number of flowers per panicle and number of first panicles

When grown under conditions of nitrogen limitation, an increase was observed for number of filled seeds and fill rate (overall increase of 6.2%, with a p-value≦0:05).

### 2. Fasciclin-Like AGP (FLAB) Polypeptides

The results of the evaluation of transgenic rice plants in the T1 generation and expressing a nucleic acid comprising the Open Reading Frame of SEQ ID NO: 171 encoding SEQ ID NO: 172 under non-stress conditions are presented below (Table G5). See previous Examples for details on the generations of the transgenic plants.

The results of the evaluation of transgenic rice plants under drought conditions (drought screen above) are presented below (Table G5). An increase of at least 5% was observed for total seed yield (totalwgseeds), number of filled seeds (nrfilledseed), fill rate (fillrate), number of flowers per panicle, harvest index (harvestindex).

TABLE G5
Data summary for transgenic rice plants; for each parameter, the overall
effect in the transgenic plant compared to the control plant is shown for
the (T1 generation), for each parameter the p-value is <0.05.

| | transgenic compared |
|---|---|
| Trait | to control plants (%) |
| RootMax | 5.4 |
| totalwgseeds | 34.9 |
| fillrate | 50.1 |
| harvestindex | 50.3 |
| nrfilledseed | 35.1 |

### 3. Small Auxin-Up RNA (SAUR) Polypeptides

The results of the evaluation of transgenic rice plants in the T1 generation and expressing a nucleic acid comprising the longest region in SEQ ID NO: 501 encoding SEQ ID NO: 502 under non-stress conditions are presented below. See previous Examples for details on the generations of the transgenic plants.

The results of the evaluation of transgenic rice plants under non-stress conditions are presented below. An increase of at least 5% was observed for aboveground biomass (AreaMax), early vigour (EmerVigor), total seed yield (totalwgseeds), number of filled seeds (nrfilledseed), number of first panicles and (firstpan fill rate, number of flowers per panicle (nrtotalseed) (Table G6).

TABLE G6
Data summary for transgenic rice plants compared to the control plants
and expressed in percentage; for each parameter, the overall percent
increase is shown for the confirmation (T2 generation), for each
parameter the p-value is <0.05.

| Parameter: | |
|---|---|
| Yield trait | Overall |
| AreaMax | 13.0 |
| EmerVigor | 11.2 |
| totalwgseeds | 12.5 |
| nrfilledseed | 14.6 |
| firstpan | 20.2 |
| nrtotalseed | 18.2 |

When the plants were grown under Nitrogen limiting conditions as described in the Nitrogen screen above the transgenic plants showed enhanced production of first panicles (11.3% increase) relative to the control plants.

The results of the evaluation of transgenic rice plants expressing a nucleic acid comprising the longest region in SEQ ID NO: 2210 encoding SEQ ID NO: 2211 under low nitrogen conditions gave an increase of at least 5% for TKW with a p-value in the F-test of 0. Most events also showed an increase in plant height compared to corresponding nulizygotes.

The results of the evaluation of transgenic rice plants expressing a nucleic acid comprising the longest region in SEQ ID NO: 2210 encoding SEQ ID NO: 2211 under non-stress conditions gave an increase of at least 5% for total seed weight and TKW. The following parameters also showed an increase compared to corresponding nulizygotes: aboveground biomass, root biomass, harvest index, fill rate, total number of seeds and number of first panicles.

### 4. SAUR Polypeptides, or SYNP Polypeptides, or Fusion Proteins of SAUR Polypeptides and SYNP Polypeptides

The results of the evaluation of transgenic rice plants in the T1 generation and expressing a nucleic acid comprising the longest region in SEQ ID NO: 1163 encoding SEQ ID NO: 1164 under non-stress conditions are presented below. See previous Examples for details on the generations of the transgenic plants.

The results of the evaluation of transgenic rice plants under non-stress conditions are presented below. An increase of at least 5% was observed for aboveground biomass (AreaMax), early vigour (EmerVigor), total seed yield (totalwgseeds), number of filled seeds (nrfilledseed), number of first panicles and (firstpan fill rate, number of flowers per panicle (nrtotalseed) (Table G7).

TABLE G7
Data summary for transgenic rice plants compared to the control plants
and expressed in percentage; for each parameter, the overall percent
increase is shown for the confirmation (T2 generation), for each
parameter the p-value is <0.05.

| Parameter: Yield trait | Overall |
|---|---|
| Area Max | 13.0 |
| EmerVigor | 11.2 |
| totalwgseeds | 12.5 |
| nrfilledseed | 14.6 |
| firstpan | 20.2 |
| nrtotalseed | 18.2 |

When the plants were grown under Nitrogen limiting conditions as described in the Nitrogen screen above the transgenic plants showed enhanced production of first panicles (11.3% increase) relative to the control plants.

### 5. Dehydroascorbate Reductase (DHAR) Polypeptides

The results of the evaluation of transgenic rice plants in the T2 generation and expressing a nucleic acid comprising the longest Open Reading Frame in SEQ ID NO: 1957 under non-stress conditions are presented below. See previous Examples for details on the generations of the transgenic plants.

The results of the evaluation of transgenic rice plants in the T2 generation and expressing a nucleic acid encoding the polypeptide of SEQ ID NO: 1958 under non-stress conditions are presented below in Table G8. When grown under non-stress conditions, an increase of at least 5% was observed for root biomass (RootMax—total root biomass and RootThickMax—amount of thick roots), and for seed yield (Totalwgseeds—total weight of seeds, Nrfilledseed—number of filled seeds, Harvestindex—harvest index, EmerVigor—vigour of the seedlings, Nrtotalseed—number of florets of a plant, and Firstpan—the number of panicles in the first flush).

TABLE G8
Data summary for transgenic rice plants; for each parameter, the overall
percent increase is shown for the confirmation (T2 generation), for
each parameter the p-value is <0.05 and above the 5% threshold.

| Parameter | Overall |
|---|---|
| EmerVigor | 20.8 |
| RootMax | 12.3 |
| totalwgseeds | 26.3 |
| nrtotalseed | 20.3 |
| harvestindex | 15.0 |
| firstpan | 20.9 |
| nrfilledseed | 28.9 |
| RootThickMax | 9.1 |

### Example 12

### Transgenic Plants Transformed with GDH of Physcomitrella patens 126976 Show Increased Yield Related Traits

Rice plants are transformed with a construct comprising a nucleic acid encoding PpGDH (SEQ ID NO: 77) essentially as described in Examples 7 and 8, and are evaluated as described in Example 10. The transformed rice plants show increased yield related traits, compared to the control plants.

### Example 13

### Identification of SAUR Interacting Proteins

The SAUR polypeptides of Table A originating from Arabidopsis thaliana, as represented by SEQ ID NO: 2n, wherein “n” is any numeral from 1 to 81, were used to identified proteins that interact with any of said SAUR proteins by means of the silico interaction techniques “AtPID” (ian Cui, Peng Li, Guang Li, Feng Xu, Chen Zhao, Yuhua Li, Zhongnan Yang, Guang Wang, Qingbo Yu, Yixue Li, and Tieliu Shi AtPID: Arabidopsis thaliana protein interactome database an integrative platform for plant systems biology. Nucleic Acids Research, 2008, Vol. 36, Database issue D999-D1008). Version3.00 AtPID was used. This database includes 28,062 protein-protein interaction pairs involving 12,506 proteins with 23,396 pairs from prediction methods, while the other 4,666 pairs involving 2,285 proteins are manually curated from literatures. In addition, subcellular localizations of 5,562 proteins are also included. A number of SAUR interacting proteins was identified: “total SAUR inteactome”

“In silico interaction technique” as used herein refers to any method to identify proteins that interact with a query sequence mediated by a computer support. Such interactions may be experimentally verified by biochemical methods or may be computer predicted using specialized algorithms. An example of “In silico interaction technique” is that of that encompassing searches on the AtPID database. The AtPID (Arabidopsis thaliana Protein Interactome Database) represents a centralized platform to depict and integrate the information pertaining to protein-protein interaction networks, domain architecture, ortholog information and GO (Gene onthology annotation in the Arabidopsis thaliana proteome. The Protein-protein interaction pairs are predicted by integrating several methods with the Naive Baysian Classifier. All other related information curated in the AtPID is manually extracted from published literatures and other resources from some expert biologists.

### Example 14

### Identification of SAUR Co-Expressed Genes

The SAUR polynucleotides of Table A4 originating from Arabidopsis thaliana, as represented by SEQ ID NO: 2n+1, wherein “n” is any numeral from 1 to 80, were used to identified co-regulated genes in Arabidopsis thaliana using the ATTED-II platform (Obayashi 2007. Nucleic Acids Res. 2007 January; 35(Database issue):D863-9). Atted-II platform refers to a database ATTED-II as described by Obayashi et al. 2007 comprising Arabidopsis thaliana trans-factor and cis-element prediction database (ATTED-II) that provides co-regulated gene relationships based on co-expressed genes deduced from experimentally reported microarray data and the predicted cis elements. ATTED-II (http://www.atted.bio.titech.ac.jp) includes the following features: (i) lists and networks of co-expressed genes calculated from 58 publicly available experimental series, which are composed of 1388 GeneChip data in A. thaliana; (ii) prediction of cis-regulatory elements in the 200 bp region upstream of the transcription start site to predict co-regulated genes amongst the co-expressed genes; and (iii) visual representation of expression patterns for individual genes. A number of SAUR co-regulated genes were identified: “total SAUR co-regulated genes”.

### Example 15

### Identification of SAUR Yield Network Proteins

The term “SAUR yield network proteins” as used herein refer to proteins having the capacity to enhance yield related traits, said capacity mediated by the effect of a SAUR gene or SAUR polypeptide.

On a selection step, a selection of genes and/or proteins out of the two groups of data sets, “total SAUR co-regulated genes” and the “total SAUR inteactome” and having the capacity to enhanced yield related trait mediated by a SAUR gene or SAUR protein was made (Table E). The selected group of genes and proteins represent the SAUR yield network proteins (SYNP) of Arabidopsis thaliana.

TABLE E
SAUR yield network proteins (SYNP) of Arabidopsis thaliana.

| | Nucleic acid | Polypeptide |
|---|---|---|
| Name, AGI code | SEQ ID NO: | SEQ ID NO: |
| AT1G04880 | 664 | 665 |
| AT1G26480 | 666 | 667 |
| AT1G68090 | 668 | 669 |
| AT1G68750 | 670 | 671 |
| AT1G74500 | 672 | 673 |
| AT1G77980 | 674 | 675 |
| AT2G24450 | 676 | 677 |
| AT2G32460 | 678 | 679 |
| AT2G45610 | 680 | 681 |
| AT3G04700 | 682 | 683 |
| AT3G06260 | 684 | 685 |
| AT3G46520 | 686 | 687 |
| AT3G61160 | 688 | 689 |
| AT4G00870 | 690 | 691 |
| AT4G36540 | 692 | 693 |
| AT5G01700 | 694 | 695 |
| AT5G04180 | 696 | 697 |
| AT5G20390 | 698 | 699 |
| AT5G48650 | 700 | 701 |
| AT5G67110 | 702 | 703 |

### Example 16

### Identification of Homologues of SAUR Yield Network Proteins (SYNP) of Arabidopsis thaliana: Paralogous and Orthologous Proteins and Genes Encoding the Same

Paralogous and orthologous genes of those of Table E were identified using the method described in Example 1. Paralogous and orthologous proteins and genes encoding the same were selected amongst those originating from organisms of the viridiplantae kingdom (Table F).

| | Nucleic acid | Polypeptide |
|---|---|---|
| Name SYNP homologue | SEQ ID NO: | SEQ ID NO: |
| Homologue 1 | 704 | 705 |
| Homologue 2 | 706 | 707 |
| Homologue 3 | 708 | 709 |
| Homologue 4 | 710 | 711 |
| Homologue 5 | 712 | 713 |
| Homologue 6 | 714 | 715 |
| Homologue 7 | 716 | 717 |
| Homologue 8 | 718 | 719 |
| Homologue 9 | 720 | 721 |
| Homologue 10 | 722 | 723 |
| Homologue 11 | 724 | 725 |
| Homologue 12 | 726 | 727 |
| Homologue 13 | 728 | 729 |
| Homologue 14 | 730 | 731 |
| Homologue 15 | 732 | 733 |
| Homologue 16 | 734 | 735 |
| Homologue 17 | 736 | 737 |
| Homologue 18 | 738 | 739 |
| Homologue 19 | 740 | 741 |
| Homologue 20 | 742 | 743 |
| Homologue 21 | 744 | 745 |
| Homologue 22 | 746 | 747 |
| Homologue 23 | 748 | 749 |

1-115. (canceled)

116. A method for enhancing a yield-related trait in a plant relative to a corresponding control plant, comprising modulating expression in a plant of a nucleotide sequence comprising one of:
a. a first nucleic acid encoding at least a small auxin-up RNA (SAUR) polypeptide and a second nucleic acid encoding one or more SAUR Yield Network Protein (SYNP) polypeptides, or a nucleic acid encoding a protein fusion between at least a SAUR polypeptide and one or more SYNP polypeptides, wherein the first and second nucleic acids are comprised in a single nucleic acid molecule or in at least two nucleic acid molecules;
b. a nucleic acid encoding a glutamate dehydrogenase (GDH) polypeptide, wherein said GDH polypeptide is a NAD-dependent GDH having glutamate dehydrogenase activity;
c. a nucleic acid encoding a polypeptide comprising a Fasciclin-like (FLA-like) polypeptide;
d. a nucleic acid encoding a SAUR polypeptide, wherein said SAUR polypeptide comprises an Auxin inducible domain; or
e. a nucleic acid encoding a dehydroascorbate reductase (DHAR) polypeptide, wherein said DHAR polypeptide comprises at least a DHAR domain with accession number PTHR11260:SF15.

117. The method of claim 116,
a. wherein
(i) the SAUR polypeptide has at least 25% overall sequence identity to any one of the polypeptide sequences of Table A4, or to SEQ ID NO: 1164 or a variant thereof,
(ii) the SYNP polypeptide has at least 25% overall sequence identity to any one of the polypeptide sequences of Table E or Table F;
b. wherein said GDH polypeptide comprises one or more of the motifs 1 to 20 (SEQ ID NO: 3 to SEQ ID NO: 22);
c. wherein said FLA-like polypeptide comprises at least one, two, three, or four fasciclin-like domains having at least 25% sequence identity to the amino acid sequence:
(i) TITVCAVDNAGMSDLLSKQLSIYTIKNVLSFRVLLDYFDAKKLHQITNG TALAATMFQATGSATGSSGFVNITDLRGGKVGLSPADYNGPPPAKFVK SIAEIPYNISVIQISTIL (SEQ ID NO: 487) which corresponds to the N-term FLA_like domain in SEQ ID NO: 172, or
(ii) VDGGVTIFCPRDDAMKKFLPKFKNLTAEGKQSLLEYHGIPIYQSISNLKS NNGDMNTLATDGAKKYAVVIQNDGEDVTIKTKIVTAKITATVVDKLPL AIYSLDKVL (SEQ ID NO: 488) which corresponds to the C-term FLA_like domain in SEQ ID NO: 172;
d. wherein said SAUR polypeptide comprises one or more of the following motifs:
(i)
Motif 23:
(SEQ ID NO: 1155)
LAVYVGEMMQKRRFVVPVTYLSHPCFQKLLRKAEEEFGFDHPMGGLTI
PC,
(ii)
Motif 24:
(SEQ ID NO: 1156)
KHxxGVYTAEKxxYxxxIxxxxxxxxxAxxxxSxxxYxxxxPMPIxLx
xC,
(iii)
Motif 25:
(SEQ ID NO: 1157)
LQSSKQLLKSLSHSSNNVAIP,
(iv)
Motif 26:
(SEQ ID NO: 1158)
VxxxKIAxKSQ,
(v)
Motif 27:
(SEQ ID NO: 1159)
EQIFIDLASRL,
(vi)
Motif 28:
(SEQ ID NO: 1159)
VExxxVxxxxL,
wherein X represents any amino acid;
e. wherein said SAUR polypeptide is a SAUR33-like polypeptide and comprises Motifs 47 and 48 and optionally also one or both of Motifs 49 and 50 or any sequence having at least 50% sequence identity to Motifs 47 to 50, wherein Motif 47 is CEVVLFEHLLWMLENADPQ (SEQ ID NO: 2284), Motif 48 is PESLDELVEYYAC (SEQ ID NO: 2285), Motif 49 is GLSKLRCMIRRHSSSRI (SEQ ID NO: 2286) and Motif 50 is SFHGADEVPKGLHPVYVGKSRRRYLIAEELVGHPLFQNLVDRT (SEQ ID NO: 2287);
f. wherein the DHAR domain of the DHAR polypeptide has at least 49% sequence identity to the amino acid sequence located between amino acid 19 and 210 of SEQ ID NO 1958;
g. wherein said DHAR polypeptide comprises motifs having at least 49% sequence identity to any one of the following motifs:
(i)
Motif 35:
(SEQ ID NO: 2239)
P[DN]KLGDCPF[SC]QRVLLTLEEK[KH][VL]PY[KD][ML][KH]
L[IV],
(ii)
Motif 36:
(SEQ ID NO: 2240)
D[DEG]KW[VI][PAS]DSDVI[TV][QG][IL][LI]EEK[YF]PEP
[SP]L[VA]TPPE,
(iii)
Motif 37:
(SEQ ID NO: 2241)
P[FY][IV][NA]GE[KN][IV][ST]A[VA]DLSL[AG]PKLYHLE[V
I]ALGH[FY]K[KN]W[ST][VI]P;
h. wherein said DHAR polypeptide comprises any one or more sequence motifs having at least 49% sequence identity to any one of the following motifs:
(i)
Motif 38:
(SEQ ID NO: 2242)
E[VI]CVKA[AS]V[GT]AP[DN][KV]LGDCPF[SC]QRVLLTLEE,
(ii)
Motif 39:
(SEQ ID NO: 2243)
PPE[FK]ASVGSKIF[PS][TS]F[VI][GT]FLKSKD[PA][NS]DG
[TS]EQ,
(iii)
Motif 40:
(SEQ ID NO: 2243)
[IV][ST]A[VA]DLSL[AG]PKLYHL[EQ][VI]ALGH[FY]K[KN]
W[ST][VI]P[ED]S L[TP]HV[HK][NS]Y[MT]K[ALS][LI]FS
[RL][ED]SF[EV]KT;
i. wherein said DHAR polypeptide comprises any one or more of the following sequence motifs or an amino acid sequence having at least 49% sequence identity to any one of the following sequence motifs:
(i)
Motif 41:
(SEQ ID NO: 2244)
E[IV]CVKAA[VT]GAPD[VIT]LGDCPF[SC]QRVLLTLEE,
(ii)
Motif 42:
(SEQ ID NO: 2245)
PPE[FY]ASVGSKIF[PG][ST]FV[TK]FLKSKD[AP][NS]DG
[TS]E[QK]
(iii)
Motif 43:
(SEQ ID NO: 2246)
[IV][TS]AVDLSLAPKLYHL[EQ]VAL[GE]HFK[KG]W[TSK]
[VI]PE[SN]LTHVH[NA]Y[TM]K[LAS]LFSRESFEKT;
j. wherein said DHAR polypeptide comprises any one or more of the following sequence motifs or an amino acid sequence having at least 49% sequence identity to any one of the following motifs:
(i)
Motif 44:
(SEQ ID NO: 2247)
PLE[VI]C[VA]KAS[ILV]T[TV]P[ND][KR]LGDCPF[TC]QRVLL
TLEEKHLPY[DE][ML]KLVDL[SG]NKP[ED]WF,
(ii)
Motif 45:
(SEQ ID NO: 2248)
PPE[VI][PA]DSDVITQ[AST]LEEK[YF]P[ED]P[PS]L[AV]TPP
EKASVGSKIFSTF[IV]GFLKSKDP[SN]DG,
(iii)
Motif 46:
(SEQ ID NO: 2249)
QALL[ND]EL[ST][SA]FNDY[LI]KENGPFING[KE][KDE][IV]S
AADLSL[GA]PKLYH[LM]EIALGH[YF]K[NK]W;
k. wherein the DHAR polypeptide has at least 25% overall sequence identity to any of the amino acid sequences depicted in Table A, or the amino acid sequence of SEQ ID NO: 1958.

118. The method of claim 116, wherein said modulated expression is effected by introducing and expressing the nucleotide sequence in a plant.

119. The method of claim 116, wherein
a. the nucleic acid encoding a SAUR polypeptide encodes any one of the proteins listed in Table A3 or A3(i), or is a portion of such a nucleic acid, or a nucleic acid capable of hybridizing with such a nucleic acid;
b. the nucleic acid encoding a GDH polypeptide encodes any one of the proteins listed in Table A1, or is a portion of such a nucleic acid, or a nucleic acid capable of hybridizing with such a nucleic acid;
c. the nucleic acid encoding a FLA-like polypeptide encodes any one of the proteins listed in Table A2, or is a portion of such a nucleic acid, or a nucleic acid capable of hybridizing with such a nucleic acid; or
d. the nucleic acid encoding a DHAR polypeptide encodes any one of the proteins listed in Table A5, or is a portion of such a nucleic acid, or a nucleic acid capable of hybridizing with such a nucleic acid.

120. The method of claim 116,
a. wherein the nucleic acid sequence encoding a SAUR polypeptide encodes an orthologue or paralogue of any of the proteins given in Table A3 or A3(i);
b. wherein the nucleic acid sequence encoding a GDH polypeptide encodes an orthologue or paralogue of any of the proteins given in Table A1;
c. wherein the nucleic acid sequence encoding a FLA-like polypeptide encodes an orthologue or paralogue of any of the proteins given in Table A2; or
d. wherein the nucleic acid sequence encoding a DHAR polypeptide encodes an orthologue or paralogue of any of the proteins given in Table A5.

121. The method of claim 116, wherein the enhanced yield-related trait comprises increased early vigour, increased yield, increased biomass, and/or increased seed yield relative to a corresponding control plant.

122. The method of claim 116, wherein the enhanced yield-related trait is obtained under non-stress conditions.

123. The method of claim 116, wherein the enhanced yield-related trait is obtained under conditions of nitrogen deficiency, under conditions of drought stress, or under conditions of salt stress.

124. The method of claim 118, wherein the nucleic acid is operably linked to one of
(i) a constitutive promoter, a GOS2 promoter, or a GOS2 promoter from rice;
(ii) a root specific promoter, a RCc3 promoter, or a RCc3 promoter from rice; or
(iii) a tissue-specific promoter, a leaf specific promoter, or a promoter comprising the nucleic acid sequence of SEQ ID NO: 1163.

125. The method of claim 116, wherein the nucleic acid is of plant origin, from a dicotyledonous plant, or from a monocotyledonous plant.

126. A transgenic plant or part thereof, including seeds, obtained by the method of claim 116, wherein said transgenic plant or part thereof comprises one of:
a. (i) a first nucleic acid encoding at least a SAUR polypeptide selected from the group consisting of the polypeptides of Table A4 or a homologue or derivative thereof, and (ii) a second nucleic acid encoding one or more SYNP polypeptides selected from the group consisting of the polypeptides of Tables E and F or a homologue or derivative thereof; or a nucleic acid encoding a protein fusion between any two polypeptides of (i) and (ii), wherein the first and second nucleic acids are comprised in a single nucleic acid molecule or in at least two nucleic acid molecules;
b. a recombinant nucleic acid encoding a GDH polypeptide;
c. a recombinant nucleic acid encoding a FLA-like polypeptide;
d. a recombinant nucleic acid encoding a SAUR polypeptide; or
e. a recombinant nucleic acid encoding a DHAR polypeptide.

127. A construct comprising:
(i) a nucleic acid encoding a polypeptide as defined in claim 116;
(ii) one or more control sequences capable of driving expression of the nucleic acid sequence of (i); and optionally
(iii) a transcription termination sequence.

128. The construct of claim 127, wherein one of said control sequences is one of:
(i) a constitutive promoter, a GOS2 promoter, or a GOS2 promoter from rice;
(ii) a root specific promoter, an RCc3 promoter, or an RCc3 promoter from rice; or
(iii) a tissue-specific promoter, a leaf specific promoter, or a promoter comprising the nucleic acid sequence of SEQ ID NO: 1163.

129. A method for making a plant having increased early vigour, increased yield, increased biomass, and/or increased seed yield relative to a control plant, comprising transforming a plant, plant part, or plant cell with the construct of claim 127.

130. A plant, plant part, or plant cell comprising the construct of claim 127.

131. A method for the production of a transgenic plant having increased early vigour, increased yield, increased biomass, and/or increased seed yield relative to a corresponding control plant, comprising:
(i) introducing and expressing in a plant a nucleic acid encoding a polypeptide as defined in claim 116; and
(ii) cultivating the plant under conditions promoting plant growth and development.

132. A transgenic plant having increased early vigour, increased yield, increased biomass, and/or increased seed yield relative to a corresponding control plant, resulting from modulated expression of a nucleic acid encoding a polypeptide as defined in claim 116, or a transgenic plant cell derived from said transgenic plant.

133. The transgenic plant of claim 126, or a transgenic plant cell derived therefrom, wherein said transgenic plant is a crop plant, a monocot, a cereal, rice, maize, wheat, barley, millet, rye, triticale, sorghum, emmer, spelt, secale, einkorn, teff, milo, or oats.

134. Harvestable parts of the plant of claim 133, wherein said harvestable parts are shoot biomass, root biomass, and/or seeds.

135. Products derived from the transgenic plant of claim 133 and/or from harvestable parts of said plant, wherein said harvestable parts are shoot biomass, root biomass, and/or seeds.

136. An isolated nucleic acid molecule comprising one of:
(i) the nucleic acid sequence of SEQ ID NO: 99, SEQ ID NO: 1997, SEQ ID NO: 2121, or SEQ ID NO: 2193, or the complement thereof;
(ii) a nucleic acid encoding a polypeptide having at least 50% sequence identity to the amino acid sequence of SEQ ID NO: 176, and comprising an amino acid sequence having at least 50% sequence identity to any one of the motifs 4 to 6 (SEQ ID NO: 6 to 8);
(iii) a nucleic acid encoding a DHAR polypeptide comprising the amino acid sequence of any one of SEQ ID NO: 1998, SEQ ID NO: 2122 and SEQ ID NO: 2194, and conferring an enhanced yield related trait to a plant relative to a corresponding control plant;
(iv) a nucleic acid having at least 50% sequence identity to any of the nucleic acid sequences of Table A5 and conferring an enhanced yield related trait to a plant relative to a corresponding control plant;
(v) a nucleic acid encoding a DHAR polypeptide having at least 50% sequence identity to the amino acid sequence of any one of SEQ ID NO: 1998, SEQ ID NO: 2122, SEQ ID NO: 2194 and any of the other amino acid sequences in Table A5, and conferring an enhanced yield related trait to a plant relative to a corresponding control plant; or
(vi) a nucleic acid molecule which hybridizes with a nucleic acid molecule of (i) to (v) under stringent hybridization conditions and confers an enhanced yield related trait to a plant relative to a corresponding control plant.

137. An isolated polypeptide comprising one of
(i) the amino acid sequence of SEQ ID NO: 176, SEQ ID NO: 1998, SEQ ID NO: 2122 or SEQ ID NO: 2194;
(ii) an amino acid sequence having at least 50% sequence identity to the amino acid sequence of SEQ ID NO: 176, and comprising an amino acid sequence having at least 50% sequence identity to any one of the motifs 4 to 6 (SEQ ID NO: 6 to 8);
(iii) an amino acid sequence having at least 50% sequence identity to SEQ ID NO: 1998, SEQ ID NO: 2122, or SEQ ID NO: 2194; or
(iv) derivatives of any of the amino acid sequences of (i) or (iii) above.
